(19) United States
(12) Patent Application Publication
(10) Pub. No.: US 2025/0066792 A1
(43) Pub. Date: Feb. 27, 2025

(54) COMPOUNDS AND METHODS FOR MODULATION OF DYSTROPHIA MYOTONICA-PROTEIN KINASE (DMPK) EXPRESSION

(71) Applicant: Ionis Pharmaceuticals, Inc., Carlsbad, CA (US)

(72) Inventors: Eric E. Swayze, Encinitas, CA (US); Susan M. Freier, San Diego, CA (US)

(73) Assignee: Ionis Pharmaceuticals, Inc., Carlsbad, CA (US)

(21) Appl. No.: 18/627,154

(22) Filed: Apr. 4, 2024

Related U.S. Application Data
(63) Continuation of application No. 17/173,139, filed on Feb. 10, 2021, now Pat. No. 11,981,897.
Application No. 17/173,139 is a continuation of application No. 16/167,783, filed on Oct. 23, 2018, now Pat. No. 10,954,519.
Application No. 16/167,783 is a continuation of application No. 14/911,248, filed on Feb. 9, 2016, now abandoned.
(60) Provisional application No. 61/889,337, filed on Oct. 10, 2013.
(60) Provisional application No. 61/864,439, filed on Aug. 9, 2013.

(51) Int. Cl. C12N 15/113

(52) U.S. Cl. CPC C12N 15/1137; C12Y 207/11001; C12N 2310/11; C12N 2310/315; C12N 2310/32; C12N 2310/3231; C12N 2310/3341; C12N 2320/30

(57) ABSTRACT

Provided herein are methods, compounds, and compositions for reducing expression of a DMPK mRNA and protein in an animal. Also provided herein are methods, compounds, and compositions for preferentially reducing CUGexp DMPK RNA, reducing myotonia or reducing spliceopathy in an animal. Such methods, compounds, and compositions are useful to treat, prevent, delay, or ameliorate type 1 myotonic dystrophy, or a symptom thereof.

### SEQUENCE LISTING

The present application is being filed along with a Sequence Listing in electronic format. The Sequence Listing is provided as a file entitled BIOL0171SEQ.xml created Sep. 1, 2023, which is approximately 5268 Kb in size. The information in the electronic format of the sequence listing is incorporated herein by reference in its entirety.

### FIELD

Provided herein are methods, compounds, and compositions for reducing expression of DMPK mRNA and protein in an animal. Also, provided herein are methods, compounds, and compositions comprising a DMPK inhibitor for preferentially reducing CUGexp DMPK RNA, reducing myotonia, or reducing spliceopathy in an animal. Such methods, compounds, and compositions are useful, for example, to treat, prevent, or ameliorate type 1 myotonic dystrophy (DM1) in an animal.

### BACKGROUND

Myotonic dystrophy type 1 (DM1) is the most common form of muscular dystrophy in adults with an estimated frequency of 1 in 7,500 (Harper P S., Myotonic Dystrophy. London: W.B. Saunders Company; 2001). DM1 is an autosomal dominant disorder caused by expansion of a non-coding CTG repeat in DMPK1. DMPK1 is a gene encoding a cytosolic serine/threonine kinase (Brook J D, et al., Cell., 1992, 68(4):799-808). The physiologic functions and substrates of this kinase have not been fully determined. The expanded CTG repeat is located in the 3′ untranslated region (UTR) of DMPK1. This mutation leads to RNA dominance, a process in which expression of RNA containing an expanded CUG repeat (CUGexp) induces cell dysfunction (Osborne R J and Thornton C A., Human Molecular Genetics., 2006, 15(2): R162-R169).

The DMPK gene normally has 5-37 CTG repeats in the 3′ untranslated region. In myotonic dystrophy type I, this number is significantly expanded and is, for example, in the range of 50 to greater than 3,500 (Harper, Myotonic Dystrophy (Saunders, London, ed.3, 2001); Annu. Rev. Neurosci. 29: 259, 2006; EMBO J. 19: 4439, 2000; Curr Opin Neurol. 20: 572, 2007).

The CUGexp tract interacts with RNA binding proteins including muscleblind-like (MBNL) protein, a splicing factor, and causes the mutant transcript to be retained in nuclear foci. The toxicity of this RNA stems from sequestration of RNA binding proteins and activation of signaling pathways. Studies in animal models have shown that phenotypes of DM1 can be reversed if toxicity of CUGexp RNA is reduced (Wheeler T M, et al., Science., 2009, 325(5938):336-339; Mulders S A, et al., Proc Natl Acad Sci USA., 2009, 106(33):13915-13920).

In DM1, skeletal muscle is the most severely affected tissue, but the disease also has important effects on cardiac and smooth muscle, ocular lens, and brain. The cranial, distal limb, and diaphragm muscles are preferentially affected. Manual dexterity is compromised early, which causes several decades of severe disability. The median age at death is 55 years, usually from respiratory failure (de Die-Smulders C E, et al., Brain., 1998, 121(Pt 8):1557-1563).

Antisense technology is emerging as an effective means for modulating expression of certain gene products and may therefore prove to be uniquely useful in a number of therapeutic, diagnostic, and research applications for the modulation of DMPK1. Intramuscular injection of fully modified oligonucleotides targeting with the CAG-repeat were shown in mice to block formation of CUGexp-MBNL1 complexes, disperse nuclear foci of CUGexp transcripts, enhance the nucleocytoplasmic transport and translation of CUGexp transcripts, release MBNL proteins to the nucleoplasm, normalize alternative splicing of MBNL-dependent exons, and eliminate myotonia in CUGexp-expressing transgenic mice (Wheeler T M, et al., Science., 2009, 325(5938):336-339; WO2008/036406).

Presently there is no treatment that can modify the course of DM1. The burden of disease, therefore, is significant. It is, therefore, an object herein to provide compounds, compositions, and methods for treating DM1

### SUMMARY

Provided herein are methods, compounds, and compositions for inhibiting expression of DMPK and treating, preventing, delaying or ameliorating a DMPK related disease and or a symptom thereof. In certain embodiments, the compounds and compositions disclosed herein inhibit mutant DMPK or CUGexp DMPK.

Certain embodiments provide a method of reducing DMPK expression in an animal comprising administering to the animal a compound comprising a modified oligonucleotide as further described herein targeted to DMPK.

Certain embodiments provide a method of preferentially reducing CUGexp DMPK relative to wild-type DMPK, reducing myotonia, or reducing spliceopathy in an animal comprising administering to the animal a compound comprising a modified oligonucleotide, as further described herein, targeted to CUGexp DMPK. In certain instances, CUGexp DMPK transcripts are believed to be particularly sensitive to antisense knockdown via nuclear ribonucleases (such as RNase H), because of their longer residence time in the nucleus, and this sensitivity is thought to permit effective antisense inhibition of CUGexp DMPK transcripts in relevant tissues such as muscle despite the biodistribution barriers to tissue uptake of antisense oligonucleotides. Antisense mechanisms that do not elicit cleavage via nuclear ribonucleases, such as the CAG-repeat ASOs described in, for example, Wheeler T M, et al., Science., 2009, 325(5938):336-339 and WO2008/036406, do not provide the same therapeutic advantage.

Certain embodiments provide a method of treating an animal having type 1 myotonic dystrophy. In certain embodiments, the method includes administering to the animal a therapeutically effective amount of a compound comprising a modified oligonucleotide as further described herein targeted to DMPK. In certain embodiments, the method includes identifying an animal with type 1 myotonic dystrophy.

Certain embodiments provide a method of treating, preventing, delaying, or ameliorating symptoms and outcomes associated with development of DM1 including muscle stiffness, myotonia, disabling distal weakness, weakness in face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts. Certain embodiments provide a method of treating, preventing, delaying, or ameliorating symptoms and outcomes associated with development of DM1 in children, including, developmental delays, learning problems, language and speech issues, and personality development issues.

Certain embodiments provide a method of administering an antisense oligonucleotide to counteract RNA dominance by directing the cleavage of pathogenic transcripts.

In certain embodiments, the DMPK has a sequence as set forth in GenBank Accession No. NM_001081560.1 (incorporated herein as SEQ ID NO: 1). In certain embodiments, the DMPK has the sequence as set forth in GenBank Accession No. NT_011109.15 truncated from nucleotides 18540696 to 18555106 (incorporated herein as SEQ ID NO: 2). In certain embodiments, the DMPK has the sequence as set forth in GenBank Accession No. NT_039413.7 truncated from nucleotides 16666001 to 16681000 (incorporated herein as SEQ ID NO: 3). In certain embodiments, the DMPK has the sequence as set forth in GenBank Accession No. NM_032418.1 (incorporated herein as SEQ ID NO: 4). In certain embodiments, the DMPK has the sequence as set forth in GenBank Accession No. AI007148.1 (incorporated herein as SEQ ID NO: 5). In certain embodiments, the DMPK has the sequence as set forth in GenBank Accession No. AI304033.1 (incorporated herein as SEQ ID NO: 6). In certain embodiments, the DMPK has the sequence as set forth in GenBank Accession No. BC024150.1 (incorporated herein as SEQ ID NO: 7). In certain embodiments, the DMPK has the sequence as set forth in GenBank Accession No. BC056615.1 (incorporated herein as SEQ ID NO: 8). In certain embodiments, the DMPK has the sequence as set forth in GenBank Accession No. BC075715.1 (incorporated herein as SEQ ID NO: 9). In certain embodiments, the DMPK has the sequence as set forth in GenBank Accession No. BU519245.1 (incorporated herein as SEQ ID NO: 10). In certain embodiments, the DMPK has the sequence as set forth in GenBank Accession No. CB247909.1 (incorporated herein as SEQ ID NO: 11). In certain embodiments, the DMPK has the sequence as set forth in GenBank Accession No. CX208906.1 (incorporated herein as SEQ ID NO: 12). In certain embodiments, the DMPK has the sequence as set forth in GenBank Accession No. CX732022.1 (incorporated herein as SEQ ID NO: 13). In certain embodiments, the DMPK has the sequence as set forth in GenBank Accession No. S60315.1 (incorporated herein as SEQ ID NO: 14). In certain embodiments, the DMPK has the sequence as set forth in GenBank Accession No. S60316.1 (incorporated herein as SEQ ID NO: 15). In certain embodiments, the DMPK has the sequence as set forth in GenBank Accession No. NM_001081562.1 (incorporated herein as SEQ ID NO: 16). In certain embodiments, the DMPK has the sequence as set forth in GenBank Accession No. NM_001100.3 (incorporated herein as SEQ ID NO: 17).

The present disclosure provides the following non-limiting numbered embodiments:
Embodiment 1. A compound comprising a modified oligonucleotide consisting of 10-30 linked nucleosides and having a nucleobase sequence comprising a complementary region comprising at least 8 contiguous nucleobases complementary to a target region of equal length of a DMPK nucleic acid.
Embodiment 2. The compound of embodiment 1, wherein at least one nucleoside of the modified oligonucleotide comprises a bicyclic sugar selected from among cEt, LNA, α-L-LNA, ENA and 2′-thio LNA.
Embodiment 3. The compound of any of embodiments 1 to 2, wherein the target region is exon 9 of a DMPK nucleic acid.
Embodiment 4. The compound of any of embodiments 1 to 3, wherein the complementary region comprises at least 10 contiguous nucleobases complementary to a target region of equal length of a DMPK transcript.
Embodiment 5. The compound of any of embodiments 1 to 3, wherein the complementary region comprises at least 12 contiguous nucleobases complementary to a target region of equal length of a DMPK nucleic acid.
Embodiment 6. The compound of any of embodiments 1 to 3, wherein the complementary region comprises at least 14 contiguous nucleobases complementary to a target region of equal length of a DMPK nucleic acid.
Embodiment 7. The compound of any of embodiments 1 to 3, wherein the complementary region comprises at least 16 contiguous nucleobases complementary to a target region of equal length of a DMPK nucleic acid.
Embodiment 8. The compound of any of embodiments 1 to 7, wherein the DMPK nucleic acid is a DMPK pre-mRNA Embodiment 9. The compound of any of embodiments 1 to 7, wherein the DMPK nucleic acid is a DMPK mRNA.
Embodiment 10. The compound of any of embodiments 1 to 9, wherein the DMPK nucleic acid has a nucleobase sequence selected from among SEQ ID NO: 1 and SEQ ID NO: 2.
Embodiment 11. The compound of any of embodiments 1 to 10, wherein the modified oligonucleotide has a nucleobase sequence comprising a complementary region comprising at least 10 contiguous nucleobases complementary to a target region of equal length of SEQ ID NO: 1 or SEQ ID NO: 2.
Embodiment 12. The compound of embodiments 1 to 10, wherein the modified oligonucleotide has a nucleobase sequence comprising a complementary region comprising at least 12 contiguous nucleobases complementary to a target region of equal length of SEQ ID NO: 1 or SEQ ID NO: 2.
Embodiment 13. The compound of embodiments 1 to 10, wherein the modified oligonucleotide has a nucleobase sequence comprising a complementary region comprising at least 14 contiguous nucleobases complementary to a target region of equal length of SEQ ID NO: 1 or SEQ ID NO: 2.
Embodiment 14. The compound of embodiments 1 to 10, wherein the modified oligonucleotide has a nucleobase sequence comprising a complementary region comprising at least 16 contiguous nucleobases complementary to a target region of equal length of SEQ ID NO: 1 or SEQ ID NO: 2.
Embodiment 15. The compound of any of embodiments 1 to 14, wherein the target region is from nucleobase 1343 to nucleobase 1368 of SEQ ID NO.: 1.
Embodiment 16. The compound of any of embodiments 1 to 14, wherein the target region is from nucleobase 1317 to nucleobase 1366 of SEQ ID NO.: 1.
Embodiment 17. The compound of any of embodiments 1 to 14, wherein the target region is from nucleobase 2748 to nucleobase 2791 of SEQ ID NO.: 1.
Embodiment 18. The compound of any of embodiments 1 to 14, wherein the target region is from nucleobase 730 to nucleobase 748 of SEQ ID NO.: 1.
Embodiment 19. The compound of any of embodiments 1 to 14, wherein the target region is from nucleobase 10195 to nucleobase 10294 of SEQ ID NO.: 2.
Embodiment 20. The compound of any of embodiments 1 to 14, wherein the target region is from nucleobase 10195 to nucleobase 10294 of SEQ ID NO.: 2.
Embodiment 21. The compound of any of embodiments 1 to 14, wherein the target region is from nucleobase 10201 to nucleobase 10216 of SEQ ID NO.: 2.
Embodiment 22. The compound of any of embodiments 1 to 14, wherein the target region is from nucleobase 10202 to nucleobase 10218 of SEQ ID NO.: 2.
Embodiment 23. The compound of any of embodiments 1 to 22, wherein the modified oligonucleotide has a nucleobase sequence that is at least 80% complementary to the target region over the entire length of the oligonucleotide.
Embodiment 24. The compound of any of embodiments 1 to 22, wherein the modified oligonucleotide has a nucleobase sequence that is at least 90% complementary to the target region over the entire length of the oligonucleotide.
Embodiment 25. The compound of any of embodiments 1 to 22, wherein the modified oligonucleotide has a nucleobase sequence that is at least 100% complementary to the target region over the entire length of the oligonucleotide.
Embodiment 26. The compound of any of embodiments 1-25 having a nucleobase sequence comprising at least 8 contiguous nucleobases of a sequence recited in any of SEQ ID NOs: 23-874.
Embodiment 27. The compound of any of embodiments 1 to 25, wherein the modified oligonucleotide has a nucleobase sequence comprising at least 10 contiguous nucleobases of sequence recited in SEQ ID NOs: 23-32.
Embodiment 28. The compound of any of embodiments 1 to 25, wherein the modified oligonucleotide has a nucleobase sequence comprising at least 12 contiguous nucleobases of sequence recited in SEQ ID NOs: 23-32.
Embodiment 29. The compound of any of embodiments 1 to 25, wherein the modified oligonucleotide has a nucleobase sequence comprising at least 14 contiguous nucleobases of sequence recited in SEQ ID NOs: 23-32.
Embodiment 30. The compound of any of embodiments 1 to 25, wherein the modified oligonucleotide has a nucleobase sequence comprising at least 16 contiguous nucleobases of sequence recited in SEQ ID NOs: 23-32.
Embodiment 31. The compound of any of embodiments 1 to 30, wherein the modified oligonucleotide has a nucleobase sequence that consists of the sequence recited in SEQ ID NO: 23.
Embodiment 32. The compound of any of embodiments 1 to 14, wherein the modified oligonucleotide has a nucleobase sequence that consists of the sequence recited in SEQ ID NO: 25.
Embodiment 33. The compound of any of embodiments 1 to 14, wherein the modified oligonucleotide has a nucleobase sequence that consists of the sequence recited in SEQ ID NO: 26.
Embodiment 34. The compound of any of embodiments 1 to 14, wherein the modified oligonucleotide has a nucleobase sequence that consists of the sequence recited in SEQ ID NO: 27.
Embodiment 35. The compound of any of embodiments 1 to 14, wherein the modified oligonucleotide has a nucleobase sequence that consists of the sequence recited in SEQ ID NO: 28.
Embodiment 36. The compound of any of embodiments 1 to 14, wherein the modified oligonucleotide has a nucleobase sequence that consists of the sequence recited in SEQ ID NO: 29.
Embodiment 37. The compound of any of embodiments 1 to 14, wherein the modified oligonucleotide has a nucleobase sequence that consists of the sequence recited in SEQ ID NO: 30.
Embodiment 38. The compound of any of embodiments 1 to 14, wherein the modified oligonucleotide has a nucleobase sequence that consists of the sequence recited in SEQ ID NO: 31.
Embodiment 39. The compound of any of embodiments 1 to 14, wherein the modified oligonucleotide has a nucleobase sequence that consists of the sequence recited in SEQ ID NO: 32.
Embodiment 40. The compound of any of embodiments 1 to 14, wherein the modified oligonucleotide has a nucleobase sequence comprising the sequence recited in SEQ ID NO: 23, 24, 25, 26, 27, 28, 29, 30, 31, or 32.
Embodiment 41. The compound of any of embodiments 1 to 14, wherein the modified oligonucleotide has a nucleobase sequence comprising the sequence recited in SEQ ID NO: 23, 25, 26, 27, 28, 29, 30, 31, or 32.
Embodiment 42. The compound of any of embodiments 1 to 14, wherein the modified oligonucleotide has a nucleobase sequence comprising the sequence recited in SEQ ID NO: 33-874.
Embodiment 43. The compound of any of embodiments 1 to 42, wherein the nucleobase sequence of the modified oligonucleotide is at least 90% complementary to SEQ ID NOs: 1-19.
Embodiment 44. The compound of any of embodiments 1 to 34, wherein the nucleobase sequence of the modified oligonucleotide is 100% complementary to SEQ ID NOs: 1-19.
Embodiment 45. The compound of any of embodiments 1 to 30, wherein the modified oligonucleotide consists of 16 linked nucleosides.
Embodiment 46. The compound of any of embodiments 1 to 30, wherein the modified oligonucleotide consists of 17 linked nucleosides.
Embodiment 47. The compound of any of embodiments 1 to 30, wherein the modified oligonucleotide consists of 18 linked nucleosides.
Embodiment 48. The compound of any of embodiments 1 to 30, wherein the modified oligonucleotide consists of 19 linked nucleosides.
Embodiment 49. The compound of any of embodiments 1 to 30, wherein the modified oligonucleotide consists of 20 linked nucleosides.
Embodiment 50. The compound of any of embodiments 1 to 49, wherein the modified oligonucleotide is a single-stranded oligonucleotide.
Embodiment 51. The compound of any of embodiments 1 to 50 wherein at least one nucleoside comprises a modified sugar.
Embodiment 52. The compound of any of embodiments 1 to 51 wherein at least two nucleosides comprise a modified sugar.
Embodiment 53. The compound of embodiment 52, wherein each of the modified sugars have the same modification.
Embodiment 54. The compound of embodiment 52, wherein at least one the modified sugars has a different modification.
Embodiment 55. The compound of any of embodiments 51 to 54, wherein at least one modified sugar is a bicyclic sugar.
Embodiment 56. The compound of embodiment 55, wherein the bicyclic sugar is selected from among cEt, LNA, α-L-LNA, ENA and 2′-thio LNA.
Embodiment 57. The compound of embodiment 56, wherein the bicyclic sugar comprises cEt.
Embodiment 58. The compound of embodiment 56, wherein the bicyclic sugar comprises LNA.
Embodiment 59. The compound of embodiment 56, wherein the bicyclic sugar comprises α-L-LNA.
Embodiment 60. The compound of embodiment 56, wherein the bicyclic sugar comprises ENA.
Embodiment 61. The compound of embodiment 56, wherein the bicyclic sugar comprises 2′-thio LNA.
Embodiment 62. The compound of any of embodiments 1 to 61, wherein at least one modified sugar comprises a 2′-substituted nucleoside.
Embodiment 63. The compound of embodiment 62, wherein the 2′-substituted nucleoside is selected from among: 2′-OCH₃, 2′-F, and 2′-O-methoxyethyl.
Embodiment 64. The compound of any of embodiments 1 to 63, wherein at least one modified sugar comprises a 2′-O-methoxyethyl.
Embodiment 65. The compound of any of embodiments 1 to 64, wherein at least one nucleoside comprises a modified nucleobase.
Embodiment 66. The compound of embodiment 65, wherein the modified nucleobase is a 5-methylcytosine.
Embodiment 67. The compound of any of embodiments 1 to 67, wherein each cytosine is a 5-methylcytosine.
Embodiment 68. The compound of any of embodiments 1 to 67, wherein the modified oligonucleotide comprises:
   a. a gap segment consisting of linked deoxynucleosides;
   b. a 5′ wing segment consisting of linked nucleosides;
   c. a 3′ wing segment consisting of linked nucleosides;
   d. wherein the gap segment is positioned between the 5′ wing segment and the 3′ wing segment and wherein each nucleoside of each wing segment comprises a modified sugar.
Embodiment 69. The compound of embodiment 68, wherein the modified oligonucleotide consists of 16 linked nucleosides.
Embodiment 70. The compound of embodiment 68, wherein the modified oligonucleotide consists of 17 linked nucleosides.
Embodiment 71. The compound of embodiment 68, wherein the modified oligonucleotide consists of 18 linked nucleosides.
Embodiment 72. The compound of embodiment 68, wherein the modified oligonucleotide consists of 19 linked nucleosides.
Embodiment 73. The compound of embodiment 68, wherein the modified oligonucleotide consists of 20 linked nucleosides.
Embodiment 74. The compound of any of embodiments 68 to 73, wherein the 5′-wing segment consists of two linked nucleosides.
Embodiment 75. The compound of any of embodiments 68 to 73, wherein the 5′-wing segment consists of three linked nucleosides.
Embodiment 76. The compound of any of embodiments 68 to 73, wherein the 5′-wing segment consists of four linked nucleosides.
Embodiment 77. The compound of any of embodiments 68 to 73, wherein the 5′-wing segment consists of five linked nucleosides.
Embodiment 78. The compound of any of embodiments 68 to 73, wherein the 5′-wing segment consists of six linked nucleosides.
Embodiment 79. The compound of any of embodiments 68 to 78, wherein the 3′-wing segment consists of two linked nucleosides.
Embodiment 80. The compound of any of embodiments 68 to 78, wherein the 3′-wing segment consists of three linked nucleosides.
Embodiment 81. The compound of any of embodiments 68 to 78, wherein the 3′-wing segment consists of four linked nucleosides.
Embodiment 82. The compound of any of embodiments 68 to 78, wherein the 3′-wing segment consists of five linked nucleosides.
Embodiment 83. The compound of any of embodiments 68 to 78, wherein the 3′-wing segment consists of six linked nucleosides.
Embodiment 84. The compound of any of embodiments 68 to 83, wherein the gap segment consists of six linked deoxynucleosides.
Embodiment 85. The compound of any of embodiments 68 to 83, wherein the gap segment consists of seven linked deoxynucleosides.
Embodiment 86. The compound of any of embodiments 68 to 83, wherein the gap segment consists of eight linked deoxynucleosides.
Embodiment 87. The compound of any of embodiments 68 to 83, wherein the gap segment consists of nine linked deoxynucleosides.
Embodiment 88. The compound of any of embodiments 68 to 83, wherein the gap segment consists of ten linked deoxynucleosides.
Embodiment 89. The compound of any of embodiments 1 to 31, 34, 37 to 45, or 53 to 88, wherein the modified oligonucleotide consists of 16 linked nucleosides and comprises:
   a. a gap segment consisting of ten linked deoxynucleosides;
   b. a 5′ wing segment consisting of three linked nucleosides;
   c. a 3′ wing segment consisting of three linked nucleosides;
   d. wherein the gap segment is positioned between the 5′ wing segment and the 3′ wing segment, and wherein each nucleoside of each wing segment comprises a bicyclic sugar.
Embodiment 90. The compound of any of embodiments 1 to 31, 34, 37 to 45, or 53 to 88, wherein the modified oligonucleotide consists of 16 linked nucleosides and comprises:
   a. a gap segment consisting of eight linked deoxynucleosides;
   b. a 5′ wing segment consisting of four linked nucleosides and having an AABB 5′-wing motif;
   c. a 3′ wing segment consisting of four linked nucleosides and having a BBAA 3′-wing motif;
   d. wherein the gap segment is positioned between the 5′ wing segment and the 3′ wing segment.
Embodiment 91. The compound of any of embodiments 1 to 30, 35, 36, 46, or 50 to 88, wherein the modified oligonucleotide consists of 17 linked nucleosides and comprises:
   a. a gap segment consisting of seven linked deoxynucleosides;
   b. a 5′ wing segment consisting of five linked nucleosides and having an AAABB 5′-wing motif;
   c. a 3′ wing segment consisting of five linked nucleosides and having a BBAAA 3′-wing motif;
   d. wherein the gap segment is positioned between the 5′ wing segment and the 3′ wing segment.
Embodiment 92. The compound of any of embodiments 1 to 31, 34, 37 to 45, or 53 to 88, wherein the modified oligonucleotide consists of 16 linked nucleosides and comprises:
   a. a gap segment consisting of eight linked deoxynucleosides;
   b. a 5′ wing segment consisting of four linked nucleosides and having a E-E-K-K 5′-wing motif;
   c. a 3′ wing segment consisting of four linked nucleosides and having a K-K-E-E 3′-wing motif;
   d. wherein the gap segment is positioned between the 5′ wing segment and the 3′ wing segment, and wherein each E represents 2′-O-methoxyethyl sugar and each K represents a cEt sugar.
Embodiment 93. The compound of any of embodiments 1 to 30, 35, 36, 46, or 50 to 88, wherein the modified oligonucleotide consists of 17 linked nucleosides and comprises:
   a. a gap segment consisting of seven linked deoxynucleosides;
   b. a 5′ wing segment consisting of five linked nucleosides and having an E-E-E-K-K 5′-wing motif;
   c. a 3′ wing segment consisting of five linked nucleosides and having a K-K-E-E-E 3′-wing motif;
   d. wherein the gap segment is positioned between the 5′ wing segment and the 3′ wing segment, and wherein each E represents 2′-O-methoxyethyl sugar and each K represents a cEt sugar.
Embodiment 94. The compound of any of embodiments 1 to 30, 32, 33, or 49 to 88, wherein the modified oligonucleotide consists of 20 linked nucleosides and comprises:
   a. a gap segment consisting of ten linked deoxynucleosides;
   b. a 5′ wing segment consisting of five linked nucleosides;
   c. a 3′ wing segment consisting of five linked nucleosides;
   d. wherein the gap segment is positioned between the 5′ wing segment and the 3′ wing segment, and wherein each nucleoside of each wing segment comprises a 2′-O-methoxyethyl sugar.
Embodiment 95. The compound of any of embodiments 1 to 31, 34, 37 to 45, or 53 to 88, wherein the modified oligonucleotide consists of 16 linked nucleosides and comprises:
   a. a gap segment consisting of ten linked deoxynucleosides;
   b. a 5′ wing segment consisting of three linked nucleosides;
   c. a 3′ wing segment consisting of three linked nucleosides;
   d. wherein the gap segment is positioned between the 5′ wing segment and the 3′ wing segment, and wherein each nucleoside of each wing segment comprises a cEt sugar.
Embodiment 96. The compound of any of embodiments 1 to 67, wherein the modified oligonucleotide comprises at least 8 contiguous nucleobases complementary to a target region within nucleobase 1343 and nucleobase 1368 of SEQ ID NO.: 1, and wherein the modified oligonucleotide comprises:
   a. a gap segment consisting of linked deoxynucleosides;
   b. a 5′ wing segment consisting of linked nucleosides;
   c. a 3′ wing segment consisting of linked nucleosides;
   d. wherein the gap segment is positioned between the 5′ wing segment and the 3′ wing segment and wherein each nucleoside of each wing segment comprises a modified sugar.
Embodiment 97. The compound of embodiment 96, wherein each modified sugar in the 5′-wing segment has the same modifications.
Embodiment 98. The compound of embodiment 96, wherein at least two modified sugars in the 5′-wing segment have different modifications.
Embodiment 99. The compound of any of embodiments 96 to 98 wherein each modified sugar in the 3′-wing segment has the same modifications.
Embodiment 100. The compound of any of embodiments 96 to 98, wherein at least two modified sugars in the 3′-wing segment have different modification.
Embodiment 101. The compound of embodiment 96, wherein at least one modified sugar is a bicyclic sugar selected from among cEt, LNA, α-L-LNA, ENA and 2′-thio LNAs.
Embodiment 102. The compound of embodiment 90 to 91, wherein each B represents a bicyclic sugar selected from among cEt, LNA, α-L-LNA, ENA and 2′-thio LNA.
Embodiment 103. The compound of embodiment 102, wherein the bicyclic sugar comprises BNA.
Embodiment 104. The compound of embodiment 102, wherein the bicyclic sugar comprises cEt.
Embodiment 105. The compound of embodiment 102, wherein the bicyclic sugar comprises LNA.
Embodiment 106. The compound of embodiment 102, wherein the bicyclic sugar comprises α-L-LNA.
Embodiment 107. The compound of embodiment 102, wherein the bicyclic sugar comprises ENA.
Embodiment 108. The compound of embodiment 102, wherein the bicyclic sugar comprises 2′-thio LNA.
Embodiment 109. The compound of embodiment 90 or 91, wherein each A represents a 2′-substituted nucleoside is selected from among: 2′-OCH₃, 2′-F, and 2′-O-methoxyethyl.
Embodiment 110. The compound of embodiment 109, wherein the 2′-substituted nucleoside comprises 2′-O-methoxyethyl.
Embodiment 111. The compound of any of embodiments 1 to 111, wherein at least one internucleoside linkage is a modified internucleoside linkage.
Embodiment 112. The compound of any of embodiments 1 to 111, wherein each internucleoside linkage is a phosphorothioate internucleoside linkage.
Embodiment 113. A compound consisting of ISIS 486178.
Embodiment 114. A compound consisting of ISIS 512497.
Embodiment 115. A compound consisting of ISIS 598768.
Embodiment 116. A compound consisting of ISIS 594300.
Embodiment 117. A compound consisting of ISIS 594292.
Embodiment 118. A compound consisting of ISIS 569473.
Embodiment 119. A compound consisting of ISIS 598769.
Embodiment 120. A compound consisting of ISIS 570808.
Embodiment 121. A compound consisting of ISIS 598777.
Embodiment 122. A compound having a nucleobase sequence as set forth in SEQ ID NO: 23, wherein the modified oligonucleotide consists of 16 linked nucleosides and comprises:
   a. a gap segment consisting of ten linked deoxynucleosides;
   b. a 5′ wing segment consisting of three linked nucleosides;
   c. a 3′ wing segment consisting of three linked nucleosides;
   d. wherein the gap segment is positioned between the 5′ wing segment and the 3′ wing segment;
   e. wherein each nucleoside of each wing segment comprises a bicyclic sugar;
   f. wherein each internucleoside linkage is a phosphorothioate internucleoside linkage; and
   g. wherein each cytosine residue is a 5-methyl cytosine.
Embodiment 123. A compound having a nucleobase sequence as set forth in SEQ ID NO: 29, wherein the modified oligonucleotide consists of 16 linked nucleosides and comprises:
   a. a gap segment consisting of ten linked deoxynucleosides;
   b. a 5′ wing segment consisting of three linked nucleosides;
   c. a 3′ wing segment consisting of three linked nucleosides;
   d. wherein the gap segment is positioned between the 5′ wing segment and the 3′ wing segment;
   e. wherein each nucleoside of each wing segment comprises a bicyclic sugar;
   f. wherein each internucleoside linkage is a phosphorothioate internucleoside linkage; and
   g. wherein each cytosine residue is a 5-methyl cytosine.
Embodiment 124. A compound having a nucleobase sequence as set forth in SEQ ID NO: 31, wherein the modified oligonucleotide consists of 16 linked nucleosides and comprises:
   a. a gap segment consisting of ten linked deoxynucleosides;
   b. a 5′ wing segment consisting of three linked nucleosides;
   c. a 3′ wing segment consisting of three linked nucleosides;
   d. wherein the gap segment is positioned between the 5′ wing segment and the 3′ wing segment;
   e. wherein each nucleoside of each wing segment comprises a bicyclic sugar;
   f. wherein each internucleoside linkage is a phosphorothioate internucleoside linkage; and
   g. wherein each cytosine residue is a 5-methyl cytosine.
Embodiment 125. A compound having a nucleobase sequence as set forth in SEQ ID NO: 26, wherein the modified oligonucleotide consists of 16 linked nucleosides and comprises:
   a. a gap segment consisting of eight linked deoxynucleosides;
   b. a 5′ wing segment consisting of four linked nucleosides and having a E-E-K-K 5′-wing motif;
   c. a 3′ wing segment consisting of four linked nucleosides and having a K-K-E-E 3′-wing motif;
   d. wherein the gap segment is positioned between the 5′ wing segment and the 3′ wing segment;
   e. wherein each E represents 2′-O-methoxyethyl sugar and each K represents a cEt sugar;
   f. wherein each internucleoside linkage is a phosphorothioate internucleoside linkage; and
   g. wherein each cytosine residue is a 5-methyl cytosine.
Embodiment 126. A compound having a nucleobase sequence as set forth in SEQ ID NO: 30, wherein the modified oligonucleotide consists of 16 linked nucleosides and comprises:
   a. a gap segment consisting of eight linked deoxynucleosides;
   b. a 5′ wing segment consisting of four linked nucleosides and having a E-E-K-K 5′-wing motif;
   c. a 3′ wing segment consisting of four linked nucleosides and having a K-K-E-E 3′-wing motif;
   d. wherein the gap segment is positioned between the 5′ wing segment and the 3′ wing segment;
   e. wherein each E represents 2′-O-methoxyethyl sugar and each K represents a cEt sugar;
   f. wherein each internucleoside linkage is a phosphorothioate internucleoside linkage; and
   g. wherein each cytosine residue is a 5-methyl cytosine.
Embodiment 127. A compound having a nucleobase sequence as set forth in SEQ ID NO: 32, wherein the modified oligonucleotide consists of 16 linked nucleosides and comprises:
   a. a gap segment consisting of eight linked deoxynucleosides;
   b. a 5′ wing segment consisting of four linked nucleosides and having a E-E-K-K 5′-wing motif;
   c. a 3′ wing segment consisting of four linked nucleosides and having a K-K-E-E 3′-wing motif;
   d. wherein the gap segment is positioned between the 5′ wing segment and the 3′ wing segment;
   e. wherein each E represents 2′-O-methoxyethyl sugar and each K represents a cEt sugar;
   f. wherein each internucleoside linkage is a phosphorothioate internucleoside linkage; and
   g. wherein each cytosine residue is a 5-methyl cytosine.
Embodiment 128. A compound having a nucleobase sequence as set forth in SEQ ID NO: 27, wherein the modified oligonucleotide consists of 17 linked nucleosides and comprises:
   a. a gap segment consisting of seven linked deoxynucleosides;
   b. a 5′ wing segment consisting of five linked nucleosides and having an E-E-E-K-K 5′-wing motif;
   c. a 3′ wing segment consisting of five linked nucleosides and having a K-K-E-E-E 3′-wing motif;
   d. wherein the gap segment is positioned between the 5′ wing segment and the 3′ wing segment;
   e. wherein each E represents 2′-O-methoxyethyl sugar and each K represents a cEt sugar;
   f. wherein each internucleoside linkage is a phosphorothioate internucleoside linkage; and
   g. wherein each cytosine residue is a 5-methyl cytosine.
Embodiment 129. A compound having a nucleobase sequence as set forth in SEQ ID NO: 28, wherein the modified oligonucleotide consists of 17 linked nucleosides and comprises:
   a. a gap segment consisting of seven linked deoxynucleosides;
   b. a 5′ wing segment consisting of five linked nucleosides and having an E-E-E-K-K 5′-wing motif;
   c. a 3′ wing segment consisting of five linked nucleosides and having a K-K-E-E-E 3′-wing motif;
   d. wherein the gap segment is positioned between the 5′ wing segment and the 3′ wing segment;
   e. wherein each E represents 2′-O-methoxyethyl sugar and each K represents a cEt sugar;
   f. wherein each internucleoside linkage is a phosphorothioate internucleoside linkage; and
   g. wherein each cytosine residue is a 5-methyl cytosine.
Embodiment 130. A compound having a nucleobase sequence as set forth in SEQ ID NO: 25, wherein the modified oligonucleotide consists of 20 linked nucleosides and comprises:
   a. a gap segment consisting of ten linked deoxynucleosides;
   b. a 5′ wing segment consisting of five linked nucleosides;
   c. a 3′ wing segment consisting of five linked nucleosides;
   d. wherein the gap segment is positioned between the 5′ wing segment and the 3′ wing segment;
   e. wherein each nucleoside of each wing segment comprises a 2′-O-methoxyethyl sugar;
   f. wherein each internucleoside linkage is a phosphorothioate internucleoside linkage; and
   g. wherein each cytosine residue is a 5-methyl cytosine.
Embodiment 131. The compound of any of embodiments 1 to 130 comprising a conjugate.
Embodiment 132. A composition comprising the compound of any of embodiments 1 to 131, and a pharmaceutically acceptable carrier or diluent.
Embodiment 133. A method of treating DM1 in an animal comprising administering to an animal in need thereof a compound according to any of embodiments 1 to 130, or a composition according to embodiment 132.
Embodiment 134. The method of embodiment 133, wherein the compound reduces DMPK mRNA levels.
Embodiment 135. The method of embodiment 133, wherein the compound reduces DMPK protein expression.
Embodiment 136. The method of embodiment 133, wherein the compound reduces CUGexp DMPK.
Embodiment 137. The method of embodiment 133, wherein the compound preferentially reduces CUGexp DMPK.
Embodiment 138. The method of embodiment 133, wherein the compound reduces CUGexp DMPK mRNA.
Embodiment 139. The method of embodiment 133, wherein the compound preferentially reduces CUGexp DMPK mRNA.
Embodiment 140. The method of embodiment 138 or 139, wherein the preferential reduction of CUGexp is in muscle tissue.
Embodiment 141. A method of reducing myotonia in an animal comprising administering to an animal in need thereof a compound according to any of embodiments 1 to 131, or a composition according to embodiment 132.
Embodiment 142. A method of reducing MBLN dependent spliceopathy in an animal comprising administering to an animal in need thereof a compound according to any of embodiments 1 to 131, or a composition according to embodiment 132.
Embodiment 143. The method of embodiment 138, wherein splicing of any of Sercal, m-Titin, Clcn1, and Zasp is corrected.
Embodiment 144. The method of any of embodiments 133 to 143, wherein the administering is systemic administration.
Embodiment 145. The method of any of embodiments 133 to 143, wherein the administering is parenteral administration.
Embodiment 146. The method of embodiment 144, wherein the systemic administration is any of subcutaneous administration, intravenous administration, intracerebroventricular administration, and intrathecal administration.
Embodiment 147. The method of any of embodiments 133 to 143, wherein the administration is not intramuscular administration.
Embodiment 148. The method of any of embodiments 133 to 143, wherein the animal is a human.
Embodiment 149. A method of reducing spliceopathy of Sercal in an animal in need thereof by administering a compound according to any of embodiments 1 to 131, or a composition according to embodiment 132, and thereby causing Sercal exon 22 inclusion.
Embodiment 150. A method of reducing spliceopathy of m-Titin in an animal in need thereof by administering a compound according to any of embodiments 1 to 131, or a composition according to embodiment 132, and thereby causing m-Titin exon 5 inclusion.
Embodiment 151. A method of reducing spliceopathy of Clcn1 in an animal in need thereof by administering a compound according to any of embodiments 1 to 131, or a composition according to embodiment 132, and thereby causing Clcn1 exon 7a inclusion.
Embodiment 152. A method of reducing spliceopathy of Zasp in an animal in need thereof by administering a compound according to any of embodiments 1 to 131, or a composition according to embodiment 132, and thereby causing Zasp exon 11 inclusion.
Embodiment 153. A method of reducing DMPK mRNA in a cell, comprising contacting a cell with a compound according to any of embodiments 1 to 131, or a composition according to embodiment 132.
Embodiment 154. A method of reducing DMPK protein in a cell, comprising contacting a cell with a compound according to any of embodiments 1 to 131, or a composition according to embodiment 132.
Embodiment 155. A method of reducing CUGexp mRNA in a cell, comprising contacting a cell with a compound according to any of embodiments 1 to 131, or a composition according to embodiment 132.
Embodiment 156. The method of any of embodiments 149 to 151, wherein the cell is in an animal.
Embodiment 157. The method of embodiment 156, wherein the animal is a human.
Embodiment 158. A method of achieving a preferential reduction of CUGexp DMPK RNA, comprising:
   a. selecting a subject having type 1 myotonic dystrophy or having a CUGexp DMPK RNA; and
   b. administering to said subject a compound according to any of embodiments 1 to 131, or a composition according to embodiment 132;
wherein said compound according to any of embodiments 1 to 131, or a composition according to embodiment 132, when bound to said CUGexp DMPK RNA, activates a ribonuclease, thereby achieving a preferential reduction of said CUGexp DMPK RNA.
Embodiment 159. A method of achieving a preferential reduction of CUGexp DMPK RNA, comprising:
   a. selecting a subject having type 1 myotonic dystrophy or having a CUGexp DMPK RNA; and
   b. systemically administering to said subject a compound according to any of embodiments 1 to 131, or a composition according to embodiment 132;
wherein said chemically-modified antisense oligonucleotide, when bound to said CUGexp DMPK RNA, achieves a preferential reduction of said CUGexp DMPK RNA.
Embodiment 160. A method of reducing spliceopathy in a subject suspected of having type 1 myotonic dystrophy or having a nuclear retained CUGexp DMPK RNA, comprising: administering to said subject a compound according to any of embodiments 1 to 131, or a composition according to embodiment 132,
wherein the compound according to any of embodiments 1 to 131, or a composition according to embodiment 132, when bound to said mutant DMPK RNA, activates a ribonuclease, thereby reducing spliceopathy.
Embodiment 161. A method of preferentially reducing CUGexp DMPK RNA, reducing myotonia or reducing spliceopathy in an animal comprising administering to the animal a compound according to any of embodiments 1 to 131 or a pharmaceutical composition of embodiment 132, wherein the compound reduces DMPK expression in the animal, thereby preferentially reducing CUGexp DMPK RNA, reducing myotonia, or reducing spliceopathy in the animal.
Embodiment 162. A method for treating an animal with type 1 myotonic dystrophy comprising
   identifying said animal with type 1 myotonic dystrophy,
   administering to said animal a therapeutically effective amount of a compound according to any of embodiments 1 to 131 or a pharmaceutical composition of embodiment 132,
   wherein said animal with type 1 myotonic dystrophy is treated.
Embodiment 163. A method of reducing DMPK expression comprising administering to an animal a compound according to any of embodiments 1 to 131 or a pharmaceutical composition of embodiment 132, wherein expression of DMPK is reduced.
Embodiment 164. A compound according to any of embodiments 1 to 131 or a pharmaceutical composition of embodiment 132, for use in treating DM1 in an animal.
Embodiment 165. A compound according to any of embodiments 1 to 131 or a pharmaceutical composition of embodiment 132, for use in reducing myotonia in an animal.
Embodiment 166. A compound according to any of embodiments 1 to 131 or a pharmaceutical composition of embodiment 132, for use in reducing MBLN dependent spliceopathy in an animal.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. Herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques can be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furanosyl ring. A 2′-O-methoxyethyl modified sugar is a modified sugar.

“2′-O-methoxyethyl nucleotide” means a nucleotide comprising a 2′-O-methoxyethyl modified sugar moiety.

“5-methylcytosine” means a cytosine modified with a methyl group attached to position 5. A 5-methylcytosine is a modified nucleobase.

“About” means within ±7% of a value. For example, if it is stated, “the compound affected at least about 70% inhibition of DMPK”, it is implied that the DMPK levels are inhibited within a range of 63% and 77%.

“Active pharmaceutical agent” means the substance or substances in a pharmaceutical composition that provide a therapeutic benefit when administered to an animal. For example, in certain embodiments an antisense oligonucleotide targeted to DMPK is an active pharmaceutical agent.

“Active target region” or “target region” means a region to which one or more active antisense compounds is targeted. “Active antisense compounds” means antisense compounds that reduce target nucleic acid levels or protein levels.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing an agent to an animal, and includes, but is not limited to, administering by a medical professional and self-administering.

“Agent” means an active substance that can provide a therapeutic benefit when administered to an animal. “First Agent” means a therapeutic compound of the invention. For example, a first agent can be an antisense oligonucleotide targeting DMPK. “Second agent” means a second therapeutic compound of the invention (e.g. a second antisense oligonucleotide targeting DMPK) and/or a non-DMPK therapeutic compound.

“Amelioration” refers to a lessening of at least one indicator, sign, or symptom of an associated disease, disorder, or condition. The severity of indicators can be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, snoRNAs, miRNAs, and satellite repeats.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

“Bicyclic sugar” means a furanosyl ring modified by the bridging of two non-geminal carbon ring atoms. A bicyclic sugar is a modified sugar.

“Bicyclic nucleic acid” or “BNA” refers to a nucleoside or nucleotide wherein the furanose portion of the nucleoside or nucleotide includes a bridge connecting two carbon atoms on the furanose ring, thereby forming a bicyclic ring system.

“Cap structure” or “terminal cap moiety” means chemical modifications, which have been incorporated at either terminus of an antisense compound.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Co-administration” means administration of two or more agents to an individual. The two or more agents can be in a single pharmaceutical composition, or can be in separate pharmaceutical compositions. Each of the two or more agents can be administered through the same or different routes of administration. Co-administration encompasses parallel or sequential administration.

“Complementarity” means the capacity for pairing between nucleobases of a first nucleic acid and a second nucleic acid.

“Contiguous nucleobases” means nucleobases immediately adjacent to each other.

“CUGexp DMPK” means mutant DMPK RNA containing an expanded CUG repeat (CUGexp). The wild-type DMPK gene has 5-37 CTG repeats in the 3′ untranslated region. In a “CUGexp DMPK” (such as in a myotonic dystrophy type I patient) this number is significantly expanded and is, for example, in the range of 50 to greater than 3,500 (Harper, Myotonic Dystrophy (Saunders, London, ed.3, 2001); Annu. Rev. Neurosci. 29: 259, 2006; EMBO J. 19: 4439, 2000; Curr Opin Neurol. 20: 572, 2007).

“Diluent” means an ingredient in a composition that lacks pharmacological activity, but is pharmaceutically necessary or desirable. For example, the diluent in an injected composition can be a liquid, e.g. saline solution.

“DMPK” means any nucleic acid or protein of distrophia myotonica protein kinase. DMPK can be a mutant DMPK including CUGexp DMPK nucleic acid.

“DMPK expression” means the level of mRNA transcribed from the gene encoding DMPK or the level of protein translated from the mRNA. DMPK expression can be determined by art known methods such as a Northern or Western blot.

“DMPK nucleic acid” means any nucleic acid encoding DMPK. For example, in certain embodiments, a DMPK nucleic acid includes a DNA sequence encoding DMPK, an RNA sequence transcribed from DNA encoding DMPK (including genomic DNA comprising introns and exons), and an mRNA or pre-mRNA sequence encoding DMPK. “DMPK mRNA” means an mRNA encoding a DMPK protein.

“Dose” means a specified quantity of a pharmaceutical agent provided in a single administration, or in a specified time period. In certain embodiments, a dose can be administered in one, two, or more boluses, tablets, or injections. For example, in certain embodiments where subcutaneous administration is desired, the desired dose requires a volume not easily accommodated by a single injection, therefore, two or more injections can be used to achieve the desired dose. In certain embodiments, the pharmaceutical agent is administered by infusion over an extended period of time or continuously. Doses can be stated as the amount of pharmaceutical agent per hour, day, week, or month.

“Effective amount” or “therapeutically effective amount” means the amount of active pharmaceutical agent sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

“Fully complementary” or “100% complementary” means each nucleobase of a nucleobase sequence of a first nucleic acid has a complementary nucleobase in a second nucleobase sequence of a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region can be referred to as a “gap segment” and the external regions can be referred to as “wing segments.”

“Gap-widened” means a chimeric antisense compound having a gap segment of 12 or more contiguous 2′-deoxyribonucleosides positioned between and immediately adjacent to 5′ and 3′ wing segments having from one to six nucleosides.

“Hybridization” means the annealing of complementary nucleic acid molecules. In certain embodiments, complementary nucleic acid molecules include an antisense compound and a target nucleic acid.

“Identifying an animal with type 1 myotonic dystrophy” means identifying an animal having been diagnosed with a type 1 myotonic dystrophy, disorder or condition or identifying an animal predisposed to develop a type 1 myotonic dystrophy, disorder or condition. For example, individuals with a familial history can be predisposed to type 1 myotonic dystrophy, disorder or condition. Such identification can be accomplished by any method including evaluating an individual's medical history and standard clinical tests or assessments.

“Immediately adjacent” means there are no intervening elements between the immediately adjacent elements.

“Individual” means a human or non-human animal selected for treatment or therapy.

“Internucleoside linkage” refers to the chemical bond between nucleosides.

“Linked nucleosides” means adjacent nucleosides which are bonded or linked together by an internucleoside linkage.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e. a phosphodiester internucleoside bond).

“Modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleotide” means a nucleotide having, independently, a modified sugar moiety, modified internucleoside linkage, or modified nucleobase. A “modified nucleoside” means a nucleoside having, independently, a modified sugar moiety or modified nucleobase.

“Modified oligonucleotide” means an oligonucleotide comprising at least one modified nucleoside and/or modified internucleoside linkage.

“Modified sugar” refers to a substitution or change from a natural sugar moiety. Modified sugars include substituted sugar moeities and surrogate sugar moieties.

“Motif” means the pattern of chemically distinct regions in an antisense compound.

“Myotonia” means an abnormally slow relaxation of a muscle after voluntary contraction or electrical stimulation.

“Nuclear ribonuclease” means a ribonuclease found in the nucleus. Nuclear ribonucleases include, but are not limited to, RNase H including RNase H1 and RNase H2, the double stranded RNase drosha and other double stranded RNases.

“Naturally occurring internucleoside linkage” means a 3′ to 5′ phosphodiester linkage.

“Natural sugar moiety” means a sugar found in DNA (2′-H) or RNA (2′-OH).

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA). A nucleic acid can also comprise a combination of these elements in a single molecule.

“Nucleobase” means a heterocyclic moiety capable of pairing with a base of another nucleic acid.

“Nucleobase sequence” means the order of contiguous nucleobases independent of any sugar, linkage, or nucleobase modification.

“Nucleoside” means a nucleobase linked to a sugar. In certain embodiments, a nucleoside is linked to a phosphate group.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics e.g. non furanose sugar units.

“Nucleotide” means a nucleoside having a phosphate group covalently linked to the sugar portion of the nucleoside.

“Nucleotide mimetic” includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage).

“Oligomeric compound” or “oligomer” means a polymer of linked monomeric subunits which is capable of hybridizing to at least a region of a nucleic acid molecule.

“Oligonucleotide” means a polymer of linked nucleosides, wherein each nucleoside and each internucleoside linkage may be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration. Administration can be continuous, or chronic, or short or intermittent.

“Peptide” means a molecule formed by linking at least two amino acids by amide bonds. Peptide refers to polypeptides and proteins.

“Pharmaceutical composition” means a mixture of substances suitable for administering to an individual. For example, a pharmaceutical composition can comprise one or more active agents and a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Preferentially reducing CUG exp DMPK RNA” refers to a preferential reduction of RNA transcripts from a CUGexp DMPK allele relative to RNA transcripts from a normal DMPK allele.

“Prevent” refers to delaying or forestalling the onset or development of a disease, disorder, or condition for a period of time from minutes to indefinitely. Prevent also means reducing risk of developing a disease, disorder, or condition.

“Prodrug” means a therapeutic agent that is prepared in an inactive form that is converted to an active form within the body or cells thereof by the action of endogenous enzymes or other chemicals or conditions.

“Side effects” means physiological responses attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum can indicate liver toxicity or liver function abnormality. For example, increased bilirubin can indicate liver toxicity or liver function abnormality.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e. under physiological conditions in the case of in vivo assays and therapeutic treatments.

“Spliceopathy” means a change in the alternative splicing of one or more RNAs that leads to the expression of altered splice products in a particular tissue.

“Subcutaneous administration” means administration just below the skin.

“Substituted sugar moiety” means a furanosyl other than a natural sugar of RNA or DNA.

“Sugar” or “Sugar moiety” means a natural sugar moiety or a modified sugar.

“Sugar surrogate” overlaps with the slightly broader term “nucleoside mimetic” but is intended to indicate replacement of the sugar unit (furanose ring) only A sugar surrogate is capable of replacing the naturally occurring sugar moiety of a nucleoside, such that the resulting nucleoside sub-units are capable of linking together and/or linking to other nucleosides to form an oligomeric compound which is capable of hybridizing to a complementary oligomeric compound. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds. In certain embodiments, a target nucleic acid comprises a region of a DMPK nucleic acid.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutically effective amount” means an amount of an agent that provides a therapeutic benefit to an individual.

“Treat” refers to administering a pharmaceutical composition to effect an alteration or improvement of a disease, disorder, or condition.

“Type 1 myotonic dystrophy” or “DM1” means an autosomal dominant disorder caused by expansion of a non-coding CTG repeat in DMPK. This mutation leads to RNA dominance, a process in which expression of RNA containing an expanded CUG repeat (CUGexp) induced cell dysfunction. The CUGexp tract interacts with RNA binding proteins and causes the mutant transcript to be retained in nuclear foci. The toxicity of this RNA stems from sequestration of RNA binding proteins and activation of signaling pathways.

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

### Certain Embodiments

Certain embodiments provide methods, compounds, and compositions for inhibiting DMPK expression.

Certain embodiments provide a method of reducing DMPK expression in an animal comprising administering to the animal a compound comprising a modified oligonucleotide targeting DMPK.

Certain embodiments provide a method of preferentially reducing CUGexp DMPK RNA, reducing myotonia or reducing spliceopathy in an animal comprising administering to the animal a compound comprising a modified oligonucleotide targeted to DMPK, wherein the modified oligonucleotide preferentially reduces CUGexp DMPK RNA, reduces myotonia or reduces spliceopathy in the animal.

Certain embodiments provide a method of administering an antisense oligonucleotide to counteract RNA dominance by directing the cleavage of pathogenic transcripts.

Certain embodiments provide a method of reducing spliceopathy of Sercal. In certain embodiments, methods provided herein result in exon 22 inclusion. In certain embodiments, the corrective splicing occurs in the tibialis anterior, gastrocnemius, and quadriceps muscles.

Certain embodiments provide a method of reducing spliceopathy of m-Titin. In certain embodiments, methods provided herein result in exon 5 inclusion. In certain embodiments, the corrective splicing occurs in the tibialis anterior, gastrocnemius, and quadriceps muscles.

Certain embodiments provide a method of reducing spliceopathy of Clcn1. In certain embodiments, methods provided herein result in exon 7a inclusion. In certain embodiments, the corrective splicing occurs in the tibialis anterior, gastrocnemius, and quadriceps muscles.

Certain embodiments provide a method of reducing spliceopathy of Zasp. In certain embodiments, methods provided herein result in exon 11 inclusion. In certain embodiments, the corrective splicing occurs in the tibialis anterior, gastrocnemius, and quadriceps muscles.

Certain embodiments provide a method for treating an animal with type 1 myotonic dystrophy comprising: a) identifying said animal with type 1 myotonic dystrophy, and b) administering to said animal a therapeutically effective amount of a compound comprising a modified oligonucleotide targeted to DMPK. In certain embodiments, the therapeutically effective amount of the compound administered to the animal preferentially reduces CUGexp DMPK RNA, reduces myotonia or reduces spliceopathy in the animal.

Certain embodiments provide a method of achieving a preferential reduction of CUGexp DMPK RNA, including administering to the subject suspected of having type 1 myotonic dystrophy or having a CUGexp DMPK RNA a modified antisense oligonucleotide complementary to a non-repeat region of said CUGexp DMPK RNA. The modified antisense oligonucleotide, when bound to said CUGexp DMPK RNA, achieves a preferential reduction of the CUGexp DMPK RNA.

Certain embodiments provide a method of achieving a preferential reduction of CUGexp DMPK RNA, including selecting a subject having type 1 myotonic dystrophy or having a CUGexp DMPK RNA and administering to said subject a modified antisense oligonucleotide complementary to a non-repeat region of said CUGexp DMPK RNA. The modified antisense oligonucleotide, when bound to the CUGexp DMPK RNA, activates a ribonuclease or nuclear ribonuclease, thereby achieving a preferential reduction of the CUGexp DMPK RNA in the nucleus.

Certain embodiments provide a method of achieving a preferential reduction of CUGexp DMPK RNA, including selecting a subject having type 1 myotonic dystrophy or having a mutant or CUGexp DMPK RNA and systemically administering to said subject a modified antisense oligonucleotide complementary to a non-repeat region of said CUGexp DMPK RNA. The modified antisense oligonucleotide, when bound to the mutant or CUGexp DMPK RNA, achieves a preferential reduction of the mutant or CUGexp DMPK RNA.

Certain embodiments provide a method of reducing myotonia in a subject in need thereof. The method includes administering to the subject a modified antisense oligonucleotide complementary to a non-repeat region of a DMPK RNA, wherein the modified antisense oligonucleotide, when bound to the DMPK RNA, activates a ribonuclease or nuclear ribonuclease, thereby reducing myotonia. In certain embodiments, the subject has or is suspected of having type 1 myotonic dystrophy or having a mutant DMPK RNA or CUGexp DMPK RNA. In certain embodiments, the DMPK RNA is nuclear retained.

Certain embodiments provide a method of reducing spliceopathy in a subject in need thereof. The method includes administering to the subject a modified antisense oligonucleotide complementary to a non-repeat region of a DMPK RNA, wherein the modified antisense oligonucleotide, when bound to the DMPK RNA, activates a ribonuclease or nuclear ribonuclease, thereby reducing spliceopathy. In certain embodiments, the subject has or is suspected of having type 1 myotonic dystrophy or having a nuclear retained CUGexp DMPK RNA. In certain embodiments, the DMPK RNA is nuclear retained. In certain embodiments, the spliceopathy is MBNL dependent spliceopathy.

In certain embodiments, the modified antisense oligonucleotide of the methods is chimeric. In certain embodiments, the modified antisense oligonucleotide of the methods is a gapmer.

In certain embodiments of the methods provided herein, the administering is subcutaneous. In certain embodiments, the administering is intravenous.

In certain embodiments, the modified antisense oligonucleotide of the methods targets a non-coding sequence within the non-repeat region of a DMPK RNA. In certain embodiments, the oligonucleotide targets a coding region, an intron, a 5′UTR, or a 3′UTR of the mutant DMPK RNA.

In certain embodiments of the methods provided herein, the nuclear ribonuclease is RNase H1.

In certain embodiments of the methods, the DMPK RNA is reduced in muscle tissue. In certain embodiments, the mutant DMPK RNA CUGexp DMPK RNA is preferentially reduced.

In certain embodiments, the DMPK has the sequence as set forth in GenBank Accession No. NM_001081560.1 (incorporated herein as SEQ ID NO: 1). In certain embodiments, the DMPK has the sequence as set forth in GenBank Accession No. NT_011109.15 truncated from nucleotides 18540696 to 18555106 (incorporated herein as SEQ ID NO: 2). In certain embodiments, the DMPK has the sequence as set forth in GenBank Accession No. NT_039413.7 truncated from nucleotides 16666001 to 16681000 (incorporated herein as SEQ ID NO: 3). In certain embodiments, the DMPK has the sequence as set forth in GenBank Accession No. NM_032418.1 (incorporated herein as SEQ ID NO: 4). In certain embodiments, the DMPK has the sequence as set forth in GenBank Accession No. AI007148.1 (incorporated herein as SEQ ID NO: 5). In certain embodiments, the DMPK has the sequence as set forth in GenBank Accession No. AI304033.1 (incorporated herein as SEQ ID NO: 6). In certain embodiments, the DMPK has the sequence as set forth in GenBank Accession No. BC024150.1 (incorporated herein as SEQ ID NO: 7). In certain embodiments, the DMPK has the sequence as set forth in GenBank Accession No. BC056615.1 (incorporated herein as SEQ ID NO: 8). In certain embodiments, the DMPK has the sequence as set forth in GenBank Accession No. BC075715.1 (incorporated herein as SEQ ID NO: 9). In certain embodiments, the DMPK has the sequence as set forth in GenBank Accession No. BU519245.1 (incorporated herein as SEQ ID NO: 10). In certain embodiments, the DMPK has the sequence as set forth in GenBank Accession No. CB247909.1 (incorporated herein as SEQ ID NO: 11). In certain embodiments, the DMPK has the sequence as set forth in GenBank Accession No. CX208906.1 (incorporated herein as SEQ ID NO: 12). In certain embodiments, the DMPK has the sequence as set forth in GenBank Accession No. CX732022.1 (incorporated herein as SEQ ID NO: 13). In certain embodiments, the DMPK has the sequence as set forth in GenBank Accession No. S60315.1 (incorporated herein as SEQ ID NO: 14). In certain embodiments, the DMPK has the sequence as set forth in GenBank Accession No. S60316.1 (incorporated herein as SEQ ID NO: 15). In certain embodiments, the DMPK has the sequence as set forth in GenBank Accession No. NM_001081562.1 (incorporated herein as SEQ ID NO: 16). In certain embodiments, the DMPK has the sequence as set forth in GenBank Accession No. NM_001100.3 (incorporated herein as SEQ ID NO: 17).

In certain embodiments, the modified oligonucleotide has a nucleobase sequence comprising at least 8 contiguous nucleobases of a nucleobase sequence recited in any one of SEQ ID NOs: 23, 24, 25, 26, 27, 28, 29, 30, 31, 32, or 33-874. In certain embodiments, the modified oligonucleotide has a nucleobase sequence comprising at least 9, at least 10, or at least 11, contiguous nucleobases of a nucleobase sequence recited in any one of SEQ ID NOs: 23, 24, 25, 26, 27, 28, 29, 30, 31, 32, or 33-874.

In certain embodiments, the modified oligonucleotide has a nucleobase sequence comprising at least 12 contiguous nucleobases of a nucleobase sequence recited in any one of SEQ ID NOs: 23, 24, 25, 26, 27, 28, 29, 30, 31, 32, or 33-874. In certain embodiments, the modified oligonucleotide has a nucleobase sequence comprising at least 13, or at least 14, contiguous nucleobases of a nucleobase sequence recited in any one of SEQ ID NOs: 23, 24, 25, 26, 27, 28, 29, 30, 31, 32, or 33-874.

In certain embodiments, the modified oligonucleotide has a nucleobase sequence comprising at least 15 contiguous nucleobases of a nucleobase sequence recited in any one of SEQ ID NOs: 23, 24, 25, 26, 27, 28, 29, 30, 31, 32, or 33-874. In certain embodiments, the modified oligonucleotide has a nucleobase sequence comprising at least 16 contiguous nucleobases of a nucleobase sequence recited in any one of SEQ ID NOs: 23, 24, 25, 26, 27, 28, 29, 30, 31, 32, or 33-874.

In certain embodiments, the modified oligonucleotide has a nucleobase sequence comprising at least 17 contiguous nucleobases of a nucleobase sequence recited in any one of SEQ ID NOs: 24, 25, 27, or 28.

In certain embodiments, the modified oligonucleotide has a nucleobase sequence comprising at least 18 contiguous nucleobases of a nucleobase sequence recited in any one of SEQ ID NOs: 24 or 25. In certain embodiments, the modified oligonucleotide has a nucleobase sequence comprising at least 19 contiguous nucleobases of a nucleobase sequence recited in any one of SEQ ID NOs: 24 or 25.

In certain embodiments, the modified oligonucleotides provided herein are targeted to any one of the following regions of SEQ ID NO: 1: 1343-1368, 1317-1366, 2748-2791, 2155-2208, 2748-2791, 730-748, 528-547, 531-567, 636-697, 1311-1331, 1314-1339, 1446-1475, 1635-1670, 1610-1638, 1457-1486, 2773-1788, 931-948, 934-949, 937-952, 942-957, 937-957, 943-958, 937-953, 1346-1363, 1346-1361, 1347-1363, 2162-2179, 2492-2508, 2696-2717, and 2683-2703. In certain embodiments, the modified oligonucleotides provided herein are targeted to any one of the following regions of SEQ ID NO: 1: 2773-2788, 1343-1358, and 1344-1359.

In certain embodiments, the modified oligonucleotides provided herein have a nucleobase sequence comprising a complementary region comprising at least 8 contiguous nucleobases complementary to a target region. In certain embodiments, the modified oligonucleotides provided herein have a nucleobase sequence comprising a complementary region comprising at least 8 contiguous nucleobases complementary to a target region, wherein the target region is targeted to nucleobases 1343-1368, 1317-1366, 2748-2791, 2155-2208, 2748-2791, 730-748, 528-547, 531-567, 636-697, 1311-1331, 1314-1339, 1446-1475, 1635-1670, 1610-1638, 1457-1486, 2773-1788, 931-948, 934-949, 937-952, 942-957, 937-957, 943-958, 937-953, 1346-1363, 1346-1361, 1347-1363, 2162-2179, 2492-2508, 2696-2717, or 2683-2703 of SEQ ID NO: 1. In certain embodiments, the modified oligonucleotides provided herein have a nucleobase sequence comprising a complementary region comprising at least 8 contiguous nucleobases complementary to a target region, wherein the target region is targeted to nucleobases 2773-2788, 1343-1358, or 1344-1359 of SEQ ID NO: 1.

In certain embodiments, the modified oligonucleotides provided herein have a nucleobase sequence comprising a complementary region comprising at least 10 contiguous nucleobases complementary to a target region. In certain embodiments, the modified oligonucleotides provided herein have a nucleobase sequence comprising a complementary region comprising at least 10 contiguous nucleobases complementary to a target region, wherein the target region is targeted to nucleobases 1343-1368, 1317-1366, 2748-2791, 2155-2208, 2748-2791, 730-748, 528-547, 531-567, 636-697, 1311-1331, 1314-1339, 1446-1475, 1635-1670, 1610-1638, 1457-1486, 2773-1788, 931-948, 934-949, 937-952, 942-957, 937-957, 943-958, 937-953, 1346-1363, 1346-1361, 1347-1363, 2162-2179, 2492-2508, 2696-2717, or 2683-2703 of SEQ ID NO: 1. In certain embodiments, the modified oligonucleotides provided herein have a nucleobase sequence comprising a complementary region comprising at least 10 contiguous nucleobases complementary to a target region, wherein the target region is targeted to nucleobases 2773-2788, 1343-1358, or 1344-1359 of SEQ ID NO: 1.

In certain embodiments, the modified oligonucleotides provided herein have a nucleobase sequence comprising a complementary region comprising at least 12 contiguous nucleobases complementary to a target region. In certain embodiments, the modified oligonucleotides provided herein have a nucleobase sequence comprising a complementary region comprising at least 12 contiguous nucleobases complementary to a target region, wherein the target region is targeted to nucleobases 1343-1368, 1317-1366, 2748-2791, 2155-2208, 2748-2791, 730-748, 528-547, 531-567, 636-697, 1311-1331, 1314-1339, 1446-1475, 1635-1670, 1610-1638, 1457-1486, 2773-1788, 931-948, 934-949, 937-952, 942-957, 937-957, 943-958, 937-953, 1346-1363, 1346-1361, 1347-1363, 2162-2179, 2492-2508, 2696-2717, or 2683-2703 of SEQ ID NO: 1. In certain embodiments, the modified oligonucleotides provided herein have a nucleobase sequence comprising a complementary region comprising at least 12 contiguous nucleobases complementary to a target region, wherein the target region is targeted to nucleobases 2773-2788, 1343-1358, or 1344-1359 of SEQ ID NO: 1.

In certain embodiments, the modified oligonucleotides provided herein have a nucleobase sequence comprising a complementary region comprising at least 14 contiguous nucleobases complementary to a target region. In certain embodiments, the modified oligonucleotides provided herein have a nucleobase sequence comprising a complementary region comprising at least 14 contiguous nucleobases complementary to a target region, wherein the target region is targeted to nucleobases 1343-1368, 1317-1366, 2748-2791, 2155-2208, 2748-2791, 730-748, 528-547, 531-567, 636-697, 1311-1331, 1314-1339, 1446-1475, 1635-1670, 1610-1638, 1457-1486, 2773-1788, 931-948, 934-949, 937-952, 942-957, 937-957, 943-958, 937-953, 1346-1363, 1346-1361, 1347-1363, 2162-2179, 2492-2508, 2696-2717, or 2683-2703 of SEQ ID NO: 1. In certain embodiments, the modified oligonucleotides provided herein have a nucleobase sequence comprising a complementary region comprising at least 14 contiguous nucleobases complementary to a target region, wherein the target region is targeted to nucleobases 2773-2788, 1343-1358, or 1344-1359 of SEQ ID NO: 1.

In certain embodiments, the modified oligonucleotides provided herein have a nucleobase sequence comprising a complementary region comprising at least 16 contiguous nucleobases complementary to a target region. In certain embodiments, the modified oligonucleotides provided herein have a nucleobase sequence comprising a complementary region comprising at least 16 contiguous nucleobases complementary to a target region, wherein the target region is targeted to nucleobases 1343-1368, 1317-1366, 2748-2791, 2155-2208, 2748-2791, 730-748, 528-547, 531-567, 636-697, 1311-1331, 1314-1339, 1446-1475, 1635-1670, 1610-1638, 1457-1486, 2773-1788, 931-948, 934-949, 937-952, 942-957, 937-957, 943-958, 937-953, 1346-1363, 1346-1361, 1347-1363, 2162-2179, 2492-2508, 2696-2717, or 2683-2703 of SEQ ID NO: 1. In certain embodiments, the modified oligonucleotides provided herein have a nucleobase sequence comprising a complementary region comprising at least 16 contiguous nucleobases complementary to a target region, wherein the target region is targeted to nucleobases 2773-2788, 1343-1358, or 1344-1359 of SEQ ID NO: 1.

In certain embodiments, the modified oligonucleotides provided herein are targeted to any one of the following regions of SEQ ID NO: 2: 10195-10294, 13553-13572, 13748-13767, 13455-13475, 13628-13657, 13735-13760, 13746-13905, 13836-13851, 13553-13568, 13563-13578, 13624-13639, 13686-13701, 13760-13775, 13763-13779, 13765-13780, 2580-2595, 6446-6461, 11099-11115, 11082-11099, 1974-1993, 4435-4456, 6035-6052, 6360-6385, 6445-6468, 6807-6824, 6789-6806, and 6596-6615. In certain embodiments, the modified oligonucleotides provided herein are targeted to any one of the following regions of SEQ ID NO: 2: 13836-13831, 8603-8618, and 8604-8619.

In certain embodiments, the modified oligonucleotides provided herein have a nucleobase sequence comprising a complementary region comprising at least 8 contiguous nucleobases complementary to a target region. In certain embodiments, the modified oligonucleotides provided herein have a nucleobase sequence comprising a complementary region comprising at least 8 contiguous nucleobases complementary to a target region, wherein the target region is targeted to nucleobases 10195-10294, 13553-13572, 13748-13767, 13455-13475, 13628-13657, 13735-13760, 13746-13905, 13836-13851, 13553-13568, 13563-13578, 13624-13639, 13686-13701, 13760-13775, 13763-13779, 13765-13780, 2580-2595, 6446-6461, 11099-11115, 11082-11099, 1974-1993, 4435-4456, 6035-6052, 6360-6385, 6445-6468, 6807-6824, 6789-6806, or 6596-6615 of SEQ ID NO: 2. In certain embodiments, the modified oligonucleotides provided herein have a nucleobase sequence comprising a complementary region comprising at least 8 contiguous nucleobases complementary to a target region, wherein the target region is targeted to nucleobases 13836-13831, 8603-8618, or 8604-8619 of SEQ ID NO: 2.

In certain embodiments, the modified oligonucleotides provided herein have a nucleobase sequence comprising a complementary region comprising at least 10 contiguous nucleobases complementary to a target region. In certain embodiments, the modified oligonucleotides provided herein have a nucleobase sequence comprising a complementary region comprising at least 10 contiguous nucleobases complementary to a target region, wherein the target region is targeted to nucleobases 10195-10294, 13553-13572, 13748-13767, 13455-13475, 13628-13657, 13735-13760, 13746-13905, 13836-13851, 13553-13568, 13563-13578, 13624-13639, 13686-13701, 13760-13775, 13763-13779, 13765-13780, 2580-2595, 6446-6461, 11099-11115, 11082-11099, 1974-1993, 4435-4456, 6035-6052, 6360-6385, 6445-6468, 6807-6824, 6789-6806, or 6596-6615 of SEQ ID NO: 2. In certain embodiments, the modified oligonucleotides provided herein have a nucleobase sequence comprising a complementary region comprising at least 10 contiguous nucleobases complementary to a target region, wherein the target region is targeted to nucleobases 13836-13831, 8603-8618, or 8604-8619 of SEQ ID NO: 2.

In certain embodiments, the modified oligonucleotides provided herein have a nucleobase sequence comprising a complementary region comprising at least 12 contiguous nucleobases complementary to a target region. In certain embodiments, the modified oligonucleotides provided herein have a nucleobase sequence comprising a complementary region comprising at least 12 contiguous nucleobases complementary to a target region, wherein the target region is targeted to nucleobases 10195-10294, 13553-13572, 13748-13767, 13455-13475, 13628-13657, 13735-13760, 13746-13905, 13836-13851, 13553-13568, 13563-13578, 13624-13639, 13686-13701, 13760-13775, 13763-13779, 13765-13780, 2580-2595, 6446-6461, 11099-11115, 11082-11099, 1974-1993, 4435-4456, 6035-6052, 6360-6385, 6445-6468, 6807-6824, 6789-6806, or 6596-6615 of SEQ ID NO: 2. In certain embodiments, the modified oligonucleotides provided herein have a nucleobase sequence comprising a complementary region comprising at least 12 contiguous nucleobases complementary to a target region, wherein the target region is targeted to nucleobases 13836-13831, 8603-8618, or 8604-8619 of SEQ ID NO: 2.

In certain embodiments, the modified oligonucleotides provided herein have a nucleobase sequence comprising a complementary region comprising at least 14 contiguous nucleobases complementary to a target region. In certain embodiments, the modified oligonucleotides provided herein have a nucleobase sequence comprising a complementary region comprising at least 14 contiguous nucleobases complementary to a target region, wherein the target region is targeted to nucleobases 10195-10294, 13553-13572, 13748-13767, 13455-13475, 13628-13657, 13735-13760, 13746-13905, 13836-13851, 13553-13568, 13563-13578, 13624-13639, 13686-13701, 13760-13775, 13763-13779, 13765-13780, 2580-2595, 6446-6461, 11099-11115, 11082-11099, 1974-1993, 4435-4456, 6035-6052, 6360-6385, 6445-6468, 6807-6824, 6789-6806, or 6596-6615 of SEQ ID NO: 2. In certain embodiments, the modified oligonucleotides provided herein have a nucleobase sequence comprising a complementary region comprising at least 14 contiguous nucleobases complementary to a target region, wherein the target region is targeted to nucleobases 13836-13831, 8603-8618, or 8604-8619 of SEQ ID NO: 2.

In certain embodiments, the modified oligonucleotides provided herein have a nucleobase sequence comprising a complementary region comprising at least 16 contiguous nucleobases complementary to a target region. In certain embodiments, the modified oligonucleotides provided herein have a nucleobase sequence comprising a complementary region comprising at least 16 contiguous nucleobases complementary to a target region, wherein the target region is targeted to nucleobases 10195-10294, 13553-13572, 13748-13767, 13455-13475, 13628-13657, 13735-13760, 13746-13905, 13836-13851, 13553-13568, 13563-13578, 13624-13639, 13686-13701, 13760-13775, 13763-13779, 13765-13780, 2580-2595, 6446-6461, 11099-11115, 11082-11099, 1974-1993, 4435-4456, 6035-6052, 6360-6385, 6445-6468, 6807-6824, 6789-6806, or 6596-6615 of SEQ ID NO: 2. In certain embodiments, the modified oligonucleotides provided herein have a nucleobase sequence comprising a complementary region comprising at least 16 contiguous nucleobases complementary to a target region, wherein the target region is targeted to nucleobases 13836-13831, 8603-8618, or 8604-8619 of SEQ ID NO: 2.

In certain embodiments, the animal is a human.

In certain embodiments, the compounds or compositions of the invention are designated as a first agent and the methods of the invention further comprise administering a second agent. In certain embodiments, the first agent and the second agent are co-administered. In certain embodiments the first agent and the second agent are co-administered sequentially or concomitantly.

In certain embodiments, administration comprises parenteral administration.

In certain embodiments, the compound is a single-stranded modified oligonucleotide. In certain embodiments, the nucleobase sequence of the modified oligonucleotide is at least 95% complementary to any one of SEQ ID NOs: 1-19 as measured over the entirety of said modified oligonucleotide. In certain embodiments, the nucleobase sequence of the modified oligonucleotide is 100% complementary to any one of SEQ ID NOs: 1-19 as measured over the entirety of said modified oligonucleotide. In certain embodiments, the compound is a single-stranded modified oligonucleotide. In certain embodiments, the nucleobase sequence of the modified oligonucleotide is at least 95% complementary to any one of SEQ ID NO: 1 as measured over the entirety of said modified oligonucleotide. In certain embodiments, the nucleobase sequence of the modified oligonucleotide is 100% complementary to any one of SEQ ID NO: 1 as measured over the entirety of said modified oligonucleotide.

In certain embodiments, the nucleobase sequence of the modified oligonucleotide is at least 90% complementary to any one of SEQ ID NO: 1 as measured over the entirety of said modified oligonucleotide. In certain embodiments, the nucleobase sequence of the modified oligonucleotide is 85% complementary to any one of SEQ ID NOs: 1 as measured over the entirety of said modified oligonucleotide.

In certain embodiments, the nucleobase sequence of the modified oligonucleotide is at least 90% complementary to any one of SEQ ID NO: 2 as measured over the entirety of said modified oligonucleotide. In certain embodiments, the nucleobase sequence of the modified oligonucleotide is 85% complementary to any one of SEQ ID NO: 2 as measured over the entirety of said modified oligonucleotide.

In certain embodiments, at least one internucleoside linkage of said modified oligonucleotide is a modified internucleoside linkage. In certain embodiments, each internucleoside linkage is a phosphorothioate internucleoside linkage.

In certain embodiments, at least one nucleoside of said modified oligonucleotide comprises a modified sugar. In certain embodiments, at least one modified sugar is a bicyclic sugar. In certain embodiments, at least one modified sugar comprises a 2′-O-methoxyethyl or a 4′-(CH₂)ₙ—O-2′ bridge, wherein n is 1 or 2.

In certain embodiments, at least one nucleoside of said modified oligonucleotide comprises a modified nucleobase. In certain embodiments, the modified nucleobase is a 5-methylcytosine.

In certain embodiments, the modified oligonucleotide comprises: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar.

In certain embodiments, the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, and each cytosine in said modified oligonucleotide is a 5′-methylcytosine.

In certain embodiments, the modified oligonucleotide consists of 20 linked nucleosides. In certain embodiments, the modified oligonucleotide consists of 19 linked nucleosides. In certain embodiments, the modified oligonucleotide consists of 18 linked nucleosides. In certain embodiments, the modified oligonucleotide consists of 17 linked nucleosides. In certain embodiments, the modified oligonucleotide consists of 16 linked nucleosides.

Certain embodiments provide a method of preferentially reducing CUGexp DMPK RNA, reducing myotonia or reducing spliceopathy in an animal comprising administering to the animal a compound comprising a modified oligonucleotide having a gap segment consisting of ten linked deoxynucleosides, a 5′ wing segment consisting of five linked nucleosides and a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine.

In certain embodiments, the modified oligonucleotide comprises: a) a gap segment consisting of eight linked deoxynucleosides; b) a 5′ wing segment consisting of four linked nucleosides and having a E-E-K-K 5′-wing motif, c) a 3′ wing segment consisting of four linked nucleosides and having a K-K-E-E 3′-wing motif, and d) wherein the gap segment is positioned between the 5′ wing segment and the 3′ wing segment, and wherein each E represents 2′-O-methoxyethyl sugar and each K represents a cEt sugar.

In certain embodiments, the modified oligonucleotide comprises: a) a gap segment consisting of seven linked deoxynucleosides; b) a 5′ wing segment consisting of five linked nucleosides and having an E-E-E-K-K 5′-wing motif, c) a 3′ wing segment consisting of five linked nucleosides and having a K-K-E-E-E 3′-wing motif, and d) wherein the gap segment is positioned between the 5′ wing segment and the 3′ wing segment, and wherein each E represents 2′-O-methoxyethyl sugar and each K represents a cEt sugar.

In certain embodiments, the modified oligonucleotide comprises: a) a gap segment consisting of ten linked deoxynucleosides; b) a 5′ wing segment consisting of five linked nucleosides; c) a 3′ wing segment consisting of five linked nucleosides; and d) wherein the gap segment is positioned between the 5′ wing segment and the 3′ wing segment, and wherein each nucleoside of each wing segment comprises a 2′-O-methoxyethyl sugar.

In certain embodiments, the modified oligonucleotide comprises: a) a gap segment consisting of ten linked deoxynucleosides; b) a 5′ wing segment consisting of three linked nucleosides; c) a 3′ wing segment consisting of three linked nucleosides; and d) wherein the gap segment is positioned between the 5′ wing segment and the 3′ wing segment, and wherein each nucleoside of each wing segment comprises a cEt sugar.

Certain embodiments provide a method of preferentially reducing CUGexp DMPK RNA, reducing myotonia or reducing spliceopathy in an animal comprising administering to the animal a compound comprising a modified oligonucleotide having: a) a gap segment consisting of eight linked deoxynucleosides; b) a 5′ wing segment consisting of four linked nucleosides and having a E-E-K-K 5′-wing motif, c) a 3′ wing segment consisting of four linked nucleosides and having a K-K-E-E 3′-wing motif, and d) wherein the gap segment is positioned between the 5′ wing segment and the 3′ wing segment, and wherein each E represents 2′-O-methoxyethyl sugar and each K represents a cEt sugar.

Certain embodiments provide a method of preferentially reducing CUGexp DMPK RNA, reducing myotonia or reducing spliceopathy in an animal comprising administering to the animal a compound comprising a modified oligonucleotide having: a) a gap segment consisting of seven linked deoxynucleosides; b) a 5′ wing segment consisting of five linked nucleosides and having an E-E-E-K-K 5′-wing motif, c) a 3′ wing segment consisting of five linked nucleosides and having a K-K-E-E-E 3′-wing motif, and d) wherein the gap segment is positioned between the 5′ wing segment and the 3′ wing segment, and wherein each E represents 2′-O-methoxyethyl sugar and each K represents a cEt sugar.

Certain embodiments provide a method of preferentially reducing CUGexp DMPK RNA, reducing myotonia or reducing spliceopathy in an animal comprising administering to the animal a compound comprising a modified oligonucleotide having: a) a gap segment consisting of ten linked deoxynucleosides; b) a 5′ wing segment consisting of five linked nucleosides; c) a 3′ wing segment consisting of five linked nucleosides; and d) wherein the gap segment is positioned between the 5′ wing segment and the 3′ wing segment, and wherein each nucleoside of each wing segment comprises a 2′-O-methoxyethyl sugar.

Certain embodiments provide a method of preferentially reducing CUGexp DMPK RNA, reducing myotonia or reducing spliceopathy in an animal comprising administering to the animal a compound comprising a modified oligonucleotide having: a) a gap segment consisting of ten linked deoxynucleosides; b) a 5′ wing segment consisting of three linked nucleosides; c) a 3′ wing segment consisting of three linked nucleosides; and d) wherein the gap segment is positioned between the 5′ wing segment and the 3′ wing segment, and wherein each nucleoside of each wing segment comprises a cEt sugar.

Certain embodiments provide the use of any compound as described herein in the manufacture of a medicament for use in any of the therapeutic methods described herein. For example, certain embodiments provide the use of a compound as described herein in the manufacture of a medicament for treating, ameliorating, or preventing type 1 myotonic dystrophy. Certain embodiments provide the use of a compound as described herein in the manufacture of a medicament for inhibiting expression of DMPK and treating, preventing, delaying or ameliorating a DMPK related disease and or a symptom thereof. Certain embodiments provide the use of a compound as described herein in the manufacture of a medicament for reducing DMPK expression in an animal. Certain embodiments provide the use of a compound as described herein in the manufacture of a medicament for preferentially reducing CUGexp DMPK, reducing myotonia, or reducing spliceopathy in an animal. Certain embodiments provide the use of a compound as described herein in the manufacture of a medicament for treating an animal with type 1 myotonic dystrophy. Certain embodiments provide the use of a compound as described herein in the manufacture of a medicament for treating, preventing, delaying, or ameliorating symptoms and outcomes associated with development of DM1 including muscle stiffness, myotonia, disabling distal weakness, weakness in face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts. Certain embodiments provide the use of a compound as described herein in the manufacture of a medicament for counteracting RNA dominance by directing the cleavage of pathogenic transcripts.

Certain embodiments provide a kit for treating, preventing, or ameliorating type 1 myotonic dystrophy as described herein wherein the kit comprises: a) a compound as described herein; and optionally b) an additional agent or therapy as described herein. The kit can further include instructions or a label for using the kit to treat, prevent, or ameliorate type 1 myotonic dystrophy.

Certain embodiments provide any compound or composition as described herein, for use in any of the therapeutic methods described herein. For example, certain embodiments provide a compound or composition as described herein for inhibiting expression of DMPK and treating, preventing, delaying or ameliorating a DMPK related disease and or a symptom thereof. Certain embodiments provide a compound or composition as described herein for use in reducing DMPK expression in an animal. Certain embodiments provide a compound or composition as described herein for use in preferentially reducing CUGexp DMPK, reducing myotonia, or reducing spliceopathy in an animal. Certain embodiments provide a compound or composition as described herein for use in treating an animal with type 1 myotonic dystrophy. Certain embodiments provide a compound or composition as described herein for use in treating, preventing, delaying, or ameliorating symptoms and outcomes associated with development of DM1 including muscle stiffness, myotonia, disabling distal weakness, weakness in face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts. Certain embodiments provide a compound or composition as described herein for use in counteracting RNA dominance by directing the cleavage of pathogenic transcripts. Certain embodiments provide compounds comprising a modified oligonucleotide consisting of 12 to 30 linked nucleosides having a nucleobase sequence comprising at least 12 contiguous nucleobases of any of the nucleobase sequences of SEQ ID NOs: 23, 24, 25, 26, 27, 28, 29, 30, 31, 32, or 33-874.

Other compounds which can be used in the methods described herein are also provided.

For example, certain embodiments provide compounds comprising a modified oligonucleotide consisting of 10 to 80, 12 to 50, 12 to 30, 15 to 30, 18 to 24, 19 to 22, or 20 linked nucleosides having a nucleobase sequence comprising at least 8, at least 9, at least 10, at least 11, at least 12, at least 13, at least 14, at least 15, at least 16, at least 17, at least 18, or at least 19, contiguous nucleobases of any of the nucleobase sequences of SEQ ID NOs: 23, 24, 25, 26, 27, 28, 29, 30, 31, 32, or 33-874.

Certain embodiments provide compounds comprising a modified oligonucleotide consisting of 10 to 80, 12 to 50, 12 to 30, 15 to 30, 18 to 24, 19 to 22, or 20, linked nucleosides having a nucleobase sequence comprising at least 8, at least 9, at least 10, at least 11, at least 12, at least 13, at least 14, at least 15, at least 16, at least 17, at least 18, at least 19, contiguous nucleobases of any of the nucleobase sequences of SEQ ID NOs: 23, 24, 25, 26, 27, 28, 29, 30, 31, 32, or 33-874.

Certain embodiments provide compounds comprising a modified oligonucleotide consisting of 10 to 80, 12 to 50, 12 to 30, 15 to 30, or 15 to 17, linked nucleosides having a nucleobase sequence comprising a portion of at least 8, at least 9, at least 10, at least 11, at least 12, at least 13, at least 14, at least 15, at least 16, at least 17, at least 18, or at least 19, or more, contiguous nucleobases complementary to an equal length portion of nucleobases 1343-1368, 1317-1366, 2748-2791, 2155-2208, 2748-2791, 730-748, 528-547, 531-567, 636-697, 1311-1331, 1314-1339, 1446-1475, 1635-1670, 1610-1638, 1457-1486, 2773-1788, 931-948, 934-949, 937-952, 942-957, 937-957, 943-958, 937-953, 1346-1363, 1346-1361, 1347-1363, 2162-2179, 2492-2508, 2696-2717, or 2683-2703 of SEQ ID NO: 1.

Certain embodiments provide compounds comprising a modified oligonucleotide consisting of 10 to 80, 12 to 50, 12 to 30, 15 to 30, 18 to 24, 19 to 22, or 20, linked nucleosides having a nucleobase sequence comprising a portion of at least 8, at least 9, at least 10, at least 11, at least 12, at least 13, at least 14, at least 15, at least 16, at least 17, at least 18, or at least 19, or more, contiguous nucleobases complementary to an equal length portion of nucleobases 10195-10294, 13553-13572, 13748-13767, 13455-13475, 13628-13657, 13735-13760, 13746-13905, 13836-13851, 13553-13568, 13563-13578, 13624-13639, 13686-13701, 13760-13775, 13763-13779, 13765-13780, 2580-2595, 6446-6461, 11099-11115, 11082-11099, 1974-1993, 4435-4456, 6035-6052, 6360-6385, 6445-6468, 6807-6824, 6789-6806, or 6596-6615 of SEQ ID NO: 2.

In certain embodiments, the modified oligonucleotide is a single-stranded oligonucleotide.

In certain embodiments, the nucleobase sequence of the modified oligonucleotide is at least 70%, at least 75%, at least 80%, at least 85%, at least 90%, at least 95%, or 100%, complementary to any of SEQ ID NOs: 23, 24, 25, 26, 27, 28, 29, 30, 31, 32, or 33-874.

In certain embodiments, at least one internucleoside linkage is a modified internucleoside linkage.

In certain embodiments, each internucleoside linkage is a phosphorothioate internucleoside linkage.

In certain embodiments, at least one nucleoside comprises a modified sugar.

In certain embodiments, at least one modified sugar is a bicyclic sugar.

In certain embodiments, at least one modified sugar is a cEt.

In certain embodiments, at least one modified sugar comprises a 2′-O-methoxyethyl.

In certain embodiments, at least one nucleoside comprises a modified nucleobase.

In certain embodiments, the modified nucleobase is a 5-methylcytosine. In certain embodiments, each cytosine residue comprises a 5-methylcytosine.

In certain embodiments, the modified oligonucleotide consists of 16 linked nucleosides.

In certain embodiments, the modified oligonucleotide consists of 17 linked nucleosides.

In certain embodiments, the modified oligonucleotide consists of 20 linked nucleosides.

Antisense Compounds Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound can be “antisense” to a target nucleic acid, meaning that is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to DMPK as described herein is 10 to 30 nucleotides in length. In other words, the antisense compounds are in some embodiments from 10 to 30 linked nucleobases. In other embodiments, the antisense compound comprises a modified oligonucleotide consisting of 8 to 80, 10 to 80, 12 to 30, 12 to 50, 15 to 30, 15 to 18, 15 to 17, 16 to 16, 18 to 24, 19 to 22, or 20 linked nucleobases. In certain such embodiments, the antisense compound comprises a modified oligonucleotide consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked nucleobases in length, or a range defined by any two of the above values. In certain embodiments, antisense compounds of any of these lengths contain at least 8, at least 9, at least 10, at least 11, at least 12, at least 13, at least 14, at least 15, at least 16, at least 17, at least 18, or at least 19, contiguous nucleobases of the nucleobase sequence of any of the exemplary antisense compounds described herein (e.g., at least 8 contiguous nucleobases of a nucleobase sequence recited in any one of SEQ ID NOs: 23, 24, 25, 26, 27, 28, 29, 30, 31, 32, or 33-874.

In certain embodiments, the antisense compound comprises a shortened or truncated modified oligonucleotide. The shortened or truncated modified oligonucleotide can have a single nucleoside deleted from the 5′ end (5′ truncation), or alternatively from the 3′ end (3′ truncation). A shortened or truncated oligonucleotide can have two nucleosides deleted from the 5′ end, or alternatively can have two subunits deleted from the 3′ end. Alternatively, the deleted nucleosides can be dispersed throughout the modified oligonucleotide, for example, in an antisense compound having one nucleoside deleted from the 5′ end and one nucleoside deleted from the 3′ end.

When a single additional nucleoside is present in a lengthened oligonucleotide, the additional nucleoside can be located at the 5′ or 3′ end of the oligonucleotide. When two or more additional nucleosides are present, the added nucleosides can be adjacent to each other, for example, in an oligonucleotide having two nucleosides added to the 5′ end (5′ addition), or alternatively to the 3′ end (3′ addition), of the oligonucleotide. Alternatively, the added nucleoside can be dispersed throughout the antisense compound, for example, in an oligonucleotide having one nucleoside added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Target Nucleic Acids, Target Regions and Nucleotide Sequences

Nucleotide sequences that encode DMPK include, without limitation, the following sequences as set forth in GenBank Accession No. NM_001081560.1 (incorporated herein as SEQ ID NO: 1), GenBank Accession No. NT_011109.15 truncated from nucleotides 18540696 to U.S. Pat. No. 18,555,106 (incorporated herein as SEQ ID NO: 2), GenBank Accession No. NT_039413.7 truncated from nucleotides 16666001 to 16681000 (incorporated herein as SEQ ID NO: 3), GenBank Accession No. NM_032418.1 (incorporated herein as SEQ ID NO: 4), GenBank Accession No. AI007148.1 (incorporated herein as SEQ ID NO: 5), GenBank Accession No. AI304033.1 (incorporated herein as SEQ ID NO: 6), GenBank Accession No. BC024150.1 (incorporated herein as SEQ ID NO: 7), GenBank Accession No. BC056615.1 (incorporated herein as SEQ ID NO: 8), GenBank Accession No. BC075715.1 (incorporated herein as SEQ ID NO: 9), GenBank Accession No. BU519245.1 (incorporated herein as SEQ ID NO: 10), GenBank Accession No. CB247909.1 (incorporated herein as SEQ ID NO: 11), GenBank Accession No. CX208906.1 (incorporated herein as SEQ ID NO: 12), GenBank Accession No. CX732022.1 (incorporated herein as SEQ ID NO: 13), GenBank Accession No. S60315.1 (incorporated herein as SEQ ID NO: 14), GenBank Accession No. S60316.1 (incorporated herein as SEQ ID NO: 15), GenBank Accession No. NM_001081562.1 (incorporated herein as SEQ ID NO: 16), and GenBank Accession No. NM_001100.3 (incorporated herein as SEQ ID NO: 17). It is understood that the sequence set forth in each SEQ ID NO in the Examples contained herein is independent of any modification to a sugar moiety, an internucleoside linkage, or a nucleobase. As such, antisense compounds defined by a SEQ ID NO can comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region can encompass a 3′ UTR, a 5′ UTR, an exon, an intron, an exon/intron junction, a coding region, a translation initiation region, translation termination region, or other defined nucleic acid region. The structurally defined regions for DMPK can be obtained by accession number from sequence databases such as NCBI and such information is incorporated herein by reference. In certain embodiments, a target region can encompass the sequence from a 5′ target site of one target segment within the target region to a 3′ target site of another target segment within the target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region can contain one or more target segments. Multiple target segments within a target region can be overlapping. Alternatively, they can be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments can be found within a 5′ UTR, a coding region, a 3′ UTR, an intron, an exon, or an exon/intron junction. Target segments containing a start codon or a stop codon are also suitable target segments. A suitable target segment can specifically exclude a certain structurally defined region such as the start codon or stop codon.

The determination of suitable target segments can include a comparison of the sequence of a target nucleic acid to other sequences throughout the genome. For example, the BLAST algorithm can be used to identify regions of similarity amongst different nucleic acids. This comparison can prevent the selection of antisense compound sequences that can hybridize in a non-specific manner to sequences other than a selected target nucleic acid (i.e., non-target or off-target sequences).

There can be variation in activity (e.g., as defined by percent reduction of target nucleic acid levels) of the antisense compounds within an active target region. In certain embodiments, reductions in DMPK mRNA levels are indicative of inhibition of DMPK protein expression. Reductions in levels of a DMPK protein are also indicative of inhibition of target mRNA expression. Further, phenotypic changes, such as a reducing myotonia or reducing spliceopathy, can be indicative of inhibition of DMPK mRNA and/or protein expression.

### Hybridization

In some embodiments, hybridization occurs between an antisense compound disclosed herein and a DMPK nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art (Sambrooke and Russell, Molecular Cloning: A Laboratory Manual, 3^{rd} Ed., 2001). In certain embodiments, the antisense compounds provided herein are specifically hybridizable with a DMPK nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a DMPK nucleic acid).

An antisense compound can hybridize over one or more segments of a DMPK nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a DMPK nucleic acid, a target region, target segment, or specified portion thereof. In certain embodiments, the antisense compounds are at least 70%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to a DMPK nucleic acid, a target region, target segment, or specified portion thereof, and contain at least 8, at least 9, at least 10, at least 11, at least 12, at least 13, at least 14, at least 15, at least 16, at least 17, at least 18, or at least 19, contiguous nucleobases of the nucleobase sequence of any of the exemplary antisense compounds described herein (e.g., at least 8 contiguous nucleobases of a nucleobase sequence recited in any one of SEQ ID NOs: 23, 24, 25, 26, 27, 28, 29, 30, 31, 32, or 33-874). Percent complementarity of an antisense compound with a target nucleic acid can be determined using routine methods, and is measured over the entirety of the antisense compound.

For example, an antisense compound in which 18 of 20 nucleobases of the antisense compound are complementary to a target region, and would therefore specifically hybridize, would represent 90 percent complementarity. In this example, the remaining noncomplementary nucleobases can be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, antisense compound can be fully complementary to a DMPK nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound can be fully complementary to the target sequence, depending on whether the remaining 10 nucleobases of the antisense compound are also complementary to the target sequence.

The location of a non-complementary nucleobase can be at the 5′ end or 3′ end of the antisense compound. Alternatively, the non-complementary nucleobase or nucleobases can be at an internal position of the antisense compound. When two or more non-complementary nucleobases are present, they can be either contiguous (i.e. linked) or non-contiguous. In one embodiment, a non-complementary nucleobase is located in the wing segment of a gapmer antisense oligonucleotide.

In certain embodiments, antisense compounds that are, or are up to 10, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a DMPK nucleic acid, or specified portion thereof.

In certain embodiments, antisense compounds that are, or are up to 10, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a DMPK nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 10 nucleobase portion of a target segment. In certain embodiments, the antisense compounds are complementary to at least a 15 nucleobase portion of a target segment. Also contemplated are antisense compounds that are complementary to at least an 8, at least a 9, at least a 10, at least an 11, at least a 12, at least a 13, at least a 14, at least a 15, at least a 16, at least a 17, at least an 18, at least a 19, at least a 20, or more nucleobase portion of a target segment, or a range defined by any two of these values.

### Identity

The antisense compounds provided herein can also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases can be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, at least 75%, at least 80%, at least 85%, at least 90%, at least 95%, at least 96%, at least 97%, at least 98%, at least 99% or 100% identical to one or more of the exemplary antisense compounds or SEQ ID NOs, or a portion thereof, disclosed herein.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides can also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a DMPK nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds of the invention can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(Rₗ)—(CH₂)₂—N(Rₘ)(Rₙ), where each Rₗ, Rₘ and Rₐ is, independently, H or substituted or unsubstituted C₁-C₁₀ alkyl.

Examples of bicyclic nucleic acids (BNAs) include without limitation nucleosides comprising a bridge between the 4′ and the 2′ ribosyl ring atoms. In certain embodiments, antisense compounds provided herein include one or more BNA nucleosides wherein the bridge comprises one of the formulas: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see PCT/US2008/068922 published as WO/2009/006478, published Jan. 8, 2009); 4′-CH₂—N(OCH₃)-2′ (and analogs thereof see PCT/US2008/064591 published as WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C(═CH₂)-2′ (and analogs thereof see PCT/US2008/066154 published as WO 2008/154401, published on Dec. 8, 2008).

Further bicyclic nucleosides have been reported in published literature (see for example: Srivastava et al., J. Am. Chem. Soc., 2007, 129(26) 8362-8379; Frieden et al., Nucleic Acids Research, 2003, 21, 6365-6372; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wahlestedt et al., Proc. Natl. Acad. Sci. U.S.A, 2000, 97, 5633-5638; Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; U.S. Pat. Nos. 7,399,845; 7,053,207; 7,034,133; 6,794,499; 6,770,748; 6,670,461; 6,525,191; 6,268,490; U.S. Patent Publication Nos.: US2008-0039618; US2007-0287831; US2004-0171570; U.S. patent applications, Ser. Nos. 12/129,154; 61/099,844; 61/097,787; 61/086,231; 61/056,564; 61/026,998; 61/026,995; 60/989,574; International applications WO 2007/134181; WO 2005/021570; WO 2004/106356; WO 94/14226; and PCT International Applications Nos.: PCT/US2008/068922; PCT/US2008/066154; and PCT/US2008/064591). Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic nucleosides comprise a bridge between the 4′ and the 2′ carbon atoms of the pentofuranosyl sugar moiety including without limitation, bridges comprising 1 or from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is, —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)-2′ and 4′-CH₂—N(R)—O-2′- wherein each R is, independently, H, a protecting group or C₁-C₁₂ alkyl.

In certain embodiments, bicyclic nucleosides are further defined by isomeric configuration. For example, a nucleoside comprising a 4′-(CH₂)—O-2′ bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include those having a 4′ to 2′ bridge wherein such bridges include without limitation, α-L-4′-(CH₂)—O-2′, β-D-4′-CH₂—O-2′, 4′-(CH₂)₂—O-2′, 4′-CH₂—O—N(R)-2′, 4′-CH₂—N(R)—O-2′, 4′-CH(CH₃)—O-2′, 4′-CH₂—S-2′, 4′-CH₂—N(R)-2′, 4′-CH₂—CH(CH₃)-2′, and 4′-(CH₂)₃-2′, wherein R is H, a protecting group or C₁-C₁₂ alkyl.

In certain embodiments, bicyclic nucleosides have the formula:

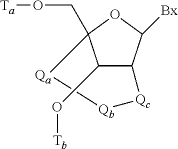

wherein:
Bx is a heterocyclic base moiety;
-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;
R_{c} is C₁-C₁₂ alkyl or an amino protecting group; and
Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides have the formula:

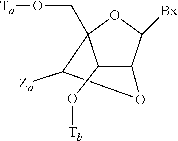

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thiol.

In one embodiment, each of the substituted groups, is, independently, mono or poly substituted with substituent groups independently selected from halogen, oxo, hydroxyl, OJ_{c}, NJ_{c}J_{d}, SJ_{c}, N₃, OC(═X)J_{c}, and NJ_{c}C(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleosides have the formula:

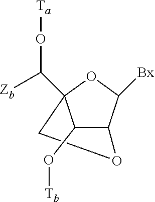

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleosides have the formula:

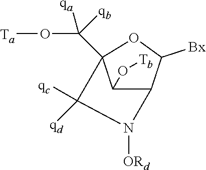

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleosides have the formula:

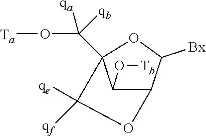

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;
or qₑ and q_{f} together are ═C(q_{g})(qₕ);
q_{g} and qₕ are each, independently, H, halogen, C₁-C₁₂ alkyl or substituted C₁-C₁₂ alkyl.

The synthesis and preparation of adenine, cytosine, guanine, 5-methyl-cytosine, thymine and uracil bicyclic nucleosides having a 4′-CH₂—O-2′ bridge, along with their oligomerization, and nucleic acid recognition properties have been described (Koshkin et al., Tetrahedron, 1998, 54, 3607-3630). The synthesis of bicyclic nucleosides has also been described in WO 98/39352 and WO 99/14226.

Analogs of various bicyclic nucleosides that have 4′ to 2′ bridging groups such as 4′-CH₂—O-2′ and 4′-CH₂—S-2′, have also been prepared (Kumar et al., Bioorg. Med. Chem. Lett., 1998, 8, 2219-2222). Preparation of oligodeoxyribonucleotide duplexes comprising bicyclic nucleosides for use as substrates for nucleic acid polymerases has also been described (Wengel et al., WO 99/14226). Furthermore, synthesis of 2′-amino-BNA, a novel conformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleosides have the formula:

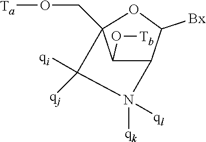

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
each qᵢ, qⱼ, qₖ and qᵢ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and
qᵢ and qⱼ or qᵢ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Frier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc. 2007, 129(26), 8362-8379).

In certain embodiments, bicyclic nucleosides include, but are not limited to, (A) α-L-methyleneoxy (4′-CH₂—O-2′) BNA, (B) β-D-methyleneoxy (4′-CH₂—O-2′) BNA, (C) ethyleneoxy (4′-(CH₂)₂—O-2′) BNA, (D) aminooxy (4′-CH₂—O—N(R)-2′) BNA, (E) oxyamino (4′-CH₂—N(R)—O-2′) BNA, (F) methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃-2′) BNA, and (K) vinyl BNA as depicted below.

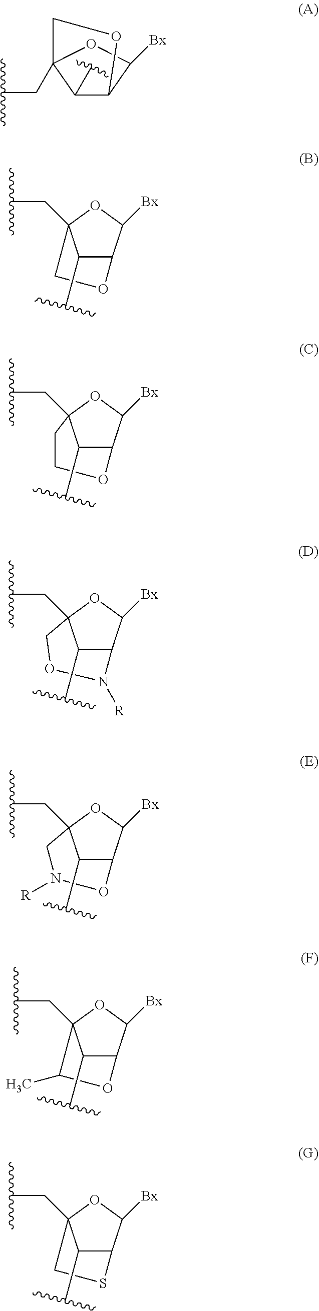

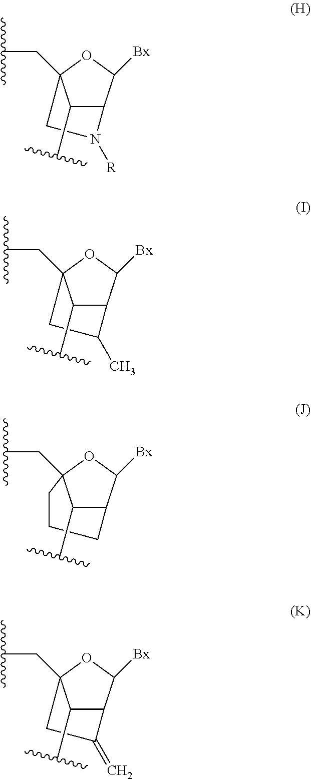

wherein Bx is the base moiety and R is, independently, H, a protecting group, C₁-C₆ alkyl or C₁-C₆ alkoxy.

In certain embodiments, nucleosides are modified by replacement of the ribosyl ring with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring such as one having one of the formula:

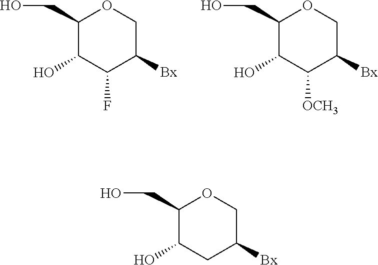

In certain embodiments, sugar surrogates are selected having the formula:

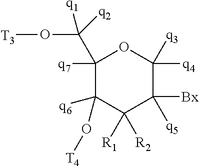

wherein:
Bx is a heterocyclic base moiety;
T₃ and T₄ are each, independently, an internucleoside linking group linking the tetrahydropyran nucleoside analog to the oligomeric compound or one of T₃ and T₄ is an internucleoside linking group linking the tetrahydropyran nucleoside analog to an oligomeric compound or oligonucleotide and the other of T₃ and T₄ is H, a hydroxyl protecting group, a linked conjugate group or a 5′ or 3-terminal group;
   q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and
one of R₁ and R₂ is hydrogen and the other is selected from halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, qᵢ, q₂, q₃, q₄, q₅, q₆ and q₇ are each H. In certain embodiments, at least one of qᵢ, q₂, q₃, q₄, q₅, q₆ and q₇ is other than H. In certain embodiments, at least one of qᵢ, q₂, q₃, q₄, q₅, q₆ and q₇ is methyl. In certain embodiments, THP nucleosides are provided wherein one of R₁ and R₂ is F. In certain embodiments, R₁ is fluoro and R₂ is H; R₁ is methoxy and R₂ is H, and R₁ is methoxyethoxy and R₂ is H.

Such sugar surrogates include, but are not limited to, what is referred to in the art as hexitol nucleic acid (HNA), altritol nucleic acid (ANA), and mannitol nucleic acid (MNA) (see Leumann, C. J., Bioorg. & Med. Chem., 2002, 10, 841-854).

In certain embodiments, sugar surrogates comprise rings having more than 5 atoms and more than one heteroatom. For example nucleosides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506).

As used here, the term “morpholino” means a sugar surrogate having the following structure:

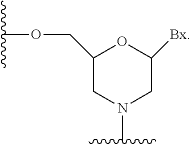

In certain embodiments, morpholinos may be modified, for example by adding or altering various substituent groups from the above morpholino structure. Such sugar surrogates are referred to herein as “modified morpholinos.”

In certain embodiments, antisense compounds comprise one or more modified cyclohexenyl nucleosides, which is a nucleoside having a six-membered cyclohexenyl in place of the pentofuranosyl residue in naturally occurring nucleosides. Modified cyclohexenyl nucleosides include, but are not limited to those described in the art (see for example commonly owned, published PCT Application WO 2010/036696, published on Apr. 10, 2010, Robeyns et al., J. Am. Chem. Soc., 2008, 130(6), 1979-1984; Horvith et al., Tetrahedron Letters, 2007, 48, 3621-3623; Nauwelaerts et al., J. Am. Chem. Soc., 2007, 129(30), 9340-9348; Gu et al., Nucleosides, Nucleotides & Nucleic Acids, 2005, 24(5-7), 993-998; Nauwelaerts et al., Nucleic Acids Research, 2005, 33(8), 2452-2463; Robeyns et al., Acta Crystallographica, Section F: Structural Biology and Crystallization Communications, 2005, F61(6), 585-586; Gu et al., Tetrahedron, 2004, 60(9), 2111-2123; Gu et al., Oligonucleotides, 2003, 13(6), 479-489; Wang et al., J. Org. Chem., 2003, 68, 4499-4505; Verbeure et al., Nucleic Acids Research, 2001, 29(24), 4941-4947; Wang et al., J Org. Chem., 2001, 66, 8478-82; Wang et al., Nucleosides, Nucleotides & Nucleic Acids, 2001, 20(4-7), 785-788; Wang et al., J. Am. Chem., 2000, 122, 8595-8602; Published PCT application, WO 06/047842; and Published PCT Application WO 01/049687; the text of each is incorporated by reference herein, in their entirety). Certain modified cyclohexenyl nucleosides have the formula:

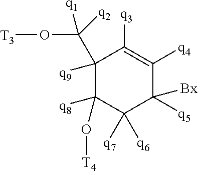

wherein:
Bx is a heterocyclic base moiety;
T₃ and T₄ are each, independently, an internucleoside linking group linking the cyclohexenyl nucleoside analog to an antisense compound or one of T₃ and T₄ is an internucleoside linking group linking the tetrahydropyran nucleoside analog to an antisense compound and the other of T₃ and T₄ is H, a hydroxyl protecting group, a linked conjugate group, or a 5′- or 3′-terminal group; and
   qᵢ, q₂, q₃, q₄, q₅, q₆, q₇, q₈ and q₉ are each, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or other sugar substituent group.

Many other bicyclic and tricyclic sugar surrogate ring systems are also known in the art that can be used to modify nucleosides for incorporation into antisense compounds (see for example review article: Leumann, Christian J., Bioorg. & Med. Chem., 2002, 10, 841-854). Such ring systems can undergo various additional substitutions to enhance activity.

Methods for the preparations of modified sugars are well known to those skilled in the art. Some representative U.S. patents that teach the preparation of such modified sugars include without limitation, U.S.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,670,633; 5,700,920; 5,792,847 and 6,600,032 and International Application PCT/US2005/019219, filed Jun. 2, 2005 and published as WO 2005/121371 on Dec. 22, 2005, and each of which is herein incorporated by reference in its entirety.

In nucleotides having modified sugar moieties, the nucleobase moieties (natural, modified or a combination thereof) are maintained for hybridization with an appropriate nucleic acid target.

In certain embodiments, antisense compounds targeted to a DMPK nucleic acid comprise one or more nucleotides having modified sugar moieties. In certain embodiments, the modified sugar moiety is 2′-MOE. In certain embodiments, the 2′-MOE modified nucleotides are arranged in a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties can also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and 0-6 substituted purines, including 2 aminopropyladenine, 5-propynyluracil and 5-propynylcytosine.

In certain embodiments, antisense compounds targeted to a DMPK nucleic acid comprise one or more modified nucleobases. In certain embodiments, gap-widened antisense oligonucleotides targeted to a DMPK nucleic acid comprise one or more modified nucleobases. In certain embodiments, the modified nucleobase is 5-methylcytosine. In certain embodiments, each cytosine is a 5-methylcytosine.

### Certain Antisense Compound Motifs

In certain embodiments, antisense compounds targeted to a DMPK nucleic acid have chemically modified subunits arranged in patterns, or motifs, to confer to the antisense compounds properties such as enhanced the inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound can optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer can in some embodiments include β-D-ribonucleosides, β-D-deoxyribonucleosides, 2′-modified nucleosides (such 2′-modified nucleosides can include 2′-MOE, and 2′-O—CH₃, among others), and bicyclic sugar modified nucleosides (such bicyclic sugar modified nucleosides can include those having a 4′-(CH₂)ₙ—O-2′ bridge, where n=1 or n=2). The wing-gap-wing motif is frequently described as “X—Y—Z”, where “X” represents the length of the 5′ wing region, “Y” represents the length of the gap region, and “Z” represents the length of the 3′ wing region. As used herein, a gapmer described as “X—Y—Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30 or more nucleotides. Thus, gapmers include, but are not limited to, for example 5-10-5, 4-8-4, 4-12-3, 4-12-4, 3-14-3, 2-13-5, 2-16-2, 1-18-1, 3-10-3, 2-10-2, 1-10-1, 2-8-2, 6-8-6, 5-8-5, 5-7-5, 1-8-1, or 2-6-2.

In certain embodiments, the antisense compound as a “wingmer” motif, having a wing-gap or gap-wing configuration, i.e. an X-Y or Y-Z configuration as described above for the gapmer configuration. Thus, wingmer configurations include, but are not limited to, for example 5-10, 8-4, 4-12, 12-4, 3-14, 16-2, 18-1, 10-3, 2-10, 1-10, 8-2, 2-13, or 5-13.

In certain embodiments, antisense compounds targeted to a DMPK nucleic acid possess a 5-10-5 gapmer motif. In certain embodiments, antisense compounds targeted to a DMPK nucleic acid possess a 5-7-5 gapmer motif. In certain embodiments, antisense compounds targeted to a DMPK nucleic acid possess a 3-10-3 gapmer motif. In certain embodiments, antisense compounds targeted to a DMPK nucleic acid possess a 4-8-4 gapmer motif.

In certain embodiments, an antisense compound targeted to a DMPK nucleic acid has a gap-widened motif.

In certain embodiments, antisense compounds of any of these gapmer or wingmer motifs contain at least 8, at least 9, at least 10, at least 11, at least 12, at least 13, at least 14, at least 15, at least 16, at least 17, at least 18, or at least 19, contiguous nucleobases of the nucleobase sequence of any of the exemplary antisense compounds described herein (e.g., at least 8 contiguous nucleobases of a nucleobase sequence recited in any one of SEQ ID NOs: 23, 24, 25, 26, 27, 28, 29, 30, 31, 32, or 33-874.

In certain embodiments, the present invention provides oligomeric compounds comprising oligonucleotides. In certain embodiments, such oligonucleotides comprise one or more chemical modification. In certain embodiments, chemically modified oligonucleotides comprise one or more modified sugars. In certain embodiments, chemically modified oligonucleotides comprise one or more modified nucleobases. In certain embodiments, chemically modified oligonucleotides comprise one or more modified internucleoside linkages. In certain embodiments, the chemically modifications (sugar modifications, nucleobase modifications, and/or linkage modifications) define a pattern or motif. In certain embodiments, the patterns of chemical modifications of sugar moieties, internucleoside linkages, and nucleobases are each independent of one another. Thus, an oligonucleotide may be described by its sugar modification motif, internucleoside linkage motif and/or nucleobase modification motif (as used herein, nucleobase modification motif describes the chemical modifications to the nucleobases independent of the sequence of nucleobases).

### Certain Sugar Motifs

In certain embodiments, oligonucleotides comprise one or more type of modified sugar moieties and/or naturally occurring sugar moieties arranged along an oligonucleotide or region thereof in a defined pattern or sugar modification motif. Such motifs may include any of the sugar modifications discussed herein and/or other known sugar modifications.

In certain embodiments, the oligonucleotides comprise or consist of a region having a gapmer sugar modification motif, which comprises two external regions or “wings” and an internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap. In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar modification motifs of the two wings are the same as one another (symmetric gapmer). In certain embodiments, the sugar modification motifs of the 5′-wing differs from the sugar modification motif of the 3′-wing (asymmetric gapmer).

### Certain 5′-Wings

In certain embodiments, the 5′-wing of a gapmer consists of 1 to 5 linked nucleosides. In certain embodiments, the 5′-wing of a gapmer consists of 2 to 5 linked nucleosides. In certain embodiments, the 5′-wing of a gapmer consists of 3 to 5 linked nucleosides. In certain embodiments, the 5′-wing of a gapmer consists of 4 or 5 linked nucleosides. In certain embodiments, the 5′-wing of a gapmer consists of 1 to 4 linked nucleosides. In certain embodiments, the 5′-wing of a gapmer consists of 1 to 3 linked nucleosides. In certain embodiments, the 5′-wing of a gapmer consists of 1 or 2 linked nucleosides. In certain embodiments, the 5′-wing of a gapmer consists of 2 to 4 linked nucleosides. In certain embodiments, the 5′-wing of a gapmer consists of 2 or 3 linked nucleosides. In certain embodiments, the 5′-wing of a gapmer consists of 3 or 4 linked nucleosides. In certain embodiments, the 5′-wing of a gapmer consists of 1 nucleoside. In certain embodiments, the 5′-wing of a gapmer consists of 2 linked nucleosides. In certain embodiments, the 5′-wing of a gapmer consists of 3linked nucleosides. In certain embodiments, the 5′-wing of a gapmer consists of 4 linked nucleosides. In certain embodiments, the 5′-wing of a gapmer consists of 5 linked nucleosides.

In certain embodiments, the 5′-wing of a gapmer comprises at least one bicyclic nucleoside. In certain embodiments, the 5′-wing of a gapmer comprises at least two bicyclic nucleosides. In certain embodiments, the 5′-wing of a gapmer comprises at least three bicyclic nucleosides. In certain embodiments, the 5′-wing of a gapmer comprises at least four bicyclic nucleosides. In certain embodiments, the 5′-wing of a gapmer comprises at least one constrained ethyl nucleoside. In certain embodiments, the 5′-wing of a gapmer comprises at least one LNA nucleoside. In certain embodiments, each nucleoside of the 5′-wing of a gapmer is a bicyclic nucleoside. In certain embodiments, each nucleoside of the 5′-wing of a gapmer is a constrained ethyl nucleoside. In certain embodiments, each nucleoside of the 5′-wing of a gapmer is a LNA nucleoside.

In certain embodiments, the 5′-wing of a gapmer comprises at least one non-bicyclic modified nucleoside. In certain embodiments, the 5′-wing of a gapmer comprises at least one 2′-substituted nucleoside. In certain embodiments, the 5′-wing of a gapmer comprises at least one 2′-MOE nucleoside. In certain embodiments, the 5′-wing of a gapmer comprises at least one 2′-OMe nucleoside. In certain embodiments, each nucleoside of the 5′-wing of a gapmer is a non-bicyclic modified nucleoside. In certain embodiments, each nucleoside of the 5′-wing of a gapmer is a 2′-substituted nucleoside. In certain embodiments, each nucleoside of the 5′-wing of a gapmer is a 2′-MOE nucleoside. In certain embodiments, each nucleoside of the 5′-wing of a gapmer is a 2′-OMe nucleoside.

In certain embodiments, the 5′-wing of a gapmer comprises at least one bicyclic nucleoside and at least one non-bicyclic modified nucleoside. In certain embodiments, the 5′-wing of a gapmer comprises at least one bicyclic nucleoside and at least one 2′-substituted nucleoside. In certain embodiments, the 5′-wing of a gapmer comprises at least one bicyclic nucleoside and at least one 2′-MOE nucleoside. In certain embodiments, the 5′-wing of a gapmer comprises at least one bicyclic nucleoside and at least one 2′-OMe nucleoside. In certain embodiments, the 5′-wing of a gapmer comprises at least one bicyclic nucleoside and at least one 2′-deoxynucleoside.

In certain embodiments, the 5′-wing of a gapmer comprises at least one constrained ethyl nucleoside and at least one non-bicyclic modified nucleoside. In certain embodiments, the 5′-wing of a gapmer comprises at least one constrained ethyl nucleoside and at least one 2′-substituted nucleoside. In certain embodiments, the 5′-wing of a gapmer comprises at least one constrained ethyl nucleoside and at least one 2′-MOE nucleoside. In certain embodiments, the 5′-wing of a gapmer comprises at least one constrained ethyl nucleoside and at least one 2′-OMe nucleoside. In certain embodiments, the 5′-wing of a gapmer comprises at least one constrained ethyl nucleoside and at least one 2′-deoxynucleoside.

In certain embodiments, the 5′-wing of a gapmer comprises at least one LNA nucleoside and at least one non-bicyclic modified nucleoside. In certain embodiments, the 5′-wing of a gapmer comprises at least one LNA nucleoside and at least one 2′-substituted nucleoside. In certain embodiments, the 5′-wing of a gapmer comprises at least one LNA nucleoside and at least one 2′-MOE nucleoside. In certain embodiments, the 5′-wing of a gapmer comprises at least one LNA nucleoside and at least one 2′-OMe nucleoside. In certain embodiments, the 5′-wing of a gapmer comprises at least one LNA nucleoside and at least one 2′-deoxynucleoside.

In certain embodiments, the 5′-wing of a gapmer comprises three constrained ethyl nucleosides. In certain embodiments, the 5′-wing of a gapmer comprises two bicyclic nucleosides and two non bicyclic modified nucleosides. In certain embodiments, the 5′-wing of a gapmer comprises two constrained ethyl nucleosides and two 2′-MOE nucleosides. In certain embodiments, the 5′-wing of a gapmer comprises two bicyclic nucleosides and two non bicyclic modified nucleosides. In certain embodiments, the 5′-wing of a gapmer comprises two constrained ethyl nucleosides and two 2′-MOE nucleosides. In certain embodiments, the 5′-wing of a gapmer comprises two constrained ethyl nucleosides and three 2′-MOE nucleosides.

In certain embodiments, the 5′-wing of a gapmer comprises three LNA nucleosides. In certain embodiments, the 5′-wing of a gapmer comprises two LNA nucleosides and two non bicyclic modified nucleosides. In certain embodiments, the 5′-wing of a gapmer comprises two LNA nucleosides and two 2′-MOE nucleosides. In certain embodiments, the 5′-wing of a gapmer comprises two LNA and two non bicyclic modified nucleosides. In certain embodiments, the 5′-wing of a gapmer comprises two LNA nucleosides and two 2′-MOE nucleosides. In certain embodiments, the 5′-wing of a gapmer comprises two LNA nucleosides and three 2′-MOE nucleosides.

In certain embodiments, the 5′-wing of a gapmer comprises three constrained ethyl nucleosides. In certain embodiments, the 5′-wing of a gapmer comprises two bicyclic nucleosides and two non bicyclic modified nucleosides. In certain embodiments, the 5′-wing of a gapmer comprises two constrained ethyl nucleosides and two 2′-OMe nucleosides. In certain embodiments, the 5′-wing of a gapmer comprises two bicyclic nucleosides and two non bicyclic modified nucleosides. In certain embodiments, the 5′-wing of a gapmer comprises two constrained ethyl nucleosides and two 2′-OMe nucleosides. In certain embodiments, the 5′-wing of a gapmer comprises two constrained ethyl nucleosides and three 2′-OMe nucleosides.

In certain embodiments, the 5′-wing of a gapmer comprises three LNA nucleosides. In certain embodiments, the 5′-wing of a gapmer comprises two LNA nucleosides and two non bicyclic modified nucleosides. In certain embodiments, the 5′-wing of a gapmer comprises two LNA nucleosides and two 2′-OMe nucleosides. In certain embodiments, the 5′-wing of a gapmer comprises two LNA and two non bicyclic modified nucleosides. In certain embodiments, the 5′-wing of a gapmer comprises two LNA nucleosides and two 2′-OMe nucleosides. In certain embodiments, the 5′-wing of a gapmer comprises two LNA nucleosides and three 2′-OMe nucleosides.

In certain embodiments, the 5′-wing of a gapmer has an AABB motif, wherein each A is selected from among a 2′-MOE nucleoside and a 2′OMe nucleoside. In certain embodiments, the 5′-wing of a gapmer has an AABB motif, wherein each B is selected from among a cEt, LNA, α-L-LNA, ENA and 2′-thio LNA nucleoside. In certain embodiments, the 5′-wing of a gapmer has an AABB motif, wherein each A represents a 2′-MOE nucleoside and each B represents a constrained ethyl nucleoside.

In certain embodiments, the 5′-wing of a gapmer has an AAABB motif, wherein each A is selected from among a 2′-MOE nucleoside and a 2′OMe nucleoside. In certain embodiments, the 5′-wing of a gapmer has an AABB motif, wherein each B is selected from among a cEt, LNA, α-L-LNA, ENA and 2′-thio LNA nucleoside. In certain embodiments, the 5′-wing of a gapmer has an AABB motif, wherein each A represents a 2′-MOE nucleoside and each B represents a constrained ethyl nucleoside.

### Certain 3′-Wings

In certain embodiments, the 3′-wing of a gapmer consists of 1 to 5 linked nucleosides. In certain embodiments, the 3′-wing of a gapmer consists of 2 to 5 linked nucleosides. In certain embodiments, the 3′-wing of a gapmer consists of 3 to 5 linked nucleosides. In certain embodiments, the 3′-wing of a gapmer consists of 4 or 5 linked nucleosides. In certain embodiments, the 3′-wing of a gapmer consists of 1 to 4 linked nucleosides. In certain embodiments, the 3′-wing of a gapmer consists of 1 to 3 linked nucleosides. In certain embodiments, the 3′-wing of a gapmer consists of 1 or 2 linked nucleosides. In certain embodiments, the 3′-wing of a gapmer consists of 2 to 4 linked nucleosides. In certain embodiments, the 3′-wing of a gapmer consists of 2 or 3 linked nucleosides. In certain embodiments, the 3′-wing of a gapmer consists of 3 or 4 linked nucleosides. In certain embodiments, the 3′-wing of a gapmer consists of 1 nucleoside. In certain embodiments, the 3′-wing of a gapmer consists of 2 linked nucleosides. In certain embodiments, the 3′-wing of a gapmer consists of 3linked nucleosides. In certain embodiments, the 3′-wing of a gapmer consists of 4 linked nucleosides. In certain embodiments, the 3′-wing of a gapmer consists of 5 linked nucleosides.

In certain embodiments, the 3′-wing of a gapmer comprises at least one bicyclic nucleoside. In certain embodiments, the 3′-wing of a gapmer comprises at least one constrained ethyl nucleoside. In certain embodiments, the 3′-wing of a gapmer comprises at least one LNA nucleoside. In certain embodiments, each nucleoside of the 3′-wing of a gapmer is a bicyclic nucleoside. In certain embodiments, each nucleoside of the 3′-wing of a gapmer is a constrained ethyl nucleoside. In certain embodiments, each nucleoside of the 3′-wing of a gapmer is a LNA nucleoside.

In certain embodiments, the 3′-wing of a gapmer comprises at least one non-bicyclic modified nucleoside. In certain embodiments, the 3′-wing of a gapmer comprises at least two non-bicyclic modified nucleosides. In certain embodiments, the 3′-wing of a gapmer comprises at least three non-bicyclic modified nucleosides. In certain embodiments, the 3′-wing of a gapmer comprises at least four non-bicyclic modified nucleosides. In certain embodiments, the 3′-wing of a gapmer comprises at least one 2′-substituted nucleoside. In certain embodiments, the 3′-wing of a gapmer comprises at least one 2′-MOE nucleoside. In certain embodiments, the 3′-wing of a gapmer comprises at least one 2′-OMe nucleoside. In certain embodiments, each nucleoside of the 3′-wing of a gapmer is a non-bicyclic modified nucleoside. In certain embodiments, each nucleoside of the 3′-wing of a gapmer is a 2′-substituted nucleoside. In certain embodiments, each nucleoside of the 3′-wing of a gapmer is a 2′-MOE nucleoside. In certain embodiments, each nucleoside of the 3′-wing of a gapmer is a 2′-OMe nucleoside.

In certain embodiments, the 3′-wing of a gapmer comprises at least one bicyclic nucleoside and at least one non-bicyclic modified nucleoside. In certain embodiments, the 3′-wing of a gapmer comprises at least one bicyclic nucleoside and at least one 2′-substituted nucleoside. In certain embodiments, the 3′-wing of a gapmer comprises at least one bicyclic nucleoside and at least one 2′-MOE nucleoside. In certain embodiments, the 3′-wing of a gapmer comprises at least one bicyclic nucleoside and at least one 2′-OMe nucleoside. In certain embodiments, the 3′-wing of a gapmer comprises at least one bicyclic nucleoside and at least one 2′-deoxynucleoside.

In certain embodiments, the 3′-wing of a gapmer comprises at least one constrained ethyl nucleoside and at least one non-bicyclic modified nucleoside. In certain embodiments, the 3′-wing of a gapmer comprises at least one constrained ethyl nucleoside and at least one 2′-substituted nucleoside. In certain embodiments, the 3′-wing of a gapmer comprises at least one constrained ethyl nucleoside and at least one 2′-MOE nucleoside. In certain embodiments, the 3′-wing of a gapmer comprises at least one constrained ethyl nucleoside and at least one 2′-OMe nucleoside. In certain embodiments, the 3′-wing of a gapmer comprises at least one constrained ethyl nucleoside and at least one 2′-deoxynucleoside.

In certain embodiments, the 3′-wing of a gapmer comprises at least one LNA nucleoside and at least one non-bicyclic modified nucleoside. In certain embodiments, the 3′-wing of a gapmer comprises at least one LNA nucleoside and at least one 2′-substituted nucleoside. In certain embodiments, the 3′-wing of a gapmer comprises at least one LNA nucleoside and at least one 2′-MOE nucleoside. In certain embodiments, the 3′-wing of a gapmer comprises at least one LNA nucleoside and at least one 2′-OMe nucleoside. In certain embodiments, the 3′-wing of a gapmer comprises at least one LNA nucleoside and at least one 2′-deoxynucleoside.

In certain embodiments, the 3′-wing of a gapmer comprises three constrained ethyl nucleosides. In certain embodiments, the 3′-wing of a gapmer comprises two bicyclic nucleosides and two non bicyclic modified nucleosides. In certain embodiments, the 3′-wing of a gapmer comprises two constrained ethyl nucleosides and two 2′-MOE nucleosides. In certain embodiments, the 3′-wing of a gapmer comprises two bicyclic nucleosides and two non bicyclic modified nucleosides. In certain embodiments, the 3′-wing of a gapmer comprises two constrained ethyl nucleosides and two 2′-MOE nucleosides. In certain embodiments, the 5′-wing of a gapmer comprises two constrained ethyl nucleosides and three 2′-MOE nucleosides.

In certain embodiments, the 3′-wing of a gapmer comprises three LNA nucleosides. In certain embodiments, the 3′-wing of a gapmer comprises two LNA nucleosides and two non bicyclic modified nucleosides. In certain embodiments, the 3′-wing of a gapmer comprises two LNA nucleosides and two 2′-MOE nucleosides. In certain embodiments, the 3′-wing of a gapmer comprises two LNA and two non bicyclic modified nucleosides. In certain embodiments, the 3′-wing of a gapmer comprises two LNA nucleosides and two 2′-MOE nucleosides. In certain embodiments, the 3′-wing of a gapmer comprises two LNA nucleosides and three 2′-MOE nucleosides.

In certain embodiments, the 3′-wing of a gapmer comprises three constrained ethyl nucleosides. In certain embodiments, the 3′-wing of a gapmer comprises two bicyclic nucleosides and two non bicyclic modified nucleosides. In certain embodiments, the 3′-wing of a gapmer comprises two constrained ethyl nucleosides and two 2′-OMe nucleosides. In certain embodiments, the 3′-wing of a gapmer comprises two bicyclic nucleosides and two non bicyclic modified nucleosides. In certain embodiments, the 3′-wing of a gapmer comprises two constrained ethyl nucleosides and two 2′-OMe nucleosides. In certain embodiments, the 3′-wing of a gapmer comprises two constrained ethyl nucleosides and three 2′-OMe nucleosides.

In certain embodiments, the 3′-wing of a gapmer comprises three LNA nucleosides. In certain embodiments, the 3′-wing of a gapmer comprises two LNA nucleosides and two non bicyclic modified nucleosides. In certain embodiments, the 3′-wing of a gapmer comprises two LNA nucleosides and two 2′-OMe nucleosides. In certain embodiments, the 3′-wing of a gapmer comprises two LNA and two non bicyclic modified nucleosides. In certain embodiments, the 3′-wing of a gapmer comprises two LNA nucleosides and two 2′-OMe nucleosides. In certain embodiments, the 3′-wing of a gapmer comprises two LNA nucleosides and three 2′-OMe nucleosides.

In certain embodiments, the 3′-wing of a gapmer has a BBAA motif, wherein each A is selected from among a 2′-MOE nucleoside and a 2′OMe nucleoside. In certain embodiments, the 3′-wing of a gapmer has an BBAA motif, wherein each B is selected from among a cEt, LNA, α-L-LNA, ENA and 2′-thio LNA nucleoside. In certain embodiments, the 3′-wing of a gapmer has a BBAA motif, wherein each A represents a 2′-MOE nucleoside and each B represents a constrained ethyl nucleoside.

In certain embodiments, the 3′-wing of a gapmer has a BBAAA motif, wherein each A is selected from among a 2′-MOE nucleoside and a 2′OMe nucleoside. In certain embodiments, the 3′-wing of a gapmer has a BBAA motif, wherein each B is selected from among a cEt, LNA, α-L-LNA, ENA and 2′-thio LNA nucleoside. In certain embodiments, the 3′-wing of a gapmer has a BBAA motif, wherein each A represents a 2′-MOE nucleoside and each B represents a constrained ethyl nucleoside.

### Compositions and Methods for Formulating Pharmaceutical Compositions

Antisense oligonucleotides can be admixed with pharmaceutically acceptable active or inert substance for the preparation of pharmaceutical compositions or formulations. Compositions and methods for the formulation of pharmaceutical compositions are dependent upon a number of criteria, including, but not limited to, route of administration, extent of disease, or dose to be administered.

Antisense compound targeted to a DMPK nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a DMPK nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds can be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of DMPK nucleic acids can be tested in vitro in a variety of cell types. Cell types used for such analyses are available from commercial vendors (e.g. American Type Culture Collection, Manassas, VA; Zen-Bio, Inc., Research Triangle Park, NC; Clonetics Corporation, Walkersville, MD) and cells are cultured according to the vendor's instructions using commercially available reagents (e.g. Invitrogen Life Technologies, Carlsbad, CA). Illustrative cell types include, but are not limited to, HepG2 cells, Hep3B cells, primary hepatocytes, A549 cells, GM04281 fibroblasts and LLC-MK2 cells.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluence in culture.

One reagent commonly used to introduce antisense oligonucleotides into cultured cells includes the cationic lipid transfection reagent LIPOFECTIN® (Invitrogen, Carlsbad, CA). Antisense oligonucleotides are mixed with LIPOFECTIN® in OPTI-MEM® 1 (Invitrogen, Carlsbad, CA) to achieve the desired final concentration of antisense oligonucleotide and a LIPOFECTIN® concentration that typically ranges 2 to 12 μg/mL per 100 nM antisense oligonucleotide.

Another reagent used to introduce antisense oligonucleotides into cultured cells includes LIPOFECTAMINE 2000® (Invitrogen, Carlsbad, CA). Antisense oligonucleotide is mixed with LIPOFECTAMINE 2000® in OPTI-MEM® 1 reduced serum medium (Invitrogen, Carlsbad, CA) to achieve the desired concentration of antisense oligonucleotide and a LIPOFECTAMINE® concentration that typically ranges 2 to 12 μg/mL per 100 nM antisense oligonucleotide.

Another reagent used to introduce antisense oligonucleotides into cultured cells includes Cytofectin® (Invitrogen, Carlsbad, CA). Antisense oligonucleotide is mixed with Cytofectin® in OPTI-MEM® 1 reduced serum medium (Invitrogen, Carlsbad, CA) to achieve the desired concentration of antisense oligonucleotide and a Cytofectin® concentration that typically ranges 2 to 12 μg/mL per 100 nM antisense oligonucleotide.

Another technique used to introduce antisense oligonucleotides into cultured cells includes electroporation.

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE2000®, Lipofectin or Cytofectin. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL® Reagent (Invitrogen, Carlsbad, CA) according to the manufacturer's recommended protocols.

### Analysis of Inhibition of Target Levels or Expression

Inhibition of levels or expression of a DMPK nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, CA and used according to manufacturer's instructions.

### Quantitative Real-Time PCR Analysis of Target RNA Levels

Quantitation of target RNA levels can be accomplished by quantitative real-time PCR using the ABI PRISM® 7600, 7700, or 7900 Sequence Detection System (PE-Applied Biosystems, Foster City, CA) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, CA). RT, real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN® (Invitrogen, Inc. Carlsbad, CA). Cyclophilin A expression is quantified by real time PCR, by being run simultaneously with the target, multiplexing, or separately. Total RNA is quantified using RIBOGREEN® RNA quantification reagent (Invitrogen, Inc. Eugene, OR). Methods of RNA quantification by RIBOGREEN® are taught in Jones, L. J., et al, (Analytical Biochemistry, 1998, 265, 368-374). A CYTOFLUOR® 4000 instrument (PE Applied Biosystems) is used to measure RIBOGREEN® fluorescence.

Probes and primers are designed to hybridize to a DMPK nucleic acid. Methods for designing real-time PCR probes and primers are well known in the art, and can include the use of software such as PRIMER EXPRESS® Software (Applied Biosystems, Foster City, CA).

### Analysis of Protein Levels

Antisense inhibition of DMPK nucleic acids can be assessed by measuring DMPK protein levels. Protein levels of DMPK can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, MI), or can be prepared via conventional monoclonal or polyclonal antibody generation methods well known in the art.

### In Vivo Testing of Antisense Compounds

Antisense compounds, for example, antisense oligonucleotides, are tested in animals to assess their ability to inhibit expression of DMPK and produce phenotypic changes. Testing can be performed in normal animals, or in experimental disease models, for example, the HSA^{LR} mouse model of myotonic dystrophy (DM1).

The HSA^{LR} mouse model is an established model for DM1 (Mankodi, A. et al. Science. 289: 1769, 2000). The mice carry a human skeletal actin (hACTA1) transgene with 220 CTG repeats inserted in the 3′ UTR of the gene. The hACTA1-CUG^{exp} transcript accumulates in nuclear foci in skeletal muscles and results in myotonia similar to that in human DM1 (Mankodi, A. et al. Mol. Cell 10: 35, 2002; Lin, X. et al. Hum. Mol. Genet. 15: 2087, 2006). Hence, it is expected that amelioration of DM1 symptoms in the HSA^{LR} mouse by antisense inhibition of the hACTA1 transgene would predict amelioration of similar symptoms in human patients by antisense inhibition of the DMPK transcript.

Expression of CUG^{exp} RNA in mice causes extensive remodeling of the muscle transcriptome, much of which is reproduced by ablation of MBNL1. Hence, it is expected that normalization of the transcriptome in HSA^{LR} mice would predict normalization of the human transcriptome in DM1 patients by antisense inhibition of the DMPK transcript.

For administration to animals, antisense oligonucleotides are formulated in a pharmaceutically acceptable diluent, such as phosphate-buffered saline. Administration includes parenteral routes of administration. Following a period of treatment with antisense oligonucleotides, RNA is isolated from tissue and changes in DMPK nucleic acid expression are measured. Changes in DMPK protein levels are also measured.

### Splicing

Myotonic dystrophy (DM1) is caused by CTG repeat expansions in the 3′ untranslated region of the DMPK gene (Brook, J. D. et al. Cell. 68: 799, 1992). This mutation leads to RNA dominance, a process in which expression of RNA containing an expanded CUG repeat (CUGexp) induces cell dysfunction (Osborne R J and Thornton C A., Human Molecular Genetics., 2006, 15(2): R162-R169). Such CUGexp are retained in the nuclear foci of skeletal muscles (Davis, B. M. et al. Proc. Natl. Acad. Sci. U.S.A. 94:7388, 1997). The accumulation of CUGexp in the nuclear foci leads to the sequestration of poly(CUG)-binding proteins, such as, Muscleblind-like 1 (MBLN1) (Miller, J. W. et al. EMBO J. 19: 4439, 2000). MBLN1 is a splicing factor and regulates the splicing of genes such as Sercal, CIC-1, Titin, and Zasp. Therefore, sequestration of MBLN1 by CUGexp triggers misregulated alternative splicing of the exons of genes that MBLN1 normally controls (Lin, X. et al. Hum. Mol. Genet. 15: 2087, 2006). Correction of alternative splicing in an animal displaying such disregulation, such as, for example, in a DM1 patient and the HSALR mouse model, is a useful indicator for the efficacy of a treatment, including treatment with an antisense oligonucleotide.

### Certain Antisense Mechanisms

Myotonic dystrophy (DM1) is caused by CTG repeat expansions in the 3′ untranslated region of the DMPK gene. In certain embodiments, expansions in the 3′ untranslated region of the DMPK gene results in the transcription of RNA containing an expanded CUG repeat, and RNA containing an expanded CUG repeat (CUGexp) is retained in the nuclear foci of skeletal muscles. In certain instances, the cellular machinery responsible for exporting mRNA from the nucleus into the cytoplasm does not export RNA containing an expanded CUG repeat from the nucleus or does so less efficiently. In certain embodiments, cells do not export DMPK CUGexp mRNA from the nucleus or such export is reduced. Accordingly, in certain embodiments, DMPK CUGexp mRNA accumulates in the nucleus. In certain embodiments, more copies of DMPK CUGexp mRNA are present in the nucleus of a cell than are copies of wild-type DMPK mRNA, which is exported normally. In such embodiments, antisense compounds that reduce target in the nucleus will preferentially reduce mutant DMPK CUGexp mRNA relative to wild type DMPK mRNA, due to their relative abundances in the nucleus, even if the antisense compound does not otherwise distinguish between mutant and wild type. Since RNase H dependent antisense compounds are active in the nucleus, such compounds are particularly well suited for such use.

In certain instances, wild-type DMPK pre-mRNA and mutant CUGexp DMPK pre-mRNA are expected to be processed into mRNA at similar rate. Accordingly, approximately the same amount of wild-type DMPK pre-mRNA and mutant CUGexp DMPK pre-mRNA are expected to be present in the nucleus of a cell. However, after processing, wild type DMPK mRNA is exported from the nucleus relatively quickly, and mutant CUGexp DMPK mRNA is exported slowly or not at all. In certain such embodiments, mutant CUGexp DMPK mRNA accumulates in the nucleus in greater amounts than wild-type DMPK mRNA. In certain such embodiments, an antisense oligonucleotide targeted to the mRNA, will preferentially reduce the expression of the mutant CUGexp DMPK mRNA compared to the wild-type DMPK mRNA because more copies of the mutant CUGexp DMPK mRNA are present in the nucleus of the cell. In certain embodiments, antisense compounds targeted to pre-mRNA and not mRNA (e.g., targeting an intron) are not expected to preferentially reduce mutant DMPK relative to wild type, because the nuclear abundance of the two pre-mRNAs is likely to be similar. In certain embodiments, antisense compounds described herein are not targeted to introns of DMPK pre-mRNA. In certain embodiments, antisense compounds described herein are targeted to exons or exon-exon junctions present in DMPK mRNA. In certain embodiments, use of an antisense oligonucleotide to target the mRNA is therefore preferred because an antisense oligonucleotide having one or more features described herein (i) has activity in the nucleus of a cell and (2) will preferentially reduce mutant CUGexp DMPK mRNA compared to wild-type DMPK mRNA.

### Certain Biomarkers

DM1 severity in mouse models is determined, at least in part, by the level of CUG^{exp} transcript accumulation in the nucleus or nuclear foci. A useful physiological marker for DM1 severity is the development of high-frequency runs of involuntary action potentials (myotonia).

### Certain Indications

In certain embodiments, provided herein are methods of treating an individual comprising administering one or more pharmaceutical compositions as described herein. In certain embodiments, the individual has type 1 myotonic dystrophy (DM1).

Accordingly, provided herein are methods for ameliorating a symptom associated with type 1 myotonic dystrophy in a subject in need thereof. In certain embodiments, provided is a method for reducing the rate of onset of a symptom associated with type 1 myotonic dystrophy. In certain embodiments, provided is a method for reducing the severity of a symptom associated with type 1 myotonic dystrophy. In certain embodiments, symptoms associated with DM1 include muscle stiffness, myotonia, disabling distal weakness, weakness in face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts. In children, the symptoms may also be developmental delays, learning problems, language and speech issues, and personality development issues.

In certain embodiments, the methods comprise administering to an individual in need thereof a therapeutically effective amount of a compound targeted to a DMPK nucleic acid.

In certain embodiments, administration of an antisense compound targeted to a DMPK nucleic acid results in reduction of DMPK expression by at least about 15%, by at least about 20%, by at least about 25%, by at least about 30%, by at least about 35%, by at least about 40%, by at least about 45%, by at least about 50%, by at least about 55%, by at least about 60%, by least about 65%, by least about 70%, by least about 75%, by least about 80%, by at least about 85%, by at least about 90%, by at least about 95% or by at least about 99%, or a range defined by any two of these values.

In certain embodiments, pharmaceutical compositions comprising an antisense compound targeted to DMPK are used for the preparation of a medicament for treating a patient suffering or susceptible to type 1 myotonic dystrophy.

In certain embodiments, the methods described herein include administering a compound comprising a modified oligonucleotide having a contiguous nucleobases portion as described herein of a sequence recited in SEQ ID NOs: 23, 24, 25, 26, 27, 28, 29, 30, 31, 32, or 33-874.

### Administration

In certain embodiments, the compounds and compositions as described herein are administered parenterally.

In certain embodiments, parenteral administration is by infusion. Infusion can be chronic or continuous or short or intermittent. In certain embodiments, infused pharmaceutical agents are delivered with a pump. In certain embodiments, parenteral administration is by injection (e.g., bolus injection). The injection can be delivered with a syringe.

Parenteral administration includes subcutaneous administration, intravenous administration, intramuscular administration, intraarterial administration, intraperitoneal administration, or intracranial administration, e.g., intrathecal or intracerebroventricular administration. Administration can be continuous, or chronic, or short, or intermittent.

In certain embodiments, the administering is subcutaneous, intravenous, intracerebral, intracerebroventricular, intrathecal or another administration that results in a systemic effect of the oligonucleotide (systemic administration is characterized by a systemic effect, i.e., an effect in more than one tissue) or delivery to the CNS or to the CSF.

The duration of action as measured by inhibition of alpha 1 actin and reduction of myotonia in the HSA^{LR} mouse model of DM1 is prolonged in muscle tissue including quadriceps, gastrocnemius, and the tibialis anterior (see Examples, below). Subcutaneous injections of antisense oligonucleotide for 4 weeks results in inhibition of alpha 1 actin by at least 70% in quadriceps, gastrocnemius, and the tibialis anterior in HSA^{LR} mice for at least 11 weeks (77 days) after termination of dosing. Subcutaneous injections of antisense oligonucleotide for 4 weeks results in elimination of myotonia in quadriceps, gastrocnemius, and the tibialis anterior in HSA^{LR} mice for at least 11 weeks (77 days) after termination of dosing.

In certain embodiments, delivery of a compound of composition, as described herein, results in at least 70% down-regulation of a target mRNA and/or target protein for at least 77 days. In certain embodiments, delivery of a compound or composition, as described herein, results in 50%, 55%, 60%, 65%, 70%, 75%, 80%, 85%, 90%, 95%, or 100% down-regulation of a target mRNA and/or target protein for at least 30 days, at least 35 days, at least 40 days, at least 45 days, at least 50 days, at least 55 days, at least 60 days, at least 65 days, at least 70 days, at least 75 days, at least 76 days, at least 77 days, at least 78 days, at least 79 days, at least 80 days, at least 85 days, at least 90 days, at least 95 days, at least 100 days, at least 105 days, at least 110 days, at least 115 days, at least 120 days, at least 1 year.

In certain embodiments, an antisense oligonucleotide is delivered by injection or infusion once every 77 days. In certain embodiments, an antisense oligonucleotide is delivered by injection or infusion once every month, every two months, every three months, every 6 months, twice a year or once a year.

### Certain Combination Therapies

In certain embodiments, a first agent comprising the modified oligonucleotide of the invention is co-administered with one or more secondary agents. In certain embodiments, such second agents are designed to treat the same type 1 myotonic dystrophy as the first agent described herein. In certain embodiments, such second agents are designed to treat a different disease, disorder, or condition as the first agent described herein. In certain embodiments, such second agents are designed to treat an undesired side effect of one or more pharmaceutical compositions as described herein. In certain embodiments, second agents are co-administered with the first agent to treat an undesired effect of the first agent. In certain embodiments, second agents are co-administered with the first agent to produce a combinational effect. In certain embodiments, second agents are co-administered with the first agent to produce a synergistic effect.

In certain embodiments, a first agent and one or more second agents are administered at the same time. In certain embodiments, the first agent and one or more second agents are administered at different times. In certain embodiments, the first agent and one or more second agents are prepared together in a single pharmaceutical formulation. In certain embodiments, the first agent and one or more second agents are prepared separately.

### Certain Comparator Compounds

In certain embodiments, the compounds disclosed herein benefit from one or more improved in vitro and/or in vivo properties relative to an appropriate comparator compound.

In certain embodiments, ISIS 445569, a 5-10-5 MOE gapmer, having a sequence of (from 5′ to 3′) CGGAGCGGTTGTGAACTGGC (incorporated herein as SEQ ID NO: 24), wherein each internucleoside linkage is a phosphorothioate linkage, each cytosine is a 5-methylcytosine, and each of nucleosides 1-5 and 16-20 comprise a 2′-O-methoxyethyl moiety, which was previously described in WO 2012/012443, incorporated herein by reference, is a comparator compound.

ISIS 445569 is an appropriate representative comparator compound because ISIS 445569 demonstrates statistically significant reduction of human DMPK in vitro as measured using a plurality of primer probe sets (see e.g. Example 1 and Example 2 of WO 2012/012443). Additionally, ISIS 445569 demonstrates statistically significant dose-dependent inhibition of human DMPK in vitro in both human skeletal muscle cells and DM1 fibroblasts (see e.g. Example 4 and Example 5 of WO 2012/012443 and Example 28 of WO 2012/012467). ISIS 445569 also reduces human DMPK transcript expression in transgenic mice (Examples 23 and 24 of WO 2012/012443 and Examples 29 and 30 of WO 2012/012467). ISIS 445569 was a preferred human DMPK antisense compound in WO 2012/012443 and WO 2012/012467.

### Certain Compounds

In certain embodiments, the compounds disclosed herein benefit from improved activity and/or improved tolerability relative to appropriate comparator compounds, such as ISIS 445569. For example, in certain embodiments, ISIS 598769, ISIS 598768, and/or ISIS 486178 have more activity and/or tolerability than appropriate comparator compounds, such as ISIS 445569.

In certain embodiments, the compounds disclosed herein are more potent than appropriate comparator compounds, such as ISIS 445569. For example, as provided in Example 10 (described herein), ISIS 598769 achieved an IC₅₀ of 1.9 μM, ISIS 598768 achieved an IC₅₀ of 1.2 μM, and ISIS 486178 achieved an IC₅₀ of 0.7 μM in a 6 point dose response curve (61.7 nM, 185.2 nM, 555.6 nM, 1666.7 nM, 5000.0 nM, and 15000.0 nM) in cultured in HepG2 cells when transfected using electroporation, whereas ISIS 445569 achieved an IC₅₀ of 2.3 μM. Thus, ISIS 598769, ISIS 598768, and ISIS 486178 are more potent than the comparator compound, ISIS 445569.

In certain embodiments, the compounds disclosed herein have greater activity than appropriate comparator compounds, such as ISIS 445569, at achieving dose-dependent inhibition of DMPK across multiple different muscle tissues. In another example, as provided in Example 16 (described herein), ISIS 598768 and ISIS 598769 achieved greater dose-dependent inhibition than the comparator compound ISIS 445569 across several different muscle tissues when administered subcutaneously to DMSXL transgenic mice twice a week for 4 weeks with 25 mg/kg/week, 50 mg/kg/wk, or 100 mg/kg/wk. In some muscle tissues, for example, in the tibialis anterior, both ISIS 598768 and ISIS 598769 achieved greater inhibition of DMPK at 25, 50 and 100 mg/kg/wk than ISIS 445569 achieved at 200 mg/kg/wk. In the quadriceps and gastrocnemius, both ISIS 598768 and ISIS 598769 achieved equal or greater inhibition of DMPK at 50 mg/kg/wk than ISIS 445569 achieved at 100 or 200 mg/kg/wk. Thus, ISIS 598768 and ISIS 598769 have greater activity than ISIS 445569 at achieving dose-dependent inhibition of DMPK across multiple different muscle tissues.

In certain embodiments, the compounds disclosed herein are more tolerable than appropriate comparator compounds, such as ISIS 445569, when administered to CD-1 mice. In another example, as provided in Example 17 (described herein), ISIS 598769, ISIS 598768, and ISIS 486178 exhibited more favorable tolerability markers than ISIS 445569 when administered to CD-1 mice. ISIS 598769, ISIS 598768, and ISIS 486178 were administered subcutaneously twice a week for 6 weeks at 50 mg/kg/wk. ISIS 445569 was administered subcutaneously twice a week for 6 weeks at 100 mg/kg/wk. After treatment, ALT, AST, and BUN levels were lower in ISIS 486178 and ISIS 598768 treated mice than in ISIS 445569 treated mice. After treatment, ALT and AST levels were lower in ISIS 598769 treated mice than in ISIS 445569 treated mice. Therefore, ISIS 598769, ISIS 598768, and ISIS 486178 are more tolerable than the comparator compound, ISIS 445569 in CD-1 mice.

In certain embodiments, the compounds disclosed herein are more tolerable than appropriate comparator compounds, such as ISIS 445569, when administered to Sprague-Dawley rats. In another example, as provided in Example 18 (described herein), ISIS 598769, ISIS 598768, and ISIS 486178 exhibited more favorable tolerability markers than ISIS 445569 when administered to Sprague-Dawley rats. ISIS 598769, ISIS 598768, and ISIS 486178 were administered subcutaneously twice a week for 6 weeks at 50 mg/kg/wk. ISIS 445569 was administered subcutaneously twice a week for 6 weeks at 100 mg/kg/wk. After treatment, ALT and AST levels were lower in ISIS 486178, ISIS 598769, and ISIS 598768 treated mice than in ISIS 445569 treated mice. Therefore ISIS 598769, ISIS 598768, and ISIS 486178 are more tolerable than the comparator compound, ISIS 445569 in Sprague-Dawley rats.

In certain embodiments, the compounds disclosed herein exhibit more favorable tolerability markers in cynomolgous monkeys than appropriate comparator compounds, such as ISIS 445569. In another example, as provided in Example 19 (described herein), ISIS 598769, ISIS 598768, and ISIS 486178 exhibited more favorable tolerability markers in cynomolgous monkeys including Alanine aminotransferase (ALT), aspartate aminotransferase (AST), lactate dehydrogenase (LDH), and creatine kinase (CK) assessment. In certain embodiments, ALT and AST levels are used as indicators of hepatotoxicity. For example, in certain embodiments, elevated ALT and AST levels indicate trauma to liver cells. In certain embodiments, elevated CK levels are associated with damage to cells in muscle tissue. In certain embodiments, elevated LDH levels are associated with cellular tissue damage.

In certain embodiments, the compounds disclosed herein are more tolerable than appropriate comparator compounds, such as ISIS 445569, when administered to cynomolgous monkeys. As provided in Example 19, groups of cynomolgous monkeys were treated with 40 mg/kg/wk of ISIS 598769, ISIS 598768, ISIS 486178, and ISIS 445569. Treatment with ISIS 445569 resulted in elevated ALT and AST levels at 93 days into treatment. Treatment with ISIS 598768, and ISIS 486178 resulted in lower ALT and AST levels at 58 and 93 days into treatment compared to ISIS 445569. Treatment with ISIS 598769, resulted in lower AST levels at 58 and 93 days into treatment and lower ALT levels at 93 days of treatment compared to ISIS 445569. Furthermore, the ALT and AST levels of monkeys receiving doses of ISIS 598769, ISIS 598768, and ISIS 486178 were consistent with the ALT and AST levels of monkeys given saline. Treatment with ISIS 445569 resulted in elevated LDH levels compared to the LDH levels measured in animals given ISIS 598769, ISIS 598768, and ISIS 486178 at 93 days into treatment. Additionally, treatment with ISIS 445569 resulted in elevated CK levels compared to the CK levels measured in animals given ISIS 598769, ISIS 598768, and ISIS 486178 at 93 days into treatment. Therefore, ISIS 598769, ISIS 598768, and ISIS 486178 are more tolerable than the comparator compound, ISIS 445569.

As the data discussed above demonstrate, ISIS 598769, ISIS 598768, and ISIS 486178 possess a wider range of well-tolerated doses at which ISIS 598769, ISIS 598768, and ISIS 486178 are active compared to the comparator compound, ISIS 445569. Additionally, the totality of the data presented in the examples herein and discussed above demonstrate that each of ISIS 598769, ISIS 598768, and ISIS 486178 possess a number of safety and activity advantages over the comparator compound, ISIS 445569. In other words, each of ISIS 598769, ISIS 598768, and ISIS 486178 are likely to be safer and more active drugs in humans than ISIS 445569.

In certain embodiments, ISIS 445569 is likely to be a safer and more active drug in humans for reducing CUGexp DMPK mRNA and\or treating conditions or symptoms associated with having myotonic dystrophy type 1 than the other compounds disclosed in WO 2012/012443 and/or WO 2012/012467.

In certain embodiments, ISIS 512497 has a better safety profile in primates and CD-1 mice than ISIS 445569. In certain embodiments, ISIS 512497 achieves greater knockdown of human DMPK nucleic acid in multiple muscle tissues when administered at the same dose and at lower doses than ISIS 445569.

In certain embodiments, ISIS 486178 has a better safety profile in mice, rats, and primates than ISIS 445569. In certain embodiments, ISIS 486178 achieves greater knockdown of human DMPK nucleic acid in one or more muscle tissues when administered at the same dose and at lower doses than ISIS 445569.

In certain embodiments, ISIS 570808 achieves much greater knockdown of human DMPK nucleic acid at least five different muscle tissues when administered at the same dose and at lower dose than ISIS 445569.

In certain embodiments, ISIS 594292 achieves greater knockdown of human DMPK nucleic acid in one or more muscle tissues when administered at the same dose as ISIS 445569. In certain embodiments, ISIS 486178 has a better safety profile in primates than ISIS 445569.

In certain embodiments, ISIS 569473 achieves greater knockdown of human DMPK nucleic acid in one or more muscle tissues when administered at the same dose as ISIS 445569. In certain embodiments, ISIS 569473 has a better safety profile in primates than ISIS 445569.

In certain embodiments, ISIS 594300 achieves greater knockdown of human DMPK nucleic acid in one or more muscle tissues when administered at the same dose as ISIS 445569. In certain embodiments, ISIS 594300 has a better safety profile in primates than ISIS 445569.

In certain embodiments, ISIS 598777 achieves greater knockdown of human DMPK nucleic acid in one or more muscle tissues when administered at the same dose as ISIS 445569. In certain embodiments, ISIS 598777 has a better safety profile in primates than ISIS 445569.

In certain embodiments, ISIS 598768 achieves greater knockdown of human DMPK nucleic acid in one or more muscle tissues when administered at the same dose as ISIS 445569. In certain embodiments, ISIS 598768 has a better safety profile in primates than ISIS 445569.

In certain embodiments, ISIS 598769 achieves greater knockdown of human DMPK nucleic acid in one or more muscle tissues when administered at the same dose as ISIS 445569. In certain embodiments, ISIS 598769 has a better safety profile in primates than ISIS 445569.

### Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified or naturally occurring bases, such as “AT^{me}CGAUCG,” wherein ^{me}C indicates a cytosine base comprising a methyl group at the 5-position.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Design of Antisense Oligonucleotides Targeting Human Dystrophia Myotonica Protein Kinase (hDMPK)

A series of antisense oligonucleotides (ASOs) were designed to target hDMPK. The newly designed ASOs were prepared using standard oligonucleotide synthesis well known in the art and are described in Tables 1 and 2, below. Subscripts “s” indicate phosphorothioate internucleoside linkages; subscripts “k” indicate 6′-(S)—CH₃ bicyclic nucleosides (cEt); subscripts “e” indicate 2′-O-methoxyethyl (MOE) modified nucleosides; and subscripts “d” indicate β-D-2′-deoxyribonucleosides. “mC” indicates 5-methylcytosine nucleosides.

The antisense oligonucleotides are targeted to either SEQ ID NO: 1 (GENBANK Accession No. NM_001081560.1) and/or SEQ ID NO: 2 (the complement of GENBANK Accession No. NT_011109.15 truncated from nucleotides 18540696 to 18555106). “Start site” indicates the 5′-most nucleoside to which the gapmer is targeted in the human gene sequence. “Stop site” indicates the 3′-most nucleoside to which the gapmer is targeted human gene sequence.

TABLE 1
Design of antisense oligonucleotides targeting hDMPK and targeted
to SEQ ID NO 2

| | | | | | | Unmodi- | Modi- |
|---|---|---|---|---|---|---|---|
| | | | | | | fied | fied |
| ISIS | | | | Start | Stop | SEQ ID | SEQ ID |
| No | Composition (5′ to 3′) | Motif | Length | Site | Site | No | No. |
| 486178 | Aₖₛ^{m}CₖₛAₖₛA_{ds}T_{ds}A_{ds}A_{ds}A_{ds} | kkk-10- | 16 | 13836 | 13851 | 23 | 881 |
| | T_{ds}A_{ds}^{m}C_{ds}^{m}C_{ds}G_{ds}AₖₛGₖₛGk | kkk | | | | | |
| 445569 | ^{m}CₑₛGₑₛGₑₛAₑₛGₑₛ^{m}C_{ds}G_{ds}G_{ds}T_{ds}T_{ds}G_{ds} | e5-d10- | 20 | 13226 | 13245 | 24 | 1443 |
| | T_{ds}G_{ds}A_{ds}A_{ds}^{m}CₑₛTₑₛGₑₛGₑₛ^{m}Cₑ | e5 | | | | | |
| 512497 | Gₑₛ^{m}CₑₛGₑₛ^{m}Cₑₛ^{m}AₑₛC_{ds}^{m}C_{ds}T_{ds}T_{ds}^{m}C_{ds} | e5-d10- | 20 | 8608 | 8627 | 25 | 1444 |
| | ^{m}C_{ds}^{m}C_{ds}G_{ds}A_{ds}A_{ds}TₑₛGₑₛTₑₛ^{m}Cₑₛ^{m}Cₑ | e5 | | | | | |
| 598768 | ^{m}Cₑₛ^{m}Cₑₛ^{m}CₖₛGₖₛA_{ds}A_{ds}T_{ds}G_{ds} | eekk-d8- | 16 | 8603 | 8618 | 26 | 1342 |
| | T_{ds}^{m}C_{ds}^{m}C_{ds}G_{ds}Aₖₛ^{m}CₖₛAₑₛGₑ | kkee | | | | | |
| 594300 | ^{m}CₑₛGₑₛGₑₛAₖₛGₖₛ^{m}C_{ds}G_{ds}G_{ds} | eeekk- | 17 | 13229 | 13245 | 27 | 1441 |
| | T_{ds}T_{ds}G_{ds}T_{ds}GₖₛAₖₛAₑₛ^{m}CₑₛTₑ | d7-kkeee | | | | | |
| 594292 | Aₑₛ^{m}CₑₛAₑₛAₖₛTₖₛA_{ds}A_{ds}A_{ds}T_{ds} | eeekk- | 17 | 13835 | 13851 | 28 | 1442 |
| | A_{ds}^{m}C_{ds}^{m}C_{ds}GₖₛAₖₛGₑₛGₑₛAₑ | d7-kkeee | | | | | |
| 569473 | GₖₛAₖₛ^{m}CₖₛA_{ds}A_{ds}T_{ds}^{m}C_{ds}T_{ds} | kkk-d10- | 16 | 5082 | 5097 | 29 | 882 |
| | ^{m}C_{ds}^{m}C_{ds}G_{ds}^{m}C_{ds}^{m}C_{ds}AₖₛGₖₛGₖ | kkk | | | | | |
| 598769 | Tₑₛ^{m}Cₑₛ^{m}Cₖₛ^{m}CₖₛG_{ds}A_{ds}A_{ds}T_{ds} | eekk-d8- | 16 | 8604 | 8619 | 30 | 1343 |
| | G_{ds}T_{ds}^{m}C_{ds}^{m}C_{ds}GₖₛAₖₛ^{m}CₑₛAₑ | kkee | | | | | |
| 570808 | TₖₛGₖₛTₖₛA_{ds}A_{ds}T_{ds}G_{ds}T_{ds} | kkk-d10- | 16 | 10201 | 10216 | 31 | 883 |
| | T_{ds}G_{ds}T_{ds}^{m}C_{ds}^{m}C_{ds}AₖₛGₖₛTₖ | kkk | | | | | |
| 598777 | GₑₛTₑₛGₖₛTₖₛA_{ds}A_{ds}T_{ds}G_{ds} | eekk-d8- | 16 | 10202 | 10217 | 32 | 1344 |
| | T_{ds}T_{ds}G_{ds}T_{ds}^{m}Cₖₛ^{m}CₖₛAₑₛGₑ | kkee | | | | | |

TABLE 2
Design of antisense oligonucleotides targeting hDMPK and targeted
to SEQ ID NO 1

| ISIS | | | | Start | Stop |
|---|---|---|---|---|---|
| No. | Composition (5′ to 3′) | Motif | Length | Site | Site |
| 486178 | Aₖₛ^{m}CₖₛAₖₛA_{ds}T_{ds}A_{ds}A_{ds}A_{ds} | kkk-10-kkk | 16 | 2773 | 2788 |
| | T_{ds}^{m}A_{ds}^{m}C_{ds}^{m}C_{ds}G_{ds}AₖₛGₖₛGₖ | | | | |
| 445569 | CₑₛGₑₛGₑₛAₑₛGₑₛ^{m}C_{ds}G_{ds}G_{ds}T_{ds}T_{ds}G_{ds} | e5-d10-e5 | 20 | 2163 | 2182 |
| | T_{ds}G_{ds}A_{ds}A_{ds}^{m}CₑₛTₑₛGₑₛGₑₛ^{m}Cₑ | | | | |
| 512497 | Gₑₛ^{m}CₑₛGₑₛ^{m}CₑₛAₑₛ^{m}C_{ds}^{m}C_{ds}T_{ds}T_{ds}^{m}C_{ds} | e5-d10-e5 | 20 | 1348 | 1367 |
| | ^{m}C_{ds}^{m}C_{ds}G_{ds}A_{ds}A_{ds}TₑₛGₑₛTₑₛ^{m}Cₑₛ^{m}Cₑ | | | | |
| 598768 | ^{m}Cₑₛ^{m}Cₑₛ^{m}CₖₛGₖₛA_{ds}A_{ds}T_{ds}G_{ds} | eekk-d8-kkee | 16 | 1343 | 1358 |
| | T_{ds}^{m}C_{ds}^{m}C_{ds}G_{ds}Aₖₛ^{m}CₖₛAₑₛGₑ | | | | |
| 594300 | ^{m}CₑₛGₑₛGₑₛAₖₛGₖₛ^{m}C_{ds}G_{ds}G_{ds} | eeekk-d7-kkeee | 17 | 2166 | 2182 |
| | T_{ds}T_{ds}G_{ds}T_{ds}GₖₛAₖₛAₑₛ^{m}CₑₛTₑ | | | | |
| 594292 | Aₑₛ^{m}CₑₛAₑₛAₖₛTₖₛA_{ds}A_{ds}A_{ds}T_{ds} | eeekk-d7-kkeee | 17 | 2772 | 2788 |
| | A_{ds}^{m}C_{ds}^{m}C_{ds}GₖₛAₖₛGₑₛGₑₛAₑ | | | | |
| 569473 | GₖₛAₖₛ^{m}CₖₛA_{ds}A_{ds}T_{ds}^{m}C_{ds}T_{ds} | kkk-d10-kkk | 16 | 730 | 745 |
| | ^{m}C_{ds}^{m}C_{ds}G_{ds}^{m}C_{ds}^{m}C_{ds}AₖₛGₖₛGₖ | | | | |
| 598769 | Tₑₛ^{m}Cₑₛ^{m}Cₖₛ^{m}CₖₛG_{ds}A_{ds}A_{ds}T_{ds} | eekk-d8-kkee | 16 | 1344 | 1359 |
| | G_{ds}T_{ds}^{m}C_{ds}^{m}C_{ds}GₖₛAₖₛ^{m}CₑₛAₑ | | | | |

### Example 2: Antisense Inhibition of Human DMPK in Human Skeletal Muscle Cells (hSKMc)

Antisense oligonucleotides targeted to a human DMPK nucleic acid were tested for their effect on DMPK RNA transcript in vitro. Cultured hSKMc cells at a density of 20,000 cells per well were transfected using electroporation with 10,000 nM antisense oligonucleotide. After approximately 24 hours, RNA was isolated from the cells and DMPK transcript levels were measured by quantitative real-time PCR. DMPK RNA transcript levels were adjusted according to total RNA content, as measured by RIBOGREEN®. Results are presented as percent expression of DMPK, relative to untreated control cells.

The antisense oligonucleotides in Tables 3, 4, 5, and 6 are 5-10-5 gapmers, where the gap segment comprises ten 2′-deoxynucleosides and each wing segment comprises five 2′-MOE nucleosides. The internucleoside linkages throughout each gapmer are phosphorothioate (P═S) linkages. All cytsoine residues throughout each gapmer are 5-methylcytosines. ‘Target start site’ indicates the 5′-most nucleoside to which the antisense oligonucleotide is targeted in the human genomic gene sequence. ‘Target stop site’ indicates the 3′-most nucleoside to which the antisense oligonucleotide is targeted in the human genomic sequence. All the antisense oligonucleotides listed in Table 3, 4, or 5 target SEQ ID NO: 1 (GENBANK Accession No. NM_001081560.1). All the antisense oligonucleotides listed in Table 6 target SEQ ID NO: 2 (the complement of GENBANK Accession No. NT_011109.15 truncated from nucleotides 18540696 to 18555106).

Several of the antisense oligonucleotides in Tables 2, 3, 4, and 5 demonstrated significant inhibition of DMPK mRNA levels under the conditions specified above.

TABLE 3
Inhibition of human DMPK RNA transcript in hSKMc by 5-10-5 gapmers targeting
SEQ ID NO: 1

| | | | Start Site | Stop Site | SEQ |
|---|---|---|---|---|---|
| ISIS | | % Target | on Seq ID: | on Seq | ID |
| No. | Sequence | Expression | 1 | ID: 1 | NO. |
| UTC | N/A | 100.0 | N/A | N/A | 33 |
| 444401 | TTGCACTTTGCGAACCAACG | 7.3 | 2490 | 2509 | 34 |
| 512326 | CGACACCTCGCCCCTCTTCA | 13.4 | 528 | 547 | 35 |
| 512327 | ACGACACCTCGCCCCTCTTC | 40.8 | 529 | 548 | 36 |
| 512328 | CACGACACCTCGCCCCTCTT | 27.8 | 530 | 549 | 37 |
| 512329 | GCACGACACCTCGCCCCTCT | 16.5 | 531 | 550 | 38 |
| 512330 | AGCACGACACCTCGCCCCTC | 17.9 | 532 | 551 | 39 |
| 512331 | AAGCACGACACCTCGCCCCT | 18.8 | 533 | 552 | 40 |
| 512332 | GAAGCACGACACCTCGCCCC | 23.3 | 534 | 553 | 41 |
| 512333 | GGAAGCACGACACCTCGCCC | 28.1 | 535 | 554 | 42 |
| 512334 | CGGAAGCACGACACCTCGCC | 16.3 | 536 | 555 | 43 |
| 512335 | ACGGAAGCACGACACCTCGC | 28.7 | 537 | 556 | 44 |
| 512336 | CACGGAAGCACGACACCTCG | 15.9 | 538 | 557 | 45 |
| 512337 | TCACGGAAGCACGACACCTC | 18.8 | 539 | 558 | 46 |
| 512338 | CTCACGGAAGCACGACACCT | 16.4 | 540 | 559 | 47 |
| 512339 | CCTCACGGAAGCACGACACC | 20.2 | 541 | 560 | 48 |
| 512340 | TCCTCACGGAAGCACGACAC | 19.3 | 542 | 561 | 49 |
| 512341 | CTCCTCACGGAAGCACGACA | 15.2 | 543 | 562 | 50 |
| 512342 | TCTCCTCACGGAAGCACGAC | 16.2 | 544 | 563 | 51 |
| 512343 | CTCTCCTCACGGAAGCACGA | 16.4 | 545 | 564 | 52 |
| 512344 | CCTCTCCTCACGGAAGCACG | 15.7 | 546 | 565 | 53 |
| 512345 | CCCTCTCCTCACGGAAGCAC | 14.7 | 547 | 566 | 54 |
| 512346 | TCCCTCTCCTCACGGAAGCA | 20.6 | 548 | 567 | 55 |
| 512347 | GTCCCTCTCCTCACGGAAGC | 32.6 | 549 | 568 | 56 |
| 512348 | CGTCCCTCTCCTCACGGAAG | 31.5 | 550 | 569 | 57 |
| 512349 | GGTCCCCATTCACCAACACG | 41.6 | 568 | 587 | 58 |
| 512350 | CGGTCCCCATTCACCAACAC | 31.6 | 569 | 588 | 59 |
| 512351 | CCGGTCCCCATTCACCAACA | 38.1 | 570 | 589 | 60 |
| 512352 | GCCGGTCCCCATTCACCAAC | 55.5 | 571 | 590 | 61 |
| 512353 | CGCCGGTCCCCATTCACCAA | 42.9 | 572 | 591 | 62 |
| 512354 | CCGCCGGTCCCCATTCACCA | 35.7 | 573 | 592 | 63 |
| 512355 | ACCGCCGGTCCCCATTCACC | 51.4 | 574 | 593 | 64 |
| 512356 | CACCGCCGGTCCCCATTCAC | 34.4 | 575 | 594 | 65 |
| 512357 | CCACCGCCGGTCCCCATTCA | 40.4 | 576 | 595 | 66 |
| 512358 | TCCACCGCCGGTCCCCATTC | 35.5 | 577 | 596 | 67 |
| 512359 | ATCCACCGCCGGTCCCCATT | 41.7 | 578 | 597 | 68 |
| 512360 | GATCCACCGCCGGTCCCCAT | 51.0 | 579 | 598 | 69 |
| 512361 | TGATCCACCGCCGGTCCCCA | 35.9 | 580 | 599 | 70 |
| 512362 | GTGATCCACCGCCGGTCCCC | 53.2 | 581 | 600 | 71 |
| 512363 | CGTGATCCACCGCCGGTCCC | 28.2 | 582 | 601 | 72 |
| 512364 | TTCTCATCCTGGAAGGCGAA | 34.6 | 611 | 630 | 73 |
| 512365 | GTTCTCATCCTGGAAGGCGA | 57.1 | 612 | 631 | 74 |
| 512366 | AGTTCTCATCCTGGAAGGCG | 72.1 | 613 | 632 | 75 |
| 512367 | GTAGTTCTCATCCTGGAAGG | 47.1 | 615 | 634 | 76 |
| 512368 | GGTAGTTCTCATCCTGGAAG | 56.0 | 616 | 635 | 77 |
| 512369 | AGGTAGTTCTCATCCTGGAA | 48.3 | 617 | 636 | 78 |
| 512370 | CAGGTAGTTCTCATCCTGGA | 20.2 | 618 | 637 | 79 |
| 512371 | TACAGGTAGTTCTCATCCTG | 44.0 | 620 | 639 | 80 |
| 512372 | GTACAGGTAGTTCTCATCCT | 64.1 | 621 | 640 | 81 |
| 512373 | GGTACAGGTAGTTCTCATCC | 54.2 | 622 | 641 | 82 |
| 512374 | AGGTACAGGTAGTTCTCATC | 65.6 | 623 | 642 | 83 |
| 512375 | CCAGGTACAGGTAGTTCTCA | 45.7 | 625 | 644 | 84 |
| 512376 | ACCAGGTACAGGTAGTTCTC | 60.4 | 626 | 645 | 85 |
| 512377 | GACCAGGTACAGGTAGTTCT | 62.2 | 627 | 646 | 86 |
| 512378 | TGACCAGGTACAGGTAGTTC | 64.9 | 628 | 647 | 87 |
| 512379 | CATGACCAGGTACAGGTAGT | 39.2 | 630 | 649 | 88 |
| 512380 | CCATGACCAGGTACAGGTAG | 27.7 | 631 | 650 | 89 |
| 512381 | TCCATGACCAGGTACAGGTA | 21.6 | 632 | 651 | 90 |
| 512382 | CTCCATGACCAGGTACAGGT | 25.7 | 633 | 652 | 91 |
| 512383 | ACTCCATGACCAGGTACAGG | 28.6 | 634 | 653 | 92 |
| 512384 | TACTCCATGACCAGGTACAG | 23.7 | 635 | 654 | 93 |
| 512385 | ATACTCCATGACCAGGTACA | 20.8 | 636 | 655 | 94 |
| 512386 | AATACTCCATGACCAGGTAC | 22.0 | 637 | 656 | 95 |
| 512387 | TAATACTCCATGACCAGGTA | 14.7 | 638 | 657 | 96 |
| 512388 | CGTAATACTCCATGACCAGG | 10.4 | 640 | 659 | 97 |
| 512389 | AGCAGTGTCAGCAGGTCCCC | 15.0 | 665 | 684 | 98 |
| 512390 | CAGCAGTGTCAGCAGGTCCC | 13.0 | 666 | 685 | 99 |
| 512391 | TCAGCAGTGTCAGCAGGTCC | 22.3 | 667 | 686 | 100 |
| 512392 | CTCAGCAGTGTCAGCAGGTC | 16.4 | 668 | 687 | 101 |
| 512393 | GCTCAGCAGTGTCAGCAGGT | 22.2 | 669 | 688 | 102 |
| 512394 | TGCTCAGCAGTGTCAGCAGG | 26.2 | 670 | 689 | 103 |
| 512395 | TTGCTCAGCAGTGTCAGCAG | 27.4 | 671 | 690 | 104 |
| 512396 | CTTGCTCAGCAGTGTCAGCA | 15.7 | 672 | 691 | 105 |
| 512397 | ACTTGCTCAGCAGTGTCAGC | 43.5 | 673 | 692 | 106 |
| 512398 | AACTTGCTCAGCAGTGTCAG | 26.9 | 674 | 693 | 107 |
| 512399 | AAACTTGCTCAGCAGTGTCA | 20.0 | 675 | 694 | 108 |
| 512400 | CAAACTTGCTCAGCAGTGTC | 23.1 | 676 | 695 | 109 |
| 512401 | CCAAACTTGCTCAGCAGTGT | 20.5 | 677 | 696 | 110 |
| 512402 | CCCAAACTTGCTCAGCAGTG | 13.5 | 678 | 697 | 33 |

TABLE 4
Inhibition of human DMPK RNA transcript in hSKMc by 5-10-5 gapmers
targeting SEQ ID NO: 1

| | | | Start Site | Stop Site | SEQ |
|---|---|---|---|---|---|
| ISIS | | % Target | on Seq ID: | on Seq | ID |
| No. | Sequence | Expression | 1 | ID: 1 | NO. |
| UTC | N/A | 100 | N/A | N/A | |
| 444401 | TTGCACTTTGCGAACCAACG | 13.4 | 2490 | 2509 | 33 |
| 512480 | GTGAGCCCGTCCTCCACCAA | 29.8 | 1310 | 1329 | 111 |
| 512481 | AGTGAGCCCGTCCTCCACCA | 15.6 | 1311 | 1330 | 112 |
| 512482 | CAGTGAGCCCGTCCTCCACC | 10.7 | 1312 | 1331 | 113 |
| 512483 | GCAGTGAGCCCGTCCTCCAC | 33.3 | 1313 | 1332 | 114 |
| 512484 | GGCAGTGAGCCCGTCCTCCA | 9.6 | 1314 | 1333 | 115 |
| 512485 | TGGCAGTGAGCCCGTCCTCC | 8.8 | 1315 | 1334 | 116 |
| 512486 | CATGGCAGTGAGCCCGTCCT | 10.5 | 1317 | 1336 | 117 |
| 512487 | CCATGGCAGTGAGCCCGTCC | 10.1 | 1318 | 1337 | 118 |
| 512488 | TCCATGGCAGTGAGCCCGTC | 13.7 | 1319 | 1338 | 119 |
| 512489 | CTCCATGGCAGTGAGCCCGT | 16.9 | 1320 | 1339 | 120 |
| 512490 | TCTCCATGGCAGTGAGCCCG | 29.1 | 1321 | 1340 | 121 |
| 512491 | GTCTCCATGGCAGTGAGCCC | 41.3 | 1322 | 1341 | 122 |
| 512492 | CCTTCCCGAATGTCCGACAG | 8.8 | 1343 | 1362 | 123 |
| 512493 | ACCTTCCCGAATGTCCGACA | 12.1 | 1344 | 1363 | 124 |
| 512494 | CACCTTCCCGAATGTCCGAC | 6 | 1345 | 1364 | 125 |
| 512495 | GCACCTTCCCGAATGTCCGA | 8.5 | 1346 | 1365 | 126 |
| 512496 | CGCACCTTCCCGAATGTCCG | 5.6 | 1347 | 1366 | 127 |
| 512497 | GCGCACCTTCCCGAATGTCC | 7.7 | 1348 | 1367 | 25 |
| 512498 | GGCGCACCTTCCCGAATGTC | 15 | 1349 | 1368 | 128 |
| 512499 | ACAAAAGGCAGGTGGACCCC | 22.8 | 1373 | 1392 | 129 |
| 512500 | CACAAAAGGCAGGTGGACCC | 22 | 1374 | 1393 | 130 |
| 512501 | CCACAAAAGGCAGGTGGACC | 16.4 | 1375 | 1394 | 131 |
| 512502 | CCCACAAAAGGCAGGTGGAC | 15.8 | 1376 | 1395 | 132 |
| 512503 | GCCCACAAAAGGCAGGTGGA | 25.1 | 1377 | 1396 | 133 |
| 512504 | AGCCCACAAAAGGCAGGTGG | 24.7 | 1378 | 1397 | 134 |
| 512505 | TAGCCCACAAAAGGCAGGTG | 20.7 | 1379 | 1398 | 135 |
| 512506 | GTAGCCCACAAAAGGCAGGT | 20.7 | 1380 | 1399 | 136 |
| 512507 | AGTAGCCCACAAAAGGCAGG | 27.8 | 1381 | 1400 | 137 |
| 512508 | GAGTAGCCCACAAAAGGCAG | 43.9 | 1382 | 1401 | 138 |
| 512509 | GGAGTAGCCCACAAAAGGCA | 29.9 | 1383 | 1402 | 139 |
| 512510 | AGGAGTAGCCCACAAAAGGC | 31.9 | 1384 | 1403 | 140 |
| 512511 | TAGGAGTAGCCCACAAAAGG | 59.9 | 1385 | 1404 | 141 |
| 512512 | GTAGGAGTAGCCCACAAAAG | 40.1 | 1386 | 1405 | 142 |
| 512513 | AGTAGGAGTAGCCCACAAAA | 48.1 | 1387 | 1406 | 143 |
| 512514 | GAGTAGGAGTAGCCCACAAA | 53.3 | 1388 | 1407 | 144 |
| 512515 | GGAGTAGGAGTAGCCCACAA | 24.7 | 1389 | 1408 | 145 |
| 512516 | AGGAGTAGGAGTAGCCCACA | 22.1 | 1390 | 1409 | 146 |
| 512517 | CAGGAGTAGGAGTAGCCCAC | 15.4 | 1391 | 1410 | 147 |
| 512518 | GCAGGAGTAGGAGTAGCCCA | 32.8 | 1392 | 1411 | 148 |
| 512519 | TGCAGGAGTAGGAGTAGCCC | 37.6 | 1393 | 1412 | 149 |
| 512520 | ATGCAGGAGTAGGAGTAGCC | 47.4 | 1394 | 1413 | 150 |
| 512521 | CATGCAGGAGTAGGAGTAGC | 67.2 | 1395 | 1414 | 151 |
| 512522 | CCATGCAGGAGTAGGAGTAG | 58.8 | 1396 | 1415 | 152 |
| 512523 | GCCATGCAGGAGTAGGAGTA | 42.4 | 1397 | 1416 | 153 |
| 512524 | GGCCATGCAGGAGTAGGAGT | 34.1 | 1398 | 1417 | 154 |
| 512525 | GGGCCATGCAGGAGTAGGAG | 44.5 | 1399 | 1418 | 155 |
| 512526 | AGGGCCATGCAGGAGTAGGA | 42 | 1400 | 1419 | 156 |
| 512527 | GAGGGCCATGCAGGAGTAGG | 46.3 | 1401 | 1420 | 157 |
| 512528 | CTGAGGGCCATGCAGGAGTA | 25.3 | 1403 | 1422 | 158 |
| 512529 | CCTGAGGGCCATGCAGGAGT | 28.1 | 1404 | 1423 | 159 |
| 512530 | CCCTGAGGGCCATGCAGGAG | 22.8 | 1405 | 1424 | 160 |
| 512531 | TCCCTGAGGGCCATGCAGGA | 25.7 | 1406 | 1425 | 161 |
| 512532 | GTCCCTGAGGGCCATGCAGG | 17 | 1407 | 1426 | 162 |
| 512533 | TGTCCCTGAGGGCCATGCAG | 18.9 | 1408 | 1427 | 163 |
| 512534 | CTGTCCCTGAGGGCCATGCA | 27.3 | 1409 | 1428 | 164 |
| 512535 | ACTGTCCCTGAGGGCCATGC | 16.5 | 1410 | 1429 | 165 |
| 512536 | CACTGTCCCTGAGGGCCATG | 26 | 1411 | 1430 | 166 |
| 512537 | TCACTGTCCCTGAGGGCCAT | 22.7 | 1412 | 1431 | 167 |
| 512538 | CTCACTGTCCCTGAGGGCCA | 20.2 | 1413 | 1432 | 168 |
| 512539 | CCTCACTGTCCCTGAGGGCC | 19.3 | 1414 | 1433 | 169 |
| 512540 | ACCTCACTGTCCCTGAGGGC | 31 | 1415 | 1434 | 170 |
| 512541 | GACCTCACTGTCCCTGAGGG | 51.4 | 1416 | 1435 | 171 |
| 512542 | GGACCTCACTGTCCCTGAGG | 28 | 1417 | 1436 | 172 |
| 512543 | GGGACCTCACTGTCCCTGAG | 42.6 | 1418 | 1437 | 173 |
| 512544 | CCTCCAGTTCCATGGGTGTG | 16.7 | 1444 | 1463 | 174 |
| 512545 | GCCTCCAGTTCCATGGGTGT | 21.9 | 1445 | 1464 | 175 |
| 512546 | GGCCTCCAGTTCCATGGGTG | 19 | 1446 | 1465 | 176 |
| 512547 | CGGCCTCCAGTTCCATGGGT | 14.9 | 1447 | 1466 | 177 |
| 512548 | TCGGCCTCCAGTTCCATGGG | 23 | 1448 | 1467 | 178 |
| 512549 | CTCGGCCTCCAGTTCCATGG | 15.7 | 1449 | 1468 | 179 |
| 512550 | GCTCGGCCTCCAGTTCCATG | 16.2 | 1450 | 1469 | 180 |
| 512551 | TGCTCGGCCTCCAGTTCCAT | 17.7 | 1451 | 1470 | 181 |
| 512552 | CTGCTCGGCCTCCAGTTCCA | 18.4 | 1452 | 1471 | 182 |
| 512553 | GCTGCTCGGCCTCCAGTTCC | 22 | 1453 | 1472 | 183 |
| 512554 | AGCTGCTCGGCCTCCAGTTC | 32.4 | 1454 | 1473 | 184 |
| 512555 | CAGCTGCTCGGCCTCCAGTT | 15.7 | 1455 | 1474 | 185 |
| 512556 | GCAGCTGCTCGGCCTCCAGT | 16.3 | 1456 | 1475 | 186 |

TABLE 5
Inhibition of human DMPK RNA transcript in hSKMc by 5-10-5 gapmers targeting
SEQ ID NO: 1

| | | | Start Site | Stop Site | SEQ |
|---|---|---|---|---|---|
| ISIS | | % Target | on Seq ID: | on Seq | ID |
| No. | Sequence | Expression | 1 | ID: 1 | NO. |
| UTC | N/A | 100.0 | N/A | N/A | |
| 444401 | TTGCACTTTGCGAACCAACG | 7.0 | 2490 | 2509 | 33 |
| 512557 | AGCAGCTGCTCGGCCTCCAG | 25.2 | 1457 | 1476 | 187 |
| 512558 | AAGCAGCTGCTCGGCCTCCA | 16.1 | 1458 | 1477 | 188 |
| 512559 | CAAGCAGCTGCTCGGCCTCC | 21.9 | 1459 | 1478 | 189 |
| 512560 | TCAAGCAGCTGCTCGGCCTC | 24.8 | 1460 | 1479 | 190 |
| 512561 | CTCAAGCAGCTGCTCGGCCT | 19.8 | 1461 | 1480 | 191 |
| 512562 | GCTCAAGCAGCTGCTCGGCC | 11.6 | 1462 | 1481 | 192 |
| 512563 | GGCTCAAGCAGCTGCTCGGC | 19.8 | 1463 | 1482 | 193 |
| 512564 | TGGCTCAAGCAGCTGCTCGG | 31.9 | 1464 | 1483 | 194 |
| 512565 | GTGGCTCAAGCAGCTGCTCG | 27.5 | 1465 | 1484 | 195 |
| 512566 | TGTGGCTCAAGCAGCTGCTC | 35.4 | 1466 | 1485 | 196 |
| 512567 | GTGTGGCTCAAGCAGCTGCT | 24.8 | 1467 | 1486 | 197 |
| 512568 | CCACTTCAGCTGTTTCATCC | 43.1 | 1525 | 1544 | 198 |
| 512569 | TGCCACTTCAGCTGTTTCAT | 35.0 | 1527 | 1546 | 199 |
| 512570 | CTGCCACTTCAGCTGTTTCA | 27.8 | 1528 | 1547 | 200 |
| 512571 | ACTGCCACTTCAGCTGTTTC | 78.9 | 1529 | 1548 | 201 |
| 512572 | AACTGCCACTTCAGCTGTTT | 36.4 | 1530 | 1549 | 202 |
| 512573 | GAACTGCCACTTCAGCTGTT | 30.3 | 1531 | 1550 | 203 |
| 512574 | GGAACTGCCACTTCAGCTGT | 66.7 | 1532 | 1551 | 204 |
| 512575 | TGGAACTGCCACTTCAGCTG | 22.6 | 1533 | 1552 | 205 |
| 512576 | CTGGAACTGCCACTTCAGCT | 22.9 | 1534 | 1553 | 206 |
| 512577 | GCTGGAACTGCCACTTCAGC | 59.5 | 1535 | 1554 | 207 |
| 512578 | CGCTGGAACTGCCACTTCAG | 24.9 | 1536 | 1555 | 208 |
| 512579 | CCGCTGGAACTGCCACTTCA | 42.5 | 1537 | 1556 | 209 |
| 512580 | GCCGCTGGAACTGCCACTTC | 20.0 | 1538 | 1557 | 210 |
| 512581 | AGCCGCTGGAACTGCCACTT | 19.4 | 1539 | 1558 | 211 |
| 512582 | CTCAGCCTCTGCCGCAGGGA | 22.1 | 1560 | 1579 | 212 |
| 512583 | CCTCAGCCTCTGCCGCAGGG | 33.7 | 1561 | 1580 | 213 |
| 512584 | GGCCTCAGCCTCTGCCGCAG | 24.6 | 1563 | 1582 | 214 |
| 512585 | CGGCCTCAGCCTCTGCCGCA | 55.1 | 1564 | 1583 | 215 |
| 512586 | TCGGCCTCAGCCTCTGCCGC | 60.8 | 1565 | 1584 | 216 |
| 512587 | CTCGGCCTCAGCCTCTGCCG | 31.8 | 1566 | 1585 | 217 |
| 512588 | CCTCGGCCTCAGCCTCTGCC | 16.4 | 1567 | 1586 | 218 |
| 512589 | ACCTCGGCCTCAGCCTCTGC | 31.1 | 1568 | 1587 | 219 |
| 512590 | CACCTCGGCCTCAGCCTCTG | 39.7 | 1569 | 1588 | 220 |
| 512591 | TCACCTCGGCCTCAGCCTCT | 24.8 | 1570 | 1589 | 221 |
| 512592 | GTCACCTCGGCCTCAGCCTC | 28.7 | 1571 | 1590 | 222 |
| 512593 | CGTCACCTCGGCCTCAGCCT | 20.3 | 1572 | 1591 | 223 |
| 512594 | AGCACCTCCTCCTCCAGGGC | 18.4 | 1610 | 1629 | 224 |
| 512595 | GAGCACCTCCTCCTCCAGGG | 19.9 | 1611 | 1630 | 225 |
| 512596 | TGAGCACCTCCTCCTCCAGG | 15.6 | 1612 | 1631 | 226 |
| 512597 | GTGAGCACCTCCTCCTCCAG | 22.3 | 1613 | 1632 | 227 |
| 512598 | GGTGAGCACCTCCTCCTCCA | 19.4 | 1614 | 1633 | 228 |
| 512599 | GGGTGAGCACCTCCTCCTCC | 17.3 | 1615 | 1634 | 229 |
| 512600 | CGGGTGAGCACCTCCTCCTC | 12.2 | 1616 | 1635 | 230 |
| 512601 | CCGGGTGAGCACCTCCTCCT | 15.9 | 1617 | 1636 | 231 |
| 512602 | GCCGGGTGAGCACCTCCTCC | 15.7 | 1618 | 1637 | 232 |
| 512603 | TGCCGGGTGAGCACCTCCTC | 15.1 | 1619 | 1638 | 233 |
| 512604 | CTGCCGGGTGAGCACCTCCT | 24.5 | 1620 | 1639 | 234 |
| 512605 | TCTGCCGGGTGAGCACCTCC | 33.8 | 1621 | 1640 | 235 |
| 512606 | GCTCTGCCGGGTGAGCACCT | 26.1 | 1623 | 1642 | 236 |
| 512607 | GGCTCTGCCGGGTGAGCACC | 50.4 | 1624 | 1643 | 237 |
| 512608 | AGGCTCTGCCGGGTGAGCAC | 42.9 | 1625 | 1644 | 238 |
| 512609 | CAGGCTCTGCCGGGTGAGCA | 39.2 | 1626 | 1645 | 239 |
| 512610 | TCAGGCTCTGCCGGGTGAGC | 20.2 | 1627 | 1646 | 240 |
| 512611 | GCTCAGGCTCTGCCGGGTGA | 22.5 | 1629 | 1648 | 241 |
| 512612 | CGGCTCAGGCTCTGCCGGGT | 27.0 | 1631 | 1650 | 242 |
| 512613 | CCGGCTCAGGCTCTGCCGGG | 68.8 | 1632 | 1651 | 243 |
| 512614 | CCCGGCTCAGGCTCTGCCGG | 58.8 | 1633 | 1652 | 244 |
| 512615 | TCCCGGCTCAGGCTCTGCCG | 24.8 | 1634 | 1653 | 245 |
| 512616 | CTCCCGGCTCAGGCTCTGCC | 10.4 | 1635 | 1654 | 246 |
| 512617 | TCTCCCGGCTCAGGCTCTGC | 12.8 | 1636 | 1655 | 247 |
| 512618 | ATCTCCCGGCTCAGGCTCTG | 13.3 | 1637 | 1656 | 248 |
| 512619 | CATCTCCCGGCTCAGGCTCT | 7.7 | 1638 | 1657 | 249 |
| 512620 | CCATCTCCCGGCTCAGGCTC | 2.8 | 1639 | 1658 | 250 |
| 512621 | TCCATCTCCCGGCTCAGGCT | 2.6 | 1640 | 1659 | 251 |
| 512622 | CTCCATCTCCCGGCTCAGGC | 1.5 | 1641 | 1660 | 252 |
| 512623 | CCTCCATCTCCCGGCTCAGG | 1.4 | 1642 | 1661 | 253 |
| 512624 | GCCTCCATCTCCCGGCTCAG | 2.0 | 1643 | 1662 | 254 |
| 512625 | GGCCTCCATCTCCCGGCTCA | 8.3 | 1644 | 1663 | 255 |
| 512626 | TGGCCTCCATCTCCCGGCTC | 9.4 | 1645 | 1664 | 256 |
| 512627 | ATGGCCTCCATCTCCCGGCT | 6.3 | 1646 | 1665 | 257 |
| 512628 | GATGGCCTCCATCTCCCGGC | 2.7 | 1647 | 1666 | 258 |
| 512629 | GGATGGCCTCCATCTCCCGG | 1.3 | 1648 | 1667 | 259 |
| 512630 | CGGATGGCCTCCATCTCCCG | 1.5 | 1649 | 1668 | 260 |
| 512631 | GCGGATGGCCTCCATCTCCC | 2.4 | 1650 | 1669 | 261 |
| 512632 | TGCGGATGGCCTCCATCTCC | 2.2 | 1651 | 1670 | 262 |
| 512633 | GTTCCGAGCCTCTGCCTCGC | 29.2 | 1701 | 1720 | 263 |

TABLE 6
Inhibition of human DMPK RNA transcript in hSKMc by 5-10-5 gapmers targeting
SEQ ID NO: 2

| | | | Start Site | Stop Site | SEQ |
|---|---|---|---|---|---|
| ISIS | | % Target | on Seq ID: | on Seq | ID |
| No. | Sequence | Expression | 2 | ID: 2 | NO. |
| UTC | N/A | 100.0 | N/A | N/A | |
| 444401 | TTGCACTTTGCGAACCAACG | 7.0 | 13553 | 13572 | 33 |
| 444436 | GTCGGAGGACGAGGTCAATA | 9.7 | 13748 | 13767 | 264 |
| 512634 | AGGGCCTCAGCCTGGCCGAA | 31.7 | 13452 | 13471 | 265 |
| 512635 | CAGGGCCTCAGCCTGGCCGA | 39.5 | 13453 | 13472 | 266 |
| 512636 | GTCAGGGCCTCAGCCTGGCC | 20.5 | 13455 | 13474 | 267 |
| 512637 | CGTCAGGGCCTCAGCCTGGC | 23.3 | 13456 | 13475 | 268 |
| 512638 | AGCAAATTTCCCGAGTAAGC | 14.7 | 13628 | 13647 | 269 |
| 512639 | AAGCAAATTTCCCGAGTAAG | 21.2 | 13629 | 13648 | 270 |
| 512640 | AAAAGCAAATTTCCCGAGTA | 23.0 | 13631 | 13650 | 271 |
| 512641 | CAAAAGCAAATTTCCCGAGT | 19.7 | 13632 | 13651 | 272 |
| 512642 | GCAAAAGCAAATTTCCCGAG | 26.6 | 13633 | 13652 | 273 |
| 512643 | GGCAAAAGCAAATTTCCCGA | 12.8 | 13634 | 13653 | 274 |
| 512644 | TGGCAAAAGCAAATTTCCCG | 12.2 | 13635 | 13654 | 275 |
| 512645 | TTTGGCAAAAGCAAATTTCC | 24.2 | 13637 | 13656 | 276 |
| 512646 | GTTTGGCAAAAGCAAATTTC | 25.5 | 13638 | 13657 | 277 |
| 512647 | GGGTTTGGCAAAAGCAAATT | 43.0 | 13640 | 13659 | 278 |
| 512648 | CGGGTTTGGCAAAAGCAAAT | 27.2 | 13641 | 13660 | 279 |
| 512649 | AAGCGGGTTTGGCAAAAGCA | 27.0 | 13644 | 13663 | 280 |
| 512650 | AATATCCAAACCGCCGAAGC | 45.7 | 13728 | 13747 | 281 |
| 512651 | AAATATCCAAACCGCCGAAG | 56.6 | 13729 | 13748 | 282 |
| 512652 | ATAAATATCCAAACCGCCGA | 39.0 | 13731 | 13750 | 283 |
| 512653 | AATAAATATCCAAACCGCCG | 34.7 | 13732 | 13751 | 284 |
| 512654 | TCAATAAATATCCAAACCGC | 34.7 | 13734 | 13753 | 285 |
| 512655 | GTCAATAAATATCCAAACCG | 19.1 | 13735 | 13754 | 286 |
| 512656 | GGTCAATAAATATCCAAACC | 24.3 | 13736 | 13755 | 287 |
| 512657 | AGGTCAATAAATATCCAAAC | 23.5 | 13737 | 13756 | 288 |
| 512658 | GAGGTCAATAAATATCCAAA | 24.2 | 13738 | 13757 | 289 |
| 512659 | ACGAGGTCAATAAATATCCA | 28.3 | 13740 | 13759 | 290 |
| 512660 | GACGAGGTCAATAAATATCC | 17.8 | 13741 | 13760 | 291 |
| 512661 | AGGACGAGGTCAATAAATAT | 45.7 | 13743 | 13762 | 292 |
| 512662 | GAGGACGAGGTCAATAAATA | 27.6 | 13744 | 13763 | 293 |
| 512663 | CGGAGGACGAGGTCAATAAA | 15.8 | 13746 | 13765 | 294 |
| 512664 | TCGGAGGACGAGGTCAATAA | 10.8 | 13747 | 13766 | 295 |
| 512665 | AGTCGGAGGACGAGGTCAAT | 15.4 | 13749 | 13768 | 296 |
| 512666 | GAGTCGGAGGACGAGGTCAA | 18.8 | 13750 | 13769 | 297 |
| 512667 | GCGAGTCGGAGGACGAGGTC | 26.0 | 13752 | 13771 | 298 |
| 512668 | AGCGAGTCGGAGGACGAGGT | 21.7 | 13753 | 13772 | 299 |
| 512669 | CAGCGAGTCGGAGGACGAGG | 13.7 | 13754 | 13773 | 300 |
| 512670 | TCAGCGAGTCGGAGGACGAG | 16.5 | 13755 | 13774 | 301 |
| 512671 | GTCAGCGAGTCGGAGGACGA | 17.4 | 13756 | 13775 | 302 |
| 512672 | CTGTCAGCGAGTCGGAGGAC | 25.2 | 13758 | 13777 | 303 |
| 512673 | CCTGTCAGCGAGTCGGAGGA | 18.4 | 13759 | 13778 | 304 |
| 512674 | AGCCTGTCAGCGAGTCGGAG | 16.8 | 13761 | 13780 | 305 |
| 512675 | GTCTCAGTGCATCCAAAACG | 11.8 | 13807 | 13826 | 306 |
| 512676 | GGTCTCAGTGCATCCAAAAC | 17.7 | 13808 | 13827 | 307 |
| 512677 | GGGTCTCAGTGCATCCAAAA | 11.2 | 13809 | 13828 | 308 |
| 512678 | GGAGGGCCTTTTATTCGCGA | 17.8 | 13884 | 13903 | 309 |
| 512679 | TGGAGGGCCTTTTATTCGCG | 13.2 | 13885 | 13904 | 310 |
| 512680 | ATGGAGGGCCTTTTATTCGC | 19.3 | 13886 | 13905 | 311 |
| 512681 | GATGGAGGGCCTTTTATTCG | 30.5 | 13887 | 13906 | 312 |
| 512682 | AGATGGAGGGCCTTTTATTC | 50.8 | 13888 | 13907 | 313 |
| 512683 | CAGATGGAGGGCCTTTTATT | 46.1 | 13889 | 13908 | 314 |
| 512684 | GCAGATGGAGGGCCTTTTAT | 50.4 | 13890 | 13909 | 315 |
| 512685 | CCCTCAGGCTCTCTGCTTTA | 34.7 | 655 | 674 | 316 |
| 512686 | GCCCTCAGGCTCTCTGCTTT | 47.9 | 656 | 675 | 317 |
| 512687 | AGCCCTCAGGCTCTCTGCTT | 47.4 | 657 | 676 | 318 |
| 512688 | TAGCCCTCAGGCTCTCTGCT | 54.1 | 658 | 677 | 319 |
| 512689 | TTAGCCCTCAGGCTCTCTGC | 48.0 | 659 | 678 | 320 |
| 512690 | TTTAGCCCTCAGGCTCTCTG | 50.7 | 660 | 679 | 321 |
| 512691 | ATTTAGCCCTCAGGCTCTCT | 47.3 | 661 | 680 | 322 |
| 512692 | AATTTAGCCCTCAGGCTCTC | 44.8 | 662 | 681 | 323 |
| 512693 | AAATTTAGCCCTCAGGCTCT | 39.2 | 663 | 682 | 324 |
| 512694 | TAAATTTAGCCCTCAGGCTC | 48.0 | 664 | 683 | 325 |
| 512695 | TTAAATTTAGCCCTCAGGCT | 54.9 | 665 | 684 | 326 |
| 512696 | GTTAAATTTAGCCCTCAGGC | 48.1 | 666 | 685 | 327 |
| 512697 | AGTTAAATTTAGCCCTCAGG | 39.3 | 667 | 686 | 328 |
| 512698 | CAGTTAAATTTAGCCCTCAG | 47.5 | 668 | 687 | 329 |
| 512699 | ACAGTTAAATTTAGCCCTCA | 68.2 | 669 | 688 | 330 |
| 512700 | GACAGTTAAATTTAGCCCTC | 59.2 | 670 | 689 | 331 |
| 512701 | GGACAGTTAAATTTAGCCCT | 63.7 | 671 | 690 | 332 |
| 512702 | CGGACAGTTAAATTTAGCCC | 50.7 | 672 | 691 | 333 |
| 512703 | TCGGACAGTTAAATTTAGCC | 39.6 | 673 | 692 | 334 |
| 512704 | CTCGGACAGTTAAATTTAGC | 36.5 | 674 | 693 | 335 |
| 512705 | ACTCGGACAGTTAAATTTAG | 59.1 | 675 | 694 | 336 |
| 512706 | GACTCGGACAGTTAAATTTA | 50.0 | 676 | 695 | 337 |
| 512707 | CGACTCGGACAGTTAAATTT | 63.0 | 677 | 696 | 338 |
| 512708 | CCGACTCGGACAGTTAAATT | 34.3 | 678 | 697 | 339 |
| 512709 | TCCGACTCGGACAGTTAAAT | 39.5 | 679 | 698 | 340 |

### Example 3: Design of Antisense Oligonucleotides Targeting Human Dystrophia Myotonica Protein Kinase (hDMPK)

A series of antisense oligonucleotides (ASOs) were designed to target hDMPK. The newly designed ASOs were prepared using standard oligonucleotide synthesis well known in the art and are described in Table 7, below. Subscripts “s” indicate phosphorothioate internucleoside linkages; subscripts “k” indicate 6′-(S)—CH₃ bicyclic nucleosides (cEt); subscripts “e” indicate 2′-O-methoxyethyl (MOE) modified nucleosides; and subscripts “d” indicate β-D-2′-deoxyribonucleosides. “^{m}C” indicates 5-methylcytosine nucleosides.

The antisense oligonucleotides targeted to a human DMPK nucleic acid were tested for their effect on DMPK RNA transcript in vitro. Cultured HepG2 cells at a density of 20,000 cells per well were transfected using electroporation with 4,500 nM antisense oligonucleotide. After approximately 24 hours, RNA was isolated from the cells and DMPK transcript levels were measured by quantitative real-time PCR. DMPK RNA transcript levels were adjusted according to total RNA content, as measured by RIBOGREEN®. Results are presented as percent expression of DMPK, relative to untreated control cells.

‘Target start site’ indicates the 5′-most nucleoside to which the antisense oligonucleotide is targeted in the human genomic gene sequence. ‘Target stop site’ indicates the 3′-most nucleoside to which the antisense oligonucleotide is targeted in the human genomic sequence. All the antisense oligonucleotides listed in Table 7 target SEQ ID NO: 1 (GENBANK Accession No. NM_001081560.1).

Several of the antisense oligonucleotides demonstrated significant inhibition of DMPK mRNA levels under the conditions specified above.

TABLE 7
Inhibition of human DMPK RNA transcript in HepG2 cells targeting SEQ ID NO: 1

| | | | Start | Stop | | |
|---|---|---|---|---|---|---|
| | | | Site | Site | Un- | |
| | | | on | on | modified | Modified |
| ISIS | | % Target | Seq | Seq | Seq ID | Seq ID |
| No. | Sequence | Expression | ID: 1 | ID: 1 | No. | No. |
| UTC | N/A | 100 | N/A | N/A | | |
| 533424 | Tₑₛ^{m}CₑₛTₑₛ^{m}C_{ds}^{m}C_{ds}T_{ds}^{m}C_{ds}A_{ds}^{m}C_{ds}G_{ds} | 34.4 | 548 | 563 | 341 | 1345 |
| | G_{ds}A_{ds}A_{ds}Gₖₛ^{m}CₖₛAₖ | | | | | |
| 533425 | ^{m}CₑₛTₑₛ^{m}CₑₛT_{ds}^{m}C_{ds}^{m}C_{ds}T_{ds}^{m}C_{ds}A_{ds}^{m} | 32.1 | 549 | 564 | 342 | 1346 |
| | C_{ds}G_{ds}G_{ds}A_{ds}AₖₛGₖₛ^{m}Cₖ | | | | | |
| 533426 | ^{m}Cₑₛ^{m}CₑₛTₑₛ^{m}C_{ds}T_{ds}^{m}C_{ds}^{m}C_{ds}T_{ds}^{m}C_{ds} | 52.1 | 550 | 565 | 343 | 1347 |
| | A_{ds}^{m}C_{ds}G_{ds}G_{ds}AₖₛAₖₛGₖ | | | | | |
| 533427 | AₑₛAₑₛAₑₛ^{m}C_{ds}T_{ds}T_{ds}G_{ds}^{m}C_{ds}T_{ds}^{m}C_{ds} | 36.8 | 679 | 694 | 344 | 1348 |
| | A_{ds}G_{ds}^{m}C_{ds}AₖₛGₖₛTₖ | | | | | |
| 533428 | ^{m}CₑₛAₑₛAₑₛA_{ds}^{m}C_{ds}T_{ds}T_{ds}G_{ds}^{m}C_{ds}T_{ds} | 59.9 | 680 | 695 | 345 | 1349 |
| | ^{m}C_{ds}A_{ds}G_{ds}^{m}CₖₛAₖₛGₖ | | | | | |
| 533429 | ^{m}Cₑₛ^{m}CₑₛAₑₛA_{ds}A_{ds}^{m}C_{ds}T_{ds}T_{ds}G_{ds}^{m}C_{ds} | 39.3 | 681 | 696 | 346 | 1350 |
| | T_{ds}^{m}C_{ds}A_{ds}Gₖₛ^{m}CₖₛAₖ | | | | | |
| 533430 | ^{m}Cₑₛ^{m}Cₑₛ^{m}CₑₛA_{ds}A_{ds}A_{ds}^{m}C_{ds}T_{ds}T_{ds}G_{ds} | 37.6 | 682 | 697 | 347 | 1351 |
| | ^{m}C_{ds}T_{ds}^{m}C_{ds}AₖₛGₖₛ^{m}Cₖ | | | | | |
| 533431 | ^{m}Cₑₛ^{m}Cₑₛ^{m}Cₑₛ^{m}C_{ds}A_{ds}A_{ds}A_{ds}^{m}C_{ds}T_{ds}T_{ds} | 39.6 | 683 | 698 | 348 | 1352 |
| | G_{ds}^{m}C_{ds}T_{ds}^{m}CₖₛAₖₛGₖ | | | | | |
| 533432 | Tₑₛ^{m}Cₑₛ^{m}Cₑₛ^{m}C_{ds}^{m}C_{ds}A_{ds}A_{ds}A_{ds}^{m}C_{ds}T_{ds} | 52.1 | 684 | 699 | 349 | 1353 |
| | _{ds}T_{ds}G_{ds}^{m}C_{ds}Tₖₛ^{m}CₖₛAₖ | | | | | |
| 533433 | GₑₛTₑₛTₑₛT_{ds}G_{ds}A_{ds}T_{ds}G_{ds}T_{ds}^{m}C_{ds}^{m}C_{ds} | 53.9 | 782 | 797 | 350 | 1354 |
| | C_{ds}T_{ds}GₖₛTₖₛGₖ | | | | | |
| 533434 | GₑₛGₑₛTₑₛT_{ds}T_{ds}G_{ds}A_{ds}T_{ds}G_{ds}T_{ds}^{m}C_{ds} | 38.1 | 783 | 798 | 351 | 1355 |
| | ^{m}C_{ds}^{m}C_{ds}TₖₛGₖₛTₖ | | | | | |
| 533435 | GₑₛGₑₛGₑₛT_{ds}T_{ds}T_{ds}G_{ds}A_{ds}T_{ds}G_{ds}T_{ds}^{m} | 43.7 | 784 | 799 | 352 | 1356 |
| | C_{ds}^{m}C_{ds}^{m}CₖₛTₖₛGₖ | | | | | |
| 533436 | Aₑₛ^{m}CₑₛAₑₛG_{ds}^{m}C_{ds}^{m}C_{ds}T_{ds}G_{ds}^{m}C_{ds}A_{ds} | 29.5 | 927 | 942 | 353 | 1357 |
| | G_{ds}G_{ds}A_{ds}Tₖₛ^{m}CₖₛTₖ | | | | | |
| 533437 | ^{m}CₑₛAₑₛ^{m}CₑₛA_{ds}G_{ds}^{m}C_{ds}^{m}C_{ds}T_{ds}G_{ds}^{m} | 48.6 | 928 | 943 | 354 | 1358 |
| | C_{ds}A_{ds}G_{ds}G_{ds}AₖₛTₖₛ^{m}Cₖ | | | | | |
| 533438 | ^{m}Cₑₛ^{m}CₑₛAₑₛ^{m}C_{ds}A_{ds}G_{ds}^{m}C_{ds}^{m}C_{ds}T_{ds} | 46.9 | 929 | 944 | 355 | 1359 |
| | G_{ds}^{m}C_{ds}A_{ds}G_{ds}GₖₛAₖₛTₖ | | | | | |
| 533439 | ^{m}Cₑₛ^{m}Cₑₛ^{m}CₑₛA_{ds}^{m}C_{ds}A_{ds}G_{ds}^{m}C_{ds}^{m}C_{ds} | 43.6 | 930 | 945 | 356 | 1360 |
| | T_{ds}G_{ds}^{m}C_{ds}A_{ds}GₖₛGₖₛAₖ | | | | | |
| 533440 | Gₑₛ^{m}Cₑₛ^{m}Cₑₛ^{m}C_{ds}A_{ds}^{m}C_{ds}A_{ds}G_{ds}^{m}C_{ds}^{m} | 26.9 | 931 | 946 | 357 | 1361 |
| | C_{ds}T_{ds}G_{ds}^{m}C_{ds}AₖₛGₖₛGₖ | | | | | |
| 533441 | ^{m}CₑₛGₑₛ^{m}Cₑₛ^{m}C_{ds}^{m}C_{ds}A_{ds}^{m}C_{ds}A_{ds}G_{ds}^{m} | 31.3 | 932 | 947 | 358 | 1362 |
| | C_{ds}^{m}C_{ds}T_{ds}G_{ds}^{m}CₖₛAₖₛGₖ | | | | | |
| 533442 | ^{m}Cₑₛ^{m}CₑₛGₑₛ^{m}C_{ds}^{m}C_{ds}^{m}C_{ds}A_{ds}^{m}C_{ds}A_{ds} | 20.5 | 933 | 948 | 359 | 1363 |
| | G_{ds}^{m}C_{ds}^{m}C_{ds}T_{ds}Gₖₛ^{m}CₖₛAₖ | | | | | |
| 533443 | Aₑₛ^{m}Cₑₛ^{m}CₑₛG_{ds}^{m}C_{ds}^{m}C_{ds}^{m}C_{ds}A_{ds}^{m}C_{ds} | 13.7 | 934 | 949 | 360 | 1364 |
| | A_{ds}G_{ds}^{m}C_{ds}^{m}C_{ds}TₖₛGₖₛ^{m}Cₖ | | | | | |
| 533444 | ^{m}CₑₛAₑₛ^{m}CₑₛC_{ds}G_{ds}^{m}C_{ds}^{m}C_{ds}^{m}C_{ds}A_{ds} | 29.4 | 935 | 950 | 361 | 1365 |
| | ^{m}C_{ds}A_{ds}G_{ds}^{m}C_{ds}^{m}CₖₛTₖₛGₖ | | | | | |
| 533445 | ^{m}Cₑₛ^{m}CₑₛAₑₛ^{m}C_{ds}^{m}C_{ds}G_{ds}^{m}C_{ds}^{m}C_{ds}^{m}C_{ds} | 32 | 936 | 951 | 362 | 1366 |
| | A_{ds}^{m}C_{ds}A_{ds}G_{ds}^{m}Cₖₛ^{m}CₖₛTₖ | | | | | |
| 533446 | ^{m}Cₑₛ^{m}Cₑₛ^{m}CₑₛA_{ds}^{m}C_{ds}C_{ds}^{m}G_{ds}^{m}C_{ds}^{m}C_{ds} | 8.3 | 937 | 952 | 363 | 1367 |
| | ^{m}C_{ds}A_{ds}^{m}C_{ds}A_{ds}Gₖₛ^{m}Cₖₛ^{m}Cₖ | | | | | |
| 533447 | Gₑₛ^{m}Cₑₛ^{m}Cₑₛ^{m}C_{ds}A_{ds}^{m}C_{ds}^{m}C_{ds}G_{ds}^{m}C_{ds} | 18.3 | 938 | 953 | 364 | 1368 |
| | ^{m}C_{ds}^{m}C_{ds}A_{ds}^{m}C_{ds}AₖₛGₖₛ^{m}Cₖ | | | | | |
| 533448 | ^{m}Cₑₛ^{m}CₑₛAₑₛG_{ds}G_{ds}^{m}C_{ds}^{m}C_{ds}^{m}C_{ds}A_{ds}^{m} | 19.4 | 942 | 957 | 365 | 1369 |
| | C_{ds}^{m}C_{ds}G_{ds}^{m}C_{ds}^{m}Cₖₛ^{m}CₖₛAₖ | | | | | |
| 533449 | ^{m}Cₑₛ^{m}Cₑₛ^{m}CₑₛA_{ds}G_{ds}G_{ds}^{m}C_{ds}^{m}C_{ds}^{m}C_{ds} | 24.2 | 943 | 958 | 366 | 1370 |
| | A_{ds}^{m}C_{ds}^{m}C_{ds}G_{ds}^{m}Cₖₛ^{m}Cₖₛ^{m}Cₖ | | | | | |
| 533450 | Tₑₛ^{m}Cₑₛ^{m}Cₑₛ^{m}C_{ds}A_{ds}G_{ds}G_{ds}^{m}C_{ds}^{m}C_{ds}^{m} | 39.2 | 944 | 959 | 367 | 1371 |
| | C_{ds}A_{ds}^{m}C_{ds}^{m}C_{ds}Gₖₛ^{m}Cₖₛ^{m}Cₖ | | | | | |
| 533451 | TₑₛGₑₛ^{m}Cₑₛ^{m}C_{ds}T_{ds}G_{ds}T_{ds}^{m}C_{ds}^{m}C_{ds}^{m}C_{ds} | 44.2 | 950 | 965 | 368 | 1372 |
| | A_{ds}G_{ds}G_{ds}^{m}Cₖₛ^{m}Cₖₛ^{m}Cₖ | | | | | |
| 533452 | ^{m}CₑₛTₑₛGₑₛ^{m}C_{ds}^{m}C_{ds}T_{ds}G_{ds}T_{ds}^{m}C_{ds}^{m}C_{ds} | 55.6 | 951 | 966 | 369 | 1373 |
| | ^{m}C_{ds}A_{ds}G_{ds}Gₖₛ^{m}Cₖₛ^{m}Cₖ | | | | | |
| 533453 | Gₑₛ^{m}CₑₛTₑₛG_{ds}C_{ds}^{m}C_{ds}T_{ds}G_{ds}T_{ds}^{m}C_{ds} | 71.2 | 952 | 967 | 370 | 1374 |
| | ^{m}C_{ds}A_{ds}GₖₛGₖₛ^{m}Cₖ | | | | | |
| 533454 | GₑₛGₑₛTₑₛG_{ds}G_{ds}^{m}C_{ds}A_{ds}^{m}C_{ds}^{m}C_{ds}T_{ds} | 39.6 | 1276 | 1291 | 371 | 1375 |
| | T_{ds}^{m}C_{ds}G_{ds}AₖₛAₖₛAₖ | | | | | |
| 533455 | ^{m}CₑₛGₑₛGₑₛT_{ds}G_{ds}G_{ds}^{m}C_{ds}A_{ds}^{m}C_{ds}^{m}C_{ds} | 52.9 | 1277 | 1292 | 372 | 1376 |
| | T_{ds}T_{ds}^{m}C_{ds}GₖₛAₖₛAₖ | | | | | |
| 533456 | Tₑₛ^{m}CₑₛGₑₛG_{ds}T_{ds}G_{ds}G_{ds}^{m}C_{ds}A_{ds}^{m}C_{ds} | 27 | 1278 | 1293 | 373 | 1377 |
| | ^{m}C_{ds}T_{ds}T_{ds}^{m}CₖₛGₖₛAₖ | | | | | |
| 533457 | AₑₛGₑₛTₑₛG_{ds}A_{ds}G_{ds}^{m}C_{ds}^{m}C_{ds}^{m}C_{ds}G_{ds} | 51.5 | 1315 | 1330 | 374 | 1378 |
| | T_{ds}^{m}C_{ds}C_{ds}Tₖₛ^{m}Cₖₛ^{m}Cₖ | | | | | |
| 533458 | ^{m}CₑₛAₑₛGₑₛT_{ds}G_{ds}A_{ds}G_{ds}^{m}C_{ds}^{m}C_{ds}^{m}C_{ds} | 55.1 | 1316 | 1331 | 375 | 1379 |
| | G_{ds}T_{ds}^{m}C_{ds}^{m}CₖₛTₖₛ^{m}Cₖ | | | | | |
| 533459 | Gₑₛ^{m}CₑₛAₑₛG_{ds}T_{ds}G_{ds}A_{ds}G_{ds}C_{ds}^{m}C_{ds} | 33.7 | 1317 | 1332 | 376 | 1380 |
| | ^{m}C_{ds}G_{ds}T_{ds}^{m}Cₖₛ^{m}CₖₛTₖ | | | | | |
| 533460 | Tₑₛ^{m}Cₑₛ^{m}Cₑₛ^{m}C_{ds}G_{ds}A_{ds}A_{ds}T_{ds}G_{ds}T_{ds} | 28.7 | 1344 | 1359 | 377 | 1381 |
| | C_{ds}^{m}C_{ds}G_{ds}Aₖₛ^{m}CₖₛAₖ | | | | | |
| 533461 | TₑₛTₑₛ^{m}Cₑₛ^{m}C_{ds}^{m}C_{ds}G_{ds}A_{ds}A_{ds}T_{ds}G_{ds} | 36.2 | 1345 | 1360 | 378 | 1382 |
| | T_{ds}^{m}C_{ds}^{m}C_{ds}GₖₛAₖₛ^{m}Cₖ | | | | | |
| 533462 | ^{m}CₑₛTₑₛTₑₛC_{ds}^{m}C_{ds}^{m}C_{ds}G_{ds}A_{ds}A_{ds}T_{ds} | 23 | 1346 | 1361 | 379 | 1383 |
| | G_{ds}T_{ds}^{m}C_{ds}CₖₛGₖₛAₖ | | | | | |
| 533463 | ^{m}Cₑₛ^{m}CₑₛTₑₛT_{ds}C_{ds}^{m}C_{ds}^{m}C_{ds}G_{ds}A_{ds}A_{ds} | 11.5 | 1347 | 1362 | 380 | 1384 |
| | T_{ds}G_{ds}T_{ds}^{m}CₖₛCₖₛGₖ | | | | | |
| 533464 | Aₑₛ^{m}Cₑₛ^{m}CₑₛT_{ds}T_{ds}C_{ds}^{m}C_{ds}^{m}C_{ds}G_{ds}A_{ds} | 19.9 | 1348 | 1363 | 381 | 1385 |
| | A_{ds}T_{ds}G_{ds}Tₖₛ^{m}Cₖₛ^{m}Cₖ | | | | | |
| 533465 | ^{m}CₑₛAₑₛ^{m}Cₑₛ^{m}C_{ds}T_{ds}T_{ds}C_{ds}^{m}C_{ds}^{m}C_{ds} | 30.2 | 1349 | 1364 | 382 | 1386 |
| | G_{ds}A_{ds}A_{ds}T_{ds}GₖₛTₖₛCₖ | | | | | |
| 533466 | Gₑₛ^{m}CₑₛAₑₛC_{ds}^{m}C_{ds}T_{ds}T_{ds}C_{ds}^{m}C_{ds}^{m} | 30.2 | 1350 | 1365 | 383 | 1387 |
| | C_{ds}G_{ds}A_{ds}A_{ds}TₖₛGₖₛTₖ | | | | | |
| 533467 | ^{m}CₑₛGₑₛ^{m}CₑₛA_{ds}^{m}C_{ds}C_{ds}T_{ds}T_{ds}^{m}C_{ds} | 35.5 | 1351 | 1366 | 384 | 1388 |
| | C_{ds}^{m}C_{ds}G_{ds}A_{ds}AₖₛTₖₛGₖ | | | | | |
| 533468 | AₑₛTₑₛ^{m}Cₑₛ^{m}C_{ds}G_{ds}^{m}C_{ds}T_{ds}^{m}C_{ds}^{m}C_{ds}T_{ds} | 47.4 | 1746 | 1761 | 385 | 1389 |
| | G_{ds}^{m}C_{ds}A_{ds}Aₖₛ^{m}CₖₛTₖ | | | | | |
| 533469 | ^{m}CₑₛAₑₛTₑₛ^{m}C_{ds}^{m}C_{ds}G_{ds}^{m}C_{ds}T_{ds}C_{ds}^{m} | 51.2 | 1747 | 1762 | 386 | 1390 |
| | C_{ds}T_{ds}G_{ds}^{m}C_{ds}AₖₛAₖₛ^{m}Cₖ | | | | | |
| 533470 | ^{m}Cₑₛ^{m}CₑₛAₑₛT_{ds}C_{ds}^{m}C_{ds}G_{ds}^{m}C_{ds}T_{ds}^{m} | 35.5 | 1748 | 1763 | 387 | 1391 |
| | C_{ds}^{m}C_{ds}T_{ds}G_{ds}CₖₛAₖₛAₖ | | | | | |
| 533471 | Gₑₛ^{m}CₑₛTₑₛC_{ds}^{m}C_{ds}^{m}C_{ds}T_{ds}^{m}C_{ds}T_{ds}G_{ds} | 65.6 | 1770 | 1785 | 388 | 1392 |
| | ^{m}C_{ds}^{m}C_{ds}T_{ds}Gₖₛ^{m}CₖₛAₖ | | | | | |
| 533472 | AₑₛGₑₛGₑₛT_{ds}G_{ds}G_{ds}A_{ds}T_{ds}^{m}C_{ds}^{m}C_{ds}G_{ds} | 51.8 | 1816 | 1831 | 389 | 1393 |
| | T_{ds}G_{ds}Gₖₛ^{m}Cₖₛ^{m}Cₖ | | | | | |
| 533473 | GₑₛGₑₛGₑₛA_{ds}A_{ds}G_{ds}G_{ds}T_{ds}G_{ds}G_{ds}A_{ds} | 44.9 | 1820 | 1835 | 390 | 1394 |
| | T_{ds}^{m}C_{ds}^{m}CₖₛGₖₛTₖ | | | | | |
| 533474 | Aₑₛ^{m}CₑₛAₑₛG_{ds}G_{ds}A_{ds}G_{ds}^{m}C_{ds}A_{ds}G_{ds} | 80.8 | 1955 | 1970 | 391 | 1395 |
| | G_{ds}G_{ds}A_{ds}AₖₛAₖₛGₖ | | | | | |
| 533475 | ^{m}CₑₛAₑₛGₑₛA_{ds}C_{ds}T_{ds}G_{ds}^{m}C_{ds}G_{ds}G_{ds} | 95.5 | 2034 | 2049 | 392 | 1396 |
| | T_{ds}G_{ds}A_{ds}GₖₛTₖₛTₖ | | | | | |
| 533476 | GₑₛGₑₛ^{m}CₑₛT_{ds}C_{ds}^{m}C_{ds}T_{ds}G_{ds}G_{ds}G_{ds} | 55.7 | 2050 | 2065 | 393 | 1397 |
| | ^{m}C_{ds}G_{ds}G_{ds}^{m}CₖₛGₖₛCₖ | | | | | |
| 533477 | GₑₛGₑₛ^{m}CₑₛG_{ds}G_{ds}^{m}C_{ds}T_{ds}^{m}C_{ds}^{m}C_{ds}T_{ds} | 45.8 | 2053 | 2068 | 394 | 1398 |
| | G_{ds}G_{ds}G_{ds}^{m}CₖₛGₖₛGₖ | | | | | |
| 533478 | ^{m}CₑₛGₑₛ^{m}CₑₛG_{ds}G_{ds}G_{ds}^{m}C_{ds}G_{ds}G_{ds}^{m}C_{ds} | 83.7 | 2057 | 2072 | 395 | 1399 |
| | T_{ds}^{m}C_{ds}^{m}C_{ds}TₖₛGₖₛGₖ | | | | | |
| 533479 | GₑₛAₑₛGₑₛC_{ds}G_{ds}^{m}C_{ds}G_{ds}G_{ds}G_{ds}^{m}C_{ds} | 79.8 | 2060 | 2075 | 396 | 1400 |
| | G_{ds}G_{ds}^{m}C_{ds}TₖₛCₖₛ^{m}Cₖ | | | | | |
| 533480 | GₑₛGₑₛTₑₛT_{ds}^{m}C_{ds}A_{ds}G_{ds}G_{ds}G_{ds}A_{ds}G_{ds} | 49.4 | 2068 | 2083 | 397 | 1401 |
| | ^{m}C_{ds}G_{ds}^{m}CₖₛGₖₛGₖ | | | | | |
| 533481 | AₑₛGₑₛTₑₛTas^{m}C_{ds}T_{ds}A_{ds}G_{ds}G_{ds}G_{ds}T_{ds} | 37 | 2076 | 2091 | 398 | 1402 |
| | T_{ds}^{m}C_{ds}AₖₛGₖₛGₖ | | | | | |
| 533482 | ^{m}CₑₛAₑₛGₑₛT_{ds}T_{ds}C_{ds}T_{ds}A_{ds}G_{ds}G_{ds}G_{ds} | 28.5 | 2077 | 2092 | 399 | 1403 |
| | T_{ds}T_{ds}^{m}CₖₛAₖₛGₖ | | | | | |
| 533483 | AₑₛCₑₛAₑₛG_{ds}T_{ds}T_{ds}^{m}C_{ds}T_{ds}A_{ds}G_{ds}G_{ds} | 42 | 2078 | 2093 | 400 | 1404 |
| | G_{ds}T_{ds}Tₖₛ^{m}CₖₛAₖ | | | | | |
| 533484 | GₑₛAₑₛ^{m}CₑₛA_{ds}G_{ds}T_{ds}T_{ds}^{m}C_{ds}T_{ds}A_{ds}G_{ds} | 37.4 | 2079 | 2094 | 401 | 1405 |
| | G_{ds}G_{ds}TₖₛTₖₛ^{m}Cₖ | | | | | |
| 533485 | AₑₛGₑₛAₑₛ^{m}C_{ds}A_{ds}G_{ds}T_{ds}T_{ds}^{m}C_{ds}T_{ds}A_{ds} | 66.5 | 2080 | 2095 | 402 | 1406 |
| | G_{ds}G_{ds}GₖₛTₖₛTₖ | | | | | |
| 533486 | AₑₛAₑₛGₑₛA_{ds}^{m}C_{ds}A_{ds}G_{ds}T_{ds}T_{ds}C_{ds}T_{ds} | 62.4 | 2081 | 2096 | 403 | 1407 |
| | A_{ds}G_{ds}GₖₛGₖₛTₖ | | | | | |
| 533487 | GₑₛAₑₛAₑₛG_{ds}A_{ds}^{m}C_{ds}A_{ds}G_{ds}T_{ds}T_{ds}^{m}C_{ds} | 56.9 | 2082 | 2097 | 404 | 1408 |
| | T_{ds}A_{ds}GₖₛGₖₛGₖ | | | | | |
| 533488 | ^{m}CₑₛGₑₛAₑₛA_{ds}G_{ds}A_{ds}^{m}C_{ds}A_{ds}G_{ds}T_{ds}T_{ds} | 36.8 | 2083 | 2098 | 405 | 1409 |
| | ^{m}C_{ds}T_{ds}AₖₛGₖₛGₖ | | | | | |
| 533489 | Tₑₛ^{m}CₑₛGₑₛA_{ds}A_{ds}G_{ds}A_{ds}^{m}C_{ds}A_{ds}G_{ds}T_{ds} | 49.6 | 2084 | 2099 | 406 | 1410 |
| | T_{ds}^{m}C_{ds}TₖₛAₖₛGₖ | | | | | |
| 533490 | GₑₛTₑₛCₑₛG_{ds}A_{ds}A_{ds}G_{ds}A_{ds}^{m}C_{ds}A_{ds} | 40.4 | 2085 | 2100 | 407 | 1411 |
| | G_{ds}T_{ds}T_{ds}^{m}CₖₛTₖₛAₖ | | | | | |
| 533491 | AₑₛGₑₛTₑₛ^{m}C_{ds}G_{ds}A_{ds}A_{ds}G_{ds}A_{ds}^{m}C_{ds} | 37.4 | 2086 | 2101 | 408 | 1412 |
| | A_{ds}G_{ds}T_{ds}Tₖₛ^{m}CₖₛTₖ | | | | | |
| 533492 | GₑₛAₑₛGₑₛT_{ds}^{m}C_{ds}G_{ds}A_{ds}A_{ds}G_{ds}A_{ds}^{m} | 36.6 | 2087 | 2102 | 409 | 1413 |
| | C_{ds}A_{ds}G_{ds}TₖₛTₖₛ^{m}Cₖ | | | | | |
| 533493 | GₑₛGₑₛAₑₛG_{ds}T_{ds}C_{ds}G_{ds}A_{ds}A_{ds}G_{ds}A_{ds} | 33.2 | 2088 | 2103 | 410 | 1414 |
| | ^{m}C_{ds}A_{ds}GₖₛTₖₛTₖ | | | | | |
| 533494 | ^{m}CₑₛGₑₛGₑₛA_{ds}G_{ds}T_{ds}^{m}C_{ds}G_{ds}A_{ds}A_{ds} | 45.3 | 2089 | 2104 | 411 | 1415 |
| | G_{ds}A_{ds}^{m}C_{ds}AₖₛGₖₛTₖ | | | | | |
| 533495 | ^{m}Cₑₛ^{m}CₑₛGₑₛG_{ds}A_{ds}G_{ds}T_{ds}^{m}C_{ds}G_{ds}A_{ds} | 45.9 | 2090 | 2105 | 412 | 1416 |
| | A_{ds}G_{ds}A_{ds}^{m}CₖₛAₖₛGₖ | | | | | |
| 533496 | ^{m}Cₑₛ^{m}Cₑₛ^{m}CₑₛG_{ds}G_{ds}A_{ds}G_{ds}T_{ds}^{m}C_{ds}G_{ds} | 51.3 | 2091 | 2106 | 413 | 1417 |
| | A_{ds}A_{ds}G_{ds}Aₖₛ^{m}CₖₛAₖ | | | | | |
| 533497 | ^{m}Cₑₛ^{m}Cₑₛ^{m}Cₑₛ^{m}C_{ds}G_{ds}G_{ds}A_{ds}G_{ds}T_{ds}^{m} | 49.2 | 2092 | 2107 | 414 | 1418 |
| | C_{ds}G_{ds}A_{ds}A_{ds}GₖₛAₖₛ^{m}Cₖ | | | | | |
| 533498 | Gₑₛ^{m}Cₑₛ^{m}Cₑₛ^{m}C_{ds}^{m}C_{ds}G_{ds}G_{ds}A_{ds}G_{ds}T_{ds} | 52.3 | 2093 | 2108 | 415 | 1419 |
| | ^{m}C_{ds}G_{ds}A_{ds}AₖₛGₖₛAₖ | | | | | |
| 533499 | GₑₛGₑₛ^{m}Cₑₛ^{m}C_{ds}^{m}C_{ds}^{m}C_{ds}G_{ds}G_{ds}A_{ds}G_{ds} | 54.9 | 2094 | 2109 | 416 | 1420 |
| | T_{ds}^{m}C_{ds}G_{ds}AₖₛAₖₛGₖ | | | | | |
| 533500 | GₑₛGₑₛGₑₛ^{m}C_{ds}^{m}C_{ds}^{m}C_{ds}^{m}C_{ds}G_{ds}G_{ds}A_{ds} | 46.7 | 2095 | 2110 | 417 | 1421 |
| | G_{ds}T_{ds}^{m}C_{ds}GₖₛAₖₛAₖ | | | | | |
| 533809 | Aₑₛ^{m}CₑₛAₑₛA_{ds}T_{ds}A_{ds}A_{ds}A_{ds}T_{ds}A_{ds}^{m}C_{ds} | 51.4 | 2773 | 2788 | 418 | 1422 |
| | ^{m}C_{ds}G_{ds}AₖₛGₖₛGₖ | | | | | |

### Example 4: Design of Antisense Oligonucleotides Targeting Human Dystrophia Myotonica Protein Kinase (hDMPK)

### Dose Response HepG2

A series of antisense oligonucleotides (ASOs) were designed to target hDMPK The newly designed ASOs were prepared using standard oligonucleotide synthesis well known in the art and are described in Table 8, below. Subscripts “s” indicate phosphorothioate internucleoside linkages; subscripts “k” indicate 6′-(S)—CH₃ bicyclic nucleosides (cEt); subscripts “e” indicate 2′-O-methoxyethyl (MOE) modified nucleosides; and subscripts “d” indicate β-D-2′-deoxyribonucleosides. “c^{m}C” indicates 5-methylcytosine nucleosides.

The antisense oligonucleotides are targeted to SEQ ID NO: 1 (GENBANK Accession No. NM_001081560.1). “Start site” indicates the 5′-most nucleoside to which the gapmer is targeted in the human gene sequence. “Stop site” indicates the 3′-most nucleoside to which the gapmer is targeted human gene sequence.

TABLE 8
Design of antisense oligonucleotides targeting hDMPK

| | | | | Un- | |
|---|---|---|---|---|---|
| | | | | modified | Modified |
| ISIS | | Start | Stop | SEQ ID | SEQ ID |
| No. | Composition (5′ to 3′) | Site | Site | NO | NO |
| 533440 | Gₑₛ^{m}Cₑₛ^{m}Cₑₛ^{m}C_{ds}A_{ds}^{m}C_{ds}A_{ds}G_{ds}^{m}C_{ds}^{m}C_{ds}T_{ds}G_{ds}^{m}C_{ds} | 931 | 946 | 357 | 1361 |
| | AₖₛGₖₛGₖ | | | | |
| 533442 | ^{m}Cₑₛ^{m}CₑₛGₑₛ^{m}C_{ds}^{m}C_{ds}^{m}C_{ds}A_{ds}^{m}C_{ds}A_{ds}G_{ds}^{m}C_{ds}^{m}C_{ds}T_{ds} | 933 | 948 | 359 | 1363 |
| | Gₖₛ^{m}CₖₛAₖ | | | | |
| 533443 | Aₑₛ^{m}Cₑₛ^{m}CₑₛG_{ds}^{m}C_{ds}^{m}C_{ds}^{m}C_{ds}A_{ds}^{m}C_{ds}A_{ds}G_{ds}^{m}C_{ds}^{m} | 934 | 949 | 360 | 1364 |
| | C_{ds}TₖₛGₖₛ^{m}Cₖ | | | | |
| 533446 | ^{m}Cₑₛ^{m}Cₑₛ^{m}CₑₛA_{ds}^{m}C_{ds}^{m}C_{ds}G_{ds}^{m}C_{ds}^{m}C_{ds}^{m}C_{ds}A_{ds}^{m}C_{ds} | 937 | 952 | 363 | 1367 |
| | A_{ds}Gₖₛ^{m}Cₖₛ^{m}Cₖ | | | | |
| 533447 | Gₑₛ^{m}Cₑₛ^{m}Cₑₛ^{m}C_{ds}A_{ds}^{m}C_{ds}^{m}C_{ds}G_{ds}^{m}C_{ds}^{m}C_{ds}^{m}C_{ds}A_{ds}^{m} | 938 | 953 | 364 | 1368 |
| | C_{ds}AₖₛGₖₛ^{m}Cₖ | | | | |
| 533448 | ^{m}Cₑₛ^{m}CₑₛAₑₛG_{ds}G_{ds}^{m}C_{ds}^{m}C_{ds}^{m}C_{ds}A_{ds}^{m}C_{ds}^{m}C_{ds}G_{ds}^{m} | 942 | 957 | 365 | 1369 |
| | C_{ds}^{m}Cₖₛ^{m}CₖₛAₖ | | | | |
| 533449 | ^{m}Cₑₛ^{m}Cₑₛ^{m}CₑₛA_{ds}G_{ds}G_{ds}^{m}C_{ds}^{m}C_{ds}^{m}C_{ds}A_{ds}^{m}C_{ds}^{m}C_{ds} | 943 | 958 | 366 | 1370 |
| | G_{ds}^{m}Cₖₛ^{m}Cₖₛ^{m}Cₖ | | | | |
| 533462 | ^{m}CₑₛTₑₛTₑₛ^{m}C_{ds}^{m}C_{ds}^{m}C_{ds}G_{ds}A_{ds}A_{ds}T_{ds}G_{ds}T_{ds}^{m}C_{ds}^{m} | 1346 | 1361 | 379 | 1383 |
| | CₖₛGₖₛAₖ | | | | |
| 533463 | ^{m}Cₑₛ^{m}CₑₛTₑₛT_{ds}^{m}C_{ds}^{m}C_{ds}^{m}C_{ds}G_{ds}A_{ds}A_{ds}T_{ds}G_{ds}T_{ds}^{m} | 1347 | 1362 | 380 | 1384 |
| | Cₖₛ^{m}CₖₛGₖ | | | | |
| 533464 | Aₑₛ^{m}Cₑₛ^{m}CₑₛT_{ds}T_{ds}^{m}C_{ds}^{m}C_{ds}^{m}C_{ds}G_{ds}A_{ds}A_{ds}T_{ds}G_{ds}Tₖₛ | 1348 | 1363 | 381 | 1385 |
| | ^{m}Cₖₛ^{m}Cₖ | | | | |
| 533529 | ^{m}CₑₛGₑₛGₑₛT_{ds}T_{ds}G_{ds}T_{ds}G_{ds}A_{ds}A_{ds}^{m}C_{ds}T_{ds}G_{ds}Gₖₛ^{m} | 2162 | 2177 | 23 | 1423 |
| | CₖₛAₖ | | | | |
| 533530 | AₑₛGₑₛ^{m}CₑₛG_{ds}G_{ds}T_{ds}T_{ds}G_{ds}T_{ds}G_{ds}A_{ds}A_{ds}^{m}C_{ds}TₖₛGₖₛ | 2164 | 2179 | 419 | 1424 |
| | Gₖ | | | | |
| 533599 | Gₑₛ^{m}CₑₛAₑₛ^{m}C_{ds}T_{ds}T_{ds}T_{ds}G_{ds}^{m}C_{ds}G_{ds}A_{ds}A_{ds}^{m}C_{ds}^{m}Cₖₛ | 2492 | 2507 | 420 | 1425 |
| | AₖₛAₖ | | | | |
| 533600 | TₑₛGₑₛ^{m}CₑₛA_{ds}^{m}C_{ds}T_{ds}T_{ds}T_{ds}G_{ds}^{m}C_{ds}G_{ds}A_{ds}A_{ds}^{m}Cₖₛ | 2493 | 2508 | 421 | 1426 |
| | CₖₛAₖ | | | | |

### Example 5: Dose Response HepG2

Antisense oligonucleotides targeted to a human DMPK nucleic acid were tested for their effect on human DMPK RNA transcript in vitro. Cultured HepG2 cells at a density of 20,000 cells per well were transfected using electroporation with 625 nM, 1250 nM, 2500 nM, 5000 nM, and 10000.0 nM concentrations of each antisense oligonucleotide. After approximately 24 hours, RNA was isolated from the cells and DMPK RNA transcript levels were measured by quantitative real-time PCR using primer probe set RTS3164 (forward sequence AGCCTGAGCCGGGAGATG, designated herein as SEQ ID NO: 20; reverse sequence GCGTAGTTGACTGGCGAAGTT, designated herein as SEQ ID NO: 21; probe sequence AGGCCATCCGCACGGACAACCX, designated herein as SEQ ID NO: 22). Human DMPK RNA transcript levels were adjusted according to total RNA content, as measured by RIBOGREEN®. Results are presented in the table below as percent expression of human DMPK, relative to untreated control (UTC) cells. The tested antisense oligonucleotide sequences demonstrated dose-dependent inhibition of human DMPK mRNA levels under the conditions specified above.

TABLE 9
Inhibition of human DMPK RNA transcript
in HepG2 cells targeting SEQ ID NO: 1

| ISIS | Dose | % Target | Start Site | Stop Site |
|---|---|---|---|---|
| No. | (nM) | Expression | on Seq ID: 1 | on Seq ID: 1 |
| UTC | N/A | 100 | N/A | N/A |
| 486178 | 625.0 | 39.4 | 2773 | 2788 |
| 486178 | 1250.0 | 31.2 | 2773 | 2788 |
| 486178 | 2500.0 | 20.6 | 2773 | 2788 |
| 486178 | 5000.0 | 13 | 2773 | 2788 |
| 486178 | 10000.0 | 11.5 | 2773 | 2788 |
| 533440 | 625.0 | 55.4 | 931 | 946 |
| 533440 | 1250.0 | 40.4 | 931 | 946 |
| 533440 | 2500.0 | 25.4 | 931 | 946 |
| 533440 | 5000.0 | 22.6 | 931 | 946 |
| 533440 | 10000.0 | 10.3 | 931 | 946 |
| 533442 | 625.0 | 55.2 | 933 | 948 |
| 533442 | 1250.0 | 33.1 | 933 | 948 |
| 533442 | 2500.0 | 29 | 933 | 948 |
| 533442 | 5000.0 | 16.9 | 933 | 948 |
| 533442 | 10000.0 | 7.2 | 933 | 948 |
| 533443 | 625.0 | 44.8 | 934 | 949 |
| 533443 | 1250.0 | 29.4 | 934 | 949 |
| 533443 | 2500.0 | 19.9 | 934 | 949 |
| 533443 | 5000.0 | 10.8 | 934 | 949 |
| 533443 | 10000.0 | 7 | 934 | 949 |
| 533446 | 625.0 | 50.9 | 937 | 952 |
| 533446 | 1250.0 | 35.5 | 937 | 952 |
| 533446 | 2500.0 | 30.4 | 937 | 952 |
| 533446 | 5000.0 | 14.6 | 937 | 952 |
| 533446 | 10000.0 | 14 | 937 | 952 |
| 533447 | 625.0 | 53.3 | 938 | 953 |
| 533447 | 1250.0 | 31.7 | 938 | 953 |
| 533447 | 2500.0 | 16.8 | 938 | 953 |
| 533447 | 5000.0 | 11.7 | 938 | 953 |
| 533447 | 10000.0 | 4.4 | 938 | 953 |
| 533448 | 625.0 | 58.8 | 942 | 957 |
| 533448 | 1250.0 | 36.9 | 942 | 957 |
| 533448 | 2500.0 | 24.8 | 942 | 957 |
| 533448 | 5000.0 | 11.5 | 942 | 957 |
| 533448 | 10000.0 | 10.1 | 942 | 957 |
| 533449 | 625.0 | 61.1 | 943 | 958 |
| 533449 | 1250.0 | 42.8 | 943 | 958 |
| 533449 | 2500.0 | 30.4 | 943 | 958 |
| 533449 | 5000.0 | 20.2 | 943 | 958 |
| 533449 | 10000.0 | 10.1 | 943 | 958 |
| 533462 | 625.0 | 50.7 | 1346 | 1361 |
| 533462 | 1250.0 | 32.3 | 1346 | 1361 |
| 533462 | 2500.0 | 29.2 | 1346 | 1361 |
| 533462 | 5000.0 | 12.5 | 1346 | 1361 |
| 533462 | 10000.0 | 5.8 | 1346 | 1361 |
| 533463 | 625.0 | 39.1 | 1347 | 1362 |
| 533463 | 1250.0 | 23.7 | 1347 | 1362 |
| 533463 | 2500.0 | 12.6 | 1347 | 1362 |
| 533463 | 5000.0 | 9.3 | 1347 | 1362 |
| 533463 | 10000.0 | 3.2 | 1347 | 1362 |
| 533464 | 625.0 | 48.8 | 1348 | 1363 |
| 533464 | 1250.0 | 36.4 | 1348 | 1363 |
| 533464 | 2500.0 | 24.5 | 1348 | 1363 |
| 533464 | 5000.0 | 11.7 | 1348 | 1363 |
| 533464 | 10000.0 | 5 | 1348 | 1363 |
| 533529 | 625.0 | 35.8 | 2162 | 2177 |
| 533529 | 1250.0 | 26.4 | 2162 | 2177 |
| 533529 | 2500.0 | 18.3 | 2162 | 2177 |
| 533529 | 5000.0 | 14.8 | 2162 | 2177 |
| 533529 | 10000.0 | 14.7 | 2162 | 2177 |
| 533530 | 625.0 | 47.4 | 2164 | 2179 |
| 533530 | 1250.0 | 22.1 | 2164 | 2179 |
| 533530 | 2500.0 | 21.5 | 2164 | 2179 |
| 533530 | 5000.0 | 14.4 | 2164 | 2179 |
| 533530 | 10000.0 | 8 | 2164 | 2179 |
| 533599 | 625.0 | 31.3 | 2492 | 2507 |
| 533599 | 1250.0 | 21.9 | 2492 | 2507 |
| 533599 | 2500.0 | 13.1 | 2492 | 2507 |
| 533599 | 5000.0 | 8.8 | 2492 | 2507 |
| 533599 | 10000.0 | 7.3 | 2492 | 2507 |
| 533600 | 625.0 | 33.8 | 2493 | 2508 |
| 533600 | 1250.0 | 20.9 | 2493 | 2508 |
| 533600 | 2500.0 | 16.5 | 2493 | 2508 |
| 533600 | 5000.0 | 10.4 | 2493 | 2508 |
| 533600 | 10000.0 | 12.1 | 2493 | 2508 |

### Example 6: Design of Antisense Oligonucleotides Targeting Human Dystrophia Myotonica Protein Kinase (hDMPK)

A series of antisense oligonucleotides (ASOs) were designed to target hDMPK. The newly designed ASOs were prepared using standard oligonucleotide synthesis well known in the art and are described in Table 10, below. Subscripts “s” indicate phosphorothioate intemnucleoside linkages; subscripts “k” indicate 6′-(S)—CH₃ bicyclic nucleosides (cEt); subscripts “e” indicate 2′-O-methoxyethyl (MOE) modified nucleosides; and subscripts “d” indicate β-D-2′-deoxyribonucleosides. ^{m}C indicates 5-methylcytosine nucleosides.

The antisense oligonucleotides are targeted to SEQ ID NO: 2 (the complement of GENBANK Accession No. NT_011109.15 truncated from nucleotides 18540696 to 18555106). “Start site” indicates the 5′-most nucleoside to which the gapmer is targeted in the human gene sequence. “Stop site” indicates the 3′-most nucleoside to which the gapmer is targeted human gene sequence.

TABLE 10
Design of antisense oligonucleotides targeting hDMPK

| | | Start | Stop | Un- | |
|---|---|---|---|---|---|
| | | Site on | Site on | modified | Modified |
| ISIS | | Seq | Seq | Seq | Seq ID |
| No. | Sequence | ID: 2 | ID: 2 | ID No. | No. |
| UTC | N/A | N/A | N/A | | |
| 486178 | Aₖₛ^{m}CₖₛAₖₛA_{ds}T_{ds}A_{ds}A_{ds}A_{ds}T_{ds}A_{ds}^{m}C_{ds}^{m}C_{ds}G_{ds}AₖₛGₖₛ | 13836 | 13851 | 23 | 881 |
| | Gₖ | | | | |
| 533597 | Aₑₛ^{m}CₑₛTₑₛT_{ds}T_{ds}G_{ds}^{m}C_{ds}G_{ds}A_{ds}A_{ds}^{m}C_{ds}^{m}C_{ds}A_{ds}Aₖₛ^{m}Cₖₛ | 13553 | 13568 | 422 | 1427 |
| | Gₖ | | | | |
| 533603 | AₑₛAₑₛAₑₛG_{ds}^{m}C_{ds}T_{ds}T_{ds}T_{ds}G_{ds}^{m}C_{ds}A_{ds}^{m}C_{ds}T_{ds}TₖₛTₖₛ | 13563 | 13578 | 423 | 1428 |
| | Gₖ | | | | |
| 533617 | Tₑₛ^{m}Cₑₛ^{m}Cₑₛ^{m}C_{ds}G_{ds}A_{ds}G_{ds}T_{ds}A_{ds}A_{ds}G_{ds}^{m}C_{ds}A_{ds}GₖₛGₖₛ | 13624 | 13639 | 424 | 1429 |
| | ^{m}Cₖ | | | | |
| 533649 | Gₑₛ^{m}CₑₛAₑₛG_{ds}^{m}C_{ds}G_{ds}^{m}C_{ds}A_{ds}A_{ds}G_{ds}T_{ds}G_{ds}A_{ds}GₖₛGₖₛ | 13686 | 13701 | 425 | 1430 |
| | Aₖ | | | | |
| 533694 | GₑₛTₑₛ^{m}CₑₛA_{ds}G_{ds}^{m}C_{ds}G_{ds}A_{ds}G_{ds}T_{ds}^{m}C_{ds}G_{ds}G_{ds}AₖₛGₖₛ | 13760 | 13775 | 426 | 1431 |
| | Gₖ | | | | |
| 533697 | ^{m}CₑₛCₑₛTₑₛG_{ds}T_{ds}^{m}C_{ds}A_{ds}G_{ds}^{m}C_{ds}G_{ds}A_{ds}G_{ds}T_{ds}^{m}CₖₛGₖₛ | 13763 | 13778 | 427 | 1432 |
| | Gₖ | | | | |
| 533698 | Gₑₛ^{m}Cₑₛ^{m}CₑₛT_{ds}G_{ds}T_{ds}^{m}C_{ds}A_{ds}G_{ds}^{m}C_{ds}G_{ds}A_{ds}G_{ds}Tₖₛ^{m}Cₖₛ | 13764 | 13779 | 428 | 1433 |
| | Gₖ | | | | |
| 533699 | AₑₛGₑₛ^{m}Cₑₛ^{m}C_{ds}T_{ds}G_{ds}T_{ds}^{m}C_{ds}A_{ds}G_{ds}^{m}C_{ds}G_{ds}A_{ds}GₖₛTₖₛ | 13765 | 13780 | 429 | 1434 |
| | ^{m}Cₖ | | | | |
| 533711 | GₑₛGₑₛGₑₛT_{ds}^{m}C_{ds}T_{ds}^{m}C_{ds}A_{ds}G_{ds}T_{ds}G_{ds}^{m}C_{ds}A_{ds}Tₖₛ^{m}Cₖₛ | 13813 | 13828 | 430 | 1435 |
| | ^{m}Cₖ | | | | |
| 533721 | AₑₛGₑₛGₑₛT_{ds}T_{ds}T_{ds}T_{ds}T_{ds}^{m}C_{ds}^{m}C_{ds}A_{ds}G_{ds}A_{ds}GₖₛGₖₛ^{m} | 2580 | 2595 | 431 | 1436 |
| | Cₖ | | | | |
| 533722 | AₑₛAₑₛGₑₛG_{ds}T_{ds}T_{ds}T_{ds}T_{ds}T_{ds}^{m}C_{ds}^{m}C_{ds}A_{ds}G_{ds}AₖₛGₖₛGₖ | 2581 | 2596 | 432 | 1437 |
| 533751 | GₑₛGₑₛTₑₛ^{m}C_{ds}A_{ds}^{m}C_{ds}T_{ds}G_{ds}^{m}C_{ds}T_{ds}G_{ds}G_{ds}G_{ds}Tₖₛ^{m}Cₖₛ | 6446 | 6461 | 433 | 1438 |
| | ^{m}Cₖ | | | | |
| 533786 | GₑₛTₑₛGₑₛG_{ds}T_{ds}T_{ds}T_{ds}^{m}C_{ds}T_{ds}G_{ds}T_{ds}C_{ds}T_{ds}Gₖₛ^{m}CₖₛTₖ | 11099 | 11114 | 434 | 1439 |
| 533787 | ^{m}CₑₛGₑₛTₑₛG_{ds}G_{ds}T_{ds}T_{ds}T_{ds}^{m}C_{ds}T_{ds}G_{ds}T_{ds}^{m}C_{ds}TₖₛGₖₛ^{m} | 11100 | 11115 | 435 | 1440 |
| | Cₖ | | | | |

### Example 7: Dose Response for ASOs Targeted to a Human DMPK RNA Transcript in HepG2 Cells

Antisense oligonucleotides targeted to a human DMPK nucleic acid were tested for their effect on human DMPK RNA transcript in vitro. Cultured HepG2 cells at a density of 20,000 cells per well were transfected using electroporation with 625 nM, 1250 nM, 2500 nM, 5000 nM, and 10000.0 nM concentrations of each antisense oligonucleotide. After approximately 24 hours, RNA was isolated from the cells and DMPK RNA transcript levels were measured by quantitative real-time PCR using primer probe set RTS3164 (forward sequence AGCCTGAGCCGGGAGATG, designated herein as SEQ ID NO: 20; reverse sequence GCGTAGTTGACTGGCGAAGTT, designated herein as SEQ ID NO: 21; probe sequence AGGCCATCCGCACGGACAACCX, designated herein as SEQ ID NO: 22). Human DMPK RNA transcript levels were adjusted according to total RNA content, as measured by RIBOGREEN®. Results are presented as percent expression of human DMPK, relative to untreated control (UTC) cells and are shown in the table below. The tested antisense oligonucleotide sequences demonstrated dose-dependent inhibition of human DMPK mRNA levels under the conditions specified above.

TABLE 11
Inhibition of human DMPK RNA transcript
in HepG2 cells targeting SEQ ID NO: 1

| ISIS | Dose | % Target | Start Site | Stop Site |
|---|---|---|---|---|
| No. | (nM) | Expression | on Seq ID: 2 | on Seq ID: 2 |
| UTC | NA | 100 | N/A | N/A |
| 486178 | 625.000 | 39.4 | 13836 | 13851 |
| 486178 | 1250.000 | 27.3 | 13836 | 13851 |
| 486178 | 2500.000 | 14 | 13836 | 13851 |
| 486178 | 5000.000 | 16.3 | 13836 | 13851 |
| 486178 | 10000.000 | 8.3 | 13836 | 13851 |
| 533597 | 625.000 | 42.4 | 13553 | 13568 |
| 533597 | 1250.000 | 30.3 | 13553 | 13568 |
| 533597 | 2500.000 | 15.3 | 13553 | 13568 |
| 533597 | 5000.000 | 10 | 13553 | 13568 |
| 533597 | 10000.000 | 10.6 | 13553 | 13568 |
| 533603 | 625.000 | 48.2 | 13563 | 13578 |
| 533603 | 1250.000 | 31.1 | 13563 | 13578 |
| 533603 | 2500.000 | 22.4 | 13563 | 13578 |
| 533603 | 5000.000 | 15.6 | 13563 | 13578 |
| 533603 | 10000.000 | 9.9 | 13563 | 13578 |
| 533617 | 625.000 | 38.4 | 13624 | 13639 |
| 533617 | 1250.000 | 26.3 | 13624 | 13639 |
| 533617 | 2500.000 | 21.6 | 13624 | 13639 |
| 533617 | 5000.000 | 15.8 | 13624 | 13639 |
| 533617 | 10000.000 | 14.6 | 13624 | 13639 |
| 533649 | 625.000 | 52.2 | 13686 | 13701 |
| 533649 | 1250.000 | 27.8 | 13686 | 13701 |
| 533649 | 2500.000 | 24.6 | 13686 | 13701 |
| 533649 | 5000.000 | 20.5 | 13686 | 13701 |
| 533649 | 10000.000 | 14.5 | 13686 | 13701 |
| 533694 | 625.000 | 53.3 | 13760 | 13775 |
| 533694 | 1250.000 | 29.4 | 13760 | 13775 |
| 533694 | 2500.000 | 23.6 | 13760 | 13775 |
| 533694 | 5000.000 | 18.7 | 13760 | 13775 |
| 533694 | 10000.000 | 13.5 | 13760 | 13775 |
| 533697 | 625.000 | 30.6 | 13763 | 13778 |
| 533697 | 1250.000 | 14.9 | 13763 | 13778 |
| 533697 | 2500.000 | 13.8 | 13763 | 13778 |
| 533697 | 5000.000 | 9.7 | 13763 | 13778 |
| 533697 | 10000.000 | 7.1 | 13763 | 13778 |
| 533698 | 625.000 | 23.4 | 13764 | 13779 |
| 533698 | 1250.000 | 15.5 | 13764 | 13779 |
| 533698 | 2500.000 | 13.8 | 13764 | 13779 |
| 533698 | 5000.000 | 12.4 | 13764 | 13779 |
| 533698 | 10000.000 | 10.2 | 13764 | 13779 |
| 533699 | 625.000 | 38.2 | 13765 | 13780 |
| 533699 | 1250.000 | 26.9 | 13765 | 13780 |
| 533699 | 2500.000 | 17.6 | 13765 | 13780 |
| 533699 | 5000.000 | 12.9 | 13765 | 13780 |
| 533699 | 10000.000 | 9.3 | 13765 | 13780 |
| 533711 | 625.000 | 35.1 | 13813 | 13828 |
| 533711 | 1250.000 | 34.6 | 13813 | 13828 |
| 533711 | 2500.000 | 22.4 | 13813 | 13828 |
| 533711 | 5000.000 | 22 | 13813 | 13828 |
| 533711 | 10000.000 | 13 | 13813 | 13828 |
| 533721 | 625.000 | 36.3 | 2580 | 2595 |
| 533721 | 1250.000 | 29.8 | 2580 | 2595 |
| 533721 | 2500.000 | 23.2 | 2580 | 2595 |
| 533721 | 5000.000 | 17.8 | 2580 | 2595 |
| 533721 | 10000.000 | 17.2 | 2580 | 2595 |
| 533722 | 625.000 | 48.5 | 2581 | 2596 |
| 533722 | 1250.000 | 28.6 | 2581 | 2596 |
| 533722 | 2500.000 | 21.9 | 2581 | 2596 |
| 533722 | 5000.000 | 28.1 | 2581 | 2596 |
| 533722 | 10000.000 | 13.8 | 2581 | 2596 |
| 533751 | 625.000 | 37.7 | 6446 | 6461 |
| 533751 | 1250.000 | 21.6 | 6446 | 6461 |
| 533751 | 2500.000 | 12.6 | 6446 | 6461 |
| 533751 | 5000.000 | 9.7 | 6446 | 6461 |
| 533751 | 10000.000 | 8.5 | 6446 | 6461 |
| 533786 | 625.000 | 53.6 | 11099 | 11114 |
| 533786 | 1250.000 | 26.6 | 11099 | 11114 |
| 533786 | 2500.000 | 14.7 | 11099 | 11114 |
| 533786 | 5000.000 | 9.6 | 11099 | 11114 |
| 533786 | 10000.000 | 5.5 | 11099 | 11114 |
| 533787 | 625.000 | 43.8 | 11100 | 11115 |
| 533787 | 1250.000 | 27.7 | 11100 | 11115 |
| 533787 | 2500.000 | 16.3 | 11100 | 11115 |
| 533787 | 5000.000 | 7 | 11100 | 11115 |
| 533787 | 10000.000 | 4.5 | 11100 | 11115 |

### Example 8: ASOs Designed to Target a Human DMPK RNA Transcript

A series of antisense oligonucleotides (ASOs) were designed to target hDMPK. The newly designed ASOs were prepared using standard oligonucleotide synthesis well known in the art and are described in Table 12, below. Subscripts “s” indicate phosphorothioate internucleoside linkages; subscripts “k” indicate 6′-(S)—CH₃ bicyclic nucleosides (cEt); subscripts “e” indicate 2′-methoxyethyl (MOE) modified nucleosides; and subscripts “d” indicate β-D-2′-deoxyribonucleosides. “^{m}C” indicates 5-methylcytosine nucleosides.

The antisense oligonucleotides targeted to a human DMPK nucleic acid were tested for their effect on DMPK RNA transcript in vitro. Cultured hSKMC cells at a density of 20,000 cells per well were transfected using electroporation with 800 nM antisense oligonucleotide. After approximately 24 hours, RNA was isolated from the cells and DMPK transcript levels were measured by quantitative real-time PCR. DMPK RNA transcript levels were adjusted according to total RNA content, as measured by RIBOGREEN®. Results are presented as percent expression of DMPK, relative to untreated control cells.

‘Target start site’ indicates the 5′-most nucleoside to which the antisense oligonucleotide is targeted in the human genomic gene sequence. ‘Target stop site’ indicates the 3′-most nucleoside to which the antisense oligonucleotide is targeted in the human genomic sequence. All the antisense oligonucleotides listed in Table 12 target SEQ ID NO: 1 (GENBANK Accession No. NM_001081560.1).

Several of the antisense oligonucleotides demonstrated significant inhibition of DMPK mRNA levels under the conditions specified above.

TABLE 12
Inhibition of human DMPK RNA transcript in HepG2 cells using ASOs targeting SEQ ID NO: 1

| | | | Start | Stop | | |
|---|---|---|---|---|---|---|
| | | | Site | Site | | |
| | | | on | on | Unmodified | Modified |
| | | % Target | Seq | Seq | Seq ID | Seq ID |
| ISIS No. | Sequence | Expression | ID: 1 | ID: 1 | No. | No. |
| UTC | N/A | 100 | N/A | N/A | | |
| 444401 | TₑₛTₑₛGₑₛ^{m}CₑₛAₑₛ^{m}C_{ds}T_{ds}T_{ds}T_{ds}G_{ds}^{m}C_{ds}G_{ds}A_{ds}A_{ds} | 25.2 | 2490 | 2509 | 33 | 1445 |
| | ^{m}C_{ds}^{m}CₑₛAₑₛAₑₛ^{m}CₑₛGₑ | | | | | |
| 444436 | GₑₛTₑₛ^{m}CₑₛGₑₛGₑₛA_{ds}G_{ds}G_{ds}A_{ds}^{m}C_{ds}G_{ds}A_{ds}G_{ds}G_{ds} | 30.8 | 2685 | 2704 | 264 | 1446 |
| | T_{ds}^{m}CₑₛAₑₛAₑₛTₑₛAₑ | | | | | |
| 486072 | AₖₛAₖₛGₖₛA_{ds}^{m}C_{ds}A_{ds}G_{ds}T_{ds}T_{ds}^{m}C_{ds}T_{ds}A_{ds}G_{ds}Gₖₛ | 36.8 | 2081 | 2096 | 403 | 884 |
| | GₖₛTₖ | | | | | |
| 486073 | ^{m}CₖₛGₖₛAₖₛA_{ds}G_{ds}A_{ds}^{m}C_{ds}A_{ds}G_{ds}T_{ds}T_{ds}^{m}C_{ds}T_{ds}Aₖₛ | 22.4 | 2083 | 2098 | 405 | 885 |
| | GₖₛGₖ | | | | | |
| 486075 | GₖₛTₖₛ^{m}CₖₛG_{ds}A_{ds}A_{ds}G_{ds}A_{ds}^{m}C_{ds}A_{ds}G_{ds}T_{ds}T_{ds}^{m}Cₖₛ | 41.3 | 2085 | 2100 | 407 | 886 |
| | TₖₛAₖ | | | | | |
| 486076 | AₖₛGₖₛTₖₛ^{m}C_{ds}G_{ds}A_{ds}A_{ds}G_{ds}A_{ds}^{m}C_{ds}A_{ds}G_{ds}T_{ds}Tₖₛ | 22.4 | 2086 | 2101 | 408 | 887 |
| | ^{m}CₖₛTₖ | | | | | |
| 486077 | GₖₛAₖₛGₖₛT_{ds}^{m}C_{ds}G_{ds}A_{ds}A_{ds}G_{ds}A_{ds}^{m}C_{ds}A_{ds}G_{ds}Tₖₛ | 35.2 | 2087 | 2102 | 409 | 888 |
| | Tₖₛ^{m}Cₖ | | | | | |
| 486078 | ^{m}CₖₛGₖₛGₖₛA_{ds}G_{ds}T_{ds}^{m}C_{ds}G_{ds}A_{ds}A_{ds}G_{ds}A_{ds}^{m}C_{ds}Aₖₛ | 12.4 | 2089 | 2104 | 411 | 889 |
| | GₖₛTₖ | | | | | |
| 486079 | ^{m}Cₖₛ^{m}Cₖₛ^{m}CₖₛG_{ds}G_{ds}A_{ds}G_{ds}T_{ds}^{m}C_{ds}G_{ds}A_{ds}A_{ds}G_{ds} | 36.5 | 2091 | 2106 | 413 | 890 |
| | Aₖₛ^{m}CₖₛAₖ | | | | | |
| 486080 | ^{m}Cₖₛ^{m}Cₖₛ^{m}Cₖₛ^{m}C_{ds}G_{ds}G_{ds}A_{ds}G_{ds}T_{ds}^{m}C_{ds}G_{ds}A_{ds}A_{ds} | 19.9 | 2092 | 2107 | 414 | 891 |
| | GₖₛAₖₛ^{m}Cₖ | | | | | |
| 486085 | GₖₛAₖₛAₖₛ^{m}C_{ds}T_{ds}G_{ds}G_{ds}^{m}C_{ds}A_{ds}G_{ds}G_{ds}^{m}C_{ds}G_{ds}Gₖₛ | 30.1 | 2155 | 2170 | 436 | 892 |
| | TₖₛGₖ | | | | | |
| 486086 | TₖₛGₖₛTₖₛG_{ds}A_{ds}A_{ds}^{m}C_{ds}T_{ds}G_{ds}G_{ds}^{m}C_{ds}A_{ds}G_{ds}Gₖₛ | 17.2 | 2158 | 2173 | 437 | 893 |
| | ^{m}CₖₛGₖ | | | | | |
| 486087 | GₖₛGₖₛTₖₛT_{ds}G_{ds}T_{ds}G_{ds}A_{ds}A_{ds}^{m}C_{ds}T_{ds}G_{ds}G_{ds}^{m}Cₖₛ | 11.5 | 2161 | 2176 | 438 | 894 |
| | AₖₛGₖ | | | | | |
| 486088 | GₖₛAₖₛGₖₛ^{m}C_{ds}G_{ds}G_{ds}T_{ds}T_{ds}G_{ds}T_{ds}G_{ds}A_{ds}A_{ds}^{m}Cₖₛ | 21.7 | 2165 | 2180 | 439 | 895 |
| | TₖₛGₖ | | | | | |
| 486094 | Aₖₛ^{m}CₖₛTₖₛG_{ds}G_{ds}A_{ds}G_{ds}^{m}C_{ds}T_{ds}G_{ds}G_{ds}G_{ds}^{m}C_{ds}Gₖₛ | 30.2 | 2193 | 2208 | 440 | 896 |
| | GₖₛAₖ | | | | | |
| 486096 | AₖₛGₖₛGₖₛA_{ds}^{m}C_{ds}T_{ds}G_{ds}G_{ds}A_{ds}G_{ds}^{m}C_{ds}T_{ds}G_{ds}Gₖₛ | 43.5 | 2196 | 2211 | 441 | 897 |
| | Gₖₛ^{m}Cₖ | | | | | |
| 486097 | Tₖₛ^{m}CₖₛAₖₛ^{m}C_{ds}A_{ds}G_{ds}G_{ds}A_{ds}^{m}C_{ds}T_{ds}G_{ds}G_{ds}A_{ds}Gₖₛ | 54.5 | 2200 | 2215 | 442 | 898 |
| | ^{m}CₖₛTₖ | | | | | |
| 486098 | AₖₛTₖₛ^{m}CₖₛA_{ds}^{m}C_{ds}A_{ds}G_{ds}G_{ds}A_{ds}^{m}C_{ds}T_{ds}G_{ds}G_{ds}Aₖₛ | 77.3 | 2201 | 2216 | 443 | 899 |
| | Gₖₛ^{m}Cₖ | | | | | |
| 486099 | GₖₛGₖₛAₖₛT_{ds}^{m}C_{ds}A_{ds}^{m}C_{ds}A_{ds}G_{ds}G_{ds}A_{ds}^{m}C_{ds}T_{ds}Gₖₛ | 24.8 | 2203 | 2218 | 444 | 900 |
| | GₖₛAₖ | | | | | |
| 486101 | ^{m}CₖₛAₖₛGₖₛ^{m}C_{ds}^{m}C_{ds}T_{ds}G_{ds}G_{ds}^{m}C_{ds}^{m}C_{ds}G_{ds}A_{ds}A_{ds} | 31.6 | 2386 | 2401 | 445 | 901 |
| | AₖₛGₖₛAₖ | | | | | |
| 486102 | ^{m}CₖₛTₖₛ^{m}CₖₛA_{ds}G_{ds}^{m}C_{ds}^{m}C_{ds}T_{ds}G_{ds}G_{ds}^{m}C_{ds}^{m}C_{ds}G_{ds} | 35.1 | 2388 | 2403 | 446 | 902 |
| | AₖₛAₖₛAₖ | | | | | |
| 486104 | GₖₛTₖₛ^{m}CₖₛA_{ds}G_{ds}G_{ds}G_{ds}^{m}C_{ds}^{m}C_{ds}T_{ds}^{m}C_{ds}A_{ds}G_{ds}^{m} | 26.9 | 2396 | 2411 | 447 | 903 |
| | Cₖₛ^{m}CₖₛTₖ | | | | | |
| 486105 | ^{m}CₖₛGₖₛTₖₛ^{m}C_{ds}A_{ds}G_{ds}G_{ds}G_{ds}^{m}C_{ds}^{m}C_{ds}T_{ds}^{m}C_{ds}A_{ds} | 48.4 | 2397 | 2412 | 448 | 904 |
| | Gₖₛ^{m}Cₖₛ^{m}Cₖ | | | | | |
| 486110 | TₖₛTₖₛTₖₛG_{ds}^{m}C_{ds}A_{ds}^{m}C_{ds}T_{ds}T_{ds}T_{ds}G_{ds}^{m}C_{ds}G_{ds}Aₖₛ | 31.6 | 2495 | 2510 | 449 | 905 |
| | Aₖₛ^{m}Cₖ | | | | | |
| 486111 | GₖₛAₖₛAₖₛA_{ds}G_{ds}^{m}C_{ds}T_{ds}T_{ds}T_{ds}G_{ds}^{m}C_{ds}A_{ds}^{m}C_{ds}Tₖₛ | 31.9 | 2501 | 2516 | 450 | 906 |
| | TₖₛTₖ | | | | | |
| 486112 | AₖₛAₖₛTₖₛT_{ds}T_{ds}^{m}C_{ds}^{m}C_{ds}^{m}C_{ds}G_{ds}A_{ds}G_{ds}T_{ds}A_{ds}Aₖₛ | 47.4 | 2565 | 2580 | 451 | 907 |
| | Gₖₛ^{m}Cₖ | | | | | |
| 486115 | Gₖₛ^{m}CₖₛAₖₛA_{ds}A_{ds}T_{ds}T_{ds}T_{ds}^{m}C_{ds}^{m}C_{ds}^{m}C_{ds}G_{ds}A_{ds} | 20.8 | 2568 | 2583 | 452 | 90 |
| | GₖₛTₖₛAₖ | | | | | |
| 486116 | AₖₛGₖₛ^{m}CₖₛA_{ds}A_{ds}A_{ds}T_{ds}T_{ds}T_{ds}^{m}C_{ds}^{m}C_{ds}^{m}C_{ds}G_{ds} | 23.9 | 2569 | 2584 | 453 | 909 |
| | AₖₛGₖₛTₖ | | | | | |
| 486117 | AₖₛAₖₛGₖₛ^{m}C_{ds}A_{ds}A_{ds}A_{ds}T_{ds}T_{ds}T_{ds}^{m}C_{ds}^{m}C_{ds}^{m}C_{ds} | 22 | 2570 | 2585 | 454 | 910 |
| | GₖₛAₖₛGₖ | | | | | |
| 486118 | AₖₛAₖₛAₖₛG_{ds}^{m}C_{ds}A_{ds}A_{ds}A_{ds}T_{ds}T_{ds}T_{ds}^{m}C_{ds}^{m}C_{ds}^{m} | 26.7 | 2571 | 2586 | 455 | 911 |
| | CₖₛGₖₛAₖ | | | | | |
| 486119 | AₖₛAₖₛAₖₛA_{ds}G_{ds}^{m}C_{ds}A_{ds}A_{ds}A_{ds}T_{ds}T_{ds}T_{ds}^{m}C_{ds}^{m}Cₖₛ | 33.5 | 2572 | 2587 | 456 | 912 |
| | ^{m}CₖₛGₖ | | | | | |
| 486120 | Gₖₛ^{m}CₖₛAₖₛA_{ds}A_{ds}A_{ds}G_{ds}^{m}C_{ds}A_{ds}A_{ds}A_{ds}T_{ds}T_{ds}Tₖₛ | 51.4 | 2574 | 2589 | 457 | 913 |
| | ^{m}Cₖₛ^{m}Cₖ | | | | | |
| 486121 | GₖₛGₖₛ^{m}CₖₛA_{ds}A_{ds}A_{ds}A_{ds}G_{ds}^{m}C_{ds}A_{ds}A_{ds}A_{ds}T_{ds}Tₖₛ | 60.8 | 2575 | 2590 | 458 | 914 |
| | Tₖₛ^{m}Cₖ | | | | | |
| 486123 | TₖₛTₖₛGₖₛG_{ds}^{m}C_{ds}A_{ds}A_{ds}A_{ds}A_{ds}G_{ds}^{m}C_{ds}A_{ds}A_{ds}Aₖₛ | 39.8 | 2577 | 2592 | 459 | 915 |
| | TₖₛTₖ | | | | | |
| 486125 | GₖₛTₖₛTₖₛT_{ds}G_{ds}G_{ds}^{m}C_{ds}A_{ds}A_{ds}A_{ds}A_{ds}G_{ds}^{m}C_{ds}Aₖₛ | 32.7 | 2579 | 2594 | 460 | 916 |
| | AₖₛAₖ | | | | | |
| 486126 | GₖₛGₖₛTₖₛT_{ds}T_{ds}G_{ds}G_{ds}^{m}C_{ds}A_{ds}A_{ds}A_{ds}A_{ds}G_{ds}^{m}Cₖₛ | 19.2 | 2580 | 2595 | 461 | 917 |
| | AₖₛAₖ | | | | | |
| 486127 | GₖₛGₖₛGₖₛT_{ds}T_{ds}T_{ds}G_{ds}G_{ds}^{m}C_{ds}A_{ds}A_{ds}A_{ds}A_{ds}Gₖₛ^{m} | 36.1 | 2581 | 2596 | 462 | 918 |
| | CₖₛAₖ | | | | | |
| 486128 | Gₖₛ^{m}CₖₛGₖₛG_{ds}G_{ds}T_{ds}T_{ds}T_{ds}G_{ds}G_{ds}^{m}C_{ds}A_{ds}A_{ds}Aₖₛ | 39.1 | 2583 | 2598 | 463 | 919 |
| | AₖₛGₖ | | | | | |
| 486129 | AₖₛGₖₛ^{m}CₖₛG_{ds}G_{ds}G_{ds}T_{ds}T_{ds}T_{ds}G_{ds}G_{ds}^{m}C_{ds}A_{ds}Aₖₛ | 31.4 | 2584 | 2599 | 464 | 920 |
| | AₖₛAₖ | | | | | |
| 486130 | AₖₛAₖₛGₖₛ^{m}C_{ds}G_{ds}G_{ds}G_{ds}T_{ds}T_{ds}T_{ds}G_{ds}G_{ds}^{m}C_{ds}Aₖₛ | 35.7 | 2585 | 2600 | 465 | 921 |
| | AₖₛAₖ | | | | | |
| 486133 | ^{m}CₖₛTₖₛ^{m}Cₖₛ^{m}C_{ds}G_{ds}A_{ds}G_{ds}A_{ds}G_{ds}^{m}C_{ds}A_{ds}G_{ds}^{m}C_{ds} | 45.9 | 2631 | 2646 | 466 | 922 |
| | Gₖₛ^{m}CₖₛAₖ | | | | | |
| 486134 | Gₖₛ^{m}CₖₛTₖₛ^{m}C_{ds}^{m}C_{ds}G_{ds}A_{ds}G_{ds}A_{ds}G_{ds}^{m}C_{ds}A_{ds}G_{ds}^{m} | 29.5 | 2632 | 2647 | 467 | 923 |
| | CₖₛGₖₛ^{m}Cₖ | | | | | |
| 486135 | GₖₛGₖₛ^{m}CₖₛT_{ds}^{m}C_{ds}^{m}C_{ds}G_{ds}A_{ds}G_{ds}A_{ds}G_{ds}^{m}C_{ds}A_{ds} | 51.4 | 2633 | 2648 | 468 | 924 |
| | Gₖₛ^{m}CₖₛGₖ | | | | | |
| 486142 | TₖₛAₖₛAₖₛA_{ds}T_{ds}A_{ds}T_{ds}^{m}C_{ds}^{m}C_{ds}A_{ds}A_{ds}A_{ds}^{m}C_{ds}^{m} | 64.4 | 2671 | 2686 | 469 | 925 |
| | CₖₛGₖₛ^{m}Cₖ | | | | | |
| 486147 | GₖₛTₖₛ^{m}CₖₛA_{ds}A_{ds}T_{ds}A_{ds}A_{ds}A_{ds}T_{ds}A_{ds}T_{ds}^{m}C_{ds}^{m}Cₖₛ | 16.1 | 2676 | 2691 | 470 | 926 |
| | AₖₛAₖ | | | | | |
| 486148 | AₖₛGₖₛGₖₛT_{ds}^{m}C_{ds}A_{ds}A_{ds}T_{ds}A_{ds}A_{ds}A_{ds}T_{ds}A_{ds}Tₖₛ^{m} | 18.3 | 2678 | 2693 | 471 | 927 |
| | Cₖₛ^{m}Cₖ | | | | | |
| 486149 | ^{m}CₖₛGₖₛAₖₛG_{ds}G_{ds}T_{ds}^{m}C_{ds}A_{ds}A_{ds}T_{ds}A_{ds}A_{ds}A_{ds}Tₖₛ | 37.9 | 2680 | 2695 | 472 | 928 |
| | AₖₛTₖ | | | | | |
| 486150 | Aₖₛ^{m}CₖₛGₖₛA_{ds}G_{ds}G_{ds}T_{ds}^{m}C_{ds}A_{ds}A_{ds}T_{ds}A_{ds}A_{ds}Aₖₛ | 45.3 | 2681 | 2696 | 473 | 929 |
| | TₖₛAₖ | | | | | |
| 486151 | GₖₛAₖₛ^{m}CₖₛG_{ds}A_{ds}G_{ds}G_{ds}T_{ds}^{m}C_{ds}A_{ds}A_{ds}T_{ds}A_{ds}Aₖₛ | 52.2 | 2682 | 2697 | 474 | 930 |
| | AₖₛTₖ | | | | | |
| 486152 | GₖₛGₖₛAₖₛ^{m}C_{ds}G_{ds}A_{ds}G_{ds}G_{ds}T_{ds}^{m}C_{ds}A_{ds}A_{ds}T_{ds}Aₖₛ | 19.8 | 2683 | 2698 | 475 | 931 |
| | AₖₛAₖ | | | | | |
| 486153 | AₖₛGₖₛGₖₛA_{ds}^{m}C_{ds}G_{ds}A_{ds}G_{ds}G_{ds}T_{ds}^{m}C_{ds}A_{ds}A_{ds}Tₖₛ | 19.9 | 2684 | 2699 | 476 | 932 |
| | AₖₛAₖ | | | | | |
| 486154 | GₖₛAₖₛGₖₛG_{ds}A_{ds}^{m}C_{ds}G_{ds}A_{ds}G_{ds}G_{ds}T_{ds}^{m}C_{ds}A_{ds}Aₖₛ | 19.6 | 2685 | 2700 | 477 | 933 |
| | TₖₛAₖ | | | | | |
| 486155 | GₖₛGₖₛAₖₛG_{ds}G_{ds}A_{ds}^{m}C_{ds}G_{ds}A_{ds}G_{ds}G_{ds}T_{ds}^{m}C_{ds}Aₖₛ | 38.3 | 2686 | 2701 | 478 | 934 |
| | AₖₛTₖ | | | | | |
| 486156 | ^{m}CₖₛGₖₛGₖₛA_{ds}G_{ds}G_{ds}A_{ds}^{m}C_{ds}G_{ds}A_{ds}G_{ds}G_{ds}T_{ds}^{m}Cₖₛ | 14.1 | 2687 | 2702 | 479 | 935 |
| | AₖₛAₖ | | | | | |
| 486157 | Tₖₛ^{m}CₖₛGₖₛG_{ds}A_{ds}G_{ds}G_{ds}A_{ds}^{m}C_{ds}G_{ds}A_{ds}G_{ds}G_{ds}Tₖₛ | 23.2 | 2688 | 2703 | 480 | 936 |
| | ^{m}CₖₛAₖ | | | | | |
| 486158 | GₖₛTₖₛ^{m}CₖₛG_{ds}G_{ds}A_{ds}G_{ds}G_{ds}A_{ds}^{m}C_{ds}G_{ds}A_{ds}G_{ds}Gₖₛ | 34.5 | 2689 | 2704 | 481 | 937 |
| | Tₖₛ^{m}Cₖ | | | | | |
| 486159 | AₖₛGₖₛTₖₛ^{m}C_{ds}G_{ds}G_{ds}A_{ds}G_{ds}G_{ds}A_{ds}^{m}C_{ds}G_{ds}A_{ds}Gₖₛ | 23.7 | 2690 | 2705 | 482 | 938 |
| | GₖₛTₖ | | | | | |
| 486160 | GₖₛAₖₛGₖₛT_{ds}^{m}C_{ds}G_{ds}G_{ds}A_{ds}G_{ds}G_{ds}A_{ds}^{m}C_{ds}G_{ds}Aₖₛ | 14.3 | 2691 | 2706 | 483 | 939 |
| | GₖₛGₖ | | | | | |
| 486161 | ^{m}CₖₛGₖₛAₖₛG_{ds}T_{ds}^{m}C_{ds}G_{ds}G_{ds}A_{ds}G_{ds}G_{ds}A_{ds}^{m}C_{ds}Gₖₛ | 29 | 2692 | 2707 | 484 | 940 |
| | AₖₛGₖ | | | | | |
| 486162 | AₖₛGₖₛ^{m}CₖₛG_{ds}A_{ds}G_{ds}T_{ds}^{m}C_{ds}G_{ds}G_{ds}A_{ds}G_{ds}G_{ds}Aₖₛ | 20.6 | 2694 | 2709 | 485 | 941 |
| | ^{m}CₖₛGₖ | | | | | |
| 486163 | ^{m}CₖₛAₖₛGₖₛ^{m}C_{ds}G_{ds}A_{ds}G_{ds}T_{ds}^{m}C_{ds}G_{ds}G_{ds}A_{ds}G_{ds}Gₖₛ | 29 | 2695 | 2710 | 486 | 942 |
| | Aₖₛ^{m}Cₖ | | | | | |
| 486164 | Tₖₛ^{m}CₖₛAₖₛG_{ds}^{m}C_{ds}G_{ds}A_{ds}G_{ds}T_{ds}^{m}C_{ds}G_{ds}G_{ds}A_{ds}Gₖₛ | 17 | 2696 | 2711 | 487 | 943 |
| | GₖₛAₖ | | | | | |
| 486165 | GₖₛTₖₛ^{m}CₖₛA_{ds}G_{ds}^{m}C_{ds}G_{ds}A_{ds}G_{ds}T_{ds}^{m}C_{ds}G_{ds}G_{ds}Aₖₛ | 14.2 | 2697 | 2712 | 426 | 944 |
| | GₖₛGₖ | | | | | |
| 486166 | TₖₛGₖₛTₖₛ^{m}C_{ds}A_{ds}G_{ds}^{m}C_{ds}G_{ds}A_{ds}G_{ds}T_{ds}^{m}C_{ds}G_{ds}Gₖₛ | 25.1 | 2698 | 2713 | 488 | 945 |
| | AₖₛGₖ | | | | | |
| 486167 | ^{m}CₖₛTₖₛGₖₛT_{ds}^{m}C_{ds}A_{ds}G_{ds}^{m}C_{ds}G_{ds}A_{ds}G_{ds}T_{ds}^{m}C_{ds} | 15 | 2699 | 2714 | 489 | 946 |
| | GₖₛGₖₛAₖ | | | | | |
| 486168 | ^{m}Cₖₛ^{m}CₖₛTₖₛG_{ds}T_{ds}^{m}C_{ds}A_{ds}G_{ds}^{m}C_{ds}G_{ds}A_{ds}G_{ds}T_{ds}^{m} | 12.4 | 2700 | 2715 | 427 | 947 |
| | CₖₛGₖₛGₖ | | | | | |
| 486169 | Gₖₛ^{m}Cₖₛ^{m}CₖₛT_{ds}G_{ds}T_{ds}^{m}C_{ds}A_{ds}G_{ds}^{m}C_{ds}G_{ds}A_{ds}G_{ds} | 24.5 | 2701 | 2716 | 428 | 948 |
| | Tₖₛ^{m}CₖₛGₖ | | | | | |
| 486170 | AₖₛGₖₛ^{m}Cₖₛ^{m}C_{ds}T_{ds}G_{ds}T_{ds}^{m}C_{ds}A_{ds}G_{ds}^{m}C_{ds}G_{ds}A_{ds} | 16.3 | 2702 | 2717 | 429 | 949 |
| | GₖₛTₖₛ^{m}Cₖ | | | | | |
| 486171 | ^{m}CₖₛAₖₛGₖₛT_{ds}G_{ds}^{m}C_{ds}A_{ds}T_{ds}^{m}C_{ds}^{m}C_{ds}A_{ds}A_{ds}A_{ds} | 31.8 | 2744 | 2759 | 490 | 950 |
| | Aₖₛ^{m}CₖₛGₖ | | | | | |
| 486172 | Tₖₛ^{m}CₖₛAₖₛG_{ds}T_{ds}G_{ds}^{m}C_{ds}A_{ds}T_{ds}^{m}C_{ds}^{m}C_{ds}A_{ds}A_{ds} | 23.1 | 2745 | 2760 | 491 | 951 |
| | AₖₛAₖₛ^{m}Cₖ | | | | | |
| 486173 | ^{m}CₖₛTₖₛ^{m}CₖₛA_{ds}G_{ds}T_{ds}G_{ds}^{m}C_{ds}A_{ds}T_{ds}^{m}C_{ds}^{m}C_{ds}A_{ds} | 23 | 2746 | 2761 | 492 | 952 |
| | AₖₛAₖₛAₖ | | | | | |
| 486174 | Tₖₛ^{m}CₖₛTₖₛ^{m}C_{ds}A_{ds}G_{ds}T_{ds}G_{ds}^{m}C_{ds}A_{ds}T_{ds}^{m}C_{ds}^{m}C_{ds} | 50.9 | 2747 | 2762 | 493 | 953 |
| | AₖₛAₖₛAₖ | | | | | |
| 486175 | GₖₛTₖₛ^{m}CₖₛT_{ds}^{m}C_{ds}A_{ds}G_{ds}T_{ds}G_{ds}^{m}C_{ds}A_{ds}T_{ds}^{m}C_{ds}^{m} | 17.2 | 2748 | 2763 | 494 | 954 |
| | CₖₛAₖₛAₖ | | | | | |
| 486176 | GₖₛGₖₛGₖₛT_{ds}^{m}C_{ds}T_{ds}^{m}C_{ds}A_{ds}G_{ds}T_{ds}G_{ds}^{m}C_{ds}A_{ds}Tₖₛ | 37.6 | 2750 | 2765 | 430 | 955 |
| | ^{m}Cₖₛ^{m}Cₖ | | | | | |
| 486177 | ^{m}CₖₛAₖₛAₖₛT_{ds}A_{ds}A_{ds}A_{ds}T_{ds}A_{ds}^{m}C_{ds}^{m}C_{ds}G_{ds}A_{ds}Gₖₛ | 40 | 2772 | 2787 | 495 | 956 |
| | GₖₛAₖ | | | | | |
| 486178 | Aₖₛ^{m}CₖₛAₖₛA_{ds}T_{ds}A_{ds}A_{ds}A_{ds}T_{ds}A_{ds}^{m}C_{ds}^{m}C_{ds}G_{ds}Aₖₛ | 11.3 | 2773 | 2788 | 23 | 881 |
| | GₖₛGₖ | | | | | |
| 486179 | AₖₛGₖₛAₖₛ^{m}C_{ds}A_{ds}A_{ds}T_{ds}A_{ds}A_{ds}A_{ds}T_{ds}A_{ds}^{m}C_{ds}^{m}Cₖₛ | 13.5 | 2775 | 2790 | 496 | 957 |
| | GₖₛAₖ | | | | | |
| 486180 | ^{m}CₖₛAₖₛGₖₛA_{ds}^{m}C_{ds}A_{ds}A_{ds}T_{ds}A_{ds}A_{ds}A_{ds}T_{ds}A_{ds}^{m}Cₖₛ | 18.6 | 2776 | 2791 | 497 | 958 |
| | ^{m}CₖₛGk | | | | | |

### Example 9: ASOs Designed to Target a Human DMPK RNA Transcript

A series of antisense oligonucleotides (ASOs) were designed to target hDMPK. The newly designed ASOs were prepared using standard oligonucleotide synthesis well known in the art and are described in Table 13 to 18, below. Subscripts “s” indicate phosphorothioate internucleoside linkages; subscripts “k” indicate 6′-(S)—CH₃ bicyclic nucleosides (cEt); subscripts “e” indicate 2′-methoxyethyl (MOE) modified nucleosides; and subscripts “d” indicate β-D-2′-deoxyribonucleosides. “^{m}C” indicates 5-methylcytosine nucleosides.

The antisense oligonucleotides targeted to a human DMPK nucleic acid were tested for their effect on DMPK RNA transcript in vitro. Cultured HepG2 cells at a density of 20,000 cells per well were transfected using electroporation with 4,500 nM antisense oligonucleotide. After approximately 24 hours, RNA was isolated from the cells and DMPK transcript levels were measured by quantitative real-time PCR. DMPK RNA transcript levels were adjusted according to total RNA content, as measured by RIBOGREEN®. Results are presented as percent expression of DMPK, relative to untreated control cells, with “% Target Expression” representing the percent expression of DMPK relative to untreated control cells

All the antisense oligonucleotides listed in Table 13 target SEQ TD NO: 1 (GENBANK Accession No. NM_001081560.1). All the antisense oligonucleotides listed in Table 14 to 18 target SEQ ID NO: 2 (the complement of GENBANK Accession No. NT_011109.15 truncated from nucleotides 18540696 to 18555106). ‘Target start site’ indicates the 5′-most nucleoside to which the antisense oligonucleotide is targeted in the human genomic gene sequence. ‘Target stop site’ indicates the 3′-most nucleoside to which the antisense oligonucleotide is targeted in the human genomic sequence.

TABLE 13
Inhibition of human DMPK RNA transcript in HepG2 cells targeting SEQ ID NO: 1

| | | | Start | Stop | | |
|---|---|---|---|---|---|---|
| | | | Site | Site | | |
| | | | on | on | | |
| | | | Sec | Seq | | Modified |
| ISIS | | % Target | ID: | ID: | Unmodified | Seq ID |
| No. | Sequence | Expression | 1 | 1 | Seq ID No. | No |
| UTC | N/A | 100 | N/A | N/A | | |
| 445569 | ^{m}CₑₛGₑₛGₑₛAₑₛGₑₛ^{m}C_{ds}G_{ds}G_{ds}T_{ds}T_{ds}G_{ds}T_{ds}G_{ds}A_{ds}A_{ds} | 36.7 | 2163 | 2182 | 24 | 1443 |
| | ^{m}CₑₛTₑₛGₑₛGₑₛ^{m}Cₑ | | | | | |
| 486178 | Aₖₛ^{m}CₖₛAₖₛA_{ds}T_{ds}A_{ds}A_{ds}A_{ds}T_{ds}A_{ds}^{m}C_{ds}^{m}C_{ds}G_{ds}Aₖₛ | 21.3 | 2773 | 2788 | 23 | 881 |
| | GₖₛGₖ | | | | | |
| 569403 | ^{m}CₖₛAₖₛ^{m}CₖₛG_{ds}G_{ds}A_{ds}A_{ds}G_{ds}^{m}C_{ds}A_{ds}^{m}C_{ds}G_{ds}A_{ds}^{m} | 18.8 | 542 | 557 | 498 | 959 |
| | CₖₛAₖₛ^{m}Cₖ | | | | | |
| 569404 | Tₖₛ^{m}CₖₛAₖₛ^{m}C_{ds}G_{ds}G_{ds}A_{ds}A_{ds}G_{ds}^{m}C_{ds}A_{ds}^{m}C_{ds}G_{ds}Aₖₛ | 25.2 | 543 | 558 | 499 | 960 |
| | ^{m}CₖₛAₖ | | | | | |
| 569405 | ^{m}CₖₛTₖₛ^{m}CₖₛA_{ds}^{m}C_{ds}G_{ds}G_{ds}A_{ds}A_{ds}G_{ds}^{m}C_{ds}A_{ds}^{m}C_{ds}Gₖₛ | 21.2 | 544 | 559 | 500 | 961 |
| | Aₖₛ^{m}Cₖ | | | | | |
| 569406 | ^{m}Cₖₛ^{m}CₖₛTₖₛ^{m}C_{ds}T_{ds}^{m}C_{ds}^{m}C_{ds}T_{ds}^{m}C_{ds}A_{ds}^{m}C_{ds}G_{ds}G_{ds} | 27.9 | 550 | 565 | 343 | 962 |
| | AₖₛAₖₛGₖ | | | | | |
| 569407 | GₖₛTₖₛ^{m}Cₖₛ^{m}C_{ds}^{m}C_{ds}T_{ds}^{m}C_{ds}T_{ds}^{m}C_{ds}^{m}C_{ds}T_{ds}^{m}C_{ds}A_{ds}^{m} | 30.9 | 553 | 568 | 501 | 963 |
| | CₖₛGₖₛGₖ | | | | | |
| 569408 | ^{m}CₖₛGₖₛTₖₛ^{m}C_{ds}^{m}C_{ds}^{m}C_{ds}T_{ds}^{m}C_{ds}T_{ds}^{m}C_{ds}^{m}C_{ds}T_{ds}^{m}C_{ds} | 32.8 | 554 | 569 | 502 | 964 |
| | Aₖₛ^{m}CₖₛGₖ | | | | | |
| 569409 | ^{m}Cₖₛ^{m}Cₖₛ^{m}CₖₛA_{ds}T_{ds}T_{ds}^{m}C_{ds}A_{ds}^{m}C_{ds}^{m}C_{ds}A_{ds}A_{ds}^{m}C_{ds} | 33 | 568 | 583 | 503 | 965 |
| | Aₖₛ^{m}CₖₛGₖ | | | | | |
| 569410 | ^{m}Cₖₛ^{m}Cₖₛ^{m}Cₖₛ^{m}C_{ds}A_{ds}T_{ds}T_{ds}^{m}C_{ds}A_{ds}^{m}C_{ds}^{m}C_{ds}A_{ds}A_{ds} | 42.1 | 569 | 584 | 504 | 966 |
| | ^{m}CₖₛAₖₛ^{m}Cₖ | | | | | |
| 569411 | Tₖₛ^{m}Cₖₛ^{m}Cₖₛ^{m}C_{ds}^{m}C_{ds}A_{ds}T_{ds}T_{ds}^{m}C_{ds}A_{ds}^{m}C_{ds}^{m}C_{ds}A_{ds} | 68.6 | 570 | 585 | 505 | 967 |
| | Aₖₛ^{m}CₖₛAₖ | | | | | |
| 569412 | GₖₛTₖₛ^{m}Cₖₛ^{m}C_{ds}^{m}C_{ds}^{m}C_{ds}A_{ds}T_{ds}T_{ds}^{m}C_{ds}A_{ds}^{m}C_{ds}^{m}C_{ds} | 60.7 | 571 | 586 | 506 | 968 |
| | AₖₛAₖₛ^{m}Cₖ | | | | | |
| 569413 | GₖₛGₖₛTₖₛ^{m}C_{ds}^{m}C_{ds}^{m}C_{ds}^{m}C_{ds}A_{ds}T_{ds}T_{ds}^{m}C_{ds}A_{ds}^{m}C_{ds}^{m} | 65.1 | 572 | 587 | 507 | 969 |
| | CₖₛAₖₛAₖ | | | | | |
| 569414 | ^{m}CₖₛGₖₛGₖₛT_{ds}^{m}C_{ds}^{m}C_{ds}^{m}C_{ds}^{m}C_{ds}A_{ds}T_{ds}T_{ds}^{m}C_{ds}A_{ds}^{m} | 54.4 | 573 | 588 | 508 | 970 |
| | Cₖₛ^{m}CₖₛAₖ | | | | | |
| 569415 | ^{m}Cₖₛ^{m}CₖₛGₖₛG_{ds}T_{ds}^{m}C_{ds}^{m}C_{ds}^{m}C_{ds}^{m}C_{ds}A_{ds}T_{ds}T_{ds}^{m}C_{ds} | 51.3 | 574 | 589 | 509 | 971 |
| | Aₖₛ^{m}Cₖₛ^{m}Cₖ | | | | | |
| 569416 | Gₖₛ^{m}Cₖₛ^{m}CₖₛG_{ds}G_{ds}T_{ds}^{m}C_{ds}^{m}C_{ds}^{m}C_{ds}^{m}C_{ds}A_{ds}T_{ds}T_{ds}^{m} | 57.9 | 575 | 590 | 510 | 972 |
| | CₖₛAₖₛ^{m}Cₖ | | | | | |
| 569417 | ^{m}CₖₛGₖₛ^{m}Cₖₛ^{m}C_{ds}G_{ds}G_{ds}T_{ds}^{m}C_{ds}^{m}C_{ds}^{m}C_{ds}^{m}C_{ds}A_{ds}T_{ds} | 43.2 | 576 | 591 | 511 | 973 |
| | Tₖₛ^{m}CₖₛAₖ | | | | | |
| 569418 | ^{m}Cₖₛ^{m}CₖₛGₖₛ^{m}C_{ds}^{m}C_{ds}G_{ds}G_{ds}T_{ds}^{m}C_{ds}^{m}C_{ds}^{m}C_{ds}^{m}C_{ds}A_{ds} | 79.3 | 577 | 592 | 512 | 974 |
| | TₖₛTₖₛ^{m}Cₖ | | | | | |
| 569419 | Aₖₛ^{m}Cₖₛ^{m}CₖₛG_{ds}^{m}C_{ds}^{m}C_{ds}G_{ds}G_{ds}T_{ds}^{m}C_{ds}^{m}C_{ds}^{m}C_{ds}^{m}C_{ds} | 36 | 578 | 593 | 513 | 975 |
| | AₖₛTₖₛTₖ | | | | | |
| 569420 | ^{m}CₖₛAₖₛ^{m}Cₖₛ^{m}C_{ds}G_{ds}^{m}C_{ds}^{m}C_{ds}G_{ds}G_{ds}T_{ds}^{m}C_{ds}^{m}C_{ds}^{m}C_{ds} | 36.2 | 579 | 594 | 514 | 976 |
| | ^{m}CₖₛAₖₛTₖ | | | | | |
| 569421 | ^{m}Cₖₛ^{m}CₖₛAₖₛ^{m}C_{ds}^{m}C_{ds}G_{ds}^{m}C_{ds}^{m}C_{ds}G_{ds}G_{ds}T_{ds}^{m}C_{ds}^{m}C_{ds} | 34.7 | 580 | 595 | 515 | 977 |
| | ^{m}Cₖₛ^{m}CₖₛAₖ | | | | | |
| 569422 | Tₖₛ^{m}Cₖₛ^{m}CₖₛA_{ds}^{m}C_{ds}^{m}C_{ds}G_{ds}^{m}C_{ds}^{m}C_{ds}G_{ds}G_{ds}T_{ds}^{m}C_{ds} | 40 | 581 | 596 | 516 | 978 |
| | ^{m}Cₖₛ^{m}Cₖₛ^{m}Cₖ | | | | | |
| 569423 | AₖₛTₖₛ^{m}Cₖₛ^{m}C_{ds}A_{ds}^{m}C_{ds}^{m}C_{ds}G_{ds}^{m}C_{ds}^{m}C_{ds}G_{ds}G_{ds}T_{ds}^{m} | 31.6 | 582 | 597 | 517 | 979 |
| | Cₖₛ^{m}Cₖₛ^{m}Cₖ | | | | | |
| 569424 | GₖₛAₖₛTₖₛ^{m}C_{ds}^{m}C_{ds}A_{ds}^{m}C_{ds}^{m}C_{ds}G_{ds}^{m}C_{ds}C_{ds}G_{ds}G_{ds} | 56 | 583 | 598 | 518 | 980 |
| | Tₖₛ^{m}Cₖₛ^{m}Cₖ | | | | | |
| 569425 | TₖₛGₖₛAₖₛT_{ds}^{m}C_{ds}^{m}C_{ds}A_{ds}^{m}C_{ds}^{m}C_{ds}G_{ds}^{m}C_{ds}^{m}C_{ds}G_{ds} | 53.9 | 584 | 599 | 519 | 981 |
| | GₖₛTₖₛ^{m}Cₖ | | | | | |
| 569426 | GₖₛTₖₛGₖₛA_{ds}T_{ds}^{m}C_{ds}^{m}C_{ds}A_{ds}^{m}C_{ds}^{m}C_{ds}G_{ds}^{m}C_{ds}^{m}C_{ds} | 54.1 | 585 | 600 | 520 | 982 |
| | GₖₛGₖₛTₖ | | | | | |
| 569427 | ^{m}CₖₛGₖₛTₖₛG_{ds}A_{ds}T_{ds}^{m}C_{ds}^{m}C_{ds}A_{ds}^{m}C_{ds}^{m}C_{ds}G_{ds}^{m}C_{ds}^{m} | 34.8 | 586 | 601 | 521 | 983 |
| | CₖₛGₖₛGₖ | | | | | |
| 569428 | ^{m}CₖₛAₖₛTₖₛ^{m}C_{ds}^{m}C_{ds}T_{ds}G_{ds}G_{ds}A_{ds}A_{ds}G_{ds}G_{ds}^{m}C_{ds}Gₖₛ | 71 | 611 | 626 | 522 | 984 |
| | AₖₛAₖ | | | | | |
| 569429 | Tₖₛ^{m}CₖₛAₖₛT_{ds}^{m}C_{ds}^{m}C_{ds}T_{ds}G_{ds}G_{ds}A_{ds}A_{ds}G_{ds}G_{ds}^{m}Cₖₛ | 51.1 | 612 | 627 | 523 | 985 |
| | GₖₛAₖ | | | | | |
| 569430 | AₖₛGₖₛTₖₛT_{ds}^{m}C_{ds}T_{ds}^{m}C_{ds}A_{ds}T_{ds}^{m}C_{ds}^{m}C_{ds}T_{ds}G_{ds}Gₖₛ | 69.2 | 617 | 632 | 524 | 986 |
| | AₖₛAₖ | | | | | |
| 569431 | TₖₛAₖₛGₖₛT_{ds}T_{ds}^{m}C_{ds}T_{ds}^{m}C_{ds}A_{ds}T_{ds}^{m}C_{ds}^{m}C_{ds}T_{ds}Gₖₛ | 48.6 | 618 | 633 | 525 | 987 |
| | GₖₛAₖ | | | | | |
| 569432 | GₖₛTₖₛAₖₛG_{ds}T_{ds}T_{ds}^{m}C_{ds}T_{ds}^{m}C_{ds}A_{ds}T_{ds}^{m}C_{ds}^{m}C_{ds}Tₖₛ | 29.6 | 619 | 634 | 526 | 988 |
| | GₖₛGₖ | | | | | |
| 569433 | ^{m}CₖₛAₖₛGₖₛG_{ds}T_{ds}A_{ds}^{m}C_{ds}A_{ds}G_{ds}G_{ds}T_{ds}A_{ds}G_{ds}TₖₛTₖₛ | 36.5 | 628 | 643 | 527 | 989 |
| | ^{m}Cₖ | | | | | |
| 569434 | ^{m}Cₖₛ^{m}CₖₛAₖₛG_{ds}G_{ds}T_{ds}A_{ds}^{m}C_{ds}A_{ds}G_{ds}G_{ds}T_{ds}A_{ds}Gₖₛ | 51 | 629 | 644 | 528 | 990 |
| | TₖₛTₖ | | | | | |
| 569435 | GₖₛAₖₛ^{m}Cₖₛ^{m}C_{ds}A_{ds}G_{ds}G_{ds}T_{ds}A_{ds}^{m}C_{ds}A_{ds}G_{ds}G_{ds}Tₖₛ | 49.9 | 631 | 646 | 529 | 991 |
| | AₖₛGₖ | | | | | |
| 569436 | ^{m}CₖₛTₖₛ^{m}Cₖₛ^{m}C_{ds}A_{ds}T_{ds}G_{ds}A_{ds}^{m}C_{ds}^{m}C_{ds}A_{ds}G_{ds}G_{ds}Tₖₛ | 41 | 637 | 652 | 530 | 992 |
| | Aₖₛ^{m}Cₖ | | | | | |
| 569437 | Aₖₛ^{m}CₖₛTₖₛ^{m}C_{ds}^{m}C_{ds}A_{ds}T_{ds}G_{ds}A_{ds}^{m}C_{ds}^{m}C_{ds}A_{ds}G_{ds}Gₖₛ | 32.9 | 638 | 653 | 531 | 993 |
| | TₖₛAₖ | | | | | |
| 569438 | TₖₛAₖₛ^{m}CₖₛT_{ds}^{m}C_{ds}^{m}C_{ds}A_{ds}T_{ds}G_{ds}A_{ds}^{m}C_{ds}^{m}C_{ds}A_{ds}Gₖₛ | 25.7 | 639 | 654 | 532 | 994 |
| | GₖₛTₖ | | | | | |
| 569439 | AₖₛTₖₛAₖₛ^{m}C_{ds}T_{ds}^{m}C_{ds}^{m}C_{ds}A_{ds}T_{ds}G_{ds}A_{ds}^{m}C_{ds}^{m}C_{ds}Aₖₛ | 9.4 | 640 | 655 | 533 | 995 |
| | GₖₛGₖ | | | | | |
| 569440 | AₖₛAₖₛTₖₛA_{ds}^{m}C_{ds}T_{ds}^{m}C_{ds}^{m}C_{ds}A_{ds}T_{ds}G_{ds}A_{ds}^{m}C_{ds}^{m}Cₖₛ | 21.2 | 641 | 656 | 534 | 996 |
| | AₖₛGₖ | | | | | |
| 569441 | TₖₛAₖₛAₖₛT_{ds}A_{ds}^{m}C_{ds}T_{ds}^{m}C_{ds}^{m}C_{ds}A_{ds}T_{ds}G_{ds}A_{ds}^{m}Cₖₛ^{m} | 30.8 | 642 | 657 | 535 | 997 |
| | CₖₛAₖ | | | | | |
| 569442 | GₖₛTₖₛAₖₛA_{ds}T_{ds}A_{ds}^{m}C_{ds}T_{ds}^{m}C_{ds}^{m}C_{ds}A_{ds}T_{ds}G_{ds}Aₖₛ^{m} | 29.8 | 643 | 658 | 536 | 998 |
| | Cₖₛ^{m}Cₖ | | | | | |
| 569443 | ^{m}CₖₛGₖₛTₖₛA_{ds}A_{ds}T_{ds}A_{ds}^{m}C_{ds}T_{ds}^{m}C_{ds}^{m}C_{ds}A_{ds}T_{ds}Gₖₛ | 25.3 | 644 | 659 | 537 | 999 |
| | Aₖₛ^{m}Cₖ | | | | | |
| 569444 | ^{m}CₖₛTₖₛTₖₛG_{ds}^{m}C_{ds}T_{ds}^{m}C_{ds}A_{ds}G_{ds}^{m}C_{ds}A_{ds}G_{ds}T_{ds}Gₖₛ | 19.3 | 676 | 691 | 538 | 1000 |
| | Tₖₛ^{m}Cₖ | | | | | |
| 569445 | Aₖₛ^{m}CₖₛTₖₛT_{ds}G_{ds}^{m}C_{ds}T_{ds}^{m}C_{ds}A_{ds}G_{ds}^{m}C_{ds}A_{ds}G_{ds}Tₖₛ | 35 | 677 | 692 | 539 | 1001 |
| | GₖₛTₖ | | | | | |
| 569446 | AₖₛAₖₛ^{m}CₖₛT_{ds}T_{ds}G_{ds}^{m}C_{ds}T_{ds}^{m}C_{ds}A_{ds}G_{ds}^{m}C_{ds}A_{ds}Gₖₛ | 30 | 678 | 693 | 540 | 1002 |
| | TₖₛGₖ | | | | | |
| 569447 | AₖₛAₖₛAₖₛ^{m}C_{ds}T_{ds}T_{ds}G_{ds}^{m}C_{ds}T_{ds}^{m}C_{ds}A_{ds}G_{ds}^{m}C_{ds}Aₖₛ | 32.2 | 679 | 694 | 344 | 1003 |
| | GₖₛTₖ | | | | | |
| 569448 | ^{m}Cₖₛ^{m}CₖₛAₖₛA_{ds}A_{ds}^{m}C_{ds}T_{ds}T_{ds}G_{ds}^{m}C_{ds}T_{ds}^{m}C_{ds}A_{ds}Gₖₛ | 30.1 | 681 | 696 | 346 | 1004 |
| | ^{m}CₖₛAₖ | | | | | |
| 569449 | ^{m}Cₖₛ^{m}Cₖₛ^{m}CₖₛA_{ds}A_{ds}A_{ds}^{m}C_{ds}T_{ds}T_{ds}G_{ds}^{m}C_{ds}T_{ds}^{m}C_{ds} | 18.4 | 682 | 697 | 347 | 1005 |
| | AₖₛGₖₛ^{m}Cₖ | | | | | |
| 569450 | ^{m}Cₖₛ^{m}Cₖₛ^{m}Cₖₛ^{m}C_{ds}A_{ds}A_{ds}A_{ds}^{m}C_{ds}T_{ds}T_{ds}G_{ds}^{m}C_{ds}T_{ds}^{m} | 44.8 | 683 | 698 | 348 | 1006 |
| | CₖₛAₖₛGₖ | | | | | |
| 569451 | Gₖₛ^{m}CₖₛTₖₛ^{m}C_{ds}^{m}C_{ds}^{m}C_{ds}^{m}C_{ds}A_{ds}A_{ds}A_{ds}^{m}C_{ds}T_{ds}T_{ds} | 47 | 686 | 701 | 541 | 1007 |
| | Gₖₛ^{m}CₖₛTₖ | | | | | |
| 569452 | ^{m}CₖₛGₖₛ^{m}CₖₛT_{ds}^{m}C_{ds}^{m}C_{ds}^{m}C_{ds}^{m}C_{ds}A_{ds}A_{ds}A_{ds}^{m}C_{ds}T_{ds} | 35.4 | 687 | 702 | 542 | 1008 |
| | TₖₛGₖₛ^{m}Cₖ | | | | | |
| 569453 | ^{m}Cₖₛ^{m}CₖₛGₖₛ^{m}C_{ds}T_{ds}^{m}C_{ds}^{m}C_{ds}^{m}C_{ds}^{m}C_{ds}A_{ds}A_{ds}A_{ds}^{m}C_{ds} | 46.6 | 688 | 703 | 543 | 1009 |
| | TₖₛTₖₛGₖ | | | | | |
| 569454 | Tₖₛ^{m}Cₖₛ^{m}CₖₛG_{ds}^{m}C_{ds}T_{ds}^{m}C_{ds}^{m}C_{ds}^{m}C_{ds}^{m}C_{ds}A_{ds}A_{ds}A_{ds} | 29.4 | 689 | 704 | 544 | 1010 |
| | ^{m}CₖₛTₖₛTₖ | | | | | |
| 569455 | AₖₛTₖₛ^{m}Cₖₛ^{m}C_{ds}G_{ds}^{m}C_{ds}T_{ds}^{m}C_{ds}^{m}C_{ds}^{m}C_{ds}^{m}C_{ds}A_{ds}A_{ds} | 36.9 | 690 | 705 | 545 | 1011 |
| | Aₖₛ^{m}CₖₛTₖ | | | | | |
| 569456 | AₖₛAₖₛTₖₛ^{m}C_{ds}^{m}C_{ds}G_{ds}^{m}C_{ds}T_{ds}^{m}C_{ds}^{m}C_{ds}^{m}C_{ds}^{m}C_{ds}A_{ds} | 32.9 | 691 | 706 | 546 | 1012 |
| | AₖₛAₖₛ^{m}Cₖ | | | | | |
| 569457 | GₖₛAₖₛAₖₛT_{ds}^{m}C_{ds}^{m}C_{ds}G_{ds}^{m}C_{ds}T_{ds}^{m}C_{ds}^{m}C_{ds}^{m}C_{ds}^{m}C_{ds} | 41.7 | 692 | 707 | 547 | 1013 |
| | AₖₛAₖₛAₖ | | | | | |
| 569458 | GₖₛGₖₛAₖₛA_{ds}T_{ds}^{m}C_{ds}^{m}C_{ds}G_{ds}^{m}C_{ds}T_{ds}^{m}C_{ds}^{m}C_{ds}^{m}C_{ds}^{m} | 36.4 | 693 | 708 | 548 | 1014 |
| | CₖₛAₖₛAₖ | | | | | |
| 569459 | ^{m}CₖₛGₖₛGₖₛA_{ds}A_{ds}T_{ds}^{m}C_{ds}^{m}C_{ds}G_{ds}^{m}C_{ds}T_{ds}^{m}C_{ds}^{m}C_{ds}^{m} | 30 | 694 | 709 | 549 | 1015 |
| | Cₖₛ^{m}CₖₛAₖ | | | | | |
| 569460 | ^{m}Cₖₛ^{m}CₖₛGₖₛG_{ds}A_{ds}A_{ds}T_{ds}^{m}C_{ds}^{m}C_{ds}G_{ds}^{m}C_{ds}T_{ds}^{m}C_{ds}^{m} | 26.5 | 695 | 710 | 550 | 1016 |
| | Cₖₛ^{m}Cₖₛ^{m}Cₖ | | | | | |
| 569461 | Gₖₛ^{m}Cₖₛ^{m}CₖₛG_{ds}G_{ds}A_{ds}A_{ds}T_{ds}^{m}C_{ds}^{m}C_{ds}G_{ds}^{m}C_{ds}T_{ds}^{m} | 36.5 | 696 | 711 | 551 | 1017 |
| | Cₖₛ^{m}Cₖₛ^{m}Cₖ | | | | | |
| 569462 | AₖₛGₖₛAₖₛA_{ds}G_{ds}^{m}C_{ds}G_{ds}^{m}C_{ds}G_{ds}^{m}C_{ds}^{m}C_{ds}A_{ds}T_{ds}^{m}Cₖₛ | 26 | 713 | 728 | 552 | 1018 |
| | Tₖₛ^{m}Cₖ | | | | | |
| 569463 | TₖₛAₖₛGₖₛA_{ds}A_{ds}G_{ds}^{m}C_{ds}G_{ds}^{m}C_{ds}G_{ds}^{m}C_{ds}^{m}C_{ds}A_{ds}Tₖₛ | 40.3 | 714 | 729 | 553 | 1019 |
| | ^{m}CₖₛTₖ | | | | | |
| 569464 | GₖₛTₖₛAₖₛG_{ds}A_{ds}A_{ds}G_{ds}^{m}C_{ds}G_{ds}^{m}C_{ds}G_{ds}^{m}C_{ds}^{m}C_{ds}Aₖₛ | 28.9 | 715 | 730 | 554 | 1020 |
| | Tₖₛ^{m}Cₖ | | | | | |
| 569465 | GₖₛGₖₛTₖₛA_{ds}G_{ds}A_{ds}A_{ds}G_{ds}^{m}C_{ds}G_{ds}^{m}C_{ds}G_{ds}^{m}C_{ds}^{m}Cₖₛ | 35.7 | 716 | 731 | 555 | 1021 |
| | AₖₛTₖ | | | | | |
| 569466 | AₖₛGₖₛGₖₛT_{ds}A_{ds}G_{ds}A_{ds}A_{ds}G_{ds}^{m}C_{ds}G_{ds}^{m}C_{ds}G_{ds}^{m}Cₖₛ^{m} | 31.1 | 717 | 732 | 556 | 1022 |
| | CₖₛAₖ | | | | | |
| 569467 | ^{m}CₖₛAₖₛGₖₛG_{ds}T_{ds}A_{ds}G_{ds}A_{ds}A_{ds}G_{ds}^{m}C_{ds}G_{ds}^{m}C_{ds}Gₖₛ^{m} | 14.8 | 718 | 733 | 557 | 1023 |
| | Cₖₛ^{m}Cₖ | | | | | |
| 569468 | ^{m}Cₖₛ^{m}CₖₛAₖₛG_{ds}G_{ds}T_{ds}A_{ds}G_{ds}A_{ds}A_{ds}G_{ds}^{m}C_{ds}G_{ds}^{m}Cₖₛ | 32.1 | 719 | 734 | 558 | 1024 |
| | Gₖₛ^{m}Cₖ | | | | | |
| 569469 | Gₖₛ^{m}Cₖₛ^{m}CₖₛA_{ds}G_{ds}G_{ds}T_{ds}A_{ds}G_{ds}A_{ds}A_{ds}G_{ds}^{m}C_{ds}Gₖₛ^{m} | 54.5 | 720 | 735 | 559 | 1025 |
| | CₖₛGₖ | | | | | |
| 569470 | ^{m}CₖₛGₖₛ^{m}Cₖₛ^{m}C_{ds}A_{ds}G_{ds}G_{ds}T_{ds}A_{ds}G_{ds}A_{ds}A_{ds}G_{ds}^{m}Cₖₛ | 50.5 | 721 | 736 | 560 | 1026 |
| | Gₖₛ^{m}Cₖ | | | | | |
| 569471 | ^{m}Cₖₛ^{m}CₖₛGₖₛ^{m}C_{ds}^{m}C_{ds}A_{ds}G_{ds}G_{ds}T_{ds}A_{ds}G_{ds}A_{ds}A_{ds}Gₖₛ | 56.6 | 722 | 737 | 561 | 1027 |
| | ^{m}CₖₛGₖ | | | | | |
| 569472 | Tₖₛ^{m}Cₖₛ^{m}CₖₛG_{ds}^{m}C_{ds}^{m}C_{ds}A_{ds}G_{ds}G_{ds}T_{ds}A_{ds}G_{ds}A_{ds}Aₖₛ | 44.1 | 723 | 738 | 562 | 1028 |
| | Gₖₛ^{m}Cₖ | | | | | |
| 569473 | GₖₛAₖₛ^{m}CₖₛA_{ds}A_{ds}T_{ds}^{m}C_{ds}T_{ds}^{m}C_{ds}^{m}C_{ds}G_{ds}^{m}C_{ds}^{m}C_{ds} | 14.2 | 730 | 745 | 29 | 882 |
| | AₖₛGₖₛGₖ | | | | | |
| 569474 | TₖₛGₖₛAₖₛ^{m}C_{ds}A_{ds}A_{ds}T_{ds}^{m}C_{ds}T_{ds}^{m}C_{ds}^{m}C_{ds}G_{ds}^{m}C_{ds}^{m} | 25.9 | 731 | 746 | 563 | 1029 |
| | CₖₛAₖₛGₖ | | | | | |
| 569475 | AₖₛTₖₛGₖₛA_{ds}^{m}C_{ds}A_{ds}A_{ds}T_{ds}^{m}C_{ds}T_{ds}^{m}C_{ds}^{m}C_{ds}G_{ds}^{m}Cₖₛ | 28.7 | 732 | 747 | 564 | 1030 |
| | ^{m}CₖₛAₖ | | | | | |
| 569476 | ^{m}CₖₛAₖₛTₖₛG_{ds}A_{ds}^{m}C_{ds}A_{ds}A_{ds}T_{ds}^{m}C_{ds}T_{ds}^{m}C_{ds}^{m}C_{ds}Gₖₛ | 27.4 | 733 | 748 | 565 | 1031 |
| | ^{m}Cₖₛ^{m}Cₖ | | | | | |
| 569477 | ^{m}Cₖₛ^{m}CₖₛAₖₛT_{ds}G_{ds}A_{ds}^{m}C_{ds}A_{ds}A_{ds}T_{ds}^{m}C_{ds}T_{ds}^{m}C_{ds}^{m} | 52.4 | 734 | 749 | 566 | 1032 |
| | CₖₛGₖₛ^{m}Cₖ | | | | | |
| 569478 | Gₖₛ^{m}Cₖₛ^{m}CₖₛA_{ds}T_{ds}G_{ds}A_{ds}^{m}C_{ds}A_{ds}A_{ds}T_{ds}^{m}C_{ds}T_{ds}^{m}Cₖₛ | 50.5 | 735 | 750 | 567 | 1033 |
| | ^{m}CₖₛGₖ | | | | | |
| 569479 | GₖₛGₖₛ^{m}Cₖₛ^{m}C_{ds}A_{ds}T_{ds}G_{ds}A_{ds}^{m}C_{ds}A_{ds}A_{ds}T_{ds}^{m}C_{ds}Tₖₛ^{m} | 48.4 | 736 | 751 | 568 | 1034 |
| | Cₖₛ^{m}Ck | | | | | |

TABLE 14
Inhibition of human DMPK RNA transcript in
HepG2 cells targeting SEQ ID NO: 2

| | | | Start | Stop | Un- | |
|---|---|---|---|---|---|---|
| | | Sequence | Site | Site | modified | Modified |
| ISIS | | % Target | on Seq | on Seq | Seq ID | Seq ID |
| No. | | Expression | ID: 2 | ID: 2 | No. | No. |
| UTC | N/A | 100 | N/A | N/A | | |
| 445569 | ^{m}CₑₛGₑₛGₑₛAₑₛGₑₛ^{m}C_{ds}G_{ds}G_{ds}T_{ds}T_{ds}G_{ds}T_{ds}G_{ds} | 31.4 | 13226 | 13245 | 24 | 1443 |
| | A_{ds}A_{ds}^{m}CₑₛTₑₛGₑₛGₑₛ^{m}Cₑ | | | | | |
| 486178 | Aₖₛ^{m}CₖₛAₖₛA_{ds}T_{ds}A_{ds}A_{ds}A_{ds}T_{ds}A_{ds}^{m}C_{ds}^{m}C_{ds}G_{ds} | 25.3 | 13836 | 13851 | 23 | 881 |
| | AₖₛGₖₛGₖ | | | | | |
| 570801 | ^{m}Cₖₛ^{m}CₖₛAₖₛA_{ds}^{m}C_{ds}T_{ds}G_{ds}T_{ds}T_{ds}^{m}C_{ds}T_{ds}^{m}C_{ds}T_{ds} | 22.7 | 10165 | 10180 | 569 | 1035 |
| | TₖₛAₖₛGₖ | | | | | |
| 570802 | AₖₛAₖₛ^{m}Cₖₛ^{m}C_{ds}A_{ds}A_{ds}^{m}C_{ds}T_{ds}G_{ds}T_{ds}T_{ds}^{m}C_{ds}T_{ds} | 22.6 | 10167 | 10182 | 570 | 1036 |
| | ^{m}CₖₛTₖₛTₖ | | | | | |
| 570803 | ^{m}Cₖₛ^{m}CₖₛAₖₛG_{ds}T_{ds}A_{ds}A_{ds}T_{ds}A_{ds}A_{ds}A_{ds}A_{ds}G_{ds} | 37.4 | 10190 | 10205 | 571 | 1037 |
| | ^{m}CₖₛTₖₛGₖ | | | | | |
| 570804 | GₖₛTₖₛ^{m}Cₖₛ^{m}C_{ds}A_{ds}G_{ds}T_{ds}A_{ds}A_{ds}T_{ds}A_{ds}A_{ds}A_{ds}Aₖₛ | 24.9 | 10192 | 10207 | 572 | 1038 |
| | Gₖₛ^{m}Cₖ | | | | | |
| 570805 | GₖₛTₖₛTₖₛG_{ds}T_{ds}^{m}C_{ds}^{m}C_{ds}A_{ds}G_{ds}T_{ds}A_{ds}A_{ds}T_{ds}Aₖₛ | 23.8 | 10195 | 10210 | 573 | 1039 |
| | AₖₛAₖ | | | | | |
| 570806 | AₖₛTₖₛGₖₛT_{ds}T_{ds}G_{ds}T_{ds}^{m}C_{ds}^{m}C_{ds}A_{ds}G_{ds}T_{ds}A_{ds}Aₖₛ | 21.9 | 10197 | 10212 | 574 | 1040 |
| | TₖₛAₖ | | | | | |
| 570807 | TₖₛAₖₛAₖₛT_{ds}G_{ds}T_{ds}T_{ds}G_{ds}T_{ds}^{m}C_{ds}^{m}C_{ds}A_{ds}G_{ds}Tₖₛ | 20 | 10199 | 10214 | 575 | 1041 |
| | AₖₛAₖ | | | | | |
| 570808 | TₖₛGₖₛTₖₛA_{ds}A_{ds}T_{ds}G_{ds}T_{ds}T_{ds}G_{ds}T_{ds}^{m}C_{ds}^{m}C_{ds}Aₖₛ | 11.5 | 10201 | 10216 | 31 | 883 |
| | GₖₛTₖ | | | | | |
| 570809 | TₖₛTₖₛ^{m}CₖₛA_{ds}A_{ds}T_{ds}^{m}C_{ds}^{m}C_{ds}T_{ds}G_{ds}A_{ds}^{m}C_{ds}^{m}C_{ds} | 34.7 | 10279 | 10294 | 576 | 1042 |
| | ^{m}CₖₛAₖₛ^{m}Cₖ | | | | | |
| 570810 | GₖₛGₖₛTₖₛT_{ds}^{m}C_{ds}A_{ds}A_{ds}T_{ds}^{m}C_{ds}^{m}C_{ds}T_{ds}G_{ds}A_{ds} | 76.4 | 10281 | 10296 | 577 | 1043 |
| | ^{m}Cₖₛ^{m}Cₖₛ^{m}Cₖ | | | | | |
| 570811 | TₖₛGₖₛGₖₛG_{ds}T_{ds}T_{ds}^{m}C_{ds}A_{ds}A_{ds}T_{ds}^{m}C_{ds}^{m}C_{ds}T_{ds} | 72.4 | 10283 | 10298 | 578 | 1044 |
| | GₖₛAₖₛ^{m}Cₖ | | | | | |
| 570812 | GₖₛAₖₛTₖₛG_{ds}G_{ds}G_{ds}T_{ds}T_{ds}^{m}C_{ds}A_{ds}A_{ds}T_{ds}^{m}C_{ds} | 49 | 10285 | 10300 | 579 | 1045 |
| | ^{m}CₖₛTₖₛGₖ | | | | | |
| 570813 | AₖₛGₖₛGₖₛA_{ds}T_{ds}G_{ds}G_{ds}G_{ds}T_{ds}T_{ds}^{m}C_{ds}A_{ds}A_{ds}Tₖₛ | 80.8 | 10287 | 10302 | 580 | 1046 |
| 570814 | AₖₛGₖₛAₖₛG_{ds}G_{ds}A_{ds}T_{ds}G_{ds}G_{ds}G_{ds}T_{ds}T_{ds}^{m}C_{ds}Aₖₛ | 43.3 | 10289 | 10304 | 581 | 1047 |
| | AₖₛTₖ | | | | | |
| 570815 | AₖₛTₖₛAₖₛG_{ds}A_{ds}G_{ds}G_{ds}A_{ds}T_{ds}G_{ds}G_{ds}G_{ds}T_{ds}Tₖₛ | 63.2 | 10291 | 10306 | 582 | 1048 |
| | CₖₛAₖ | | | | | |
| 570816 | ^{m}Cₖₛ^{m}Cₖₛ^{m}CₖₛT_{ds}^{m}C_{ds}^{m}C_{ds}T_{ds}G_{ds}T_{ds}G_{ds}G_{ds}G_{ds}A_{ds} | 38.8 | 10349 | 10364 | 583 | 1049 |
| | Aₖₛ^{m}CₖₛAₖ | | | | | |
| 570817 | GₖₛTₖₛ^{m}Cₖₛ^{m}C_{ds}^{m}C_{ds}T_{ds}^{m}C_{ds}^{m}C_{ds}T_{ds}G_{ds}T_{ds}G_{ds}G_{ds} | 91 | 10351 | 10366 | 584 | 1050 |
| | GₖₛAₖₛAₖ | | | | | |
| 570818 | ^{m}CₖₛAₖₛGₖₛT_{ds}^{m}C_{ds}^{m}C_{ds}^{m}C_{ds}T_{ds}^{m}C_{ds}^{m}C_{ds}T_{ds}G_{ds} | 64.8 | 10353 | 10368 | 585 | 1051 |
| | T_{ds}GₖₛGₖₛGₖ | | | | | |
| 570819 | AₖₛGₖₛ^{m}CₖₛA_{ds}G_{ds}T_{ds}^{m}C_{ds}^{m}C_{ds}^{m}C_{ds}T_{ds}^{m}C_{ds}^{m}C_{ds} | 28.5 | 10355 | 10370 | 586 | 1052 |
| | T_{ds}GₖₛTₖₛGₖ | | | | | |
| 570820 | Aₖₛ^{m}CₖₛTₖₛ^{m}C_{ds}A_{ds}G_{ds}^{m}C_{ds}T_{ds}G_{ds}T_{ds}G_{ds}G_{ds}G_{ds} | 62.9 | 10417 | 10432 | 587 | 1053 |
| | AₖₛAₖₛGₖ | | | | | |
| 570821 | ^{m}Cₖₛ^{m}Cₖₛ^{m}CₖₛA_{ds}^{m}C_{ds}T_{ds}^{m}C_{ds}A_{ds}G_{ds}^{m}C_{ds}T_{ds}G_{ds} | 79.9 | 10420 | 10435 | 588 | 1054 |
| | T_{ds}GₖₛGₖₛGₖ | | | | | |
| 570822 | Aₖₛ^{m}Cₖₛ^{m}Cₖₛ^{m}C_{ds}^{m}C_{ds}A_{ds}^{m}C_{ds}T_{ds}^{m}C_{ds}A_{ds}G_{ds}^{m}C_{ds} | 47.5 | 10422 | 10437 | 589 | 1055 |
| | T_{ds}GₖₛTₖₛGₖ | | | | | |
| 570823 | Aₖₛ^{m}CₖₛAₖₛ^{m}C_{ds}^{m}C_{ds}^{m}C_{ds}^{m}C_{ds}A_{ds}^{m}C_{ds}T_{ds}^{m}C_{ds}A_{ds} | 78.1 | 10424 | 10439 | 590 | 1056 |
| | G_{ds}^{m}CₖₛTₖₛGₖ | | | | | |
| 570824 | Gₖₛ^{m}CₖₛAₖₛ^{m}C_{ds}A_{ds}^{m}C_{ds}^{m}C_{ds}^{m}C_{ds}^{m}C_{ds}A_{ds}^{m}C_{ds}T_{ds} | 82.5 | 10426 | 10441 | 591 | 1057 |
| | ^{m}C_{ds}AₖₛGₖₛ^{m}Cₖ | | | | | |
| 570825 | Tₖₛ^{m}CₖₛAₖₛG_{ds}^{m}C_{ds}A_{ds}^{m}C_{ds}A_{ds}^{m}C_{ds}^{m}C_{ds}^{m}C_{ds}^{m}C_{ds} | 52.6 | 10429 | 10444 | 592 | 1058 |
| | A_{ds}^{m}CₖₛTₖₛ^{m}Cₖ | | | | | |
| 570826 | GₖₛTₖₛGₖₛG_{ds}T_{ds}^{m}C_{ds}^{m}C_{ds}T_{ds}A_{ds}A_{ds}G_{ds}A_{ds}^{m}C_{ds} | 30.9 | 10474 | 10489 | 593 | 1059 |
| | TₖₛGₖₛGₖ | | | | | |
| 570827 | GₖₛAₖₛTₖₛG_{ds}T_{ds}G_{ds}G_{ds}T_{ds}^{m}C_{ds}^{m}C_{ds}T_{ds}A_{ds}A_{ds}Gₖₛ | 25.5 | 10477 | 10492 | 594 | 1060 |
| | Aₖₛ^{m}Cₖ | | | | | |
| 570828 | ^{m}CₖₛAₖₛGₖₛA_{ds}T_{ds}G_{ds}T_{ds}G_{ds}G_{ds}T_{ds}^{m}C_{ds}^{m}C_{ds}T_{ds} | 18.6 | 10479 | 10494 | 595 | 1061 |
| | AₖₛAₖₛGₖ | | | | | |
| 570829 | ^{m}Cₖₛ^{m}CₖₛTₖₛ^{m}C_{ds}^{m}C_{ds}A_{ds}^{m}C_{ds}A_{ds}G_{ds}A_{ds}T_{ds}G_{ds}T_{ds} | 44.5 | 10485 | 10500 | 596 | 1062 |
| | GₖₛGₖₛTₖ | | | | | |
| 570830 | ^{m}CₖₛAₖₛ^{m}Cₖₛ^{m}C_{ds}T_{ds}^{m}C_{ds}^{m}C_{ds}A_{ds}^{m}C_{ds}A_{ds}G_{ds}A_{ds} | 67.4 | 10487 | 10502 | 597 | 1063 |
| | T_{ds}GₖₛTₖₛGₖ | | | | | |
| 570831 | GₖₛGₖₛ^{m}Cₖₛ^{m}C_{ds}A_{ds}^{m}C_{ds}^{m}C_{ds}T_{ds}C_{ds}^{m}C_{ds}A_{ds}^{m}C_{ds} | 56.3 | 10490 | 10505 | 598 | 1064 |
| | A_{ds}GₖₛAₖₛTₖ | | | | | |
| 570832 | TₖₛGₖₛ^{m}CₖₛT_{ds}T_{ds}G_{ds}G_{ds}^{m}C_{ds}T_{ds}^{m}C_{ds}T_{ds}G_{ds}G_{ds} | 42.4 | 10501 | 10516 | 599 | 1065 |
| | ^{m}Cₖₛ^{m}CₖₛAₖ | | | | | |
| 570833 | Aₖₛ^{m}CₖₛTₖₛG_{ds}^{m}C_{ds}T_{ds}T_{ds}G_{ds}G_{ds}^{m}C_{ds}T_{ds}^{m}C_{ds}T_{ds} | 16 | 10503 | 10518 | 600 | 1066 |
| | GₖₛGₖₛ^{m}Cₖ | | | | | |
| 570834 | AₖₛGₖₛAₖₛ^{m}C_{ds}T_{ds}G_{ds}^{m}C_{ds}T_{ds}T_{ds}G_{ds}G_{ds}^{m}C_{ds}T_{ds} | 47.5 | 10505 | 10520 | 601 | 1067 |
| | ^{m}CₖₛTₖₛGₖ | | | | | |
| 570835 | GₖₛGₖₛAₖₛG_{ds}A_{ds}^{m}C_{ds}T_{ds}G_{ds}^{m}C_{ds}T_{ds}T_{ds}G_{ds}G_{ds} | 37.2 | 10507 | 10522 | 602 | 1068 |
| | ^{m}CₖₛTₖₛ^{m}Cₖ | | | | | |
| 570836 | TₖₛGₖₛ^{m}CₖₛA_{ds}G_{ds}A_{ds}^{m}C_{ds}^{m}C_{ds}^{m}C_{ds}^{m}C_{ds}T_{ds}^{m}C_{ds} | 63.1 | 10556 | 10571 | 603 | 1069 |
| | T_{ds}Tₖₛ^{m}CₖₛTₖ | | | | | |
| 570837 | ^{m}CₖₛTₖₛ^{m}Cₖₛ^{m}C_{ds}T_{ds}^{m}C_{ds}^{m}C_{ds}^{m}C_{ds}T_{ds}T_{ds}G_{ds}A_{ds} | 60.7 | 10579 | 10594 | 604 | 1070 |
| | ^{m}C_{ds}AₖₛTₖₛGₖ | | | | | |
| 570838 | ^{m}Cₖₛ^{m}CₖₛAₖₛG_{ds}A_{ds}^{m}C_{ds}^{m}C_{ds}^{m}C_{ds}^{m}C_{ds}C_{ds}A_{ds}T_{ds} | 42.9 | 10609 | 10624 | 605 | 1071 |
| | G_{ds}TₖₛTₖₛ^{m}Cₖ | | | | | |
| 570839 | GₖₛTₖₛ^{m}Cₖₛ^{m}C_{ds}A_{ds}G_{ds}A_{ds}^{m}C_{ds}^{m}C_{ds}^{m}C_{ds}^{m}C_{ds}^{m}C_{ds} | 64.3 | 10611 | 10626 | 606 | 1072 |
| | A_{ds}TₖₛGₖₛTₖ | | | | | |
| 570840 | GₖₛGₖₛGₖₛT_{ds}^{m}C_{ds}^{m}C_{ds}A_{ds}G_{ds}A_{ds}^{m}C_{ds}^{m}C_{ds}^{m}C_{ds} | 68.5 | 10613 | 10628 | 607 | 1073 |
| | ^{m}C_{ds}^{m}CₖₛAₖₛTₖ | | | | | |
| 570841 | Aₖₛ^{m}Cₖₛ^{m}CₖₛT_{ds}T_{ds}^{m}C_{ds}T_{ds}G_{ds}^{m}C_{ds}A_{ds}G_{ds}G_{ds}G_{ds} | 14.9 | 10631 | 10646 | 608 | 1074 |
| | Aₖₛ^{m}CₖₛTₖ | | | | | |
| 570842 | TₖₛAₖₛAₖₛA_{ds}^{m}C_{ds}^{m}C_{ds}T_{ds}T_{ds}^{mm}C_{ds}T_{ds}G_{ds}^{m}C_{ds}A_{ds} | 51.7 | 10634 | 10649 | 609 | 1075 |
| | GₖₛGₖₛGₖ | | | | | |
| 570843 | GₖₛAₖₛAₖₛA_{ds}A_{ds}G_{ds}^{m}C_{ds}^{m}C_{ds}^{m}C_{ds}T_{ds}G_{ds}^{m}C_{ds}^{m}C_{ds} | 46.3 | 10684 | 10699 | 610 | 1076 |
| | ^{m}Cₖₛ^{m}CₖₛTₖ | | | | | |
| 570844 | TₖₛAₖₛGₖₛG_{ds}A_{ds}A_{ds}A_{ds}A_{ds}G_{ds}^{m}C_{ds}^{m}C_{ds}^{m}C_{ds}T_{ds} | 52.3 | 10687 | 10702 | 611 | 1077 |
| | Gₖₛ^{m}Cₖₛ^{m}Cₖ | | | | | |
| 570845 | ^{m}CₖₛIₖₛTₖₛA_{ds}G_{ds}G_{ds}A_{ds}A_{ds}A_{ds}A_{ds}G_{ds}^{m}C_{ds}^{m}C_{ds} | 53.8 | 10689 | 10704 | 612 | 1078 |
| | ^{m}CₖₛTₖₛGₖ | | | | | |
| 570846 | TₖₛGₖₛ^{m}CₖₛT_{ds}T_{ds}A_{ds}G_{ds}G_{ds}A_{ds}A_{ds}A_{ds}A_{ds}G_{ds}^{m}Cₖₛ | 47.8 | 10691 | 10706 | 613 | 1079 |
| | ^{m}Cₖₛ^{m}Cₖ | | | | | |
| 570847 | Tₖₛ^{m}CₖₛTₖₛG_{ds}^{m}C_{ds}T_{ds}T_{ds}A_{ds}G_{ds}G_{ds}A_{ds}A_{ds}A_{ds}Aₖₛ | 43.9 | 10693 | 10708 | 614 | 1080 |
| | Gₖₛ^{m}Cₖ | | | | | |
| 570848 | ^{m}CₖₛTₖₛ^{m}Cₖₛ^{m}C_{ds}I_{ds}^{m}C_{ds}T_{ds}G_{ds}^{m}C_{ds}T_{ds}T_{ds}A_{ds}G_{ds} | 67.9 | 10697 | 10712 | 615 | 1081 |
| | GₖₛAₖₛAₖ | | | | | |
| 570849 | ^{m}Cₖₛ^{m}Cₖₛ^{m}CₖₛT_{ds}^{m}C_{ds}^{m}C_{ds}T_{ds}^{m}C_{ds}T_{ds}G_{ds}^{m}C_{ds}T_{ds} | 50.8 | 10699 | 10714 | 616 | 1082 |
| | T_{ds}AₖₛGₖₛGₖ | | | | | |
| 570850 | ^{m}CₖₛTₖₛGₖₛA_{ds}T_{ds}T_{ds}T_{ds}G_{ds}A_{ds}G_{ds}G_{ds}A_{ds}A_{ds}Gₖₛ | 41.1 | 10759 | 10774 | 617 | 1083 |
| | GₖₛGₖ | | | | | |
| 570851 | Tₖₛ^{m}Cₖₛ^{m}CₖₛT_{ds}G_{ds}A_{ds}T_{ds}T_{ds}T_{ds}G_{ds}A_{ds}G_{ds}G_{ds}Aₖₛ | 87.4 | 10761 | 10776 | 618 | 1084 |
| | AₖₛGₖ | | | | | |
| 570852 | ^{m}Cₖₛ^{m}CₖₛTₖₛ^{m}C_{ds}^{m}C_{ds}T_{ds}G_{ds}A_{ds}T_{ds}T_{ds}T_{ds}G_{ds}A_{ds} | 75.8 | 10763 | 10778 | 619 | 1085 |
| | GₖₛGₖₛAₖ | | | | | |
| 570853 | GₖₛAₖₛ^{m}Cₖₛ^{m}C_{ds}T_{ds}^{m}C_{ds}^{m}C_{ds}T_{ds}G_{ds}A_{ds}T_{ds}T_{ds}T_{ds} | 87.4 | 10765 | 10780 | 620 | 1086 |
| | GₖₛAₖₛGₖ | | | | | |
| 570854 | AₖₛAₖₛGₖₛA_{ds}^{m}C_{ds}^{m}C_{ds}T_{ds}^{m}C_{ds}^{m}C_{ds}T_{ds}G_{ds}A_{ds}T_{ds} | 60.3 | 10767 | 10782 | 621 | 1087 |
| | TₖₛTₖₛGₖ | | | | | |
| 570855 | ^{m}Cₖₛ^{m}CₖₛAₖₛA_{ds}G_{ds}A_{ds}^{m}C_{ds}^{m}C_{ds}T_{ds}^{m}C_{ds}^{m}C_{ds}T_{ds} | 61.4 | 10769 | 10784 | 622 | 1088 |
| | G_{ds}AₖₛTₖₛTₖ | | | | | |
| 570856 | ^{m}CₖₛTₖₛGₖₛ^{m}C_{ds}T_{ds}T_{ds}^{m}C_{ds}^{m}C_{ds}A_{ds}A_{ds}G_{ds}A_{ds}^{m}C_{ds} | 40.4 | 10775 | 10790 | 623 | 1089 |
| | ^{m}CₖₛTₖₛ^{m}Cₖ | | | | | |
| 570857 | AₖₛGₖₛ^{m}CₖₛT_{ds}G_{ds}^{m}C_{ds}T_{ds}T_{ds}^{m}C_{ds}^{m}C_{ds}A_{ds}A_{ds}G_{ds} | 48.5 | 10777 | 10792 | 624 | 1090 |
| | Aₖₛ^{m}Cₖₛ^{m}Cₖ | | | | | |
| 570858 | Gₖₛ^{m}CₖₛAₖₛG_{ds}^{mm}C_{ds}T_{ds}G_{ds}^{m}C_{ds}T_{ds}T_{ds}^{m}C_{ds}^{m}C_{ds} | 87.7 | 10779 | 10794 | 625 | 1091 |
| | A_{ds}AₖₛGₖₛAₖ | | | | | |
| 570859 | ^{m}CₖₛTₖₛGₖₛG_{ds}T_{ds}G_{ds}G_{ds}A_{ds}G_{ds}A_{ds}A_{ds}^{m}C_{ds}^{m}C_{ds} | 92.6 | 10816 | 10831 | 626 | 1092 |
| | AₖₛGₖₛAₖ | | | | | |
| 570860 | ^{m}CₖₛTₖₛ^{m}CₖₛT_{ds}G_{ds}G_{ds}T_{ds}G_{ds}G_{ds}A_{ds}G_{ds}A_{ds}A_{ds} | 86.6 | 10818 | 10833 | 627 | 1093 |
| | ^{m}CₖₛCₖₛAₖ | | | | | |
| 570861 | TₖₛTₖₛ^{m}CₖₛT_{ds}^{m}C_{ds}T_{ds}G_{ds}G_{ds}T_{ds}G_{ds}G_{ds}A_{ds}G_{ds}Aₖₛ | 82.6 | 10820 | 10835 | 628 | 1094 |
| | Aₖₛ^{m}Cₖ | | | | | |
| 570862 | GₖₛAₖₛTₖₛT_{ds}^{m}C_{ds}T_{ds}^{m}C_{ds}T_{ds}G_{ds}G_{ds}T_{ds}G_{ds}G_{ds}Aₖₛ | 76.1 | 10822 | 10837 | 629 | 1095 |
| | GₖₛAₖ | | | | | |
| 570863 | Aₖₛ^{m}CₖₛTₖₛT_{ds}A_{ds}^{m}C_{ds}T_{ds}G_{ds}T_{ds}T_{ds}T_{ds}^{m}C_{ds}A_{ds} | 80.6 | 10981 | 10996 | 630 | 1096 |
| | Tₖₛ^{m}Cₖₛ^{m}Cₖ | | | | | |
| 570864 | ^{m}CₖₛGₖₛGₖₛA_{ds}^{m}C_{ds}^{m}C_{ds}^{m}C_{ds}^{m}C_{ds}^{m}C_{ds}T_{ds}^{m}C_{ds}^{m}C | 58.7 | 11002 | 11017 | 631 | 1097 |
| | ^{m}C_{ds}^{m}CₖₛTₖₛ^{m}Cₖ | | | | | |
| 570865 | GₖₛAₖₛ^{m}CₖₛG_{ds}G_{ds}A_{ds}^{m}C_{ds}^{m}C_{ds}^{m}C_{ds}^{m}C_{ds}^{m}C_{ds}T_{ds} | 61.5 | 11004 | 11019 | 632 | 1098 |
| | ^{m}C_{ds}^{m}Cₖₛ^{m}Cₖₛ^{m}Cₖ | | | | | |
| 570866 | ^{m}CₖₛTₖₛGₖₛA_{ds}^{m}C_{ds}G_{ds}G_{ds}A_{ds}^{m}C_{ds}^{m}C_{ds}^{m}C_{ds}^{m}C_{ds} | 47.6 | 11006 | 11021 | 633 | 1099 |
| | ^{m}C_{ds}Tₖₛ^{m}Cₖₛ^{m}Cₖ | | | | | |
| 570867 | ^{m}Cₖₛ^{m}Cₖₛ^{m}CₖₛT_{ds}G_{ds}A_{ds}^{m}C_{ds}G_{ds}G_{ds}A_{ds}^{m}C_{ds}^{m}C_{ds} | 69.5 | 11008 | 11023 | 634 | 1100 |
| | ^{m}C_{ds}^{m}Cₖₛ^{m}CₖₛTₖ | | | | | |
| 570868 | AₖₛAₖₛGₖₛ^{m}C_{ds}^{m}C_{ds}^{m}C_{ds}T_{ds}^{m}C_{ds}A_{ds}^{m}C_{ds}^{m}C_{ds}T_{ds} | 54 | 11036 | 11051 | 635 | 1101 |
| | T_{ds}TₖₛTₖₛ^{m}Cₖ | | | | | |
| 570869 | GₖₛGₖₛAₖₛA_{ds}G_{ds}^{m}C_{ds}^{m}C_{ds}^{m}C_{ds}T_{ds}^{m}C_{ds}A_{ds}^{m}C_{ds} | 37.5 | 11038 | 11053 | 636 | 1102 |
| | ^{m}C_{ds}TₖₛTₖₛTₖ | | | | | |
| 570870 | ^{m}CₖₛGₖₛGₖₛG_{ds}A_{ds}A_{ds}G_{ds}^{m}C_{ds}^{m}C_{ds}^{m}C_{ds}T_{ds}^{m}C_{ds}A_{ds} | 70.7 | 11040 | 11055 | 637 | 1103 |
| | ^{m}Cₖₛ^{m}CₖₛTₖ | | | | | |
| 570871 | ^{m}Cₖₛ^{m}Cₖₛ^{m}CₖₛG_{ds}G_{ds}G_{ds}A_{ds}A_{ds}G_{ds}^{m}C_{ds}^{m}C_{ds}^{m}C_{ds} | 71.2 | 11042 | 11057 | 638 | 1104 |
| | T_{ds}^{m}CₖₛAₖₛ^{m}Cₖ | | | | | |
| 570872 | ^{m}CₖₛAₖₛ^{m}Cₖₛ^{m}C_{ds}^{m}C_{ds}G_{ds}G_{ds}G_{ds}A_{ds}A_{ds}G_{ds}^{m}C_{ds} | 51.6 | 11044 | 11059 | 639 | 1105 |
| | ^{m}C_{ds}^{m}CₖₛTₖₛ^{m}Cₖ | | | | | |
| 570873 | Gₖₛ^{m}Cₖₛ^{m}CₖₛA_{ds}^{m}C_{ds}^{m}C_{ds}C_{ds}G_{ds}G_{ds}G_{ds}A_{ds}A_{ds}G_{ds} | 45.8 | 11046 | 11061 | 640 | 1106 |
| | ^{m}Cₖₛ^{m}Cₖₛ^{m}Cₖ | | | | | |
| 570874 | Aₖₛ^{m}CₖₛGₖₛ^{m}C_{ds}^{m}C_{ds}A_{ds}^{m}C_{ds}^{m}C_{ds}^{m}C_{ds}G_{ds}G_{ds}G_{ds} | 31.8 | 11048 | 11063 | 641 | 1107 |
| | A_{ds}AₖₛGₖₛ^{m}Cₖ | | | | | |
| 570875 | ^{m}CₖₛTₖₛGₖₛT_{ds}T_{ds}^{m}C_{ds}A_{ds}G_{ds}G_{ds}A_{ds}A_{ds}G_{ds}T_{ds} | 14.3 | 11082 | 11097 | 642 | 1108 |
| | ^{m}Cₖₛ^{m}Cₖₛ^{m}Cₖ | | | | | |
| 570876 | TₖₛTₖₛ^{m}CₖₛT_{ds}G_{ds}T_{ds}T_{ds}^{m}C_{ds}A_{ds}G_{ds}G_{ds}A_{ds}A_{ds}Gₖₛ | 18 | 11084 | 11099 | 643 | 1109 |
| | Tₖₛ^{m}Cₖ | | | | | |
| 570877 | Gₖₛ^{m}CₖₛTₖₛT_{ds}^{m}C_{ds}T_{ds}G_{ds}T_{ds}T_{ds}^{m}C_{ds}A_{ds}G_{ds}G_{ds} | 44 | 11086 | 11101 | 644 | 1110 |
| | AₖₛAₖₛGₖ | | | | | |

TABLE 15
Inhibition of human DMPK RNA transcript in HepG2 cells targeting SEQ ID NO: 2

| | | | Start | Stop | | |
|---|---|---|---|---|---|---|
| | | | Site on | Site on | | Modified |
| ISIS | | % Target | Seq | Seq ID: | Unmodified | Seq ID |
| No. | Sequence | Expression | ID: 2 | 2 | Seq ID No. | No. |
| UTC | N/A | 100 | N/A | N/A | | |
| 445569 | ^{m}CₑₛGₑₛGₑₛAₑₛGₑₛ^{m}C_{ds}G_{ds}G_{ds}T_{ds}T_{ds}G_{ds}T_{ds}G_{ds} | 55 | 13226 | 13245 | 24 | 1443 |
| | A_{ds}A_{ds}^{m}CₑₛTₑₛGₑₛGₑₛ^{m}Cₑ | | | | | |
| 486178 | Aₖₛ^{m}CₖₛAₖₛA_{ds}T_{ds}A_{ds}A_{ds}A_{ds}T_{ds}A_{ds}^{m}C_{ds}^{m}C_{ds} | 33.9 | 13836 | 13851 | 23 | 881 |
| | G_{ds}AₖₛGₖₛGₖ | | | | | |
| 570647 | Gₖₛ^{m}CₖₛTₖₛT_{ds}G_{ds}G_{ds}G_{ds}^{m}C_{ds}^{m}C_{ds}^{m}C_{ds}A_{ds}^{m}C_{ds} | 80.3 | 5718 | 5733 | 645 | 1111 |
| | ^{m}C_{ds}^{m}Cₖₛ^{m}CₖₛTₖ | | | | | |
| 570648 | AₖₛGₖₛGₖₛ^{m}C_{ds}T_{ds}T_{ds}G_{ds}G_{ds}G_{ds}^{m}C_{ds}^{m}C_{ds}^{m}C_{ds} | 92.3 | 5720 | 5735 | 646 | 1112 |
| | A_{ds}^{m}Cₖₛ^{m}Cₖₛ^{m}Cₖ | | | | | |
| 570649 | ^{m}CₖₛGₖₛAₖₛG_{ds}G_{ds}^{m}C_{ds}T_{ds}T_{ds}G_{ds}G_{ds}G_{ds}^{m}C_{ds}^{m} | 100.7 | 5722 | 5737 | 647 | 1113 |
| | C_{ds}^{m}CₖₛAₖₛ^{m}Cₖ | | | | | |
| 570650 | AₖₛGₖₛ^{m}CₖₛG_{ds}A_{ds}G_{ds}G_{ds}^{m}C_{ds}T_{ds}T_{ds}G_{ds}G_{ds}G_{ds} | 75.8 | 5724 | 5739 | 648 | 1114 |
| | ^{m}Cₖₛ^{m}Cₖₛ^{m}Cₖ | | | | | |
| 570651 | AₖₛGₖₛAₖₛG_{ds}^{m}C_{ds}G_{ds}A_{ds}G_{ds}G_{ds}^{m}C_{ds}T_{ds}T_{ds}G_{ds} | 99.8 | 5726 | 5741 | 649 | 1115 |
| | GₖₛGₖₛ^{m}Cₖ | | | | | |
| 570652 | Gₖₛ^{m}CₖₛAₖₛG_{ds}A_{ds}G_{ds}^{m}C_{ds}G_{ds}A_{ds}G_{ds}G_{ds}^{m}C_{ds} | 135.4 | 5728 | 5743 | 650 | 1116 |
| | T_{ds}TₖₛGₖₛGₖ | | | | | |
| 570653 | GₖₛAₖₛGₖₛ^{m}C_{ds}A_{ds}G_{ds}A_{ds}G_{ds}^{m}C_{ds}G_{ds}A_{ds}G_{ds} | 111.5 | 5730 | 5745 | 651 | 1117 |
| | G_{ds}^{m}CₖₛTₖₛTₖ | | | | | |
| 570654 | AₖₛAₖₛAₖₛG_{ds}G_{ds}A_{ds}G_{ds}^{m}C_{ds}A_{ds}G_{ds}A_{ds}G_{ds}^{m} | 87.5 | 5734 | 5749 | 652 | 1118 |
| | C_{ds}GₖₛAₖₛGₖ | | | | | |
| 570655 | ^{m}CₖₛAₖₛAₖₛA_{ds}A_{ds}G_{ds}G_{ds}A_{ds}G_{ds}^{m}C_{ds}A_{ds}G_{ds} | 94.5 | 5736 | 5751 | 653 | 1119 |
| | A_{ds}Gₖₛ^{m}CₖₛGₖ | | | | | |
| 570656 | TₖₛGₖₛGₖₛA_{ds}^{m}C_{ds}^{m}C_{ds}A_{ds}A_{ds}A_{ds}A_{ds}G_{ds}G_{ds}A_{ds} | 75.4 | 5741 | 5756 | 654 | 1120 |
| | Gₖₛ^{m}CₖₛAₖ | | | | | |
| 570657 | ^{m}Cₖₛ^{m}CₖₛTₖₛG_{ds}G_{ds}A_{ds}^{m}C_{ds}^{m}C_{ds}A_{ds}A_{ds}A_{ds}A_{ds} | 87.3 | 5743 | 5758 | 655 | 1121 |
| | G_{ds}GₖₛAₖₛGₖ | | | | | |
| 570658 | ^{m}CₖₛAₖₛ^{m}Cₖₛ^{m}C_{ds}T_{ds}G_{ds}G_{ds}A_{ds}^{m}C_{ds}^{m}C_{ds}A_{ds}A_{ds} | 93.2 | 5745 | 5760 | 656 | 1122 |
| | A_{ds}AₖₛGₖₛGₖ | | | | | |
| 570659 | ^{m}CₖₛGₖₛ^{m}CₖₛA_{ds}^{m}C_{ds}^{m}C_{ds}T_{ds}G_{ds}G_{ds}A_{ds}^{m}C_{ds}^{m} | 70 | 5747 | 5762 | 657 | 1123 |
| | C_{ds}A_{ds}AₖₛAₖₛAₖ | | | | | |
| 570660 | GₖₛAₖₛ^{m}Cₖₛ^{m}C_{ds}G_{ds}^{m}C_{ds}A_{ds}^{m}C_{ds}^{m}C_{ds}T_{ds}G_{ds}G_{ds} | 46.4 | 5750 | 5765 | 658 | 1124 |
| | A_{ds}^{m}Cₖₛ^{m}CₖₛAₖ | | | | | |
| 570661 | Aₖₛ^{m}Cₖₛ^{m}CₖₛT_{ds}T_{ds}G_{ds}T_{ds}A_{ds}G_{ds}T_{ds}G_{ds}G_{ds}A_{ds} | 44 | 5951 | 5966 | 659 | 1125 |
| | ^{m}CₖₛGₖₛAₖ | | | | | |
| 570662 | Tₖₛ^{m}CₖₛAₖₛ^{m}C_{ds}^{m}C_{ds}T_{ds}T_{ds}G_{ds}T_{ds}A_{ds}G_{ds}T_{ds}G_{ds} | 76.8 | 5953 | 5968 | 660 | 1126 |
| | GₖₛAₖₛ^{m}Cₖ | | | | | |
| 570663 | Gₖₛ^{m}CₖₛTₖₛ^{m}C_{ds}A_{ds}^{m}C_{ds}^{m}C_{ds}T_{ds}T_{ds}G_{ds}T_{ds}A_{ds} | 69.5 | 5955 | 5970 | 661 | 1127 |
| | G_{ds}TₖₛGₖₛGₖ | | | | | |
| 570664 | GₖₛGₖₛAₖₛG_{ds}A_{ds}G_{ds}G_{ds}A_{ds}G_{ds}G_{ds}^{m}C_{ds}G_{ds}A_{ds} | 88.2 | 6015 | 6030 | 662 | 1111 |
| | TₖₛAₖₛGₖ | | | | | |
| 570665 | AₖₛGₖₛGₖₛG_{ds}A_{ds}G_{ds}A_{ds}G_{ds}G_{ds}A_{ds}G_{ds}G_{ds}^{m}C_{ds} | 96.9 | 6017 | 6032 | 663 | 1112 |
| | GₖₛAₖₛTₖ | | | | | |
| 570666 | ^{m}CₖₛTₖₛ^{m}Cₖₛ^{m}C_{ds}T_{ds}G_{ds}^{m}C_{ds}T_{ds}^{m}C_{ds}A_{ds}G_{ds}A_{ds} | 74.7 | 6028 | 6043 | 664 | 1113 |
| | G_{ds}GₖₛGₖₛAₖ | | | | | |
| 570667 | GₖₛTₖₛGₖₛ^{m}C_{ds}T_{ds}^{m}C_{ds}^{m}C_{ds}T_{ds}G_{ds}^{m}C_{ds}T_{ds}^{m}C_{ds} | 77.5 | 6031 | 6046 | 665 | 1114 |
| | A_{ds}GₖₛAₖₛGₖ | | | | | |
| 570668 | AₖₛGₖₛGₖₛT_{ds}G_{ds}^{m}C_{ds}T_{ds}^{m}C_{ds}^{m}C_{ds}T_{ds}G_{ds}^{m}C_{ds} | 76.7 | 6033 | 6048 | 666 | 1115 |
| | T_{ds}^{m}CₖₛAₖₛGₖ | | | | | |
| 570669 | AₖₛGₖₛAₖₛG_{ds}G_{ds}T_{ds}G_{ds}^{m}C_{ds}T_{ds}^{m}C_{ds}^{m}C_{ds}T_{ds} | 43.3 | 6035 | 6050 | 667 | 1116 |
| | G_{ds}^{m}CₖₛTₖₛ^{m}Cₖ | | | | | |
| 570670 | AₖₛGₖₛAₖₛG_{ds}A_{ds}G_{ds}G_{ds}T_{ds}G_{ds}^{m}C_{ds}T_{ds}^{m}C_{ds}^{m} | 27.1 | 6037 | 6052 | 668 | 1117 |
| | C_{ds}TₖₛGₖₛ^{m}Cₖ | | | | | |
| 570671 | Aₖₛ^{m}Cₖₛ^{m}Cₖₛ^{m}C_{ds}^{m}C_{ds}G_{ds}^{m}C_{ds}^{m}C_{ds}^{m}C_{ds}^{m}C_{ds}^{m} | 42.6 | 6291 | 6306 | 669 | 1118 |
| | C_{ds}G_{ds}^{m}C_{ds}Tₖₛ^{m}CₖₛAₖ | | | | | |
| 570672 | ^{m}CₖₛTₖₛAₖₛ^{m}C_{ds}^{m}C_{ds}^{m}C_{ds}^{m}C_{ds}G_{ds}^{m}C_{ds}^{m}C_{ds}^{m} | 44.9 | 6293 | 6308 | 670 | 1119 |
| | C_{ds}^{m}C_{ds}^{m}C_{ds}Gₖₛ^{m}CₖₛTₖ | | | | | |
| 570673 | Aₖₛ^{m}Cₖₛ^{m}CₖₛT_{ds}A_{ds}^{m}C_{ds}^{m}C_{ds}^{m}C_{ds}^{m}C_{ds}G_{ds}^{m}C_{ds} | 36.6 | 6295 | 6310 | 671 | 1120 |
| | ^{m}C_{ds}^{m}C_{ds}^{m}Cₖₛ^{m}CₖₛGₖ | | | | | |
| 570674 | GₖₛTₖₛAₖₛ^{m}C_{ds}^{m}C_{ds}T_{ds}A_{ds}^{m}C_{ds}^{m}C_{ds}^{m}C_{ds}^{m}C_{ds} | 52 | 6297 | 6312 | 672 | 1121 |
| | G_{ds}^{m}C_{ds}^{m}Cₖₛ^{m}Cₖₛ^{m}Cₖ | | | | | |
| 570675 | AₖₛGₖₛGₖₛT_{ds}A_{ds}^{m}C_{ds}^{m}C_{ds}T_{ds}A_{ds}^{m}C_{ds}^{m}C_{ds}^{m}C_{ds} | 56.4 | 6299 | 6314 | 673 | 1122 |
| | ^{m}C_{ds}Gₖₛ^{m}Cₖₛ^{m}Cₖ | | | | | |
| 570676 | GₖₛGₖₛGₖₛA_{ds}G_{ds}G_{ds}T_{ds}T_{ds}^{m}C_{ds}^{m}C_{ds}^{m}C_{ds}G_{ds}^{m} | 51.4 | 6329 | 6344 | 674 | 1123 |
| | C_{ds}AₖₛGₖₛ^{m}Cₖ | | | | | |
| 570677 | GₖₛTₖₛ^{m}Cₖₛ^{m}C_{ds}T_{ds}T_{ds}A_{ds}^{m}C_{ds}T_{ds}^{m}C_{ds}^{m}C_{ds}A_{ds} | 28 | 6360 | 6375 | 675 | 1124 |
| | A_{ds}^{m}CₖₛTₖₛTₖ | | | | | |
| 570678 | ^{m}CₖₛTₖₛGₖₛT_{ds}^{m}C_{ds}^{m}C_{ds}T_{ds}T_{ds}A_{ds}^{m}C_{ds}T_{ds}^{m}C_{ds} | 33.6 | 6362 | 6377 | 676 | 1125 |
| | C_{ds}AₖₛAₖₛ^{m}Cₖ | | | | | |
| 570679 | ^{m}CₖₛAₖₛ^{m}CₖₛT_{ds}G_{ds}T_{ds}^{m}C_{ds}^{m}C_{ds}T_{ds}T_{ds}A_{ds}^{m}C_{ds} | 7.9 | 6364 | 6379 | 677 | 1126 |
| | T_{ds}^{m}Cₖₛ^{m}CₖₛAₖ | | | | | |
| 570680 | GₖₛGₖₛ^{m}CₖₛA_{ds}^{m}C_{ds}T_{ds}G_{ds}T_{ds}^{m}C_{ds}^{m}C_{ds}T_{ds}T_{ds} | 20.2 | 6366 | 6381 | 678 | 1127 |
| | A_{ds}^{m}CₖₛTₖₛ^{m}Cₖ | | | | | |
| 570681 | TₖₛAₖₛGₖₛG_{ds}^{m}C_{ds}A_{ds}^{m}C_{ds}T_{ds}G_{ds}T_{ds}^{m}C_{ds}^{m}C_{ds} | 38.3 | 6368 | 6383 | 679 | 1145 |
| | T_{ds}TₖₛAₖₛ^{m}Cₖ | | | | | |
| 570682 | GₖₛGₖₛTₖₛA_{ds}G_{ds}G_{ds}^{m}C_{ds}A_{ds}^{m}C_{ds}T_{ds}G_{ds}T_{ds}^{m} | 13.9 | 6370 | 6385 | 680 | 1146 |
| | C_{ds}^{m}CₖₛTₖₛTₖ | | | | | |
| 570683 | GₖₛTₖₛ^{m}CₖₛA_{ds}^{m}C_{ds}T_{ds}G_{ds}^{m}C_{ds}T_{ds}G_{ds}G_{ds}G_{ds}T_{ds} | 29 | 6445 | 6460 | 681 | 1147 |
| | ^{m}Cₖₛ^{m}CₖₛTₖ | | | | | |
| 570684 | GₖₛGₖₛTₖₛ^{m}C_{ds}A_{ds}^{m}C_{ds}T_{ds}G_{ds}^{m}C_{ds}T_{ds}G_{ds}G_{ds} | 21.3 | 6446 | 6461 | 43 | 1148 |
| | G_{ds}Tₖₛ^{m}Cₖₛ^{m}Cₖ | | | | | |
| 570685 | AₖₛGₖₛGₖₛT_{ds}^{m}C_{ds}A_{ds}^{m}C_{ds}T_{ds}G_{ds}^{m}C_{ds}T_{ds}G_{ds} | 16.9 | 6447 | 6462 | 682 | 1149 |
| | G_{ds}GₖₛTₖₛ^{m}Cₖ | | | | | |
| 570686 | ^{m}CₖₛTₖₛAₖₛG_{ds}G_{ds}T_{ds}^{m}C_{ds}A_{ds}^{m}C_{ds}T_{ds}G_{ds}^{m}C_{ds} | 19.6 | 6449 | 6464 | 683 | 1150 |
| | T_{ds}GₖₛGₖₛGₖ | | | | | |
| 570687 | GₖₛTₖₛ^{m}CₖₛT_{ds}A_{ds}G_{ds}G_{ds}T_{ds}^{m}C_{ds}A_{ds}^{m}C_{ds}T_{ds} | 15.7 | 6451 | 6466 | 684 | 1151 |
| | G_{ds}^{m}CₖₛTₖₛGₖ | | | | | |
| 570688 | AₖₛAₖₛGₖₛT_{ds}^{m}C_{ds}T_{ds}A_{ds}G_{ds}G_{ds}T_{ds}^{m}C_{ds}A_{ds}^{m} | 16.6 | 6453 | 6468 | 685 | 1152 |
| | C_{ds}TₖₛGₖₛ^{m}Cₖ | | | | | |
| 570689 | Gₖₛ^{m}CₖₛAₖₛ^{m}C_{ds}T_{ds}^{m}C_{ds}^{m}C_{ds}A_{ds}T_{ds}T_{ds}G_{ds}T_{ds}^{m} | 13.2 | 6530 | 6545 | 686 | 1153 |
| | C_{ds}Tₖₛ^{m}CₖₛAₖ | | | | | |
| 570690 | ^{m}CₖₛTₖₛGₖₛ^{m}C_{ds}A_{ds}^{m}C_{ds}T_{ds}^{m}C_{ds}^{m}C_{ds}A_{ds}T_{ds}T_{ds} | 50.1 | 6532 | 6547 | 687 | 1154 |
| | G_{ds}Tₖₛ^{m}CₖₛTₖ | | | | | |
| 570691 | ^{m}Cₖₛ^{m}Cₖₛ^{m}CₖₛT_{ds}G_{ds}^{m}C_{ds}A_{ds}^{m}C_{ds}T_{ds}^{m}C_{ds}^{m}C_{ds} | 48.4 | 6534 | 6549 | 688 | 1155 |
| | A_{ds}T_{ds}TₖₛGₖₛTₖ | | | | | |
| 570692 | ^{m}Cₖₛ^{m}Cₖₛ^{m}Cₖₛ^{m}C_{ds}^{m}C_{ds}T_{ds}G_{ds}^{m}C_{ds}A_{ds}^{m}C_{ds}T_{ds} | 74 | 6536 | 6551 | 689 | 1156 |
| | ^{m}C_{ds}^{m}C_{ds}AₖₛTₖₛTₖ | | | | | |
| 570693 | ^{m}CₖₛTₖₛTₖₛG_{ds}^{m}C_{ds}T_{ds}G_{ds}A_{ds}G_{ds}T_{ds}^{m}C_{ds}A_{ds} | 25.3 | 6559 | 6574 | 690 | 1157 |
| | G_{ds}GₖₛAₖₛGₖ | | | | | |
| 570694 | Tₖₛ^{m}Cₖₛ^{m}CₖₛT_{ds}T_{ds}G_{ds}^{m}C_{ds}T_{ds}G_{ds}A_{ds}G_{ds}T_{ds}^{m} | 39.5 | 6561 | 6576 | 691 | 1158 |
| | C_{ds}AₖₛGₖₛGₖ | | | | | |
| 570695 | ^{m}CₖₛTₖₛTₖₛ^{m}C_{ds}^{m}C_{ds}T_{ds}T_{ds}G_{ds}^{m}C_{ds}T_{ds}G_{ds}A_{ds} | 22.9 | 6563 | 6578 | 692 | 1159 |
| | G_{ds}Tₖₛ^{m}CₖₛAₖ | | | | | |
| 570696 | Aₖₛ^{m}Cₖₛ^{m}CₖₛT_{ds}T_{ds}^{m}C_{ds}^{m}C_{ds}T_{ds}T_{ds}G_{ds}^{m}C_{ds}T_{ds} | 52.5 | 6565 | 6580 | 693 | 1160 |
| | G_{ds}AₖₛGₖₛTₖ | | | | | |
| 570697 | GₖₛGₖₛAₖₛ^{m}C_{ds}^{m}C_{ds}T_{ds}T_{ds}^{m}C_{ds}^{m}C_{ds}T_{ds}T_{ds}G_{ds}^{m} | 37.6 | 6567 | 6582 | 694 | 1161 |
| | C_{ds}TₖₛGₖₛAₖ | | | | | |
| 570698 | ^{m}CₖₛAₖₛGₖₛG_{ds}A_{ds}^{m}C_{ds}^{m}C_{ds}T_{ds}T_{ds}^{m}C_{ds}^{m}C_{ds}T_{ds} | 44.2 | 6569 | 6584 | 695 | 1162 |
| | T_{ds}Gₖₛ^{m}CₖₛTₖ | | | | | |
| 570699 | AₖₛGₖₛ^{m}Cₖₛ^{m}C_{ds}^{m}C_{ds}T_{ds}^{m}C_{ds}^{m}C_{ds}A_{ds}G_{ds}G_{ds}A_{ds} | 26.6 | 6576 | 6591 | 696 | 1163 |
| | ^{m}C_{ds}^{m}CₖₛTₖₛTₖ | | | | | |
| 570700 | TₖₛAₖₛGₖₛ^{m}C_{ds}T_{ds}^{m}C_{ds}^{m}C_{ds}^{m}C_{ds}^{m}C_{ds}A_{ds}^{m}C_{ds} | 33.6 | 6594 | 6609 | 697 | 1164 |
| | T_{ds}^{m}C_{ds}^{m}CₖₛAₖₛGₖ | | | | | |
| 570701 | GₖₛAₖₛTₖₛA_{ds}G_{ds}^{m}C_{ds}T_{ds}^{m}C_{ds}^{m}C_{ds}^{m}C_{ds}^{m}C_{ds}A_{ds} | 20.4 | 6596 | 6611 | 698 | 1165 |
| | ^{m}C_{ds}Tₖₛ^{m}Cₖₛ^{m}Cₖ | | | | | |
| 570702 | ^{m}CₖₛAₖₛGₖₛA_{ds}T_{ds}A_{ds}G_{ds}^{m}C_{ds}T_{ds}^{m}C_{ds}^{m}C_{ds}^{m}C_{ds} | 33.8 | 6598 | 6613 | 699 | 1166 |
| | ^{m}C_{ds}Aₖₛ^{m}CₖₛTₖ | | | | | |
| 570703 | ^{m}CₖₛTₖₛ^{m}CₖₛA_{ds}G_{ds}A_{ds}T_{ds}A_{ds}G_{ds}^{m}C_{ds}T_{ds}^{m}C_{ds} | 25.8 | 6600 | 6615 | 700 | 1167 |
| | ^{m}C_{ds}^{m}Cₖₛ^{m}CₖₛAₖ | | | | | |
| 570704 | AₖₛGₖₛ^{m}CₖₛT_{ds}^{m}C_{ds}A_{ds}G_{ds}A_{ds}T_{ds}A_{ds}G_{ds}^{m}C_{ds} | 29.1 | 6602 | 6617 | 701 | 1168 |
| | T_{ds}^{m}Cₖₛ^{m}Cₖₛ^{m}Cₖ | | | | | |
| 570705 | Tₖₛ^{m}CₖₛAₖₛG_{ds}^{m}C_{ds}T_{ds}^{m}C_{ds}A_{ds}G_{ds}A_{ds}T_{ds}A_{ds} | 47.4 | 6604 | 6619 | 702 | 1169 |
| | G_{ds}^{m}CₖₛTₖₛ^{m}Cₖ | | | | | |
| 570706 | Tₖₛ^{m}CₖₛTₖₛ^{m}C_{ds}A_{ds}G_{ds}^{m}C_{ds}T_{ds}^{m}C_{ds}A_{ds}G_{ds}A_{ds} | 33.4 | 6606 | 6621 | 703 | 1170 |
| | T_{ds}AₖₛGₖₛ^{m}Cₖ | | | | | |
| 570707 | GₖₛAₖₛGₖₛT_{ds}^{m}C_{ds}^{m}C_{ds}T_{ds}^{m}C_{ds}T_{ds}^{m}C_{ds}^{m}C_{ds}T_{ds} | 49 | 6636 | 6651 | 704 | 1171 |
| | G_{ds}^{m}CₖₛTₖₛTₖ | | | | | |
| 570708 | GₖₛGₖₛAₖₛG_{ds}G_{ds}A_{ds}G_{ds}T_{ds}^{m}C_{ds}^{m}C_{ds}T_{ds}^{m}C_{ds} | 79.2 | 6640 | 6655 | 705 | 1172 |
| | T_{ds}^{m}Cₖₛ^{m}CₖₛTₖ | | | | | |
| 570709 | GₖₛAₖₛGₖₛG_{ds}A_{ds}G_{ds}G_{ds}A_{ds}G_{ds}T_{ds}^{m}C_{ds}^{m}C_{ds}T_{ds} | 63.3 | 6642 | 6657 | 706 | 1173 |
| | ^{m}CₖₛTₖₛ^{m}Cₖ | | | | | |
| 570710 | ^{m}CₖₛAₖₛAₖₛA_{ds}A_{ds}G_{ds}G_{ds}G_{ds}^{m}C_{ds}A_{ds}^{m}C_{ds}^{m}C_{ds} | 38.8 | 6713 | 6728 | 707 | 1174 |
| | ^{m}C_{ds}AₖₛGₖₛAₖ | | | | | |
| 570711 | AₖₛGₖₛ^{m}CₖₛA_{ds}A_{ds}A_{ds}A_{ds}G_{ds}G_{ds}G_{ds}^{m}C_{ds}A_{ds}^{m} | 13.7 | 6715 | 6730 | 708 | 1175 |
| | C_{ds}^{m}Cₖₛ^{m}CₖₛAₖ | | | | | |
| 570712 | GₖₛGₖₛAₖₛT_{ds}^{m}C_{ds}^{m}C_{ds}^{m}C_{ds}^{m}C_{ds}A_{ds}G_{ds}T_{ds}A_{ds} | 45.8 | 6733 | 6748 | 709 | 1176 |
| | T_{ds}TₖₛGₖₛTₖ | | | | | |
| 570713 | ^{m}CₖₛTₖₛGₖₛG_{ds}A_{ds}T_{ds}^{m}C_{ds}^{m}C_{ds}^{m}C_{ds}^{m}C_{ds}A_{ds}G_{ds} | 45.6 | 6735 | 6750 | 710 | 1177 |
| | T_{ds}AₖₛTₖₛTₖ | | | | | |
| 570714 | TₖₛGₖₛ^{m}CₖₛT_{ds}G_{ds}G_{ds}A_{ds}T_{ds}^{m}C_{ds}^{m}C_{ds}^{m}C_{ds}^{m}C_{ds} | 43.6 | 6737 | 6752 | 711 | 1178 |
| | A_{ds}GₖₛTₖₛAₖ | | | | | |
| 570715 | AₖₛTₖₛTₖₛ^{m}C_{ds}T_{ds}^{m}C_{ds}T_{ds}A_{ds}G_{ds}A_{ds}^{m}C_{ds}T_{ds}G_{ds} | 18.3 | 6789 | 6804 | 712 | 1179 |
| | ^{m}CₖₛAₖₛAₖ | | | | | |
| 570716 | TₖₛAₖₛAₖₛT_{ds}T_{ds}^{m}C_{ds}T_{ds}^{m}C_{ds}T_{ds}A_{ds}G_{ds}A_{ds}^{m}C_{ds} | 15.1 | 6791 | 6806 | 713 | 1180 |
| | TₖₛGₖₛ^{m}Cₖ | | | | | |
| 570717 | Tₖₛ^{m}CₖₛTₖₛA_{ds}A_{ds}T_{ds}T_{ds}^{m}C_{ds}T_{ds}^{m}C_{ds}T_{ds}A_{ds}G_{ds} | 49.9 | 6793 | 6808 | 714 | 1181 |
| | Aₖₛ^{m}CₖₛTₖ | | | | | |
| 570718 | Tₖₛ^{m}CₖₛTₖₛ^{m}C_{ds}T_{ds}A_{ds}A_{ds}T_{ds}T_{ds}^{m}C_{ds}T_{ds}^{m}C_{ds} | 77.6 | 6795 | 6810 | 715 | 1182 |
| | T_{ds}AₖₛGₖₛAₖ | | | | | |
| 570719 | ^{m}CₖₛTₖₛ^{m}Cₖₛ^{m}C_{ds}A_{ds}T_{ds}A_{ds}A_{ds}T_{ds}T_{ds}^{m}C_{ds}T_{ds}^{m} | 42 | 6804 | 6819 | 716 | 1183 |
| | C_{ds}TₖₛAₖₛAₖ | | | | | |
| 570720 | Aₖₛ^{m}CₖₛTₖₛ^{m}C_{ds}T_{ds}^{m}C_{ds}^{m}C_{ds}A_{ds}T_{ds}A_{ds}A_{ds}T_{ds} | 28.5 | 6807 | 6822 | 717 | 1184 |
| | T_{ds}^{m}CₖₛTₖₛ^{m}Cₖ | | | | | |
| 570721 | Aₖₛ^{m}CₖₛAₖₛ^{m}C_{ds}T_{ds}^{m}C_{ds}T_{ds}^{m}C_{ds}^{m}C_{ds}A_{ds}T_{ds}A_{ds} | 27.4 | 6809 | 6824 | 718 | 1185 |
| | A_{ds}TₖₛTₖₛ^{m}Cₖ | | | | | |
| 570722 | ^{m}Cₖₛ^{m}CₖₛAₖₛ^{m}C_{ds}A_{ds}^{m}C_{ds}T_{ds}^{m}C_{ds}T_{ds}^{m}C_{ds}^{m}C_{ds} | 35.4 | 6811 | 6826 | 719 | 1186 |
| | A_{ds}T_{ds}AₖₛAₖₛTₖ | | | | | |
| 570723 | TₖₛGₖₛ^{m}Cₖₛ^{m}C_{ds}A_{ds}^{m}C_{ds}A_{ds}^{m}C_{ds}T_{ds}^{m}C_{ds}T_{ds}^{m} | 45 | 6813 | 6828 | 720 | 1187 |
| | C_{ds}^{m}C_{ds}AₖₛTₖₛAk | | | | | |

TABLE 16
Inhibition of human DMPK RNA transcript in HepG2 cells targeting SEQ ID NO: 2

| | | | Start | Stop | | |
|---|---|---|---|---|---|---|
| | | | Site on | Site on | | Modified |
| ISIS | | % Target | Seq | Seq | Unmodified | Seq ID |
| No. | Sequence | Expression | ID: 2 | ID: 2 | Seq ID No. | No. |
| UTC | N/A | 100 | N/A | N/A | | |
| 445569 | ^{m}CₑₛGₑₛGₑₛAₑₛGₑₛ^{m}C_{ds}G_{ds}G_{ds}T_{ds}T_{ds}G_{ds}T_{ds} | 33.9 | 13226 | 13245 | 24 | 1443 |
| | G_{ds}A_{ds}A_{ds}^{m}CₑₛTₑₛGₑₛGₑₛ^{m}Cₑ | | | | | |
| 486178 | Aₖₛ^{m}CₖₛAₖₛA_{ds}T_{ds}A_{ds}A_{ds}A_{ds}T_{ds}A_{ds}^{m}C_{ds}^{m}C_{ds} | 21.5 | 13836 | 13851 | 23 | 881 |
| | G_{ds}AₖₛGₖₛGₖ | | | | | |
| 570339 | ^{m}Cₖₛ^{m}Cₖₛ^{m}CₖₛA_{ds}T_{ds}G_{ds}^{m}C_{ds}^{m}C_{ds}^{m}C_{ds}A_{ds}T_{ds} | 56.2 | 1534 | 1549 | 721 | 1188 |
| | ^{m}C_{ds}^{m}C_{ds}TₖₛGₖₛ^{m}Cₖ | | | | | |
| 570340 | GₖₛGₖₛAₖₛ^{m}C_{ds}A_{ds}G_{ds}A_{ds}G_{ds}A_{ds}A_{ds}A_{ds}T_{ds} | 46.7 | 1597 | 1612 | 722 | 1189 |
| | G_{ds}TₖₛTₖₛGₖ | | | | | |
| 570341 | GₖₛGₖₛ^{m}CₖₛA_{ds}T_{ds}A_{ds}G_{ds}G_{ds}A_{ds}^{m}C_{ds}A_{ds}G_{ds} | 35.6 | 1603 | 1618 | 723 | 1190 |
| | A_{ds}GₖₛAₖₛAₖ | | | | | |
| 570342 | GₖₛTₖₛGₖₛG_{ds}^{m}C_{ds}A_{ds}T_{ds}A_{ds}G_{ds}G_{ds}A_{ds}^{m}C_{ds} | 34.8 | 1605 | 1620 | 724 | 1191 |
| | A_{ds}GₖₛAₖₛGₖ | | | | | |
| 570343 | TₖₛGₖₛGₖₛT_{ds}G_{ds}G_{ds}^{m}C_{ds}A_{ds}T_{ds}A_{ds}G_{ds}G_{ds}A_{ds} | 60.3 | 1607 | 1622 | 725 | 1192 |
| | ^{m}CₖₛAₖₛGₖ | | | | | |
| 570344 | ^{m}CₖₛTₖₛTₖₛA_{ds}^{m}C_{ds}T_{ds}^{m}C_{ds}T_{ds}G_{ds}^{m}C_{ds}^{m}C_{ds}^{m} | 49.6 | 1627 | 1642 | 726 | 1193 |
| | C_{ds}^{m}C_{ds}Tₖₛ^{m}Cₖₛ^{m}Cₖ | | | | | |
| 570345 | Aₖₛ^{m}Cₖₛ^{m}CₖₛT_{ds}T_{ds}A_{ds}^{m}C_{ds}T_{ds}^{m}C_{ds}T_{ds}G_{ds}^{m} | 48.6 | 1629 | 1644 | 727 | 1194 |
| | C_{ds}^{m}C_{ds}^{m}Cₖₛ^{m}CₖₛTₖ | | | | | |
| 570346 | TₖₛGₖₛAₖₛ^{m}C_{ds}^{m}C_{ds}T_{ds}T_{ds}A_{ds}^{m}C_{ds}T_{ds}^{m}C_{ds}T_{ds} | 36.8 | 1631 | 1646 | 728 | 1195 |
| | G_{ds}^{m}Cₖₛ^{m}Cₖₛ^{m}Cₖ | | | | | |
| 570347 | Gₖₛ^{m}CₖₛTₖₛG_{ds}A_{ds}^{m}C_{ds}^{m}C_{ds}T_{ds}T_{ds}A_{ds}^{m}C_{ds}T_{ds} | 53.5 | 1633 | 1648 | 729 | 1196 |
| | ^{m}C_{ds}TₖₛGₖₛ^{m}Cₖ | | | | | |
| 570348 | ^{m}CₖₛTₖₛGₖₛ^{m}C_{ds}T_{ds}G_{ds}A_{ds}^{m}C_{ds}^{m}C_{ds}T_{ds}T_{ds}A_{ds} | 59 | 1635 | 1650 | 730 | 1197 |
| | ^{m}C_{ds}Tₖₛ^{m}CₖₛTₖ | | | | | |
| 570349 | ^{m}CₖₛTₖₛ^{m}CₖₛT_{ds}G_{ds}^{m}C_{ds}T_{ds}G_{ds}A_{ds}^{m}C_{ds}^{m}C_{ds} | 70.8 | 1637 | 1652 | 731 | 1198 |
| | T_{ds}T_{ds}Aₖₛ^{m}CₖₛTₖ | | | | | |
| 570350 | Gₖₛ^{m}Cₖₛ^{m}CₖₛT_{ds}^{m}C_{ds}T_{ds}G_{ds}^{m}C_{ds}T_{ds}G_{ds}A_{ds}^{m} | 54 | 1639 | 1654 | 732 | 1199 |
| | C_{ds}^{m}C_{ds}TₖₛTₖₛAₖ | | | | | |
| 570351 | ^{m}Cₖₛ^{m}CₖₛAₖₛT_{ds}G_{ds}G_{ds}^{m}C_{ds}T_{ds}^{m}C_{ds}T_{ds}G_{ds}A_{ds} | 52.6 | 1666 | 1681 | 733 | 1200 |
| | G_{ds}Tₖₛ^{m}CₖₛAₖ | | | | | |
| 570352 | AₖₛGₖₛ^{m}Cₖₛ^{m}C_{ds}A_{ds}T_{ds}G_{ds}G_{ds}^{m}C_{ds}T_{ds}^{m}C_{ds}T_{ds} | 60.7 | 1668 | 1683 | 734 | 1201 |
| | G_{ds}AₖₛGₖₛTₖ | | | | | |
| 570353 | TₖₛAₖₛAₖₛG_{ds}^{m}C_{ds}^{m}C_{ds}A_{ds}T_{ds}G_{ds}G_{ds}^{m}C_{ds}T_{ds} | 82.3 | 1670 | 1685 | 735 | 1202 |
| | ^{m}C_{ds}TₖₛGₖₛAₖ | | | | | |
| 570354 | TₖₛAₖₛGₖₛ^{m}C_{ds}^{m}C_{ds}T_{ds}G_{ds}^{m}C_{ds}T_{ds}G_{ds}T_{ds}G_{ds} | 40.8 | 1687 | 1702 | 736 | 1203 |
| | A_{ds}^{m}CₖₛTₖₛ^{m}Cₖ | | | | | |
| 570355 | AₖₛTₖₛGₖₛG_{ds}G_{ds}A_{ds}G_{ds}G_{ds}^{m}C_{ds}T_{ds}G_{ds}T_{ds}T_{ds} | 90.7 | 1707 | 1722 | 737 | 1204 |
| | GₖₛGₖₛ^{m}Cₖ | | | | | |
| 570356 | ^{m}Cₖₛ^{m}CₖₛAₖₛT_{ds}G_{ds}G_{ds}G_{ds}A_{ds}G_{ds}G_{ds}^{m}C_{ds}T_{ds} | 73.9 | 1709 | 1724 | 738 | 1205 |
| | G_{ds}TₖₛTₖₛGₖ | | | | | |
| 570357 | GₖₛGₖₛ^{m}Cₖₛ^{m}C_{ds}A_{ds}T_{ds}G_{ds}G_{ds}G_{ds}A_{ds}G_{ds}G_{ds} | 94.9 | 1711 | 1726 | 739 | 1206 |
| | ^{m}C_{ds}TₖₛGₖₛTₖ | | | | | |
| 570358 | GₖₛTₖₛGₖₛ^{m}C_{ds}A_{ds}G_{ds}A_{ds}G_{ds}A_{ds}G_{ds}G_{ds}^{m}C_{ds} | 73.5 | 1720 | 1735 | 740 | 1207 |
| | ^{m}C_{ds}AₖₛTₖₛGₖ | | | | | |
| 570359 | GₖₛAₖₛGₖₛ^{m}C_{ds}T_{ds}^{m}C_{ds}^{m}C_{ds}^{m}C_{ds}A_{ds}G_{ds}^{m}C_{ds} | 70.2 | 1759 | 1774 | 741 | 1208 |
| | A_{ds}T_{ds}GₖₛAₖₛ^{m}Cₖ | | | | | |
| 570360 | AₖₛGₖₛGₖₛG_{ds}A_{ds}G_{ds}^{m}C_{ds}T_{ds}^{m}C_{ds}^{m}C_{ds}^{m}C_{ds} | 56.1 | 1762 | 1777 | 742 | 1209 |
| | A_{ds}G_{ds}^{m}CₖₛAₖₛTₖ | | | | | |
| 570361 | Gₖₛ^{m}Cₖₛ^{m}CₖₛA_{ds}T_{ds}A_{ds}G_{ds}A_{ds}G_{ds}^{m}C_{ds}^{m}C_{ds}^{m} | 54.9 | 1799 | 1814 | 743 | 1210 |
| | C_{ds}A_{ds}^{m}CₖₛTₖₛTₖ | | | | | |
| 570362 | GₖₛGₖₛGₖₛ^{m}C_{ds}^{m}C_{ds}A_{ds}T_{ds}A_{ds}G_{ds}A_{ds}G_{ds}^{m}C_{ds} | 78.1 | 1801 | 1816 | 744 | 1211 |
| | ^{m}C_{ds}^{m}CₖₛAₖₛ^{m}Cₖ | | | | | |
| 570363 | AₖₛTₖₛGₖₛ^{m}C_{ds}T_{ds}G_{ds}G_{ds}^{m}C_{ds}^{m}C_{ds}^{m}C_{ds}T_{ds}^{m} | 76.2 | 1848 | 1863 | 745 | 1212 |
| | C_{ds}^{m}C_{ds}TₖₛGₖₛGₖ | | | | | |
| 570364 | AₖₛGₖₛ^{m}CₖₛT_{ds}G_{ds}^{m}C_{ds}^{m}C_{ds}^{m}C_{ds}^{m}C_{ds}A_{ds}T_{ds} | 92.6 | 1857 | 1872 | 746 | 1213 |
| | G_{ds}^{m}C_{ds}TₖₛGₖₛGₖ | | | | | |
| 570365 | ^{m}CₖₛGₖₛ^{m}Cₖₛ^{m}C_{ds}^{m}C_{ds}^{m}C_{ds}T_{ds}G_{ds}G_{ds}^{m}C_{ds}A_{ds} | 73.6 | 1867 | 1882 | 747 | 1214 |
| | G_{ds}^{m}C_{ds}TₖₛGₖₛ^{m}Cₖ | | | | | |
| 570366 | TₖₛGₖₛ^{m}CₖₛG_{ds}^{m}C_{ds}^{m}C_{ds}^{m}C_{ds}^{m}C_{ds}T_{ds}G_{ds}G_{ds}^{m} | 76.6 | 1869 | 1884 | 748 | 1215 |
| | C_{ds}A_{ds}Gₖₛ^{m}CₖₛTₖ | | | | | |
| 570367 | Gₖₛ^{m}CₖₛTₖₛG_{ds}^{m}C_{ds}G_{ds}^{m}C_{ds}^{m}C_{ds}^{m}C_{ds}^{m}C_{ds}T_{ds} | 79.1 | 1871 | 1886 | 749 | 1216 |
| | G_{ds}G_{ds}^{m}CₖₛAₖₛGₖ | | | | | |
| 570368 | ^{m}CₖₛGₖₛGₖₛ^{m}C_{ds}T_{ds}G_{ds}^{m}C_{ds}G_{ds}^{m}C_{ds}^{m}C_{ds}^{m}C_{ds} | 82.9 | 1873 | 1888 | 750 | 1217 |
| | ^{m}C_{ds}T_{ds}GₖₛGₖₛ^{m}Cₖ | | | | | |
| 570369 | GₖₛTₖₛ^{m}CₖₛG_{ds}G_{ds}^{m}C_{ds}T_{ds}G_{ds}^{m}C_{ds}G_{ds}^{m}C_{ds}^{m} | 47.5 | 1875 | 1890 | 751 | 1218 |
| | C_{ds}^{m}C_{ds}^{m}CₖₛTₖₛGₖ | | | | | |
| 570370 | ^{m}CₖₛTₖₛGₖₛT_{ds}^{m}C_{ds}G_{ds}G_{ds}^{m}C_{ds}T_{ds}G_{ds}^{m}C_{ds}G_{ds} | 79.6 | 1877 | 1892 | 752 | 1219 |
| | ^{m}C_{ds}^{m}Cₖₛ^{m}Cₖₛ^{m}Cₖ | | | | | |
| 570371 | Gₖₛ^{m}Cₖₛ^{m}CₖₛT_{ds}G_{ds}T_{ds}^{m}C_{ds}G_{ds}G_{ds}^{m}C_{ds}T_{ds}G_{ds} | 58.4 | 1879 | 1894 | 753 | 1220 |
| | ^{m}C_{ds}Gₖₛ^{m}Cₖₛ^{m}Cₖ | | | | | |
| 570372 | ^{m}CₖₛTₖₛGₖₛ^{m}C_{ds}^{m}C_{ds}T_{ds}G_{ds}T_{ds}^{m}C_{ds}G_{ds}G_{ds}^{m} | 49.9 | 1881 | 1896 | 754 | 1221 |
| | C_{ds}T_{ds}Gₖₛ^{m}CₖₛGₖ | | | | | |
| 570373 | Aₖₛ^{m}Cₖₛ^{m}CₖₛT_{ds}G_{ds}^{m}C_{ds}^{m}C_{ds}T_{ds}G_{ds}T_{ds}^{m}C_{ds} | 27.4 | 1883 | 1898 | 755 | 1222 |
| | G_{ds}G_{ds}^{m}CₖₛTₖₛGₖ | | | | | |
| 570374 | Aₖₛ^{m}CₖₛAₖₛ^{m}C_{ds}^{m}C_{ds}T_{ds}G_{ds}^{m}C_{ds}^{m}C_{ds}T_{ds}G_{ds} | 54.3 | 1885 | 1900 | 756 | 1223 |
| | T_{ds}^{m}C_{ds}GₖₛGₖₛ^{m}Cₖ | | | | | |
| 570375 | GₖₛAₖₛAₖₛ^{m}C_{ds}A_{ds}^{m}C_{ds}^{m}C_{ds}T_{ds}G_{ds}^{m}C_{ds}^{m}C_{ds} | 50.5 | 1887 | 1902 | 757 | 1224 |
| | T_{ds}G_{ds}Tₖₛ^{m}CₖₛGₖ | | | | | |
| 570376 | ^{m}Cₖₛ^{m}CₖₛGₖₛA_{ds}A_{ds}^{m}C_{ds}A_{ds}^{m}C_{ds}^{m}C_{ds}T_{ds}G_{ds} | 57.7 | 1889 | 1904 | 758 | 1225 |
| | ^{m}C_{ds}^{m}C_{ds}TₖₛGₖₛTₖ | | | | | |
| 570377 | ^{m}CₖₛGₖₛ^{m}CₖₛC_{ds}G_{ds}A_{ds}A_{ds}^{m}C_{ds}A_{ds}^{m}C_{ds}^{m}C_{ds} | 69.3 | 1891 | 1906 | 759 | 1226 |
| | T_{ds}G_{ds}^{m}Cₖₛ^{m}CₖₛTₖ | | | | | |
| 570378 | ^{m}Cₖₛ^{m}CₖₛTₖₛG_{ds}G_{ds}G_{ds}^{m}C_{ds}A_{ds}^{m}C_{ds}^{m}C_{ds}T_{ds} | 188.2 | 1925 | 1940 | 760 | 1227 |
| | G_{ds}T_{ds}TₖₛGₖₛGₖ | | | | | |
| 570379 | GₖₛTₖₛGₖₛ^{m}C_{ds}^{m}C_{ds}T_{ds}G_{ds}G_{ds}G_{ds}^{m}C_{ds}A_{ds}^{m}C_{ds} | 111.5 | 1928 | 1943 | 761 | 1228 |
| | ^{m}C_{ds}TₖₛGₖₛTₖ | | | | | |
| 570380 | ^{m}CₖₛGₖₛ^{m}Cₖₛ^{m}C_{ds}^{m}C_{ds}T_{ds}^{m}C_{ds}^{m}C_{ds}^{m}C_{ds}A_{ds}G_{ds} | 78 | 1938 | 1953 | 762 | 1229 |
| | T_{ds}G_{ds}^{m}Cₖₛ^{m}CₖₛTₖ | | | | | |
| 570381 | Aₖₛ^{m}Cₖₛ^{m}CₖₛG_{ds}^{m}C_{ds}^{m}C_{ds}^{m}C_{ds}T_{ds}^{m}C_{ds}^{m}C_{ds}^{m} | 74.9 | 1940 | 1955 | 763 | 1230 |
| | C_{ds}A_{ds}G_{ds}TₖₛGₖₛ^{m}Cₖ | | | | | |
| 570382 | Tₖₛ^{m}CₖₛAₖₛ^{m}C_{ds}^{m}C_{ds}G_{ds}^{m}C_{ds}^{m}C_{ds}^{m}C_{ds}T_{ds}^{m} | 71.6 | 1942 | 1957 | 764 | 1231 |
| | C_{ds}^{m}C_{ds}^{m}C_{ds}AₖₛGₖₛTₖ | | | | | |
| 570383 | AₖₛGₖₛTₖₛ^{m}C_{ds}A_{ds}^{m}C_{ds}^{m}C_{ds}G_{ds}^{m}C_{ds}^{m}C_{ds}^{m}C_{ds} | 62.1 | 1944 | 1959 | 765 | 1232 |
| | T_{ds}^{m}C_{ds}^{m}Cₖₛ^{m}CₖₛAₖ | | | | | |
| 570384 | TₖₛGₖₛAₖₛG_{ds}T_{ds}^{m}C_{ds}A_{ds}^{m}C_{ds}^{m}C_{ds}G_{ds}^{m}C_{ds}^{m} | 65.6 | 1946 | 1961 | 766 | 1233 |
| | C_{ds}^{m}C_{ds}Tₖₛ^{m}Cₖₛ^{m}Cₖ | | | | | |
| 570385 | ^{m}CₖₛGₖₛTₖₛG_{ds}A_{ds}G_{ds}T_{ds}^{m}C_{ds}A_{ds}^{m}C_{ds}^{m}C_{ds} | 37.3 | 1948 | 1963 | 767 | 1234 |
| | G_{ds}^{m}C_{ds}^{m}Cₖₛ^{m}CₖₛTₖ | | | | | |
| 570386 | ^{m}CₖₛAₖₛAₖₛA_{ds}G_{ds}^{m}C_{ds}T_{ds}G_{ds}G_{ds}T_{ds}T_{ds}^{m}C_{ds} | 30.5 | 1974 | 1989 | 768 | 1235 |
| | T_{ds}^{m}Cₖₛ^{m}Cₖₛ^{m}Cₖ | | | | | |
| 570387 | TₖₛGₖₛ^{m}CₖₛA_{ds}A_{ds}A_{ds}G_{ds}^{m}C_{ds}T_{ds}G_{ds}G_{ds}T_{ds} | 35.8 | 1976 | 1991 | 769 | 1236 |
| | T_{ds}^{m}CₖₛTₖₛ^{m}Cₖ | | | | | |
| 570388 | Tₖₛ^{m}CₖₛTₖₛG_{ds}^{m}C_{ds}A_{ds}A_{ds}A_{ds}G_{ds}^{m}C_{ds}T_{ds}G_{ds} | 30.1 | 1978 | 1993 | 770 | 1237 |
| | G_{ds}TₖₛTₖₛ^{m}Cₖ | | | | | |
| 570389 | TₖₛGₖₛTₖₛ^{m}C_{ds}T_{ds}G_{ds}^{m}C_{ds}A_{ds}A_{ds}A_{ds}G_{ds}^{m}C_{ds} | 50.1 | 1980 | 1995 | 771 | 1238 |
| | T_{ds}GₖₛGₖₛTₖ | | | | | |
| 570390 | ^{m}Cₖₛ^{m}CₖₛTₖₛG_{ds}T_{ds}^{m}C_{ds}T_{ds}G_{ds}^{m}C_{ds}A_{ds}A_{ds}A_{ds} | 36 | 1982 | 1997 | 772 | 1239 |
| | G_{ds}^{m}CₖₛTₖₛGₖ | | | | | |
| 570391 | ^{m}CₖₛGₖₛ^{m}Cₖₛ^{m}C_{ds}T_{ds}G_{ds}T_{ds}^{m}C_{ds}T_{ds}G_{ds}^{m}C_{ds} | 31.1 | 1984 | 1999 | 773 | 1240 |
| | A_{ds}A_{ds}AₖₛGₖₛ^{m}Cₖ | | | | | |
| 570392 | TₖₛTₖₛGₖₛT_{ds}^{m}C_{ds}^{m}C_{ds}^{m}C_{ds}T_{ds}^{m}C_{ds}^{m}C_{ds}T_{ds} | 62.9 | 2022 | 2037 | 774 | 1241 |
| | G_{ds}G_{ds}AₖₛTₖₛ^{m}Cₖ | | | | | |
| 570393 | AₖₛGₖₛTₖₛT_{ds}G_{ds}T_{ds}^{m}C_{ds}^{m}C_{ds}^{m}C_{ds}T_{ds}^{m}C_{ds}^{m} | 57.1 | 2024 | 2039 | 775 | 1242 |
| | C_{ds}T_{ds}GₖₛGₖₛAₖ | | | | | |
| 570394 | AₖₛAₖₛAₖₛG_{ds}T_{ds}T_{ds}G_{ds}T_{ds}^{m}C_{ds}^{m}C_{ds}^{m}C_{ds}T_{ds} | 56.2 | 2026 | 2041 | 776 | 1243 |
| | ^{m}C_{ds}^{m}CₖₛTₖₛGₖ | | | | | |
| 570395 | ^{m}Cₖₛ^{m}CₖₛAₖₛA_{ds}A_{ds}G_{ds}T_{ds}T_{ds}G_{ds}T_{ds}^{m}C_{ds}^{m}C_{ds} | 48.9 | 2028 | 2043 | 777 | 1244 |
| | ^{m}C_{ds}Tₖₛ^{m}Cₖₛ^{m}Cₖ | | | | | |
| 570396 | Aₖₛ^{m}Cₖₛ^{m}Cₖₛ^{m}C_{ds}A_{ds}A_{ds}A_{ds}G_{ds}T_{ds}T_{ds}G_{ds}T_{ds} | 59.9 | 2030 | 2045 | 778 | 1245 |
| | ^{m}C_{ds}^{m}Cₖₛ^{m}CₖₛTₖ | | | | | |
| 570397 | GₖₛAₖₛAₖₛ^{m}C_{ds}^{m}C_{ds}^{m}C_{ds}A_{ds}A_{ds}A_{ds}G_{ds}T_{ds}T_{ds} | 47.9 | 2032 | 2047 | 779 | 1246 |
| | G_{ds}Tₖₛ^{m}Cₖₛ^{m}Cₖ | | | | | |
| 570398 | GₖₛAₖₛAₖₛG_{ds}A_{ds}A_{ds}^{m}C_{ds}^{m}C_{ds}^{m}C_{ds}A_{ds}A_{ds}A_{ds} | 60 | 2035 | 2050 | 780 | 1247 |
| | G_{ds}TₖₛTₖₛGₖ | | | | | |
| 570399 | ^{m}Cₖₛ^{m}CₖₛAₖₛG_{ds}A_{ds}A_{ds}G_{ds}A_{ds}A_{ds}^{m}C_{ds}^{m}C_{ds}^{m} | 51.2 | 2038 | 2053 | 781 | 1248 |
| | C_{ds}A_{ds}AₖₛAₖₛGₖ | | | | | |
| 570400 | ^{m}CₖₛAₖₛ^{m}Cₖₛ^{m}C_{ds}^{m}C_{ds}A_{ds}G_{ds}A_{ds}A_{ds}G_{ds}A_{ds} | 51.1 | 2041 | 2056 | 782 | 1249 |
| | A_{ds}^{m}C_{ds}^{m}Cₖₛ^{m}CₖₛAₖ | | | | | |
| 570401 | Gₖₛ^{m}CₖₛAₖₛG_{ds}A_{ds}A_{ds}^{m}C_{ds}^{m}C_{ds}T_{ds}A_{ds}^{m}C_{ds} | 44.9 | 2066 | 2081 | 783 | 1250 |
| | A_{ds}A_{ds}AₖₛAₖₛGₖ | | | | | |
| 570402 | GₖₛTₖₛGₖₛ^{m}C_{ds}A_{ds}G_{ds}A_{ds}A_{ds}^{m}C_{ds}^{m}C_{ds}T_{ds}A_{ds} | 53 | 2068 | 2083 | 784 | 1251 |
| | ^{m}C_{ds}AₖₛAₖₛAₖ | | | | | |
| 570403 | GₖₛGₖₛGₖₛT_{ds}G_{ds}^{m}C_{ds}A_{ds}G_{ds}A_{ds}A_{ds}^{m}C_{ds}^{m}C_{ds} | 51.5 | 2070 | 2085 | 785 | 1252 |
| | T_{ds}Aₖₛ^{m}CₖₛAₖ | | | | | |
| 570404 | GₖₛTₖₛGₖₛG_{ds}G_{ds}T_{ds}G_{ds}^{m}C_{ds}A_{ds}G_{ds}A_{ds}A_{ds}^{m} | 57.4 | 2072 | 2087 | 786 | 1253 |
| | C_{ds}^{m}CₖₛTₖₛAₖ | | | | | |
| 570405 | ^{m}Cₖₛ^{m}CₖₛAₖₛ^{m}C_{ds}A_{ds}^{m}C_{ds}G_{ds}G_{ds}^{m}C_{ds}T_{ds}^{m}C_{ds} | 54.3 | 2116 | 2131 | 787 | 1254 |
| | A_{ds}T_{ds}AₖₛGₖₛGₖ | | | | | |
| 570406 | Aₖₛ^{m}Cₖₛ^{m}Cₖₛ^{m}C_{ds}A_{ds}^{m}C_{ds}A_{ds}^{m}C_{ds}G_{ds}G_{ds}^{m}C_{ds} | 43.6 | 2118 | 2133 | 788 | 1255 |
| | T_{ds}^{m}C_{ds}AₖₛTₖₛAₖ | | | | | |
| 570407 | TₖₛGₖₛAₖₛ^{m}C_{ds}^{m}C_{ds}^{m}C_{ds}A_{ds}^{m}C_{ds}A_{ds}^{m}C_{ds}G_{ds} | 44 | 2120 | 2135 | 789 | 1256 |
| | G_{ds}^{m}C_{ds}Tₖₛ^{m}CₖₛAₖ | | | | | |
| 570408 | Gₖₛ^{m}CₖₛTₖₛG_{ds}A_{ds}^{m}C_{ds}^{m}C_{ds}^{m}C_{ds}A_{ds}^{m}C_{ds}A_{ds} | 56.5 | 2122 | 2137 | 790 | 1257 |
| | C_{ds}G_{ds}Gₖₛ^{m}CₖₛTₖ | | | | | |
| 570409 | TₖₛGₖₛGₖₛ^{m}C_{ds}T_{ds}G_{ds}A_{ds}^{m}C_{ds}^{m}C_{ds}^{m}C_{ds}A_{ds}^{m} | 54.8 | 2124 | 2139 | 791 | 1258 |
| | C_{ds}A_{ds}^{m}CₖₛGₖₛGₖ | | | | | |
| 570410 | GₖₛGₖₛTₖₛG_{ds}G_{ds}^{m}C_{ds}T_{ds}G_{ds}A_{ds}^{m}C_{ds}^{m}C_{ds}^{m} | 46.8 | 2126 | 2141 | 792 | 1259 |
| | C_{ds}A_{ds}^{m}CₖₛAₖₛ^{m}Cₖ | | | | | |
| 570411 | AₖₛTₖₛGₖₛG_{ds}T_{ds}G_{ds}G_{ds}^{m}C_{ds}T_{ds}G_{ds}A_{ds}^{m}C_{ds}^{m} | 73.8 | 2128 | 2143 | 793 | 1260 |
| | C_{ds}^{m}CₖₛAₖₛ^{m}Cₖ | | | | | |
| 570412 | GₖₛAₖₛAₖₛT_{ds}G_{ds}G_{ds}T_{ds}G_{ds}G_{ds}^{m}C_{ds}T_{ds}G_{ds}A_{ds} | 43.5 | 2130 | 2145 | 794 | 1261 |
| | ^{m}Cₖₛ^{m}Cₖₛ^{m}Cₖ | | | | | |
| 570413 | ^{m}CₖₛTₖₛAₖₛA_{ds}A_{ds}G_{ds}G_{ds}A_{ds}^{m}C_{ds}G_{ds}^{m}C_{ds}A_{ds} | 54.4 | 2159 | 2174 | 795 | 1262 |
| | G_{ds}GₖₛGₖₛAₖ | | | | | |
| 570414 | AₖₛAₖₛ^{m}CₖₛT_{ds}A_{ds}A_{ds}A_{ds}G_{ds}G_{ds}A_{ds}^{m}C_{ds}G_{ds} | 49.1 | 2161 | 2176 | 796 | 1263 |
| | ^{m}C_{ds}AₖₛGₖₛGₖ | | | | | |
| 570415 | GₖₛAₖₛGₖₛA_{ds}A_{ds}^{m}C_{ds}T_{ds}A_{ds}A_{ds}A_{ds}G_{ds}G_{ds} | 35.4 | 2164 | 2179 | 797 | 1264 |
| | A_{ds}^{m}CₖₛGₖₛ^{m}Ck | | | | | |

TABLE 17
Inhibition of human DMPK RNA transcript in HepG2 cell stargeting SEQ ID NO: 2

| | | | Start | Stop | | |
|---|---|---|---|---|---|---|
| | | | Site on | Site on | | Modified |
| ISIS | | % Target | Seq | Seq | Unmodified | Seq ID |
| No. | Sequence | Expression | ID: 2 | ID: 2 | Seq ID No. | No. |
| UTC | N/A | 100 | N/A | N/A | | |
| 445569 | ^{m}CₑₛGₑₛGₑₛAₑₛGₑₛ^{m}C_{ds}G_{ds}G_{ds}T_{ds}T_{ds}G_{ds}T_{ds} | 41.4 | 13226 | 13245 | 24 | 1443 |
| | G_{ds}A_{ds}A_{ds}^{m}CₑₛTₑₛGₑₛGₑₛ^{m}Cₑ | | | | | |
| 486178 | Aₖₛ^{m}CₖₛAₖₛA_{ds}T_{ds}A_{ds}A_{ds}A_{ds}T_{ds}A_{ds}^{m}C_{ds}^{m} | 24 | 13836 | 13851 | 23 | 881 |
| | C_{ds}G_{ds}AₖₛGₖₛGₖ | | | | | |
| 570493 | AₖₛTₖₛTₖₛG_{ds}G_{ds}T_{ds}^{m}C_{ds}^{m}C_{ds}^{m}C_{ds}A_{ds}A_{ds} | 112.1 | 3973 | 3988 | 798 | 1265 |
| | G_{ds}^{m}C_{ds}^{m}Cₖₛ^{m}Cₖₛ^{m}Cₖ | | | | | |
| 570494 | ^{m}Cₖₛ^{m}CₖₛAₖₛT_{ds}T_{ds}G_{ds}G_{ds}T_{ds}^{m}C_{ds}^{m}C_{ds}^{m}C_{ds} | 91.3 | 3975 | 3990 | 799 | 1266 |
| | A_{ds}A_{ds}Gₖₛ^{m}Cₖₛ^{m}Cₖ | | | | | |
| 570495 | Gₖₛ^{m}Cₖₛ^{m}Cₖₛ^{m}C_{ds}A_{ds}T_{ds}T_{ds}G_{ds}G_{ds}T_{ds}^{m}C_{ds} | 103.4 | 3977 | 3992 | 800 | 1267 |
| | ^{m}C_{ds}^{m}C_{ds}AₖₛAₖₛGₖ | | | | | |
| 570496 | Aₖₛ^{m}CₖₛGₖₛ^{m}C_{ds}^{m}C_{ds}^{m}C_{ds}A_{ds}T_{ds}T_{ds}G_{ds}G_{ds} | 67.8 | 3979 | 3994 | 801 | 1268 |
| | T_{ds}^{m}C_{ds}^{m}Cₖₛ^{m}CₖₛAₖ | | | | | |
| 570497 | ^{m}Cₖₛ^{m}CₖₛAₖₛ^{m}C_{ds}G_{ds}^{m}C_{ds}^{m}C_{ds}^{m}C_{ds}A_{ds}T_{ds} | 77.3 | 3981 | 3996 | 802 | 1269 |
| | T_{ds}G_{ds}G_{ds}Tₖₛ^{m}Cₖₛ^{m}Cₖ | | | | | |
| 570498 | ^{m}CₖₛAₖₛ^{m}Cₖₛ^{m}C_{ds}A_{ds}^{m}C_{ds}G_{ds}^{m}C_{ds}^{m}C_{ds}^{m}C_{ds} | 98.3 | 3983 | 3998 | 803 | 1270 |
| | A_{ds}T_{ds}T_{ds}GₖₛGₖₛTₖ | | | | | |
| 570499 | AₖₛGₖₛAₖₛ^{m}C_{ds}^{m}C_{ds}^{m}C_{ds}A_{ds}A_{ds}^{m}C_{ds}T_{ds}^{m} | 63.7 | 4036 | 4051 | 804 | 1271 |
| | C_{ds}^{m}C_{ds}A_{ds}^{m}Cₖₛ^{m}Cₖₛ^{m}Cₖ | | | | | |
| 570500 | Tₖₛ^{m}CₖₛAₖₛ^{m}C_{ds}^{m}C_{ds}T_{ds}^{m}C_{ds}G_{ds}^{m}C_{ds}^{m}C_{ds} | 43 | 4181 | 4196 | 805 | 1272 |
| | ^{m}C_{ds}^{m}C_{ds}T_{ds}^{m}CₖₛTₖₛTₖ | | | | | |
| 570501 | ^{m}Cₖₛ^{m}CₖₛTₖₛ^{m}C_{ds}A_{ds}^{m}C_{ds}^{m}C_{ds}T_{ds}^{m}C_{ds}G_{ds} | 38.1 | 4183 | 4198 | 806 | 1273 |
| | ^{m}C_{ds}^{m}C_{ds}^{m}C_{ds}^{m}CₖₛTₖₛ^{m}Cₖ | | | | | |
| 570502 | AₖₛGₖₛ^{m}Cₖₛ^{m}C_{ds}^{m}C_{ds}^{m}C_{ds}T_{ds}^{m}C_{ds}A_{ds}^{m}C_{ds} | 85.4 | 4187 | 4202 | 807 | 1274 |
| | ^{m}C_{ds}T_{ds}^{m}C_{ds}Gₖₛ^{m}Cₖₛ^{m}Cₖ | | | | | |
| 570503 | ^{m}CₖₛTₖₛ^{m}CₖₛA_{ds}A_{ds}A_{ds}G_{ds}^{m}C_{ds}^{m}C_{ds}^{m}C_{ds}^{m} | 115.8 | 4210 | 4225 | 808 | 1275 |
| | C_{ds}^{m}C_{ds}^{m}C_{ds}Aₖₛ^{m}CₖₛGₖ | | | | | |
| 570504 | AₖₛTₖₛ^{m}Cₖₛ^{m}C_{ds}T_{ds}^{m}C_{ds}A_{ds}A_{ds}A_{ds}G_{ds}^{m}C_{ds} | 114.5 | 4213 | 4228 | 809 | 1276 |
| | ^{m}C_{ds}^{m}C_{ds}^{m}Cₖₛ^{m}Cₖₛ^{m}Cₖ | | | | | |
| 570505 | GₖₛGₖₛAₖₛT_{ds}^{m}C_{ds}^{m}C_{ds}T_{ds}^{m}C_{ds}A_{ds}A_{ds}A_{ds} | 88.1 | 4215 | 4230 | 810 | 1277 |
| | G_{ds}^{m}C_{ds}^{m}Cₖₛ^{m}Cₖₛ^{m}Cₖ | | | | | |
| 570506 | Gₖₛ^{m}CₖₛGₖₛG_{ds}A_{ds}T_{ds}^{m}C_{ds}^{m}C_{ds}T_{ds}^{m}C_{ds}A_{ds} | 93.1 | 4217 | 4232 | 811 | 1278 |
| | A_{ds}A_{ds}Gₖₛ^{m}Cₖₛ^{m}Cₖ | | | | | |
| 570507 | Gₖₛ^{m}CₖₛGₖₛ^{m}C_{ds}G_{ds}G_{ds}A_{ds}T_{ds}^{m}C_{ds}^{m}C_{ds}T_{ds} | 102.9 | 4219 | 4234 | 812 | 1279 |
| | ^{m}C_{ds}A_{ds}AₖₛAₖₛGₖ | | | | | |
| 570508 | GₖₛGₖₛGₖₛ^{m}C_{ds}G_{ds}^{m}C_{ds}G_{ds}G_{ds}A_{ds}T_{ds}^{m}C_{ds} | 78.5 | 4221 | 4236 | 813 | 1280 |
| | ^{m}C_{ds}T_{ds}^{m}CₖₛAₖₛAₖ | | | | | |
| 570509 | GₖₛAₖₛGₖₛ^{m}C_{ds}T_{ds}G_{ds}^{m}C_{ds}A_{ds}G_{ds}^{m}C_{ds}^{m}C_{ds} | 192.2 | 4239 | 4254 | 814 | 1281 |
| | G_{ds}G_{ds}AₖₛGₖₛAₖ | | | | | |
| 570510 | AₖₛGₖₛGₖₛA_{ds}G_{ds}^{m}C_{ds}T_{ds}G_{ds}^{m}C_{ds}A_{ds}G_{ds}^{m} | 219.8 | 4241 | 4256 | 815 | 1282 |
| | C_{ds}^{m}C_{ds}GₖₛGₖₛAₖ | | | | | |
| 570511 | ^{m}CₖₛGₖₛGₖₛA_{ds}G_{ds}G_{ds}A_{ds}G_{ds}^{m}C_{ds}T_{ds}G_{ds}^{m} | 128.6 | 4244 | 4259 | 816 | 1283 |
| | C_{ds}A_{ds}Gₖₛ^{m}Cₖₛ^{m}Cₖ | | | | | |
| 570512 | Aₖₛ^{m}Cₖₛ^{m}Cₖₛ^{m}C_{ds}G_{ds}G_{ds}A_{ds}G_{ds}G_{ds}A_{ds}G_{ds} | 89.9 | 4247 | 4262 | 817 | 1284 |
| | ^{m}C_{ds}T_{ds}Gₖₛ^{m}CₖₛAₖ | | | | | |
| 570513 | Gₖₛ^{m}CₖₛAₖₛ^{m}C_{ds}^{m}C_{ds}^{m}C_{ds}G_{ds}G_{ds}A_{ds}G_{ds}G_{ds} | 96.1 | 4249 | 4264 | 818 | 1285 |
| | A_{ds}G_{ds}^{m}CₖₛTₖₛGₖ | | | | | |
| 570514 | GₖₛGₖₛGₖₛ^{m}C_{ds}A_{ds}^{m}C_{ds}^{m}C_{ds}^{m}C_{ds}G_{ds}G_{ds}A_{ds} | 67.8 | 4251 | 4266 | 819 | 1286 |
| | G_{ds}G_{ds}AₖₛGₖₛ^{m}Cₖ | | | | | |
| 570515 | ^{m}CₖₛAₖₛGₖₛG_{ds}G_{ds}^{m}C_{ds}A_{ds}^{m}C_{ds}^{m}C_{ds}^{m}C_{ds} | 64.2 | 4253 | 4268 | 820 | 1287 |
| | G_{ds}G_{ds}A_{ds}GₖₛGₖₛAₖ | | | | | |
| 570516 | TₖₛGₖₛ^{m}CₖₛA_{ds}G_{ds}G_{ds}G_{ds}^{m}C_{ds}A_{ds}^{m}C_{ds}^{m}C_{ds} | 62.2 | 4255 | 4270 | 821 | 1288 |
| | ^{m}C_{ds}G_{ds}GₖₛAₖₛGₖ | | | | | |
| 570517 | ^{m}Cₖₛ^{m}CₖₛTₖₛG_{ds}^{m}C_{ds}A_{ds}G_{ds}G_{ds}G_{ds}^{m}C_{ds}A_{ds} | 77.7 | 4257 | 4272 | 822 | 1289 |
| | ^{m}C_{ds}^{m}C_{ds}^{m}CₖₛGₖₛGₖ | | | | | |
| 570518 | ^{m}CₖₛGₖₛAₖₛ^{m}C_{ds}A_{ds}^{m}C_{ds}^{m}C_{ds}T_{ds}G_{ds}^{m}C_{ds} | 79 | 4262 | 4277 | 823 | 1290 |
| | A_{ds}G_{ds}G_{ds}Gₖₛ^{m}CₖₛAₖ | | | | | |
| 570519 | ^{m}CₖₛAₖₛ^{m}CₖₛG_{ds}A_{ds}^{m}C_{ds}A_{ds}^{m}C_{ds}^{m}C_{ds}T_{ds} | 68.5 | 4264 | 4279 | 824 | 1291 |
| | G_{ds}^{m}C_{ds}A_{ds}GₖₛGₖₛGₖ | | | | | |
| 570520 | AₖₛGₖₛ^{m}CₖₛA_{ds}^{m}C_{ds}G_{ds}A_{ds}^{m}C_{ds}A_{ds}^{m}C_{ds}^{m} | 39.8 | 4266 | 4281 | 825 | 1292 |
| | C_{ds}T_{ds}G_{ds}^{m}CₖₛAₖₛGₖ | | | | | |
| 570521 | GₖₛAₖₛAₖₛG_{ds}^{m}C_{ds}A_{ds}^{m}C_{ds}G_{ds}A_{ds}^{m}C_{ds}A_{ds} | 32.4 | 4268 | 4283 | 826 | 1293 |
| | ^{m}C_{ds}^{m}C_{ds}TₖₛGₖₛ^{m}Cₖ | | | | | |
| 570522 | ^{m}Cₖₛ^{m}CₖₛAₖₛG_{ds}G_{ds}T_{ds}A_{ds}G_{ds}T_{ds}T_{ds}^{m}C_{ds} | 41 | 4353 | 4368 | 827 | 1294 |
| | T_{ds}^{m}C_{ds}AₖₛTₖₛ^{m}Cₖ | | | | | |
| 570523 | ^{m}CₖₛAₖₛ^{m}Cₖₛ^{m}C_{ds}A_{ds}G_{ds}G_{ds}T_{ds}A_{ds}G_{ds}T_{ds} | 71.9 | 4355 | 4370 | 828 | 1295 |
| | T_{ds}^{m}C_{ds}Tₖₛ^{m}CₖₛAₖ | | | | | |
| 570524 | ^{m}CₖₛTₖₛ^{m}CₖₛA_{ds}^{m}C_{ds}^{m}C_{ds}A_{ds}G_{ds}G_{ds}T_{ds}A_{ds} | 105.9 | 4357 | 4372 | 829 | 1296 |
| | G_{ds}T_{ds}Tₖₛ^{m}CₖₛTₖ | | | | | |
| 570525 | AₖₛGₖₛ^{m}CₖₛT_{ds}^{m}C_{ds}A_{ds}^{m}C_{ds}^{m}C_{ds}A_{ds}G_{ds}G_{ds} | 99.3 | 4359 | 4374 | 830 | 1297 |
| | T_{ds}A_{ds}GₖₛTₖₛTₖ | | | | | |
| 570526 | GₖₛGₖₛAₖₛG_{ds}^{m}C_{ds}T_{ds}^{m}C_{ds}A_{ds}^{m}C_{ds}^{m}C_{ds}A_{ds} | 85.2 | 4361 | 4376 | 831 | 1298 |
| | G_{ds}G_{ds}TₖₛAₖₛGₖ | | | | | |
| 570527 | ^{m}Cₖₛ^{m}CₖₛGₖₛG_{ds}A_{ds}G_{ds}^{m}C_{ds}T_{ds}^{m}C_{ds}A_{ds}^{m} | 82.5 | 4363 | 4378 | 832 | 1299 |
| | C_{ds}^{m}C_{ds}A_{ds}GₖₛGₖₛTₖ | | | | | |
| 570528 | Gₖₛ^{m}Cₖₛ^{m}Cₖₛ^{m}C_{ds}G_{ds}G_{ds}A_{ds}G_{ds}^{m}C_{ds}T_{ds}^{m} | 60.5 | 4365 | 4380 | 833 | 1300 |
| | C_{ds}A_{ds}^{m}C_{ds}^{m}CₖₛAₖₛGₖ | | | | | |
| 570529 | TₖₛAₖₛGₖₛA_{ds}G_{ds}^{m}C_{ds}T_{ds}T_{ds}^{m}C_{ds}^{m}C_{ds}T_{ds}^{m} | 35.4 | 4435 | 4450 | 834 | 1301 |
| | C_{ds}T_{ds}^{m}Cₖₛ^{m}Cₖₛ^{m}Cₖ | | | | | |
| 570530 | ^{m}Cₖₛ^{m}CₖₛTₖₛA_{ds}G_{ds}A_{ds}G_{ds}^{m}C_{ds}T_{ds}T_{ds}^{m}C_{ds} | 29.4 | 4437 | 4452 | 835 | 1302 |
| | ^{m}C_{ds}T_{ds}^{m}CₖₛTₖₛ^{m}Cₖ | | | | | |
| 570531 | AₖₛTₖₛ^{m}Cₖₛ^{m}C_{ds}T_{ds}A_{ds}G_{ds}A_{ds}G_{ds}^{m}C_{ds}T_{ds} | 30.4 | 4439 | 4454 | 836 | 1303 |
| | T_{ds}^{m}C_{ds}^{m}CₖₛTₖₛ^{m}Cₖ | | | | | |
| 570532 | ^{m}CₖₛAₖₛAₖₛT_{ds}^{m}C_{ds}^{m}C_{ds}T_{ds}A_{ds}G_{ds}A_{ds}G_{ds}^{m} | 30.3 | 4441 | 4456 | 837 | 1304 |
| | C_{ds}T_{ds}Tₖₛ^{m}Cₖₛ^{m}Cₖ | | | | | |
| 570533 | ^{m}Cₖₛ^{m}Cₖₛ^{m}CₖₛA_{ds}A_{ds}T_{ds}^{m}C_{ds}^{m}C_{ds}T_{ds}A_{ds} | 54.1 | 4443 | 4458 | 838 | 1305 |
| | G_{ds}A_{ds}G_{ds}^{m}CₖₛTₖₛTₖ | | | | | |
| 570534 | ^{m}Cₖₛ^{m}Cₖₛ^{m}Cₖₛ^{m}C_{ds}^{m}C_{ds}A_{ds}A_{ds}T_{ds}^{m}C_{ds}^{m}C_{ds} | 60.1 | 4445 | 4460 | 839 | 1306 |
| | T_{ds}A_{ds}G_{ds}AₖₛGₖₛ^{m}Cₖ | | | | | |
| 570535 | ^{m}CₖₛAₖₛ^{m}Cₖₛ^{m}C_{ds}^{m}C_{ds}^{m}C_{ds}^{m}C_{ds}A_{ds}A_{ds}T_{ds} | 68.5 | 4447 | 4462 | 840 | 1307 |
| | ^{m}C_{ds}^{m}C_{ds}T_{ds}AₖₛGₖₛAₖ | | | | | |
| 570536 | AₖₛGₖₛ^{m}CₖₛA_{ds}^{m}C_{ds}^{m}C_{ds}^{m}C_{ds}^{m}C_{ds}^{m}C_{ds}A_{ds} | 37.5 | 4449 | 4464 | 841 | 1308 |
| | A_{ds}T_{ds}^{m}C_{ds}^{m}CₖₛTₖₛAₖ | | | | | |
| 570537 | Gₖₛ^{m}CₖₛAₖₛG_{ds}^{m}C_{ds}A_{ds}^{m}C_{ds}^{m}C_{ds}^{m}C_{ds}^{m}C_{ds} | 50.9 | 4451 | 4466 | 842 | 1309 |
| | ^{m}C_{ds}A_{ds}A_{ds}Tₖₛ^{m}Cₖₛ^{m}Cₖ | | | | | |
| 570538 | GₖₛGₖₛGₖₛ^{m}C_{ds}A_{ds}G_{ds}^{m}C_{ds}A_{ds}^{m}C_{ds}^{m}C_{ds}^{m} | 67.7 | 4453 | 4468 | 843 | 1310 |
| | C_{ds}^{m}C_{ds}^{m}C_{ds}AₖₛAₖₛTₖ | | | | | |
| 570539 | TₖₛGₖₛAₖₛ^{m}C_{ds}A_{ds}^{m}C_{ds}A_{ds}^{m}C_{ds}^{m}C_{ds}^{m}C_{ds} | 55.9 | 4498 | 4513 | 844 | 1311 |
| | T_{ds}^{m}C_{ds}T_{ds}TₖₛAₖₛ^{m}Cₖ | | | | | |
| 570540 | ^{m}Cₖₛ^{m}CₖₛTₖₛG_{ds}A_{ds}^{m}C_{ds}A_{ds}^{m}C_{ds}A_{ds}^{m}C_{ds}^{m} | 45.1 | 4500 | 4515 | 845 | 1312 |
| | C_{ds}^{m}C_{ds}T_{ds}^{m}CₖₛTₖₛTₖ | | | | | |
| 570541 | ^{m}CₖₛAₖₛ^{m}Cₖₛ^{m}C_{ds}T_{ds}G_{ds}A_{ds}^{m}C_{ds}A_{ds}^{m}C_{ds} | 30.9 | 4502 | 4517 | 846 | 1313 |
| | A_{ds}^{m}C_{ds}^{m}C_{ds}^{m}CₖₛTₖₛ^{m}Cₖ | | | | | |
| 570542 | Tₖₛ^{m}Cₖₛ^{m}CₖₛA_{ds}^{m}C_{ds}^{m}C_{ds}T_{ds}G_{ds}A_{ds}^{m}C_{ds} | 35 | 4504 | 4519 | 847 | 1314 |
| | A_{ds}^{m}C_{ds}A_{ds}^{m}Cₖₛ^{m}Cₖₛ^{m}Cₖ | | | | | |
| 570543 | ^{m}CₖₛAₖₛTₖₛ^{m}C_{ds}^{m}C_{ds}A_{ds}^{m}C_{ds}^{m}C_{ds}T_{ds}G_{ds} | 48 | 4506 | 4521 | 848 | 1315 |
| | A_{ds}^{m}C_{ds}A_{ds}^{m}CₖₛAₖₛ^{m}Cₖ | | | | | |
| 570544 | ^{m}CₖₛTₖₛ^{m}CₖₛA_{ds}T_{ds}^{m}C_{ds}^{m}C_{ds}A_{ds}^{m}C_{ds}^{m}C_{ds} | 37.1 | 4508 | 4523 | 849 | 1316 |
| | T_{ds}G_{ds}A_{ds}^{m}CₖₛAₖₛ^{m}Cₖ | | | | | |
| 570545 | ^{m}Cₖₛ^{m}Cₖₛ^{m}CₖₛT_{ds}^{m}C_{ds}A_{ds}T_{ds}^{m}C_{ds}^{m}C_{ds}A_{ds} | 46 | 4510 | 4525 | 850 | 1317 |
| | ^{m}C_{ds}^{m}C_{ds}T_{ds}GₖₛAₖₛ^{m}Cₖ | | | | | |
| 570546 | Gₖₛ^{m}Cₖₛ^{m}Cₖₛ^{m}C_{ds}^{m}C_{ds}T_{ds}^{m}C_{ds}A_{ds}T_{ds}^{m}C_{ds} | 79.2 | 4512 | 4527 | 851 | 1318 |
| | ^{m}C_{ds}A_{ds}^{m}C_{ds}^{m}CₖₛTₖₛGₖ | | | | | |
| 570547 | AₖₛGₖₛGₖₛ^{m}C_{ds}^{m}C_{ds}^{m}C_{ds}^{m}C_{ds}T_{ds}^{m}C_{ds}A_{ds} | 40.7 | 4514 | 4529 | 852 | 1319 |
| | T_{ds}^{m}C_{ds}^{m}C_{ds}Aₖₛ^{m}Cₖₛ^{m}Cₖ | | | | | |
| 570548 | GₖₛAₖₛAₖₛG_{ds}G_{ds}^{m}C_{ds}^{m}C_{ds}^{m}C_{ds}^{m}C_{ds}T_{ds}^{m} | 35.9 | 4516 | 4531 | 853 | 1320 |
| | C_{ds}A_{ds}T_{ds}^{m}Cₖₛ^{m}CₖₛAₖ | | | | | |
| 570549 | AₖₛGₖₛGₖₛT_{ds}A_{ds}A_{ds}G_{ds}A_{ds}G_{ds}A_{ds}^{m}C_{ds}^{m} | 18.8 | 4613 | 4628 | 854 | 1321 |
| | C_{ds}^{m}C_{ds}^{m}Cₖₛ^{m}Cₖₛ^{m}Cₖ | | | | | |
| 570550 | ^{m}Cₖₛ^{m}CₖₛAₖₛG_{ds}G_{ds}T_{ds}A_{ds}A_{ds}G_{ds}A_{ds}G_{ds} | 16.2 | 4615 | 4630 | 855 | 1322 |
| | A_{ds}^{m}C_{ds}^{m}Cₖₛ^{m}Cₖₛ^{m}Cₖ | | | | | |
| 570551 | TₖₛTₖₛ^{m}Cₖₛ^{m}C_{ds}A_{ds}G_{ds}G_{ds}T_{ds}A_{ds}A_{ds}G_{ds}A_{ds} | 38.9 | 4617 | 4632 | 856 | 1323 |
| | G_{ds}Aₖₛ^{m}Cₖₛ^{m}Cₖ | | | | | |
| 570552 | ^{m}Cₖₛ^{m}CₖₛAₖₛT_{ds}T_{ds}^{m}C_{ds}^{m}C_{ds}A_{ds}G_{ds}G_{ds}T_{ds} | 28.6 | 4620 | 4635 | 857 | 1324 |
| | A_{ds}A_{ds}GₖₛAₖₛGₖ | | | | | |
| 570553 | Tₖₛ^{m}Cₖₛ^{m}Cₖₛ^{m}C_{ds}A_{ds}T_{ds}T_{ds}^{m}C_{ds}^{m}C_{ds}A_{ds}G_{ds} | 42.6 | 4622 | 4637 | 858 | 1325 |
| | G_{ds}T_{ds}AₖₛAₖₛGₖ | | | | | |
| 570554 | TₖₛAₖₛTₖₛ^{m}C_{ds}^{m}C_{ds}^{m}C_{ds}A_{ds}T_{ds}T_{ds}^{m}C_{ds}^{m}C_{ds} | 31.8 | 4624 | 4639 | 859 | 1326 |
| | A_{ds}G_{ds}GₖₛTₖₛAₖ | | | | | |
| 570555 | ^{m}Cₖₛ^{m}CₖₛTₖₛA_{ds}T_{ds}^{m}C_{ds}^{m}C_{ds}^{m}C_{ds}A_{ds}T_{ds}T_{ds} | 62 | 4626 | 4641 | 860 | 1327 |
| | ^{m}C_{ds}^{m}C_{ds}AₖₛGₖₛGₖ | | | | | |
| 570556 | GₖₛAₖₛ^{m}Cₖₛ^{m}C_{ds}T_{ds}A_{ds}T_{ds}^{m}C_{ds}^{m}C_{ds}^{m}C_{ds} | 20 | 4628 | 4643 | 861 | 1328 |
| | A_{ds}T_{ds}T_{ds}^{m}Cₖₛ^{m}CₖₛAₖ | | | | | |
| 570557 | AₖₛAₖₛGₖₛA_{ds}^{m}C_{ds}^{m}C_{ds}T_{ds}A_{ds}T_{ds}^{m}C_{ds}^{m}C_{ds} | 29.8 | 4630 | 4645 | 862 | 1329 |
| | ^{m}C_{ds}A_{ds}TₖₛTₖₛ^{m}Cₖ | | | | | |
| 570558 | TₖₛGₖₛAₖₛA_{ds}G_{ds}A_{ds}^{m}C_{ds}^{m}C_{ds}T_{ds}A_{ds}T_{ds}^{m} | 45.5 | 4632 | 4647 | 863 | 1330 |
| | C_{ds}^{m}C_{ds}^{m}CₖₛAₖₛTₖ | | | | | |
| 570559 | TₖₛGₖₛGₖₛ^{m}C_{ds}^{m}C_{ds}^{m}C_{ds}^{m}C_{ds}G_{ds}T_{ds}T_{ds}A_{ds} | 72.7 | 4650 | 4665 | 864 | 1331 |
| | G_{ds}A_{ds}AₖₛTₖₛTₖ | | | | | |
| 570560 | AₖₛGₖₛTₖₛG_{ds}G_{ds}^{m}C_{ds}^{m}C_{ds}^{m}C_{ds}^{m}C_{ds}G_{ds}T_{ds} | 33.7 | 4652 | 4667 | 865 | 1332 |
| | T_{ds}A_{ds}GₖₛAₖₛAₖ | | | | | |
| 570561 | Gₖₛ^{m}CₖₛAₖₛG_{ds}T_{ds}G_{ds}G_{ds}^{m}C_{ds}^{m}C_{ds}^{m}C_{ds}^{m} | 17.5 | 4654 | 4669 | 866 | 1333 |
| | C_{ds}G_{ds}T_{ds}TₖₛAₖₛGₖ | | | | | |
| 570562 | AₖₛGₖₛGₖₛ^{m}C_{ds}A_{ds}G_{ds}T_{ds}G_{ds}G_{ds}^{m}C_{ds}^{m}C_{ds} | 27.9 | 4656 | 4671 | 867 | 1334 |
| | ^{m}C_{ds}^{m}C_{ds}GₖₛTₖₛTₖ | | | | | |
| 570563 | ^{m}CₖₛTₖₛAₖₛG_{ds}G_{ds}^{m}C_{ds}A_{ds}G_{ds}T_{ds}G_{ds}G_{ds}^{m} | 31.3 | 4658 | 4673 | 868 | 1335 |
| | C_{ds}^{m}C_{ds}^{m}Cₖₛ^{m}CₖₛGₖ | | | | | |
| 570564 | ^{m}Cₖₛ^{m}Cₖₛ^{m}CₖₛT_{ds}A_{ds}G_{ds}G_{ds}^{m}C_{ds}A_{ds}G_{ds}T_{ds} | 23.8 | 4660 | 4675 | 869 | 1336 |
| | G_{ds}G_{ds}^{m}Cₖₛ^{m}Cₖₛ^{m}Cₖ | | | | | |
| 570565 | AₖₛGₖₛGₖₛT_{ds}^{m}C_{ds}^{m}C_{ds}^{m}C_{ds}A_{ds}G_{ds}A_{ds}^{m}C_{ds} | 17.2 | 4678 | 4693 | 870 | 1337 |
| | A_{ds}^{m}C_{ds}Tₖₛ^{m}Cₖₛ^{m}Cₖ | | | | | |
| 570566 | AₖₛTₖₛAₖₛG_{ds}G_{ds}T_{ds}^{m}C_{ds}^{m}C_{ds}^{m}C_{ds}A_{ds}G_{ds} | 33.1 | 4680 | 4695 | 871 | 1338 |
| | A_{ds}^{m}C_{ds}Aₖₛ^{m}CₖₛTₖ | | | | | |
| 570567 | GₖₛAₖₛAₖₛT_{ds}A_{ds}G_{ds}G_{ds}T_{ds}^{m}C_{ds}^{m}C_{ds}^{m}C_{ds} | 51.8 | 4682 | 4697 | 872 | 1339 |
| | A_{ds}G_{ds}Aₖₛ^{m}CₖₛAₖ | | | | | |
| 570568 | GₖₛAₖₛGₖₛA_{ds}A_{ds}T_{ds}A_{ds}G_{ds}G_{ds}T_{ds}^{m}C_{ds}^{m} | 20.3 | 4684 | 4699 | 873 | 1340 |
| | C_{ds}^{m}C_{ds}AₖₛGₖₛAₖ | | | | | |
| 570569 | ^{m}CₖₛAₖₛGₖₛA_{ds}G_{ds}A_{ds}A_{ds}T_{ds}A_{ds}G_{ds}G_{ds}T_{ds} | 19 | 4686 | 4701 | 874 | 1341 |
| | ^{m}C_{ds}^{m}Cₖₛ^{m}CₖₛAk | | | | | |

TABLE 18
Inhibition of human DMPK RNA transcript in HepG2 cell stargeting SEQ ID NO: 2

| | | | Start | Stop | | |
|---|---|---|---|---|---|---|
| | | | Site on | Site on | | Modified |
| ISIS | | % Target | Seq | Seq | Unmodified | Seq ID |
| No. | Sequence | Expression | ID: 2 | ID: 2 | Seq ID No. | No. |
| UTC | N/A | 100 | N/A | N/A | | |
| 445569 | ^{m}CₑₛGₑₛGₑₛAₑₛGₑₛ^{m}C_{ds}G_{ds}G_{ds}T_{ds}T_{ds}G_{ds}T_{ds} | 33.8 | 13226 | 13245 | 24 | 1443 |
| | G_{ds}A_{ds}A_{ds}^{m}CₑₛTₑₛGₑₛGₑₛ^{m}Cₑ | | | | | |
| 486178 | Aₖₛ^{m}CₖₛAₖₛA_{ds}T_{ds}A_{ds}A_{ds}A_{ds}T_{ds}A_{ds}^{m}C_{ds}^{m} | 24.4 | 13836 | 13851 | 23 | 881 |
| | C_{ds}G_{ds}AₖₛGₖₛGₖ | | | | | |
| 570647 | Gₖₛ^{m}CₖₛTₖₛT_{ds}G_{ds}G_{ds}G_{ds}^{m}C_{ds}^{m}C_{ds}^{m}C_{ds}A_{ds} | 60.6 | 5718 | 5733 | 645 | 1111 |
| | ^{m}C_{ds}^{m}C_{ds}^{m}Cₖₛ^{m}CₖₛTₖ | | | | | |
| 570648 | AₖₛGₖₛGₖₛ^{m}C_{ds}T_{ds}T_{ds}G_{ds}G_{ds}G_{ds}^{m}C_{ds}^{m}C_{ds}^{m} | 82 | 5720 | 5735 | 646 | 1112 |
| | C_{ds}A_{ds}^{m}Cₖₛ^{m}Cₖₛ^{m}Cₖ | | | | | |
| 570649 | ^{m}CₖₛGₖₛAₖₛG_{ds}G_{ds}^{m}C_{ds}T_{ds}T_{ds}G_{ds}G_{ds}G_{ds}^{m} | 133.4 | 5722 | 5737 | 647 | 1113 |
| | C_{ds}^{m}C_{ds}^{m}CₖₛAₖₛ^{m}Cₖ | | | | | |
| 570650 | AₖₛGₖₛ^{m}CₖₛG_{ds}A_{ds}G_{ds}G_{ds}^{m}C_{ds}T_{ds}T_{ds}G_{ds}G_{ds} | 54.1 | 5724 | 5739 | 648 | 1114 |
| | G_{ds}^{m}Cₖₛ^{m}Cₖₛ^{m}Cₖ | | | | | |
| 570651 | AₖₛGₖₛAₖₛG_{ds}^{m}C_{ds}G_{ds}A_{ds}G_{ds}G_{ds}^{m}C_{ds}T_{ds}T_{ds} | 88.5 | 5726 | 5741 | 649 | 1115 |
| | G_{ds}GₖₛGₖₛ^{m}Cₖ | | | | | |
| 570652 | Gₖₛ^{m}CₖₛAₖₛG_{ds}A_{ds}G_{ds}^{m}C_{ds}G_{ds}A_{ds}G_{ds}G_{ds}^{m} | 162.9 | 5728 | 5743 | 650 | 1116 |
| | C_{ds}T_{ds}TₖₛGₖₛGₖ | | | | | |
| 570653 | GₖₛAₖₛGₖₛ^{m}C_{ds}A_{ds}G_{ds}A_{ds}G_{ds}^{m}C_{ds}G_{ds}A_{ds} | 130 | 5730 | 5745 | 651 | 1117 |
| | G_{ds}G_{ds}^{m}CₖₛTₖₛTₖ | | | | | |
| 570654 | AₖₛAₖₛAₖₛG_{ds}G_{ds}A_{ds}G_{ds}^{m}C_{ds}A_{ds}G_{ds}A_{ds}G_{ds} | 66.5 | 5734 | 5749 | 652 | 1118 |
| | ^{m}C_{ds}GₖₛAₖₛGₖ | | | | | |
| 570655 | ^{m}CₖₛAₖₛAₖₛA_{ds}A_{ds}G_{ds}G_{ds}A_{ds}G_{ds}^{m}C_{ds}A_{ds} | 79 | 5736 | 5751 | 653 | 1119 |
| | G_{ds}A_{ds}Gₖₛ^{m}CₖₛGₖ | | | | | |
| 570656 | TₖₛGₖₛGₖₛA_{ds}^{m}C_{ds}^{m}C_{ds}A_{ds}A_{ds}A_{ds}A_{ds}G_{ds} | 57.4 | 5741 | 5756 | 654 | 1120 |
| | G_{ds}A_{ds}Gₖₛ^{m}CₖₛAₖ | | | | | |
| 570657 | ^{m}Cₖₛ^{m}CₖₛTₖₛG_{ds}G_{ds}A_{ds}^{m}C_{ds}^{m}C_{ds}A_{ds}A_{ds}A_{ds} | 129.2 | 5743 | 5758 | 655 | 1121 |
| | A_{ds}G_{ds}GₖₛAₖₛGₖ | | | | | |
| 570658 | ^{m}CₖₛAₖₛ^{m}Cₖₛ^{m}C_{ds}T_{ds}G_{ds}G_{ds}A_{ds}^{m}C_{ds}^{m}C_{ds} | 66.3 | 5745 | 5760 | 656 | 1122 |
| | A_{ds}A_{ds}A_{ds}AₖₛGₖₛGₖ | | | | | |
| 570659 | ^{m}CₖₛGₖₛ^{m}CₖₛA_{ds}^{m}C_{ds}^{m}C_{ds}T_{ds}G_{ds}G_{ds}A_{ds}^{m} | 58.7 | 5747 | 5762 | 657 | 1123 |
| | C_{ds}^{m}C_{ds}A_{ds}AₖₛAₖₛAₖ | | | | | |
| 570660 | GₖₛAₖₛ^{m}Cₖₛ^{m}C_{ds}G_{ds}^{m}C_{ds}A_{ds}^{m}C_{ds}^{m}C_{ds}T_{ds} | 55.4 | 5750 | 5765 | 658 | 1124 |
| | G_{ds}G_{ds}A_{ds}^{m}Cₖₛ^{m}CₖₛAₖ | | | | | |
| 570661 | Aₖₛ^{m}Cₖₛ^{m}CₖₛT_{ds}T_{ds}G_{ds}T_{ds}A_{ds}G_{ds}T_{ds}G_{ds}G_{ds} | 45.4 | 5951 | 5966 | 659 | 1125 |
| | A_{ds}^{m}CₖₛGₖₛAₖ | | | | | |
| 570662 | Tₖₛ^{m}CₖₛAₖₛ^{m}C_{ds}^{m}C_{ds}T_{ds}T_{ds}G_{ds}T_{ds}A_{ds}G_{ds} | 63.5 | 5953 | 5968 | 660 | 1126 |
| | T_{ds}G_{ds}GₖₛAₖₛ^{m}Cₖ | | | | | |
| 570663 | Gₖₛ^{m}CₖₛTₖₛ^{m}C_{ds}A_{ds}^{m}C_{ds}^{m}C_{ds}T_{ds}T_{ds}G_{ds}T_{ds} | 56.6 | 5955 | 5970 | 661 | 1127 |
| | A_{ds}G_{ds}TₖₛGₖₛGₖ | | | | | |
| 570664 | GₖₛGₖₛAₖₛG_{ds}A_{ds}G_{ds}G_{ds}A_{ds}G_{ds}G_{ds}^{m}C_{ds}G_{ds} | 125.6 | 6015 | 6030 | 662 | 1111 |
| | A_{ds}TₖₛAₖₛGₖ | | | | | |
| 570665 | AₖₛGₖₛGₖₛG_{ds}A_{ds}G_{ds}A_{ds}G_{ds}G_{ds}A_{ds}G_{ds}G_{ds}^{m} | 64.2 | 6017 | 6032 | 663 | 1112 |
| | C_{ds}GₖₛAₖₛTₖ | | | | | |
| 570666 | ^{m}CₖₛTₖₛ^{m}Cₖₛ^{m}C_{ds}T_{ds}G_{ds}^{m}C_{ds}T_{ds}^{m}C_{ds}A_{ds}G_{ds} | 59 | 6028 | 6043 | 664 | 1113 |
| | A_{ds}G_{ds}GₖₛGₖₛAₖ | | | | | |
| 570667 | GₖₛTₖₛGₖₛ^{m}C_{ds}T_{ds}^{m}C_{ds}^{m}C_{ds}T_{ds}G_{ds}^{m}C_{ds}T_{ds} | 82.3 | 6031 | 6046 | 665 | 1114 |
| | ^{m}C_{ds}A_{ds}GₖₛAₖₛGₖ | | | | | |
| 570668 | AₖₛGₖₛGₖₛT_{ds}G_{ds}^{m}C_{ds}T_{ds}^{m}C_{ds}^{m}C_{ds}T_{ds}G_{ds}^{m} | 96.2 | 6033 | 6048 | 666 | 1115 |
| | C_{ds}T_{ds}^{m}CₖₛAₖₛGₖ | | | | | |
| 570669 | AₖₛGₖₛAₖₛG_{ds}G_{ds}T_{ds}G_{ds}^{m}C_{ds}T_{ds}^{m}C_{ds}^{m}C_{ds} | 26.2 | 6035 | 6050 | 667 | 1116 |
| | T_{ds}sG_{ds}^{m}CₖₛTₖₛ^{m}Cₖ | | | | | |
| 570670 | AₖₛGₖₛAₖₛG_{ds}A_{ds}G_{ds}G_{ds}T_{ds}G_{ds}^{m}C_{ds}T_{ds}^{m}C_{ds} | 18.2 | 6037 | 6052 | 668 | 1117 |
| | ^{m}C_{ds}TₖₛGₖₛ^{m}Cₖ | | | | | |
| 570671 | Aₖₛ^{m}Cₖₛ^{m}Cₖₛ^{m}C_{ds}^{m}C_{ds}G_{ds}^{m}C_{ds}^{m}C_{ds}^{m}C_{ds}^{m}C_{ds} | 29.2 | 6291 | 6306 | 669 | 1118 |
| | ^{m}C_{ds}G_{ds}^{m}C_{ds}Tₖₛ^{m}CₖₛAₖ | | | | | |
| 570672 | ^{m}CₖₛTₖₛAₖₛ^{m}C_{ds}^{m}C_{ds}^{m}C_{ds}^{m}C_{ds}G_{ds}^{m}C_{ds}^{m}C_{ds} | 50.3 | 6293 | 6308 | 670 | 1119 |
| | ^{m}C_{ds}^{m}C_{ds}^{m}C_{ds}Gₖₛ^{m}CₖₛTₖ | | | | | |
| 570673 | Aₖₛ^{m}Cₖₛ^{m}CₖₛT_{ds}A_{ds}^{m}C_{ds}^{m}C_{ds}^{m}C_{ds}^{m}C_{ds}G_{ds}^{m} | 26.8 | 6295 | 6310 | 671 | 1120 |
| | C_{ds}^{m}C_{ds}^{m}C_{ds}^{m}Cₖₛ^{m}CₖₛGₖ | | | | | |
| 570674 | GₖₛTₖₛAₖₛ^{m}C_{ds}^{m}C_{ds}T_{ds}A_{ds}^{m}C_{ds}^{m}C_{ds}^{m}C_{ds}^{m} | 40.8 | 6297 | 6312 | 672 | 1121 |
| | C_{ds}G_{ds}^{m}C_{ds}^{m}Cₖₛ^{m}Cₖₛ^{m}Cₖ | | | | | |
| 570675 | AₖₛGₖₛGₖₛT_{ds}A_{ds}^{m}C_{ds}^{m}C_{ds}T_{ds}A_{ds}^{m}C_{ds}^{m}C_{ds} | 56.1 | 6299 | 6314 | 673 | 1122 |
| | ^{m}C_{ds}^{m}C_{ds}Gₖₛ^{m}Cₖₛ^{m}Cₖ | | | | | |
| 570676 | GₖₛGₖₛGₖₛA_{ds}G_{ds}G_{ds}T_{ds}T_{ds}^{m}C_{ds}^{m}C_{ds}^{m}C_{ds} | 95 | 6329 | 6344 | 674 | 1123 |
| | G_{ds}^{m}C_{ds}AₖₛGₖₛ^{m}Cₖ | | | | | |
| 570677 | GₖₛTₖₛ^{m}Cₖₛ^{m}C_{ds}T_{ds}T_{ds}A_{ds}^{m}C_{ds}T_{ds}^{m}C_{ds}^{m}C_{ds} | 23 | 6360 | 6375 | 675 | 1124 |
| | A_{ds}A_{ds}^{m}CₖₛTₖₛTₖ | | | | | |
| 570678 | ^{m}CₖₛTₖₛGₖₛT_{ds}^{m}C_{ds}^{m}C_{ds}T_{ds}T_{ds}A_{ds}^{m}C_{ds}T_{ds}^{m} | 23.4 | 6362 | 6377 | 676 | 1125 |
| | C_{ds}^{m}C_{ds}AₖₛAₖₛ^{m}Cₖ | | | | | |
| 570679 | ^{m}CₖₛAₖₛ^{m}CₖₛT_{ds}G_{ds}T_{ds}^{m}C_{ds}^{m}C_{ds}T_{ds}T_{ds}A_{ds} | 7.4 | 6364 | 6379 | 677 | 1126 |
| | ^{m}C_{ds}T_{ds}^{m}Cₖₛ^{m}CₖₛAₖ | | | | | |
| 570680 | GₖₛGₖₛ^{m}CₖₛA_{ds}^{m}C_{ds}T_{ds}G_{ds}T_{ds}^{m}C_{ds}^{m}C_{ds}T_{ds} | 20.6 | 6366 | 6381 | 678 | 1127 |
| | T_{ds}A_{ds}^{m}CₖₛTₖₛ^{m}Cₖ | | | | | |
| 570681 | TₖₛAₖₛGₖₛG_{ds}^{m}C_{ds}A_{ds}^{m}C_{ds}T_{ds}G_{ds}T_{ds}^{m}C_{ds}^{m} | 29 | 6368 | 6383 | 679 | 1145 |
| | C_{ds}T_{ds}TₖₛAₖₛ^{m}Cₖ | | | | | |
| 570682 | GₖₛGₖₛTₖₛA_{ds}G_{ds}G_{ds}^{m}C_{ds}A_{ds}^{m}C_{ds}T_{ds}G_{ds}T_{ds} | 10.5 | 6370 | 6385 | 680 | 1146 |
| | ^{m}C_{ds}^{m}CₖₛTₖₛTₖ | | | | | |
| 570683 | GₖₛTₖₛ^{m}CₖₛA_{ds}^{m}C_{ds}T_{ds}G_{ds}^{m}C_{ds}T_{ds}G_{ds}G_{ds} | 23 | 6445 | 6460 | 681 | 1147 |
| | G_{ds}T_{ds}^{m}Cₖₛ^{m}CₖₛTₖ | | | | | |
| 570684 | GₖₛGₖₛTₖₛ^{m}C_{ds}A_{ds}^{m}C_{ds}T_{ds}G_{ds}^{m}C_{ds}T_{ds}G_{ds} | 22.5 | 6446 | 6461 | 433 | 1148 |
| | G_{ds}G_{ds}Tₖₛ^{m}Cₖₛ^{m}Cₖ | | | | | |
| 570685 | AₖₛGₖₛGₖₛT_{ds}^{m}C_{ds}A_{ds}^{m}C_{ds}T_{ds}G_{ds}^{m}C_{ds}T_{ds} | 10.2 | 6447 | 6462 | 682 | 1149 |
| | G_{ds}G_{ds}GₖₛTₖₛ^{m}Cₖ | | | | | |
| 570686 | ^{m}CₖₛTₖₛAₖₛG_{ds}G_{ds}T_{ds}^{m}C_{ds}A_{ds}^{m}C_{ds}T_{ds}G_{ds}^{m} | 11.1 | 6449 | 6464 | 683 | 1150 |
| | C_{ds}T_{ds}GₖₛGₖₛGₖ | | | | | |
| 570687 | GₖₛTₖₛ^{m}CₖₛT_{ds}A_{ds}G_{ds}G_{ds}T_{ds}^{m}C_{ds}A_{ds}^{m}C_{ds} | 11.7 | 6451 | 6466 | 684 | 1151 |
| | T_{ds}G_{ds}^{m}CₖₛTₖₛGₖ | | | | | |
| 570688 | AₖₛAₖₛGₖₛT_{ds}^{m}C_{ds}T_{ds}A_{ds}G_{ds}G_{ds}T_{ds}^{m}C_{ds}A_{ds} | 14.6 | 6453 | 6468 | 685 | 1152 |
| | ^{m}C_{ds}TₖₛGₖₛ^{m}Cₖ | | | | | |
| 570689 | Gₖₛ^{m}CₖₛAₖₛ^{m}C_{ds}T_{ds}^{m}C_{ds}^{m}C_{ds}A_{ds}T_{ds}T_{ds}G_{ds} | 10.1 | 6530 | 6545 | 686 | 1153 |
| | T_{ds}^{m}C_{ds}Tₖₛ^{m}CₖₛAₖ | | | | | |
| 570690 | ^{m}CₖₛTₖₛGₖₛ^{m}C_{ds}A_{ds}^{m}C_{ds}T_{ds}^{m}C_{ds}^{m}C_{ds}A_{ds}T_{ds} | 35.4 | 6532 | 6547 | 687 | 1154 |
| | T_{ds}G_{ds}Tₖₛ^{m}CₖₛTₖ | | | | | |
| 570691 | ^{m}Cₖₛ^{m}Cₖₛ^{m}CₖₛT_{ds}G_{ds}^{m}C_{ds}A_{ds}^{m}C_{ds}T_{ds}^{m}C_{ds}^{m} | 33.6 | 6534 | 6549 | 688 | 1155 |
| | C_{ds}A_{ds}T_{ds}TₖₛGₖₛTₖ | | | | | |
| 570692 | ^{m}Cₖₛ^{m}Cₖₛ^{m}Cₖₛ^{m}C_{ds}^{m}C_{ds}T_{ds}G_{ds}^{m}C_{ds}A_{ds}^{m}C_{ds} | 77.3 | 6536 | 6551 | 689 | 1156 |
| | T_{ds}^{m}C_{ds}^{m}C_{ds}AₖₛTₖₛTₖ | | | | | |
| 570693 | ^{m}CₖₛTₖₛTₖₛG_{ds}^{m}C_{ds}T_{ds}G_{ds}A_{ds}G_{ds}T_{ds}^{m}C_{ds} | 18.9 | 6559 | 6574 | 690 | 1157 |
| | A_{ds}G_{ds}GₖₛAₖₛGₖ | | | | | |
| 570694 | Tₖₛ^{m}Cₖₛ^{m}CₖₛT_{ds}T_{ds}G_{ds}^{m}C_{ds}T_{ds}G_{ds}A_{ds}G_{ds} | 30.9 | 6561 | 6576 | 691 | 1158 |
| | T_{ds}^{m}C_{ds}AₖₛGₖₛGₖ | | | | | |
| 570695 | ^{m}CₖₛTₖₛTₖₛ^{m}C_{ds}^{m}C_{ds}T_{ds}T_{ds}G_{ds}^{m}C_{ds}T_{ds}G_{ds} | 21 | 6563 | 6578 | 692 | 1159 |
| | A_{ds}G_{ds}Tₖₛ^{m}CₖₛAₖ | | | | | |
| 570696 | Aₖₛ^{m}Cₖₛ^{m}CₖₛT_{ds}T_{ds}^{m}C_{ds}^{m}C_{ds}T_{ds}T_{ds}G_{ds}^{m}C_{ds} | 50.3 | 6565 | 6580 | 693 | 1160 |
| | T_{ds}G_{ds}AₖₛGₖₛTₖ | | | | | |
| 570697 | GₖₛGₖₛAₖₛ^{m}C_{ds}^{m}C_{ds}T_{ds}T_{ds}^{m}C_{ds}^{m}C_{ds}T_{ds}T_{ds} | 28.3 | 6567 | 6582 | 694 | 1161 |
| | G_{ds}^{m}C_{ds}TₖₛGₖₛAₖ | | | | | |
| 570698 | ^{m}CₖₛAₖₛGₖₛG_{ds}A_{ds}^{m}C_{ds}^{m}C_{ds}T_{ds}Ta_{ds}^{m}C_{ds}^{m}C_{ds} | 47.6 | 6569 | 6584 | 695 | 1162 |
| | T_{ds}T_{ds}Gₖₛ^{m}CₖₛTₖ | | | | | |
| 570699 | AₖₛGₖₛ^{m}Cₖₛ^{m}C_{ds}^{m}C_{ds}T_{ds}^{m}C_{ds}^{m}C_{ds}A_{ds}G_{ds} | 17.9 | 6576 | 6591 | 696 | 1163 |
| | G_{ds}A_{ds}^{m}C_{ds}^{m}CₖₛTₖₛTₖ | | | | | |
| 570700 | TₖₛAₖₛGₖₛ^{m}C_{ds}T_{ds}^{m}C_{ds}^{m}C_{ds}^{m}C_{ds}^{m}C_{ds}A_{ds}^{m} | 24.1 | 6594 | 6609 | 697 | 1164 |
| | C_{ds}T_{ds}^{m}C_{ds}^{m}CₖₛAₖₛGₖ | | | | | |
| 570701 | GₖₛAₖₛTₖₛA_{ds}G_{ds}^{m}C_{ds}T_{ds}^{m}C_{ds}m^{m}C_{ds}^{m}C_{ds}^{m} | 12.9 | 6596 | 6611 | 698 | 1165 |
| | C_{ds}A_{ds}^{m}C_{ds}Tₖₛ^{m}Cₖₛ^{m}Cₖ | | | | | |
| 570702 | ^{m}CₖₛAₖₛGₖₛA_{ds}T_{ds}A_{ds}G_{ds}^{m}C_{ds}T_{ds}^{m}C_{ds}^{m}C_{ds} | 24 | 6598 | 6613 | 699 | 1166 |
| | ^{m}C_{ds}^{m}C_{ds}Aₖₛ^{m}CₖₛTₖ | | | | | |
| 570703 | CₖₛTₖₛ^{m}CₖₛA_{ds}G_{ds}A_{ds}T_{ds}A_{ds}G_{ds}^{m}C_{ds}T_{ds}^{m} | 22.3 | 6600 | 6615 | 700 | 1167 |
| | C_{ds}^{m}C_{ds}^{m}Cₖₛ^{m}CₖₛAₖ | | | | | |
| 570704 | AₖₛGₖₛ^{m}CₖₛT_{ds}^{m}C_{ds}A_{ds}G_{ds}A_{ds}T_{ds}A_{ds}G_{ds}^{m} | 31.8 | 6602 | 6617 | 701 | 1168 |
| | C_{ds}T_{ds}^{m}Cₖₛ^{m}Cₖₛ^{m}Cₖ | | | | | |
| 570705 | Tₖₛ^{m}CₖₛAₖₛG_{ds}^{m}C_{ds}T_{ds}^{m}C_{ds}A_{ds}G_{ds}A_{ds}T_{ds} | 33.9 | 6604 | 6619 | 702 | 1169 |
| | A_{ds}G_{ds}^{m}CₖₛTₖₛ^{m}Cₖ | | | | | |
| 570706 | Tₖₛ^{m}CₖₛTₖₛ^{m}C_{ds}A_{ds}G_{ds}^{m}C_{ds}T_{ds}^{m}C_{ds}A_{ds}G_{ds} | 28.1 | 6606 | 6621 | 703 | 1170 |
| | A_{ds}T_{ds}AₖₛGₖₛ^{m}Cₖ | | | | | |
| 570707 | GₖₛAₖₛGₖₛT_{ds}^{m}C_{ds}^{m}C_{ds}T_{ds}^{m}C_{ds}T_{ds}^{m}C_{ds}^{m}C_{ds} | 37.2 | 6636 | 6651 | 704 | 1171 |
| | T_{ds}G_{ds}^{m}CₖₛTₖₛTₖ | | | | | |
| 570708 | GₖₛGₖₛAₖₛG_{ds}G_{ds}A_{ds}G_{ds}T_{ds}^{m}C_{ds}^{m}C_{ds}T_{ds}^{m} | 66.3 | 6640 | 6655 | 705 | 1172 |
| | C_{ds}T_{ds}^{m}Cₖₛ^{m}CₖₛTₖ | | | | | |
| 570709 | GₖₛAₖₛGₖₛG_{ds}A_{ds}G_{ds}G_{ds}A_{ds}G_{ds}T_{ds}^{m}C_{ds}^{m} | 52.7 | 6642 | 6657 | 706 | 1173 |
| | C_{ds}T_{ds}^{m}CₖₛTₖₛ^{m}Cₖ | | | | | |
| 570710 | ^{m}CₖₛAₖₛAₖₛA_{ds}A_{ds}G_{ds}G_{ds}G_{ds}^{m}C_{ds}A_{ds}^{m}C_{ds} | 31.8 | 6713 | 6728 | 707 | 1174 |
| | ^{m}C_{ds}^{m}C_{ds}AₖₛGₖₛAₖ | | | | | |
| 570711 | AₖₛGₖₛ^{m}CₖₛA_{ds}A_{ds}A_{ds}A_{ds}G_{ds}G_{ds}G_{ds}^{m}C_{ds} | 12.3 | 6715 | 6730 | 708 | 1175 |
| | A_{ds}^{m}C_{ds}^{m}Cₖₛ^{m}CₖₛAₖ | | | | | |
| 570712 | GₖₛGₖₛAₖₛT_{ds}^{m}C_{ds}^{m}C_{ds}^{m}C_{ds}^{m}C_{ds}A_{ds}G_{ds}T_{ds} | 37.1 | 6733 | 6748 | 709 | 1176 |
| | A_{ds}T_{ds}TₖₛGₖₛTₖ | | | | | |
| 570713 | ^{m}CₖₛTₖₛGₖₛG_{ds}A_{ds}T_{ds}^{m}C_{ds}^{m}C_{ds}^{m}C_{ds}^{m}C_{ds}A_{ds} | 42.4 | 6735 | 6750 | 710 | 1177 |
| | G_{ds}T_{ds}AₖₛTₖₛTₖ | | | | | |
| 570714 | TₖₛGₖₛ^{m}CₖₛT_{ds}G_{ds}G_{ds}A_{ds}T_{ds}^{m}C_{ds}^{m}C_{ds}^{m}C_{ds} | 31.4 | 6737 | 6752 | 711 | 1178 |
| | ^{m}C_{ds}A_{ds}GₖₛTₖₛAₖ | | | | | |
| 570715 | AₖₛTₖₛTₖₛ^{m}C_{ds}T_{ds}^{m}C_{ds}T_{ds}A_{ds}G_{ds}A_{ds}^{m}C_{ds} | 12.1 | 6789 | 6804 | 712 | 1179 |
| | T_{ds}G_{ds}^{m}CₖₛAₖₛAₖ | | | | | |
| 570716 | TₖₛAₖₛAₖₛT_{ds}T_{ds}^{m}C_{ds}T_{ds}^{m}C_{ds}T_{ds}A_{ds}G_{ds}A_{ds} | 9 | 6791 | 6806 | 713 | 1180 |
| | ^{m}C_{ds}TₖₛGₖₛ^{m}Cₖ | | | | | |
| 570717 | Tₖₛ^{m}CₖₛTₖₛA_{ds}A_{ds}T_{ds}T_{ds}^{m}C_{ds}T_{ds}^{m}C_{ds}T_{ds}A_{ds} | 32.1 | 6793 | 6808 | 714 | 1181 |
| | G_{ds}Aₖₛ^{m}CₖₛTₖ | | | | | |
| 570718 | Tₖₛ^{m}CₖₛTₖₛ^{m}C_{ds}T_{ds}A_{ds}A_{ds}T_{ds}T_{ds}^{m}C_{ds}T_{ds}^{m} | 71.4 | 6795 | 6810 | 715 | 1182 |
| | C_{ds}T_{ds}AₖₛGₖₛAₖ | | | | | |
| 570719 | ^{m}CₖₛTₖₛ^{m}Cₖₛ^{m}C_{ds}A_{ds}T_{ds}A_{ds}A_{ds}T_{ds}T_{ds}^{m}C_{ds} | 36.9 | 6804 | 6819 | 716 | 1183 |
| | T_{ds}^{m}C_{ds}TₖₛAₖₛAₖ | | | | | |
| 570720 | Aₖₛ^{m}CₖₛTₖₛ^{m}C_{ds}T_{ds}^{m}C_{ds}^{m}C_{ds}A_{ds}T_{ds}A_{ds}A_{ds} | 17.1 | 6807 | 6822 | 717 | 1184 |
| | T_{ds}T_{ds}^{m}CₖₛTₖₛ^{m}Cₖ | | | | | |
| 570721 | Aₖₛ^{m}CₖₛAₖₛ^{m}C_{ds}T_{ds}^{m}C_{ds}T_{ds}^{m}C_{ds}^{m}C_{ds}A_{ds}T_{ds} | 23.7 | 6809 | 6824 | 718 | 1185 |
| | A_{ds}A_{ds}TₖₛTₖₛ^{m}Cₖ | | | | | |
| 570722 | ^{m}Cₖₛ^{m}CₖₛAₖₛ^{m}C_{ds}A_{ds}^{m}C_{ds}T_{ds}^{m}C_{ds}T_{ds}^{m}C_{ds}^{m} | 34.4 | 6811 | 6826 | 719 | 1186 |
| | C_{ds}A_{ds}T_{ds}AₖₛAₖₛTₖ | | | | | |
| 570723 | TₖₛGₖₛ^{m}Cₖₛ^{m}C_{ds}A_{ds}^{m}C_{ds}A_{ds}^{m}C_{ds}T_{ds}^{m}C_{ds}T_{ds} | 38.7 | 6813 | 6828 | 720 | 1187 |
| | ^{m}C_{ds}^{m}C_{ds}AₖₛTₖₛAk | | | | | |

### Example 10: Dose Response Studies with Antisense Oligonucleotides Targeting Human Dystrophia Myotonica-Protein Kinase (DMPK) in HepG2 Cells

Antisense oligonucleotides targeted to a human DMPK nucleic acid were tested for their effect on human DMPK RNA transcript in vitro. Cultured HepG2 cells at a density of 20,000 cells per well were transfected using electroporation with 61.7 nM, 185.2 nM, 555.6 nM, 1666.7 nM, 5000.0 nM, and 15000.0 nM concentrations of each antisense oligonucleotide. After approximately 24 hours, RNA was isolated from the cells and DMPK RNA transcript levels were measured by quantitative real-time PCR using primer probe set RTS3164 (forward sequence AGCCTGAGCCGGGAGATG, designated herein as SEQ ID NO: 20; reverse sequence GCGTAGTTGACTGGCGAAGTT, designated herein as SEQ ID NO: 21; probe sequence AGGCCATCCGCACGGACAACCX, designated herein as SEQ ID NO: 22). Human DMPK RNA transcript levels were adjusted according to total RNA content, as measured by RIBOGREEN®. Results are presented as percent expression of human DMPK, relative to untreated control (UTC) cells. For example, if the UTC is 100 and a dose of 5000 nM of ISIS No. 445569 yields a % Expression of human DMPK of 35 then the 5000 nM dose of ISIS reduced expression of human DMPK by 65% relative to the UTC. The half maximal inhibitory concentration (IC₅₀) of each oligonucleotide is presented in the table below and was calculated by plotting the concentrations of oligonucleotides used versus the percent inhibition of human DMPK mRNA expression achieved at each concentration, and noting the concentration of oligonucleotide at which 50% inhibition of human DMPK mlRNA expression was achieved compared to the control. The results are presented in Table 19.

The tested antisense oligonucleotide sequences demonstrated dose-dependent inhibition of human DMPK mRNA levels under the conditions specified above.

TABLE 19
Dose response studies for with antisense oligonucleotides
targeting hDMPK in HepG2 Cells

| ISIS | Dose | % Expression | |
|---|---|---|---|
| No. | (nM) | of human DMPK | IC₅₀ |
| UTC | ND | 100 | ND |
| 445569 | 61.7 | 115.3 | 2.3 |
| | 185.2 | 87.9 | |
| | 555.6 | 69.0 | |
| | 1666.7 | 57.2 | |
| | 5000.0 | 35.0 | |
| | 15000.0 | 22.6 | |
| 512497 | 61.7 | 108.6 | 2 |
| | 185.2 | 98.4 | |
| | 555.6 | 77.9 | |
| | 1666.7 | 57.2 | |
| | 5000.0 | 28.0 | |
| | 15000.0 | 12.8 | |
| 486178 | 61.7 | 88.2 | 0.7 |
| | 185.2 | 67.1 | |
| | 555.6 | 49.4 | |
| | 1666.7 | 32.8 | |
| | 5000.0 | 26.7 | |
| | 15000.0 | 11.8 | |
| 569473 | 61.7 | 107.9 | 0.6 |
| | 185.2 | 66.5 | |
| | 555.6 | 33.6 | |
| | 1666.7 | 23.5 | |
| | 5000.0 | 12.8 | |
| | 15000.0 | 9.2 | |
| 570808 | 61.7 | 77.2 | 0.2 |
| | 185.2 | 52.7 | |
| | 555.6 | 20.6 | |
| | 1666.7 | 8.1 | |
| | 5000.0 | 7.2 | |
| | 15000.0 | 5.4 | |
| 594292 | 61.7 | 96.2 | 5.5 |
| | 185.2 | 99.6 | |
| | 555.6 | 80.0 | |
| | 1666.7 | 59.0 | |
| | 5000.0 | 45.5 | |
| | 15000.0 | 42.8 | |
| 594300 | 61.7 | 101.7 | >15 |
| | 185.2 | 104.3 | |
| | 555.6 | 101.6 | |
| | 1666.7 | 93.6 | |
| | 5000.0 | 74.9 | |
| | 15000.0 | 66.8 | |
| 598768 | 61.7 | 95.5 | 1.2 |
| | 185.2 | 83.6 | |
| | 555.6 | 70.6 | |
| | 1666.7 | 40.7 | |
| | 5000.0 | 22.2 | |
| | 15000.0 | 7.3 | |
| 598769 | 61.7 | 103.9 | 1.9 |
| | 185.2 | 105.3 | |
| | 555.6 | 76.1 | |
| | 1666.7 | 50.4 | |
| | 5000.0 | 29.8 | |
| | 15000.0 | 12.1 | |
| 598777 | 61.7 | 96.4 | 0.9 |
| | 185.2 | 69.4 | |
| | 555.6 | 41.8 | |
| | 1666.7 | 42.8 | |
| | 5000.0 | 16.4 | |
| | 15000.0 | 27.1 | |

### Example 11: Dose Response Studies with Antisense Oligonucleotides Targeting Human Dystrophia Myotonica-Protein Kinase (hDMPK) in Steinert DM1 Myoblast Cells

Antisense oligonucleotides targeted to a human DMPK nucleic acid were tested for their effect on human DMPK RNA transcript in vitro. Cultured Steinert DM1 myoblast cells at a density of 20,000 cells per well were transfected using electroporation with 61.7 nM, 185.2 nM, 555.6 nM, 1666.7 nM, 5000.0 nM, and 15000.0 nM concentrations of each antisense oligonucleotide. After approximately 24 hours, RNA was isolated from the cells and DMPK RNA transcript levels were measured by quantitative real-time PCR using primer probe set RTS3164 described above. Human DMPK RNA transcript levels were adjusted according to total RNA content, as measured by RIBOGREEN®. Results are presented as percent (%) expression of human DMPK, relative to untreated control (UTC) cells. The half maximal inhibitory concentration (IC₅₀) of each oligonucleotide is presented in the table below and was calculated by plotting the concentrations of oligonucleotides used versus the percent inhibition of human DMPK mRNA expression achieved at each concentration, and noting the concentration of oligonucleotide at which 50% inhibition of human DMPK mRNA expression was achieved compared to the control. The results are presented in Table 20.

The tested antisense oligonucleotide sequences demonstrated dose-dependent inhibition of human DMPK mRNA levels under the conditions specified above.

TABLE 20
Dose response studies for with antisense oligonucleotides
targeting hDMPK in Steinert DM1 Cells

| ISIS | Dose | % Expression | |
|---|---|---|---|
| No. | (nM) | of human DMPK | IC₅₀ |
| UTC | ND | 100 | ND |
| 445569 | 61.7 | 58.3 | 0.4 |
| | 185.2 | 56.7 | |
| | 555.6 | 58.5 | |
| | 1666.7 | 40.9 | |
| | 5000.0 | 26.0 | |
| | 15000.0 | 23.5 | |
| 512497 | 61.7 | 78.1 | 5.1 |
| | 185.2 | 77.5 | |
| | 555.6 | 98.8 | |
| | 1666.7 | 71.2 | |
| | 5000.0 | 51.3 | |
| | 15000.0 | 22.8 | |
| 486178 | 61.7 | 78.0 | 0.5 |
| | 185.2 | 61.3 | |
| | 555.6 | 43.3 | |
| | 1666.7 | 27.4 | |
| | 5000.0 | 24.6 | |
| | 15000.0 | 16.9 | |
| 569473 | 61.7 | 83.3 | 0.6 |
| | 185.2 | 54.8 | |
| | 555.6 | 64.5 | |
| | 1666.7 | 26.1 | |
| | 5000.0 | 19.4 | |
| | 15000.0 | 15.4 | |
| 570808 | 61.7 | 103.6 | 0.9 |
| | 185.2 | 77.8 | |
| | 555.6 | 46.7 | |
| | 1666.7 | 25.2 | |
| | 5000.0 | 20.8 | |
| | 15000.0 | 19.3 | |
| 594292 | 61.7 | 100.1 | 5.6 |
| | 185.2 | 109.7 | |
| | 555.6 | 72.6 | |
| | 1666.7 | 66.2 | |
| | 5000.0 | 39.5 | |
| | 15000.0 | 45.7 | |
| 594300 | 61.7 | 96.2 | 5.6 |
| | 185.2 | 87.1 | |
| | 555.6 | 70.3 | |
| | 1666.7 | 66.4 | |
| | 5000.0 | 58.1 | |
| | 15000.0 | 33.2 | |
| 598768 | 61.7 | 77.0 | 0.7 |
| | 185.2 | 62.9 | |
| | 555.6 | 62.0 | |
| | 1666.7 | 35.6 | |
| | 5000.0 | 24.5 | |
| | 15000.0 | 21.0 | |
| 598769 | 61.7 | 70.3 | 0.4 |
| | 185.2 | 49.2 | |
| | 555.6 | 55.3 | |
| | 1666.7 | 33.2 | |
| | 5000.0 | 27.1 | |
| | 15000.0 | 13.4 | |
| 598777 | 61.7 | 87.7 | 1 |
| | 185.2 | 61.7 | |
| | 555.6 | 57.3 | |
| | 1666.7 | 37.9 | |
| | 5000.0 | 30.0 | |
| | 15000.0 | 29.7 | |

### Example 12: Dose Response Studies with Antisense Oligonucleotides Targeting Rhesus Monkey Dystrophia Myotonica-Protein Kinase (DMPK) in Cynomolgus Monkey Primary Hepatocytes

Antisense oligonucleotides targeted to a rhesus monkey DMPK nucleic acid were tested for their effect on rhesus monkey DMPK RNA transcript in vitro. Cultured cynomolgus monkey primary hepatocytes cells at a density of 20,000 cells per well were transfected using electroporation with 61.7 nM, 185.2 nM, 555.6 nM, 1666.7 nM, 5000.0 nM, and 15000.0 nM concentrations of each antisense oligonucleotide. After approximately 24 hours, RNA was isolated from the cells and DMPK RNA transcript levels were measured by quantitative real-time PCR using primer probe set RTS3164 described above. Rhesus monkey DMPK RNA transcript levels were adjusted according to total RNA content, as measured by RIBOGREEN®. Results are presented as percent (0) expression of rhesus monkey DMPK, relative to untreated control (UTC) cells. The half maximal inhibitory concentration (IC₅₀) of each oligonucleotide is presented in the table below and was calculated by plotting the concentrations of oligonucleotides used versus the percent inhibition of rhesus monkey DMPK mRNA expression achieved at each concentration, and noting the concentration of oligonucleotide at which 50% inhibition of rhesus monkey DMPK mRNA expression was achieved compared to the control.

The tested antisense oligonucleotide sequences demonstrated dose-dependent inhibition of rhesus monkey DMPK mRNA levels under the conditions specified above.

TABLE 21
Dose response studies for with antisense oligonucleotides targeting
rhesus monkey DMPK in cynomolgus monkey primary hepatocytes

| ISIS | Dose | % Expression | |
|---|---|---|---|
| No. | (nM) | of human DMPK | IC₅₀ |
| UTC | ND | 100 | ND |
| 445569 | 61.7 | 79.7 | 1.4 |
| | 185.2 | 41.1 | |
| | 555.6 | 58.1 | |
| | 1666.7 | 33.5 | |
| | 5000.0 | 46.9 | |
| | 15000.0 | 50.0 | |
| 512497 | 61.7 | 123.4 | 1.5 |
| | 185.2 | 63.7 | |
| | 555.6 | 44.8 | |
| | 1666.7 | 34.1 | |
| | 5000.0 | 51.2 | |
| | 15000.0 | 23.5 | |
| 486178 | 61.7 | 51.1 | <.06 |
| | 185.2 | 30.6 | |
| | 555.6 | 22.0 | |
| | 1666.7 | 23.5 | |
| | 5000.0 | 9.8 | |
| | 15000.0 | 19.2 | |
| 569473 | 61.7 | 82.1 | .2 |
| | 185.2 | 39.4 | |
| | 555.6 | 17.7 | |
| | 1666.7 | 28.5 | |
| | 5000.0 | 20.0 | |
| | 15000.0 | 15.6 | |
| 570808 | 61.7 | 74.6 | 0.1 |
| | 185.2 | 27.6 | |
| | 555.6 | 16.4 | |
| | 1666.7 | 25.6 | |
| | 5000.0 | 8.8 | |
| | 15000.0 | 21.9 | |
| 594292 | 61.7 | 93.0 | >15 |
| | 185.2 | 82.1 | |
| | 555.6 | 106.0 | |
| | 1666.7 | 91.1 | |
| | 5000.0 | 62.2 | |
| | 15000.0 | 70.4 | |
| 594300 | 61.7 | 105.5 | >15 |
| | 185.2 | 91.8 | |
| | 555.6 | 114.9 | |
| | 1666.7 | 65.7 | |
| | 5000.0 | 110.2 | |
| | 15000.0 | 118.8 | |
| 598768 | 61.7 | 70.3 | 0.4 |
| | 185.2 | 57.8 | |
| | 555.6 | 58.5 | |
| | 1666.7 | 16.5 | |
| | 5000.0 | 24.0 | |
| | 15000.0 | 13.4 | |
| 598769 | 61.7 | 76.5 | 1.1 |
| | 185.2 | 65.1 | |
| | 555.6 | 64.0 | |
| | 1666.7 | 34.4 | |
| | 5000.0 | 60.9 | |
| | 15000.0 | 8.6 | |
| 598777 | 61.7 | 161.4 | 2.1 |
| | 185.2 | 51.7 | |
| | 555.6 | 47.5 | |
| | 1666.7 | 34.6 | |
| | 5000.0 | 27.8 | |
| | 15000.0 | 52.9 | |

### Example 13: In Vivo Antisense Inhibition of hDMPK in DMSXL Transgenic Mice

To test the effect of antisense inhibition for the treatment of myotonic dystrophy type 1 (DM1), an appropriate mouse model was required. The transgenic mouse model, DMSXL carrying the hDMPK gene with large expansions of over 1000 CTG repeats was generated (Huguet et al., PLOS Genetics, 2012, 8(11), e1003034-e1003043). These DMSXL mice express the mutant hDMPK allele and display muscle weakness phenotype similar to that seen in DM1 patients.

ISIS 486178 from Table 1 was selected and tested for antisense inhibition of hDMPK transcript in vivo. ISIS 445569 was included in the study for comparison.

### Treatment

DMSXL mice were maintained on a 12-hour light/dark cycle and fed ad libitum normal Purina mouse chow. Animals were acclimated for at least 7 days in the research facility before initiation of the experiment. Antisense oligonucleotides (ASOs) were prepared in PBS and sterilized by filtering through a 0.2 micron filter. ASOs were dissolved in 0.9% PBS for injection.

DMSXL mice received subcutaneous injections of ISIS 445569 at 50 mg/kg or ISIS 486178 at 25 mg/kg twice per week for 4 weeks. The control group received subcutaneous injections of PBS twice weekly for 4 weeks. Each treatment group consisted of 4 animals.

Inhibition of hDMPK mRNA Levels

Twenty four hours after the final dose, the mice were sacrificed and tissues were collected. mRNA was isolated for real-time PCR analysis of hDMPK and normalized to 18s RNA. Human primer probe set RTS3164 was used to measure mRNA levels. The results are expressed as the average percent of hDMPK mRNA levels for each treatment group, relative to PBS control.

Human primer probe set RTS3164 (forward sequence AGCCTGAGCCGGGAGATG, designated herein as SEQ ID NO: 20; reverse sequence GCGTAGTTGACTGGCGAAGTT, designated herein as SEQ ID NO: 21; probe sequence AGGCCATCCGCACGGACAACCX, designated herein as SEQ ID NO: 22).

As presented in Table 22 below, treatment with antisense oligonucleotides reduced hDMPK transcript expression. The results indicate that treatment with ISIS 445569 and 486178 resulted in reduction of hDMPK mRNA levels in DMSXL mice.

TABLE 22
Effect of antisense oligonucleotides
on hDMPK inhibition in DMSXL mice

| | | | hDMPK | |
|---|---|---|---|---|
| ISIS | Dosage | Tissue | mRNA levels | |
| No. | (mg/kg) | Type | (% PBS) | Motif/Length |
| PBS | 0 | | | |
| 486178 | 25 | Tibialis | 70.7 | kkk-d10-kkk |
| | | Anterior | | (16 mer) |
| | | Soleus | 67.3 | |
| | | Quadriceps | 73.9 | |
| | | Latissiumus | 71.0 | |
| | | grand dorsi | | |
| | | Triceps | 67.1 | |
| | | Diaphragm | 68.9 | |
| | | Heart | 30.8 | |
| | | Brain | 11.8 | |
| 445569 | 50 | Tibialis | 38.4 | e5-d10-e5 |
| | | Anterior | | (20 mer) |
| | | Soleus | 47.5 | |
| | | Quadriceps | 41.3 | |
| | | Latissiumus | 35.7 | |
| | | grand dorsi | | |
| | | Triceps | 30.5 | |
| | | Diaphragm | 44.7 | |
| | | Heart | 7.6 | |
| | | Brain | 13.1 | |

### Example 14: Effect of ASO Treatment on Muscle Strength in DMSXL Mice Targeting hDMPK

### Griptest

Mice were assessed for grip strength performance in wild-type (WT) and DMSXL forelimb using a commercial grip strength dynamometer as described in the literature ((Huguet et al., PLOS Genetics, 2012, 8(11), e1003034-e1003043).

DMSXL mice received subcutaneous injections of ISIS 486178 at 25 mg/kg or ISIS 445569 at 50 mg/kg twice per week for 4 weeks. The control DMSXL group received subcutaneous injections of PBS twice weekly for 4 weeks. Each treatment group consisted of 4 animals. The forelimb force for each treatment group and WT was measured at day 0, 30, and 60 using the griptest. The grip strength performance was determined by measuring the force difference between day 60 and day 0. Results are presented as the average forelimb force from each group.

As illustrated in Table 23, below, treatment with ASOs targeting hDMPK improved muscle strength in DMSXL mice compared to untreated control. ISIS 486178, an ASO with cEt modifications, demonstrated substantial improvement in the forelimb strength (+3.4) compared to ISIS 445569 with MOE modifications (+0.38).

TABLE 23
Effect of ASO treatment on muscle strength
in DMSXL mice targeting hDMPK

| | Forelimb force (g) | | | |
|---|---|---|---|---|
| Treatment group | Day 0 | Day 30 | Day 60 | Δ = Day 60 − Day 0 |
| Untreated control | 72.2 | 70.2 | 67.5 | −4.6 |
| ASO 486178 | 62.3 | 65.7 | 65.6 | +3.4 |
| ASO 445569 | 64.3 | 68 | 64.7 | +0.38 |
| Wild type (WT) | 75.2 | 76.5 | 78.4 | +3.2 |

### Example 15: Effect of ASO Treatment on Muscle Fiber Distribution in DMSXL Mice Targeting hDMPK

The muscle fiber distribution in DMSXL mice targeting hDMPK in the presence and absence of ISIS 445569 and 486178 was also assessed. Both ASOs were previously described in Table 1, above.

DMSXL mice received subcutaneous injections of ISIS 486178 at 25 mg/kg or ISIS 445569 at 50 mg/kg twice per week for 4 weeks. The control DMSXL group received subcutaneous injections of PBS twice weekly for 4 weeks. Each treatment group consisted of 4 animals. The muscle fiber distribution was assessed and the results are presented Table 44, below.

As illustrated, treatment with ASOs targeting hDMPK decreased the distribution of DM1 Associated Type 2c muscle fiber in the tibialis anterior (TA) of DMSXL mice compared to untreated control. The results demonstrated that normal pattern of fiber distribution in the skeletal muscles can be restored with ASO treatment. ISIS 445569 demonstrated an improvement in the muscle fiber distribution as compared to the untreated control; however ISIS 486178, an ASO with cEt modifications, demonstrated muscle fiber distribution that was more consistent with the muscle fiber distribution found in the wild-type mice.

TABLE 24
Effect of ASO treatment on muscle fiber distribution
in DMSXL mice targeting hDMPK

| | Fiber Type Distribution in TA muscle | | |
|---|---|---|---|
| Treatment group | Fiber 1 | Fiber 2a | Fiber 2c |
| Untreated control | 4% | 25% | 5.90% |
| ASO 486178 | 3.10% | 15% | 0.70% |
| ASO 445569 | 4% | 21% | 2% |
| Wild type (WT) | 3.30% | 15% | 0.00% |

### Example 16: Dose-Dependent Antisense Inhibition of hDMPK in DMSXL Transgenic Mice

The newly designed ASOs from Table 1, above, were further evaluated in a dose-response study for antisense inhibition of hDMPK transcript in vivo. ISIS 445569 was included in the study for comparison.

### Treatment

DMSXL mice were maintained on a 12-hour light/dark cycle and fed ad libitum normal Purina mouse chow. Animals were acclimated for at least 7 days in the research facility before initiation of the experiment. Antisense oligonucleotides (ASOs) were prepared in PBS and sterilized by filtering through a 0.2 micron filter. ASOs were dissolved in 0.9% PBS for injection.

DMSXL mice received subcutaneous injections of PBS or ASOs from Table 1, above, targeting hDMPK. The ASO was dosed twice per week for 4 weeks at the indicated doses in Table 25, below. The control group received subcutaneous injections of PBS twice weekly for 4 weeks. Each treatment group consisted of 4 animals.

Inhibition of hDMPK mRNA Levels

Forty eight hours after the final dose, the mice were sacrificed and tissue from the tibialis anterior muscles, quadriceps muscles (left), gastrocnemius muscles, heart and diaphragm was isolated. mRNA was isolated for real-time PCR analysis of hDMPK and normalized to RIBOGREEN®. Human primer probe set RTS3164 was used to measure mRNA levels. The results summarized in Table 25, below, were independently generated from various dose-response studies. The results are presented as the average percent of hDMPK mRNA expression levels for each treatment group, relative to PBS control.

As presented, treatment with antisense oligonucleotides reduced hDMPK transcript expression in a dose-dependent manner.

TABLE 25
Dose-dependent inhibition of hDMPK mRNA levels in DMSXL mice

| | | hDMPK mRNA levels (% PBS) | | | | |
|---|---|---|---|---|---|---|
| ISIS No. | mg/kg/wk | TA | Quad (Left) | Gastroc | Heart | Diaphragm |
| PBS | 0 | 100 | 100 | 100 | 100 | 100 |
| 445569 | 50 | 54.7 | 80.3 | 97.1 | 55.4 | 21.7 |
| | 100 | 28.3 | 42.1 | 71.3 | 48.9 | 19.7 |
| | 200 | 22.2 | 33.9 | 45.2 | 34.2 | 10.0 |
| 512497 | 50 | 23.8 | 48.9 | 52.9 | 44.4 | 35.0 |
| | 100 | 9.7 | 28.7 | 24.8 | 43.8 | 24.2 |
| | 200 | 11.4 | 22.4 | 16.4 | 42.0 | 15.2 |
| 486178 | 25 | 59.1 | 56.1 | 63.1 | 75.3 | 39.1 |
| | 50 | 33.8 | 61.9 | 58.7 | 59.2 | 32.5 |
| | 100 | 36.6 | 65.8 | 51.6 | 47.3 | 26.2 |
| 570808 | 25 | 26.3 | 41.1 | 39.8 | 44.9 | 17.3 |
| | 50 | 12.2 | 13.0 | 36.3 | 18.4 | 8.1 |
| | 100 | 6.1 | 5.4 | 7.9 | 10.2 | 3.0 |
| 594292 | 25 | 48.8 | 32.2 | 68.8 | 70.6 | 72.7 |
| | 50 | 32.0 | 30.4 | 41.1 | 85.1 | 48.3 |
| | 100 | 31.6 | 39.6 | 53.3 | 63.9 | 40.2 |
| 598768 | 25 | 16.9 | 27.1 | 27.5 | 56.3 | 26.9 |
| | 50 | 10.2 | 33.6 | 24.1 | 30.8 | 20.2 |
| | 100 | 6.8 | 22.0 | 25.5 | 22.6 | 13.1 |
| 598769 | 25 | 21.6 | 50.8 | 48.1 | 61.0 | 30.3 |
| | 50 | 12.7 | 25.1 | 42.3 | 36.4 | 16.7 |
| | 100 | 12.8 | 18.4 | 33.2 | 32.0 | 20.2 |
| 569473 | 25 | 42.0 | 21.8 | 48.9 | 51.8 | 34.8 |
| | 50 | 41.6 | 16.2 | 47.6 | 55.6 | 23.6 |
| | 100 | 31.9 | 19.2 | 31.9 | 35.6 | 20.5 |
| 594300 | 25 | 114.5 | 56.7 | 96.2 | 91.0 | 62.6 |
| | 50 | 44.3 | 22.3 | 52.8 | 69.3 | 54.7 |
| | 100 | 73.0 | 22.6 | 56.6 | 78.3 | 44.5 |
| 598777 | 25 | 49.4 | 28.8 | 76.1 | 97.1 | 58.7 |
| | 50 | 44.8 | 13.6 | 36.5 | 87.4 | 40.8 |
| | 100 | 31.8 | 10.1 | 22.5 | 86.8 | 33.6 |

TA = Tibialis Anterior;
Quad = Quadriceps;
Gastroc = Gastrocnemius

### Example 17: Six Week In Vivo Tolerability Study in CD-1 Mice

The newly designed ASOs from Table 1, above, were further evaluated in a 6 week study to assess plasma chemistry, body/organ weights and histology. Groups of CD-1 mice were administered 100 mg/kg/wk of ISIS 445569 or ISIS 512497. Further groups of CD-1 mice were administered 50 mg/kg/wk of ISIS 486178, ISIS 570808, ISIS 594292, ISIS 598768, ISIS 598769, ISIS 569473, ISIS 594300, and ISIS 598777. After six weeks and two days after each group of mice received the last dose, the mice were sacrificed and tissues were collected for analysis. For each group of mice, analysis to measure alanine transaminase levels, aspartate aminotransferase levels, blood urea nitrogen (BUN) levels, albumin levels, total bilirubin, and creatine levels was measured. Additionally, organ weights were also measured, the results of which are presented in the tables below.

TABLE 26
Plasma Chemistry in CD-1 mice

| | | | BUN | Albumin | T. Bil | Creatinine |
|---|---|---|---|---|---|---|
| ISIS No. | ALT (U/L) | AST (U/L) | (mg/dL) | (g/dL) | (mg/dL) | (mg/dL) |
| PBS | 31.75 | 60.75 | 32.73 | 2.99 | 0.23 | 0.16 |
| 486178 | 65.00 | 103.00 | 27.18 | 2.90 | 0.19 | 0.13 |
| 445569 | 162.75 | 195.25 | 29.70 | 3.38 | 0.26 | 0.14 |
| 570808 | 313.50 | 332.50 | 32.40 | 2.81 | 0.28 | 0.15 |
| 594292 | 58.75 | 133.00 | 28.15 | 2.94 | 0.21 | 0.13 |
| 598768 | 45.50 | 92.00 | 26.85 | 2.90 | 0.21 | 0.11 |
| 598769 | 69.25 | 94.25 | 32.73 | 2.89 | 0.18 | 0.13 |
| 512497 | 101.25 | 144.50 | 26.90 | 2.90 | 0.19 | 0.12 |
| 569473 | 75.75 | 137.00 | 28.98 | 3.05 | 0.26 | 0.13 |
| 594300 | 46.00 | 76.75 | 24.70 | 2.94 | 0.18 | 0.11 |
| 598777 | 186.50 | 224.25 | 24.68 | 2.97 | 0.30 | 0.11 |

TABLE 27
Body & Organ Weights in CD-1 mice

| ISIS No. | *Kidney % BW | *Liver % BW | *Spleen % BW |
|---|---|---|---|
| PBS | 1.00 | 1.00 | 1.00 |
| 486178 | 1.05 | 1.05 | 1.03 |
| 445569 | 1.07 | 1.09 | 1.23 |
| 570808 | 0.94 | 1.27 | 1.43 |
| 594292 | 1.03 | 1.03 | 1.16 |
| 598768 | 1.14 | 1.08 | 0.97 |
| 598769 | 0.97 | 1.05 | 1.04 |
| 512497 | 0.99 | 1.17 | 1.38 |
| 569473 | 1.02 | 1.01 | 1.09 |
| 594300 | 1.14 | 1.07 | 1.02 |
| 598777 | 1.05 | 1.20 | 1.01 |

*Fold change over Saline control group

### Example 18: Six Week In Vivo Tolerability Study in Sprague-Dawley Rats

The newly designed ASOs from Table 1, above, were further evaluated in a 6 week study to assess plasma chemistry, body/organ weights and histology. Groups of Sprague-Dawley rats were administered 100 mpk/wk of ISIS 445569 or ISIS 512497. Further groups of Groups of Sprague-Dawley rats were administered 50 mpk/wk of ISIS 486178, ISIS 570808, ISIS 594292, ISIS 598768, ISIS 598769, ISIS 569473, ISIS 594300, and ISIS 598777. After six weeks and two days after each group of mice received the last dose, the mice were sacrificed and tissues were collected for analysis. For each group of mice, analysis to measure alanine transaminase levels, aspartate aminotransferase levels, blood urea nitrogen (BUN) levels, albumin levels, total bilirubin, creatine levels, and urinary creatine levels was measured. Additionally, organ weights were also measured, the results of which are presented in the tables below.

TABLE 28
Plasma Chemistry & Urine Analysis in Sprague-Dawley Rats

| | ALT | AST | BUN | Total protein | T.Bil | Creatinine | Urine |
|---|---|---|---|---|---|---|---|
| ISIS No. | (U/L) | (U/L) | (mg/dl) | (mg/dl) | (mg/dl) | (mg/dl) | MTP/Creatine |
| Saline | 59.25 | 100.35 | 18.05 | 3.47 | 0.158 | 0.30 | 1.09 |
| 569473 | 101 | 198.25 | 25.9 | 2.74 | 0.195 | 0.4025 | 4.59 |
| 512497 | 211 | 240.25 | 19.32 | 3.58 | 0.17 | 0.39 | 6.18 |
| 598768 | 78.2 | 103.5 | 20.6 | 3.36 | 0.14 | 0.38 | 3.85 |
| 598769 | 84.5 | 104.5 | 18.6 | 3.52 | 0.15 | 0.34 | 3.02 |
| 570808 | 82 | 141 | 23.8 | 3.08 | 0.21 | 0.4 | 2.71 |
| 598777 | 109 | 119.5 | 21.65 | 3.79 | 0.22 | 0.37 | 2.56 |
| 445569 | 117.5 | 163.2 | 22.45 | 3.86 | 0.18 | 0.47 | 6.4 |
| 594300 | 66 | 80.75 | 17.53 | 3.59 | 0.12 | 0.29 | 4.72 |
| 486178 | 56.8 | 80.75 | 23.3 | 5.28 | 0.08 | 3.0 | 4.5 |
| 594292 | 64.5 | 80.5 | 19.62 | 3.38 | 0.098 | 0.29 | 5.17 |

TABLE 29
Plasma Chemistry & Urine Analysis in Sprague-Dawley Rats

| ISIS No. | Kidney (fold)* | Liver (fold)* | Spleen (fold)* |
|---|---|---|---|
| Saline | 1 | 1 | 1 |
| 569473 | 1.46 | 1.20 | 0.82 |
| 512497 | 1.03 | 1.22 | 1.94 |
| 598768 | 0.92 | 0.92 | 1.49 |
| 598769 | 0.93 | 1.04 | 0.98 |
| 570808 | 1.18 | 0.98 | 2.43 |
| 598777 | 1.07 | 0.93 | 2.31 |
| 445569 | 1 | 1.13 | 3.25 |
| 594300 | 1.03 | 1.04 | 1.94 |
| 486178 | 0.87 | 0.89 | 1.45 |
| 594292 | 1.08 | 1.01 | 2.04 |

*Fold change over Saline control group

### Example 19: Thirteen (13) Week In Vivo Study in Cynomolgus Monkeys

Groups of 4 cynomolgus male monkeys were administered 40 mg/kg/wk of ISIS 445569, ISIS 512497, ISIS 486178, ISIS 570808, ISIS 594292, ISIS 598768, ISIS 598769, ISIS 569473, ISIS 594300, and ISIS 598777 via subcutaneous injection. Thirteen weeks after the first dose, the animals were sacrificed and tissue analysis was performed. mRNA was isolated for real-time PCR analysis of rhesus monkey DMPK and normalized to RIBOGREEN®. Primer probe set RTS3164 (described above) was used to measure mRNA levels and the results are shown in Table 30 below. Additionally, further mRNA was isolated for real-time PCR analysis of rhesus monkey DMPK and normalized to RIBOGREEN® using primer probe set RTS4447 and the results are shown in Table 31 below. RTS4447 (forward sequence AGCCTGAGCCGGGAGATG, designated herein as SEQ ID NO: 20; reverse sequence GCGTAGTTGACTGGCAAAGTT, designated herein as SEQ ID NO: 21; probe sequence AGGCCATCCGCATGGCCAACC, designated herein as SEQ ID NO: 22).

TABLE 30
Dose-dependent inhibition of DMPK mRNA levels in
Cynomolgus Monkeys using Primer Probe Set RTS3164

| | | hDMPK mRNA levels (% PBS) | | | | | |
|---|---|---|---|---|---|---|---|
| ISIS No. | mg/kg/wk | TA | Quad (Left) | Gastroc | Kidney | Heart | Liver |
| PBS | 0 | 100 | 100 | 100 | 100 | 100 | 100 |
| 486178 | 40 | 26.1 | 30.8 | 49.3 | 55.3 | 45.8 | 44.9 |
| 445569 | 40 | 68.5 | 82.2 | 128.9 | 65.6 | 91.2 | 113.5 |
| 512497 | 40 | 60.3 | 58.7 | 66.7 | 61.9 | 74.2 | 68.1 |
| 598768 | 40 | 69.1 | 64.9 | 80.7 | 58.1 | 70.6 | 100.8 |
| 594300 | 40 | 73.6 | 80.2 | 106.0 | 57.9 | 97.5 | 91.6 |
| 594292 | 40 | 55.6 | 52.0 | 71.9 | 46.2 | 72.1 | 81.6 |
| 569473 | 40 | 44.8 | 31.7 | 61.6 | 44.0 | 58.7 | 28.0 |
| 598769 | 40 | 31.7 | 28.9 | 49.7 | 26.8 | 45.0 | 38.6 |
| 570808 | 40 | 2.5 | 4.4 | 6.4 | 29.7 | 17.5 | 7.2 |
| 598777 | 40 | 53.3 | 31.8 | 76.4 | 42.7 | 44.6 | 111.6 |

TABLE 31
Dose-dependent inhibition of DMPK mRNA levels in
Cynomolgus Monkeys using Primer Probe Set RTS4447

| | | hDMPK mRNA levels (% PBS) | | | | | |
|---|---|---|---|---|---|---|---|
| ISIS No. | mg/kg/wk | TA | Quad (Left) | Gastroc | Kidney | Heart | Liver |
| PBS | 0 | 100.0 | 100.0 | 100.0 | 100.0 | 100.0 | 100.0 |
| 486178 | 40 | 26.7 | 29.0 | 32.9 | 57.0 | 49.4 | 58.1 |
| 445569 | 40 | 85.4 | 87.4 | 147.1 | 77.1 | 97.2 | 93.6 |
| 512497 | 40 | 66.4 | 70.4 | 94.2 | 81.9 | 87.6 | 79.5 |
| 598768 | 40 | 48.3 | 76.4 | 106.7 | 73.7 | 81.0 | 85.1 |
| 594300 | 40 | 100.9 | 113.5 | 219.6 | 96.9 | 131.0 | 118.9 |
| 594292 | 40 | 76.5 | 75.7 | 151.7 | 86.6 | 107.1 | 108.6 |
| 569473 | 40 | 52.6 | 51.7 | 114.2 | 72.9 | 87.2 | 53.7 |
| 598769 | 40 | 45.2 | 57.6 | 86.3 | 56.6 | 65.4 | 72.5 |
| 570808 | 40 | 6.6 | 8.3 | 14.8 | 60.7 | 27.9 | 35.0 |
| 598777 | 40 | 55.1 | 56.8 | 124.1 | 78.6 | 88.9 | 131.2 |

### Example 20: Thirteen (13) Week In Vivo Tolerability Study in Cynomolgus Monkeys

Groups of cynomolgus male monkeys were administered 40 mg/kg of ISIS 445569, ISIS 512497, ISIS 486178, ISIS 570808, ISIS 594292, ISIS 598768, ISIS 598769, ISIS 569473, ISIS 594300, and ISIS 598777 via subcutaneous injection on days 1, 3, 5, and 7. Following administration on day 7, each monkey was administered 40 mg/kg/wk of ISIS 445569, ISIS 512497, ISIS 486178, ISIS 570808, ISIS 594292, ISIS 598768, ISIS 598769, ISIS 569473, ISIS 594300, and ISIS 598777 via subcutaneous injection.

48 hours after each monkey received a subcutaneous dose on days 28 and 91, blood and urine samples were taken for analysis. Some of the monkeys had blood and urine taken 48 hours after the dose given on day 56. Alanine aminotransferase (ALT), aspartate aminotransferase (AST), lactate dehydrogenase (LDH), and creatine kinase (CK) were measured for each animal in a treatment group and the average values are presented in the table below. Day of Sample values with a negative represent time point before treatment began. For example, a Day of Treatment value of −7 represents a sample taken 7 days before the first dose. Thirteen weeks after the first dose, the animals were sacrificed and tissue analysis was performed.

TABLE 32
Plasma Chemistry & Urine Analysis in Cynomolgus Monkeys

| ISIS | Day of | ALT | AST | LDH | CK |
|---|---|---|---|---|---|
| No. | Sample | (U/L) | (U/L) | (mg/dl) | (mg/dl) |
| Saline | −14 | 34.2 | 25.9 | 604.0 | 160.8 |
| | −7 | 38.8 | 27.8 | 861.3 | 249.0 |
| | 30 | 43.0 | 34.4 | 1029.0 | 300.0 |
| | 93 | 66.1 | 43.0 | 1257.3 | 898.8 |
| 486178 | −14 | 37.6 | 40.5 | 670.0 | 236.8 |
| | −7 | 49.8 | 55.0 | 1039.8 | 380.8 |
| | 30 | 47.0 | 41.2 | 875.4 | 415.0 |
| | 93 | 59.7 | 43.6 | 960.6 | 809.6 |
| 594292 | −14 | 38.9 | 32.0 | 776.3 | 375.8 |
| | −7 | 37.8 | 38.4 | 877.3 | 210.0 |
| | 30 | 35.4 | 39.6 | 666.0 | 93.8 |
| | 93 | 49.8 | 46.3 | 958.5 | 339.0 |
| 569473 | −14 | 49.4 | 49.8 | 1185.3 | 365.3 |
| | −7 | 50.4 | 59.7 | 1609.5 | 261.0 |
| | 30 | 46.7 | 52.5 | 1390.8 | 107.8 |
| | 93 | 56.3 | 49.8 | 1483.3 | 524.5 |
| 570808 | −14 | 47.1 | 46.8 | 896.0 | 448.3 |
| | −7 | 44.4 | 63.6 | 913.3 | 257.3 |
| | 30 | 47.1 | 57.7 | 660.5 | 125.0 |
| | 93 | 79.8 | 92.2 | 813.5 | 294.0 |
| 598768 | −14 | 37.9 | 41.6 | 666.3 | 253.8 |
| | −7 | 41.4 | 53.5 | 754.0 | 231.5 |
| | 30 | 37.2 | 38.9 | 652.3 | 106.3 |
| | 93 | 45.8 | 41.5 | 721.3 | 238.3 |
| 598769 | −14 | 44.2 | 36.1 | 1106.8 | 456.8 |
| | −7 | 45.7 | 41.5 | 1323.3 | 214.0 |
| | 30 | 40.3 | 42.0 | 981.0 | 147.8 |
| | 58 | 56.7 | 49.9 | 1101.5 | 552.3 |
| | 93 | 69.0 | 50.3 | 1167.3 | 749.5 |
| 512497 | −14 | 31.5 | 34.3 | 689.3 | 293.8 |
| | −7 | 39.0 | 45.4 | 1110.3 | 286.0 |
| | 30 | 47.2 | 60.2 | 960.5 | 202.5 |
| | 93 | 69.6 | 87.1 | 997.0 | 1118.5 |
| 594300 | −14 | 42.0 | 34.0 | 935.5 | 459.5 |
| | −7 | 42.1 | 53.6 | 1020.5 | 272.0 |
| | 30 | 28.0 | 34.6 | 620.8 | 124.5 |
| | 58 | 42.9 | 48.5 | 883.5 | 169.8 |
| | 93 | 45.7 | 45.7 | 835.5 | 252.3 |
| 598777 | −14 | 45.6 | 37.7 | 707.0 | 558.5 |
| | −7 | 43.3 | 50.0 | 705.8 | 200.3 |
| | 30 | 50.2 | 47.3 | 585.3 | 159.3 |
| | 93 | 79.2 | 56.1 | 1029.0 | 785.0 |
| 445569 | −14 | 40.2 | 44.2 | 835.8 | 404.0 |
| | −7 | 41.0 | 46.1 | 1074.3 | 305.5 |
| | 30 | 45.9 | 61.7 | 994.8 | 283.0 |
| | 58 | 51.6 | 85.1 | 739.0 | 117.8 |
| | 93 | 99.3 | 97.5 | 1583.5 | 2114.0 |

1. A compound comprising a single-stranded modified oligonucleotide consisting of 10-30 linked nucleosides and having a nucleobase sequence comprising a portion of at least 12 contiguous nucleobases 100% complementary to a target region of equal length of a DMPK nucleic acid, wherein the modified oligonucleotide comprises at least one modified internucleoside linkage and/or at least one modified nucleoside comprising a modified sugar.

2. The compound of claim 1, wherein the modified oligonucleotide has a nucleobase sequence comprising a portion of at least 12 contiguous nucleobases 100% complementary to an equal length portion of nucleobases 636-697, 1317-1366, 1446-1486, 1610-1638, 1635-1670, or 2696-2717 of SEQ ID NO: 1 or nucleobases 6445-6468, 6789-6806, 13628-13657, 13735-13760, or 13746-13905 of SEQ ID NO: 2, and wherein the nucleobase sequence of the modified oligonucleotide is at least 90% complementary to SEQ ID NO: 1 or SEQ ID NO: 2 as measured over the entirety of the modified oligonucleotide.

3. The compound of claim 2, wherein the modified oligonucleotide comprises at least one modified internucleoside linkage.

4. The compound of claim 3, wherein each internucleoside linkage is a phosphorothioate internucleoside linkage.

5. The compound of claim 4, wherein the modified oligonucleotide comprises at least one modified nucleoside comprising a modified sugar.

6. The compound of claim 5, wherein the modified oligonucleotide comprises at least two modified nucleosides comprising a modified sugar.

7. The compound of claim 5, wherein the modified sugar is a bicyclic sugar.

8. The compound of claim 7, wherein the bicyclic sugar is selected from cEt, LNA, α-L-LNA, ENA, and 2′-thio LNA.

9. The compound of claim 5, wherein the modified oligonucleotide comprises at least one 2′-substituted nucleoside.

10. The compound of claim 9, wherein the 2′-substituted nucleoside is selected from 2′-OCH₃, 2′-F, and 2′-O-methoxyethyl.

11. The compound of claim 10, wherein the modified oligonucleotide comprises:
a gap segment consisting of 7-11 linked deoxynucleosides;
a 5′ wing segment consisting of 2-6 linked nucleosides;
a 3′ wing segment consisting of 2-6 linked nucleosides;
wherein the gap segment is positioned between the 5′ wing segment and the 3′ wing segment and wherein each nucleoside of each wing segment comprises a modified sugar.

12. The compound of claim 11, wherein the modified oligonucleotide consists of 16, 17, 18, 19, or 20 linked nucleosides.

13. The compound of claim 1, wherein the modified oligonucleotide has a nucleobase sequence comprising a portion of at least 12 contiguous nucleobases of any of SEQ ID NOs: 33, 94-110, 530-539, 25, 117-128, 376-384, 176-197, 224-233, 246-262, 426-429, 487-489, 433, 681-685, 712-713, 269-277, 286-291, 23, 264, 294-311, or 426-430, and wherein the nucleobase sequence of the modified oligonucleotide is at least 90% complementary to SEQ ID NO: 1 or SEQ ID NO: 2 as measured over the entirety of the modified oligonucleotide.

14. The compound of claim 13, wherein the modified oligonucleotide comprises at least one modified internucleoside linkage.

15. The compound of claim 14, wherein each internucleoside linkage of the modified oligonucleotide is a phosphorothioate internucleoside linkage.

16. The compound of claim 15, wherein the modified oligonucleotide comprises at least one modified nucleoside comprising a modified sugar.

17. The compound of claim 16, wherein the modified oligonucleotide comprises at least two modified nucleosides comprising a modified sugar.

18. The compound of claim 16, wherein the modified sugar is a bicyclic sugar.

19. The compound of claim 18, wherein the bicyclic sugar is selected from cEt, LNA, α-L-LNA, ENA, and 2′-thio LNA.

20. The compound of claim 16, wherein the modified oligonucleotide comprises at least one 2′-substituted nucleoside.

21. The compound of claim 20, wherein the 2′-substituted nucleoside is selected from 2′-OCH₃, 2′-F, and 2′-O-methoxyethyl.

22. The compound of claim 21, wherein the modified oligonucleotide comprises:
a gap segment consisting of 7-10 linked deoxynucleosides;
a 5′ wing segment consisting of 2-5 linked nucleosides;
a 3′ wing segment consisting of 2-5 linked nucleosides;
wherein the gap segment is positioned between the 5′ wing segment and the 3′ wing segment and wherein each nucleoside of each wing segment comprises a modified sugar.

23. The compound of claim 22, wherein the modified oligonucleotide consists of 16, 17, 18, 19, or 20 linked nucleosides.

24. A composition comprising the compound of claim 1 and a pharmaceutically acceptable carrier or diluent.

25. A composition comprising the compound of claim 13 and a pharmaceutically acceptable carrier or diluent.

26. A method of treating type 1 myotonic dystrophy (DM1) in an animal comprising administering to an animal in need thereof a composition according to claim 2.

27. A method of treating type 1 myotonic dystrophy (DM1) in an animal comprising administering to an animal in need thereof a composition according to claim 13.
